(19) United States
(12) Patent Application Publication
(10) Pub. No.: US 2025/0197374 A1
(43) Pub. Date: Jun. 19, 2025

(54) WEE1 DEGRADING COMPOUNDS AND USES THEREOF

(71) Applicant: BRISTOL-MYERS SQUIBB COMPANY, Princeton, NJ (US)

(72) Inventors: Jesus MORENO, Lemon Grove, CA (US); Eric R. WELIN, San Diego, CA (US)

(21) Appl. No.: 18/985,507

(22) Filed: Dec. 18, 2024

Related U.S. Application Data
(60) Provisional application No. 63/611,875, filed on Dec. 19, 2023.

(51) Int. Cl. C07D 401/14; A61K 31/4709; A61K 31/4725; A61K 31/496; A61K 31/498; A61K 31/499; A61K 31/517; A61K 31/519; A61K 31/5377; A61K 31/5386; C07D 405/14; C07D 471/04; C07D 491/048; C07D 491/08; C07D 491/107; C07D 493/10; C07D 498/10

(52) U.S. Cl. CPC C07D 401/14; A61K 31/4709; A61K 31/4725; A61K 31/496; A61K 31/498; A61K 31/499; A61K 31/517; A61K 31/519; A61K 31/5377; A61K 31/5386; C07D 405/14; C07D 471/04; C07D 491/048; C07D 491/08; C07D 491/107; C07D 493/10; C07D 498/10

(57) ABSTRACT

Provided herein are compounds and compositions thereof that reduce WEE1 kinase protein levels. In some embodiments, the compounds and compositions are provided for treatment WEE1 associated diseases such as cancer.

### CROSS-REFERENCE RELATED TO APPLICATION

This application claims benefit of U.S. Provisional Application No. 63/611,875 filed on Dec. 19, 2023, which is incorporated by reference herein in its entirety for any purpose.

### FIELD

The present disclosure relates generally to compounds, compositions, and methods for their preparation and use of the compounds and compositions for treating diseases and conditions associated with the WEE1 protein.

### BACKGROUND

Targeted protein degradation is a therapeutic modality whereby small molecules induce novel protein-protein interactions and enable destruction of target proteins that drive disease. CRBN E3 ligase modulator (CELMoD) small molecules are a class of targeted protein therapeutics that co-opts the CRL4-Cereblon E3 ubiquitin ligase complex, generating a new molecular “glue” interface on the surface of Cereblon that recruits and polyubiquitinates target proteins that are not normally ubiquitinated by Cereblon. The ubiquitin tagged proteins are then trafficked to and subsequently degraded by the 26S proteasome. The selection of target proteins recruited by Cereblon is determined by the specific molecular structure of the CELMoD compound.

WEE1 is a tyrosine kinase that phosphorylates the CDK1 and CDK2 cyclin-dependent kinases at their tyrosine-15 residue consequently inhibiting kinase activity and halting the cell cycle at the intra-S and G2/M cell cycle checkpoints. While halted at these checkpoints, cells repair stalled replication forks and DNA damage before entering mitosis; if the DNA damage cannot be repaired or rises above acceptable thresholds, cells undergo programmed apoptosis or mitotic catastrophe. Cancer cells frequently exhibit excessive replication stress, mutagenesis, and genomic instability prompting a reliance on cell cycle checkpoints to maintain DNA damage below apoptotic thresholds. Compared to normal cells, cancer cells frequently rely solely upon the intra-S and G2/M checkpoints because their G1 cell cycle checkpoint is disabled by a various mechanisms, for instance, by restricting p53 and pRB activation, or, by hyper-activating replication-promoting factors such as Cyclin D and Cyclin E. Tumors that rely heavily upon the WEE1-mediated intra-S and G2/M checkpoints are hypothesized to be exceptionally sensitive to WEE1 loss and therapeutics that target WEE1 are expected to exhibit antitumor activity with a favorable therapeutic window.

Certain cancer therapeutics function by inducing replication stress and DNA damage, including, for example, carboplatin, cisplatin, gemcitabine, pemetrexed, topotecan, doxorubicin, decitabine, and methotrexate. By inducing DNA damage and replication stress, these agents enhance the reliance of dividing tumor cells upon the WEE1-mediated cell cycle checkpoints. Therefore, CELMoD compounds that degrade WEE1 are proposed to synergistically combine with DNA damaging agents.

Accordingly, in one aspect, provided herein are CELMoD compounds that induce the Cereblon-mediated ubiquitination and degradation of WEE1 to elicit broad antitumor activity as a single agent or in combination with sensitizing therapeutics.

### SUMMARY

Described herein, in certain embodiments, are compounds and compositions thereof that degrade WEE1. In various embodiments, the compounds and compositions thereof may be used for treatment of diseases associated with WEE1, for example, cancer.

The present embodiments can be understood more fully by reference to the detailed description and examples, which are intended to exemplify non-limiting embodiments.

In some embodiments, provided herein are compounds selected from:

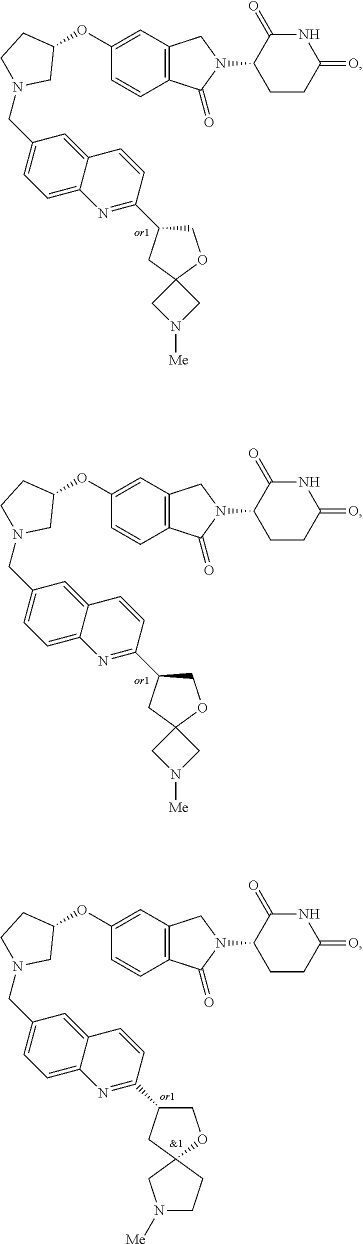 [SMILES: CN1CC2(C[C@H](c3ccc4cc(CN5CC[C@H](Oc6ccc7c(c6)CN([C@H]6CCC(=O)NC6=O)C7=O)C5)ccc4n3)CO2)C1 ; CN1CC2(C[C@@H](c3ccc4cc(CN5CC[C@H](Oc6ccc7c(c6)CN([C@H]6CCC(=O)NC6=O)C7=O)C5)ccc4n3)CO2)C1 ; CN1CC[C@]2(C[C@H](c3ccc4cc(CN5CC[C@H](Oc6ccc7c(c6)CN([C@H]6CCC(=O)NC6=O)C7=O)C5)ccc4n3)CO2)C1]

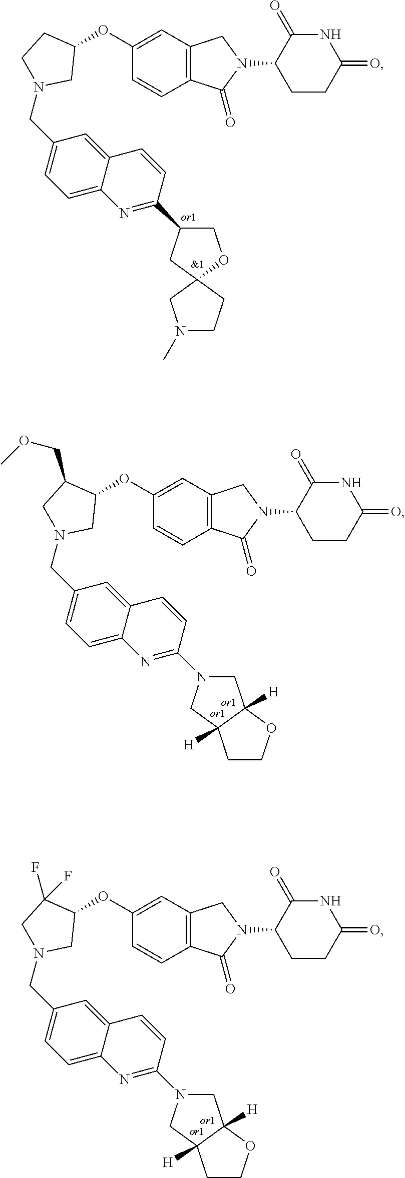 [SMILES: CN1CC[C@]2(C[C@@H](c3ccc4cc(CN5CC[C@H](Oc6ccc7c(c6)CN([C@H]6CCC(=O)NC6=O)C7=O)C5)ccc4n3)CO2)C1 ; COC[C@@H]1CN(Cc2ccc3nc(N4C[C@H]5CCO[C@H]5C4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5ccc6nc(N7C[C@H]8CCO[C@H]8C7)ccc6c5)CC4(F)F)ccc3C2=O)C(=O)N1]

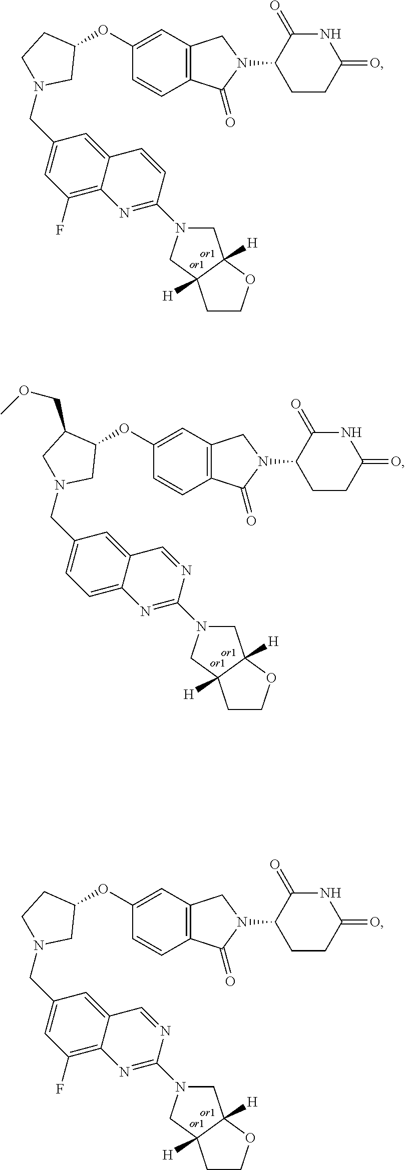 [SMILES: O=C1CC[C@H](N2Cc3cc(O[C@H]4CCN(Cc5cc(F)c6nc(N7C[C@H]8CCO[C@H]8C7)ccc6c5)C4)ccc3C2=O)C(=O)N1 ; COC[C@@H]1CN(Cc2ccc3nc(N4C[C@H]5CCO[C@H]5C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(N7C[C@H]8CCO[C@H]8C7)ncc6c5)C4)ccc3C2=O)C(=O)N1]

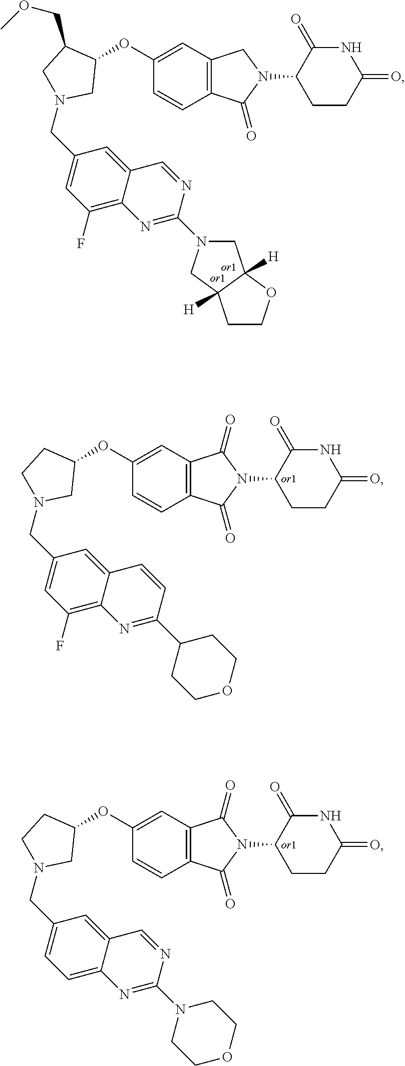 [SMILES: COC[C@@H]1CN(Cc2cc(F)c3nc(N4C[C@H]5CCO[C@H]5C4)ncc3c2)C[C@H]1Oc1ccc2c(c1)CN([C@H]1CCC(=O)NC1=O)C2=O ; O=C1CC[C@H](N2C(=O)c3ccc(O[C@H]4CCN(Cc5cc(F)c6nc(C7CCOCC7)ccc6c5)C4)cc3C2=O)C(=O)N1 ; O=C1CC[C@H](N2C(=O)c3ccc(O[C@H]4CCN(Cc5ccc6nc(N7CCOCC7)ncc6c5)C4)cc3C2=O)C(=O)N1]

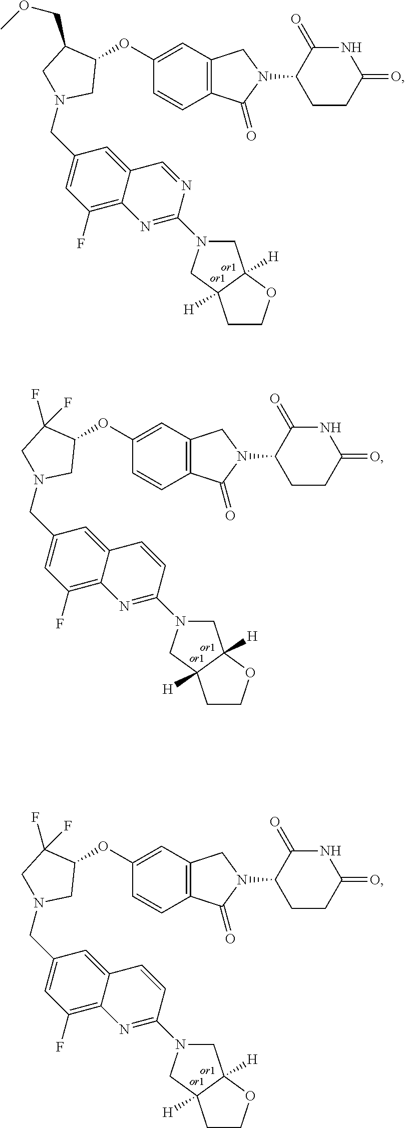 [SMILES: COC[C@@H]1CN(Cc2cc(F)c3nc(N4C[C@@H]5CCO[C@@H]5C4)ncc3c2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(N7C[C@H]8CCO[C@H]8C7)ccc6c5)CC4(F)F)ccc3C2=O)C(=O)N1 ; O=C1CC[C@H](N2Cc3cc(O[C@@H]4CN(Cc5cc(F)c6nc(N7C[C@@H]8CCO[C@@H]8C7)ccc6c5)CC4(F)F)ccc3C2=O)C(=O)N1]

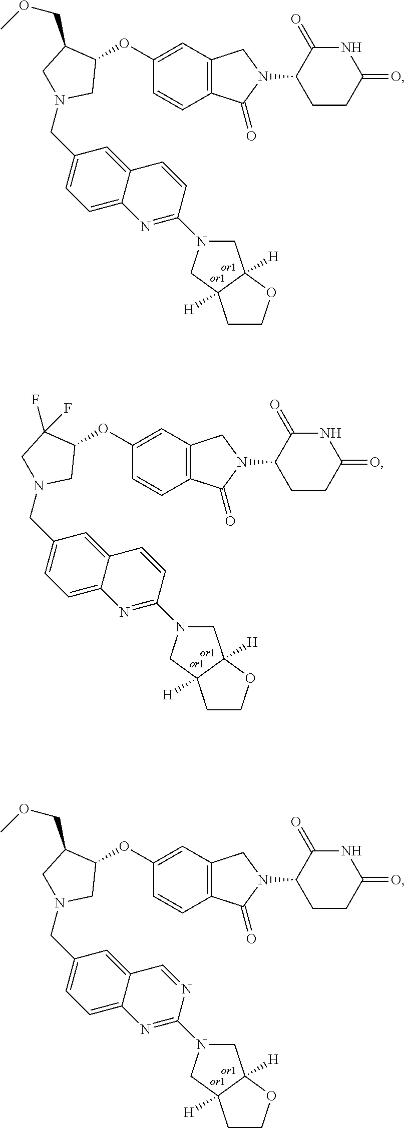 [SMILES: COC[C@@H]1CN(Cc2ccc3nc(N4C[C@@H]5CCO[C@@H]5C4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5ccc6nc(N7C[C@@H]8CCO[C@@H]8C7)ccc6c5)CC4(F)F)ccc3C2=O)C(=O)N1 ; COC[C@@H]1CN(Cc2ccc3nc(N4C[C@@H]5CCO[C@@H]5C4)ncc3c2)C[C@H]1Oc1ccc2c(c1)CN([C@H]1CCC(=O)NC1=O)C2=O]

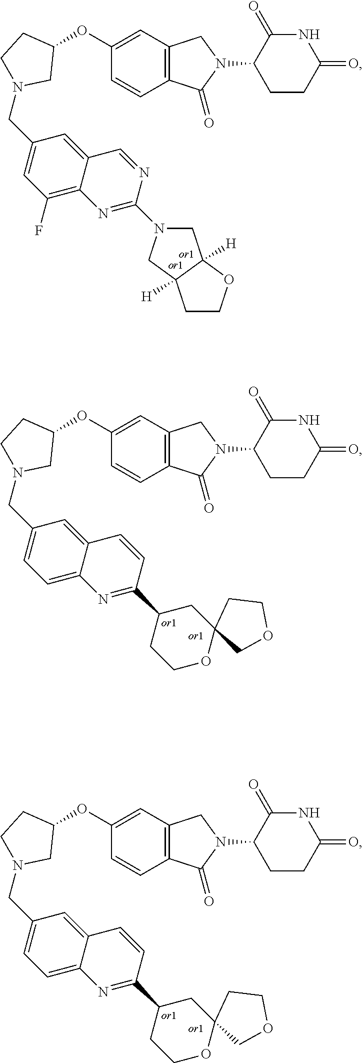 [SMILES: O=C1CC[C@H](N2Cc3cc(O[C@H]4CCN(Cc5cc(F)c6nc(N7C[C@@H]8CCO[C@@H]8C7)ncc6c5)C4)ccc3C2=O)C(=O)N1 ; O=C1CC[C@H](N2Cc3cc(O[C@H]4CCN(Cc5ccc6nc([C@@H]7CCO[C@@]8(CCOC8)C7)ccc6c5)C4)ccc3C2=O)C(=O)N1 ; O=C1CC[C@H](N2Cc3cc(O[C@H]4CCN(Cc5ccc6nc([C@@H]7CCO[C@]8(CCOC8)C7)ccc6c5)C4)ccc3C2=O)C(=O)N1]

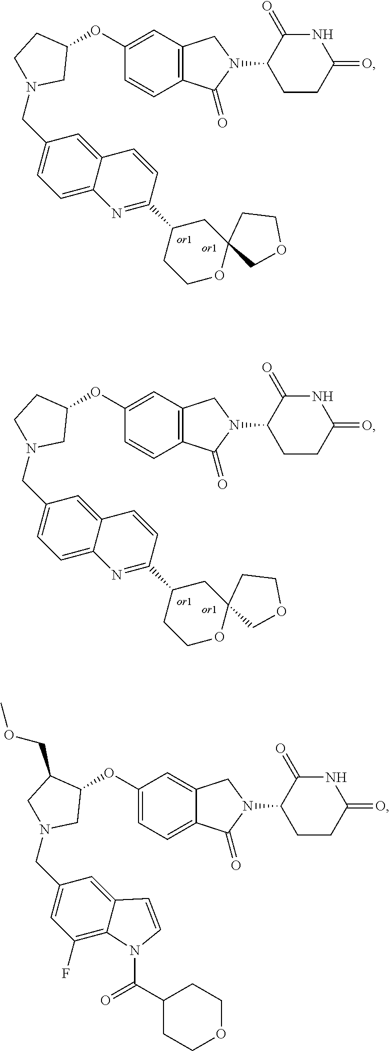 [SMILES: O=C1CC[C@H](N2Cc3cc(O[C@H]4CCN(Cc5ccc6nc([C@H]7CCO[C@@]8(CCOC8)C7)ccc6c5)C4)ccc3C2=O)C(=O)N1 ; O=C1CC[C@H](N2Cc3cc(O[C@H]4CCN(Cc5ccc6nc([C@H]7CCO[C@]8(CCOC8)C7)ccc6c5)C4)ccc3C2=O)C(=O)N1 ; COC[C@@H]1CN(Cc2cc(F)c3c(ccn3C(=O)C3CCOCC3)c2)C[C@H]1Oc1ccc2c(c1)CN([C@H]1CCC(=O)NC1=O)C2=O]

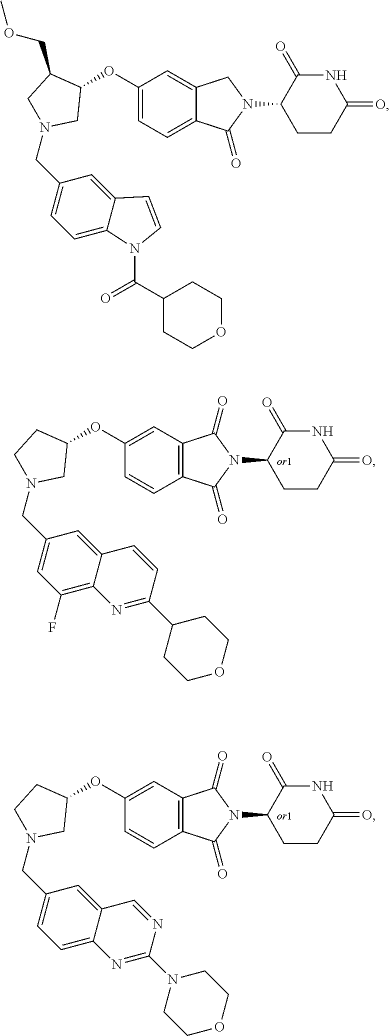 [SMILES: COC[C@@H]1CN(Cc2ccc3c(ccn3C(=O)C3CCOCC3)c2)C[C@H]1Oc1ccc2c(c1)CN([C@H]1CCC(=O)NC1=O)C2=O ; O=C1CC[C@@H](N2C(=O)c3ccc(O[C@H]4CCN(Cc5cc(F)c6nc(C7CCOCC7)ccc6c5)C4)cc3C2=O)C(=O)N1 ; O=C1CC[C@@H](N2C(=O)c3ccc(O[C@H]4CCN(Cc5ccc6nc(N7CCOCC7)ncc6c5)C4)cc3C2=O)C(=O)N1]

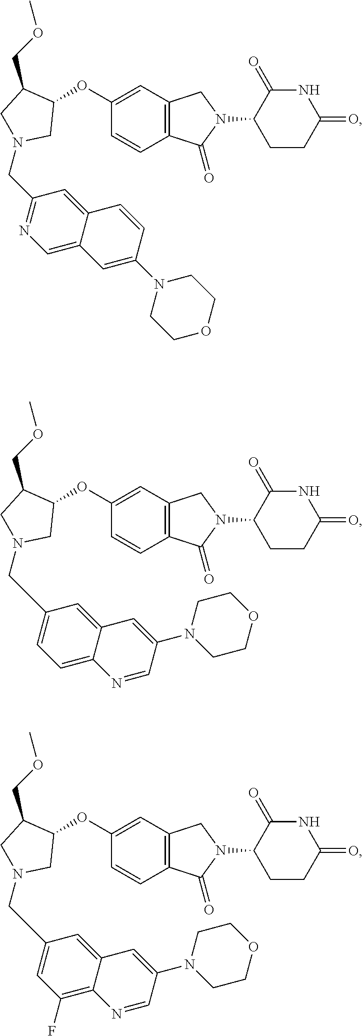 [SMILES: COC[C@@H]1CN(Cc2cc3ccc(N4CCOCC4)cc3cn2)C[C@H]1Oc1ccc2c(c1)CN([C@H]1CCC(=O)NC1=O)C2=O ; COC[C@@H]1CN(Cc2ccc3ncc(N4CCOCC4)cc3c2)C[C@H]1Oc1ccc2c(c1)CN([C@H]1CCC(=O)NC1=O)C2=O ; COC[C@@H]1CN(Cc2cc(F)c3ncc(N4CCOCC4)cc3c2)C[C@H]1Oc1ccc2c(c1)CN([C@H]1CCC(=O)NC1=O)C2=O]

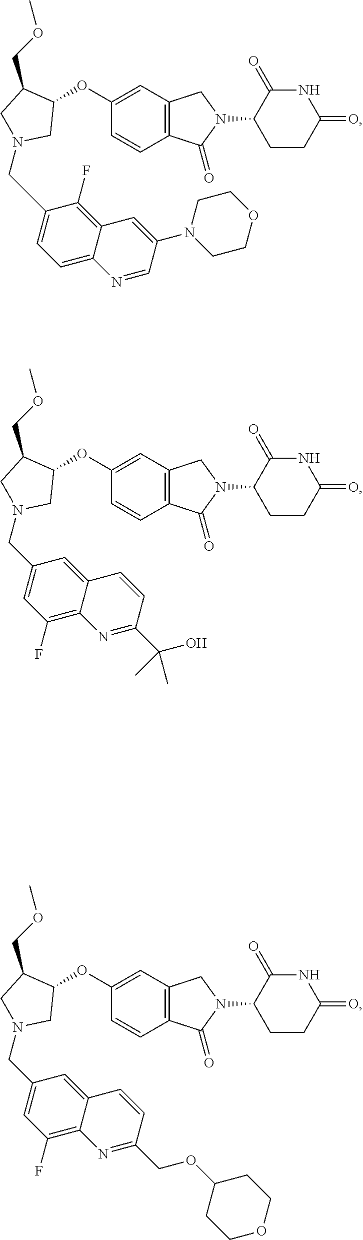 [SMILES: COC[C@@H]1CN(Cc2ccc3ncc(N4CCOCC4)cc3c2F)C[C@H]1Oc1ccc2c(c1)CN([C@H]1CCC(=O)NC1=O)C2=O ; COC[C@@H]1CN(Cc2cc(F)c3nc(C(C)(C)O)ccc3c2)C[C@H]1Oc1ccc2c(c1)CN([C@H]1CCC(=O)NC1=O)C2=O ; COC[C@@H]1CN(Cc2cc(F)c3nc(COC4CCOCC4)ccc3c2)C[C@H]1Oc1ccc2c(c1)CN([C@H]1CCC(=O)NC1=O)C2=O]

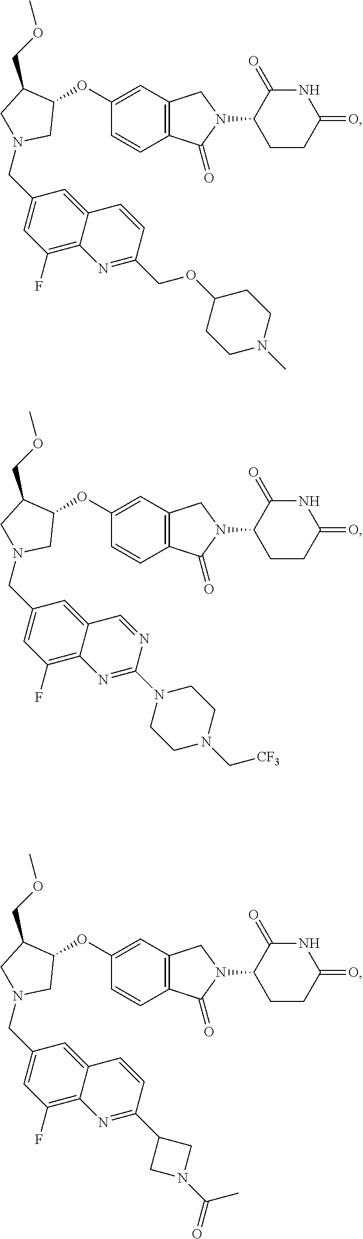 [SMILES: COC[C@@H]1CN(Cc2cc(F)c3nc(COC4CCN(C)CC4)ccc3c2)C[C@H]1Oc1ccc2c(c1)CN([C@H]1CCC(=O)NC1=O)C2=O ; COC[C@@H]1CN(Cc2cc(F)c3nc(N4CCN(CC(F)(F)F)CC4)ncc3c2)C[C@H]1Oc1ccc2c(c1)CN([C@H]1CCC(=O)NC1=O)C2=O ; COC[C@@H]1CN(Cc2cc(F)c3nc(C4CN(C(C)=O)C4)ccc3c2)C[C@H]1Oc1ccc2c(c1)CN([C@H]1CCC(=O)NC1=O)C2=O]

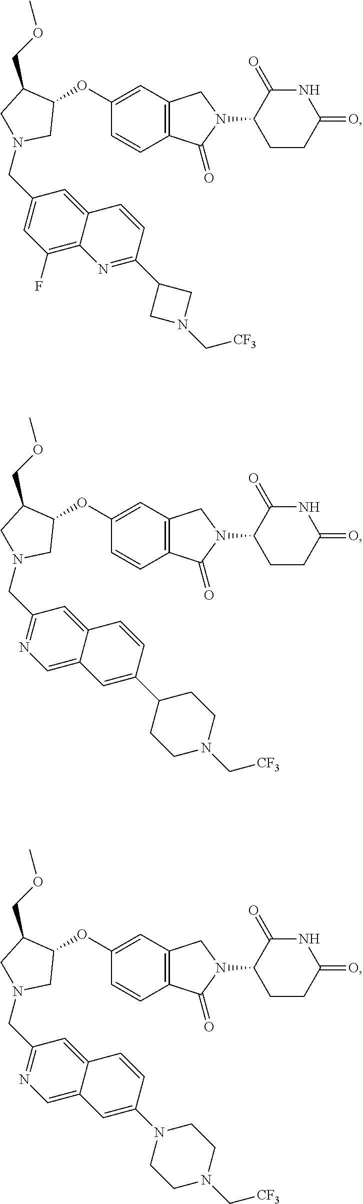 [SMILES: COC[C@@H]1CN(Cc2cc(F)c3nc(C4CN(CC(F)(F)F)C4)ccc3c2)C[C@H]1Oc1ccc2c(c1)CN([C@H]1CCC(=O)NC1=O)C2=O ; COC[C@@H]1CN(Cc2cc3ccc(C4CCN(CC(F)(F)F)CC4)cc3cn2)C[C@H]1Oc1ccc2c(c1)CN([C@H]1CCC(=O)NC1=O)C2=O ; COC[C@@H]1CN(Cc2cc3ccc(N4CCN(CC(F)(F)F)CC4)cc3cn2)C[C@H]1Oc1ccc2c(c1)CN([C@H]1CCC(=O)NC1=O)C2=O]

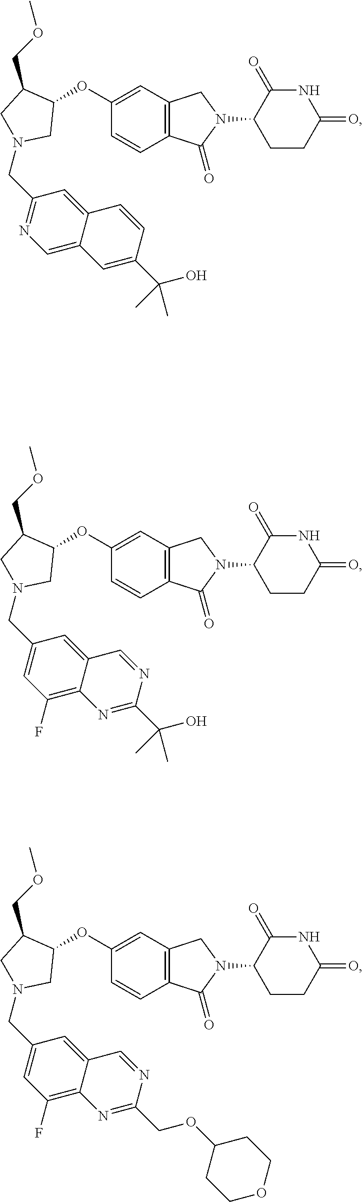 [SMILES: COC[C@@H]1CN(Cc2cc3ccc(C(C)(C)O)cc3cn2)C[C@H]1Oc1ccc2c(c1)CN([C@H]1CCC(=O)NC1=O)C2=O ; COC[C@@H]1CN(Cc2cc(F)c3nc(C(C)(C)O)ncc3c2)C[C@H]1Oc1ccc2c(c1)CN([C@H]1CCC(=O)NC1=O)C2=O ; COC[C@@H]1CN(Cc2cc(F)c3nc(COC4CCOCC4)ncc3c2)C[C@H]1Oc1ccc2c(c1)CN([C@H]1CCC(=O)NC1=O)C2=O]

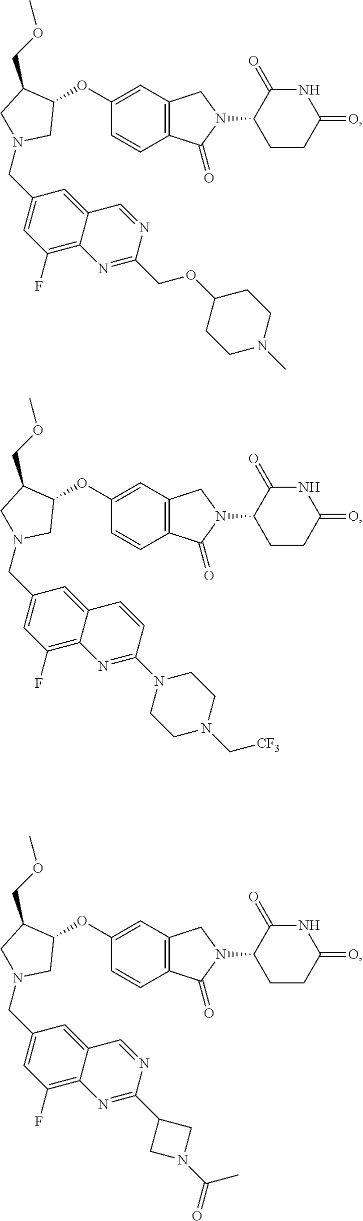 [SMILES: COC[C@@H]1CN(Cc2cc(F)c3nc(COC4CCN(C)CC4)ncc3c2)C[C@H]1Oc1ccc2c(c1)CN([C@H]1CCC(=O)NC1=O)C2=O ; COC[C@@H]1CN(Cc2cc(F)c3nc(N4CCN(CC(F)(F)F)CC4)ccc3c2)C[C@H]1Oc1ccc2c(c1)CN([C@H]1CCC(=O)NC1=O)C2=O ; COC[C@@H]1CN(Cc2cc(F)c3nc(C4CN(C(C)=O)C4)ncc3c2)C[C@H]1Oc1ccc2c(c1)CN([C@H]1CCC(=O)NC1=O)C2=O]

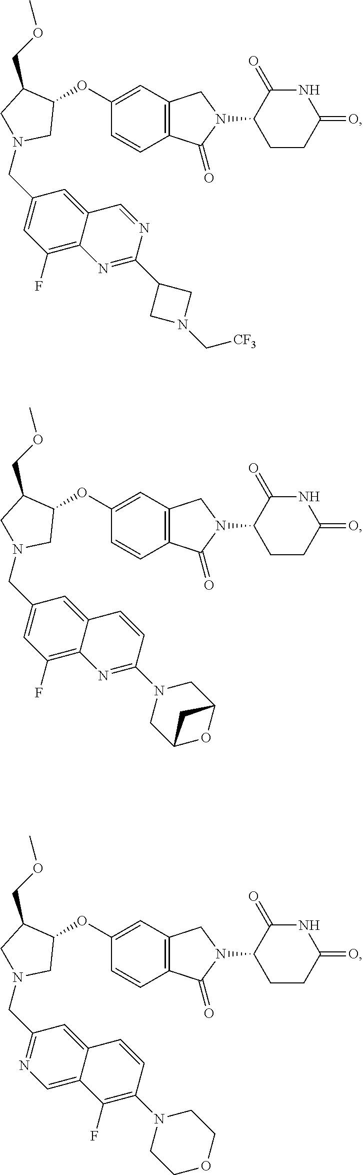 [SMILES: COC[C@@H]1CN(Cc2cc(F)c3nc(C4CN(CC(F)(F)F)C4)ncc3c2)C[C@H]1Oc1ccc2c(c1)CN([C@H]1CCC(=O)NC1=O)C2=O ; COC[C@@H]1CN(Cc2cc(F)c3nc(N4C[C@H]5C[C@@H](C4)O5)ccc3c2)C[C@H]1Oc1ccc2c(c1)CN([C@H]1CCC(=O)NC1=O)C2=O ; COC[C@@H]1CN(Cc2cc3ccc(N4CCOCC4)c(F)c3cn2)C[C@H]1Oc1ccc2c(c1)CN([C@H]1CCC(=O)NC1=O)C2=O]

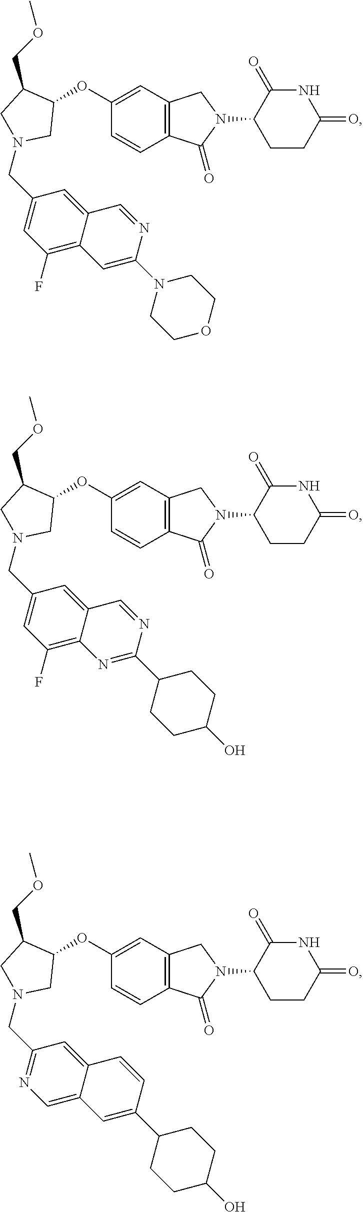 [SMILES: COC[C@@H]1CN(Cc2cc(F)c3cc(N4CCOCC4)ncc3c2)C[C@H]1Oc1ccc2c(c1)CN([C@H]1CCC(=O)NC1=O)C2=O ; COC[C@@H]1CN(Cc2cc(F)c3nc(C4CCC(O)CC4)ncc3c2)C[C@H]1Oc1ccc2c(c1)CN([C@H]1CCC(=O)NC1=O)C2=O ; COC[C@@H]1CN(Cc2cc3ccc(C4CCC(O)CC4)cc3cn2)C[C@H]1Oc1ccc2c(c1)CN([C@H]1CCC(=O)NC1=O)C2=O]

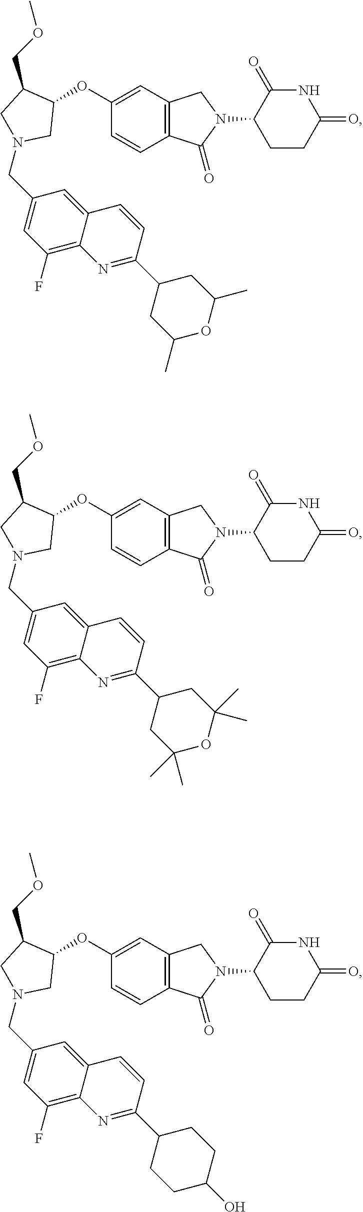 [SMILES: COC[C@@H]1CN(Cc2cc(F)c3nc(C4CC(C)OC(C)C4)ccc3c2)C[C@H]1Oc1ccc2c(c1)CN([C@H]1CCC(=O)NC1=O)C2=O ; COC[C@@H]1CN(Cc2cc(F)c3nc(C4CC(C)(C)OC(C)(C)C4)ccc3c2)C[C@H]1Oc1ccc2c(c1)CN([C@H]1CCC(=O)NC1=O)C2=O ; COC[C@@H]1CN(Cc2cc(F)c3nc(C4CCC(O)CC4)ccc3c2)C[C@H]1Oc1ccc2c(c1)CN([C@H]1CCC(=O)NC1=O)C2=O]

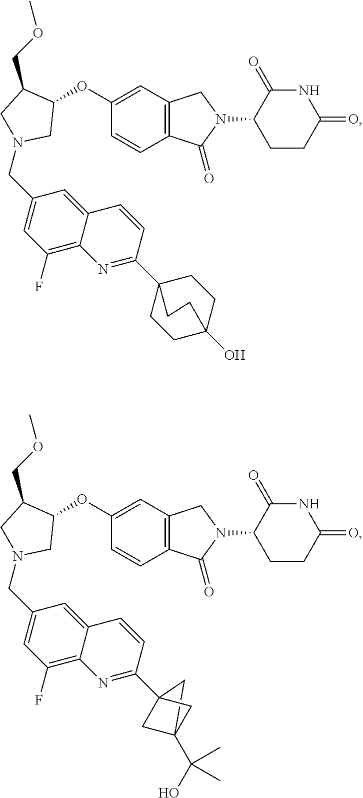 [SMILES: COC[C@@H]1CN(Cc2cc(F)c3nc(C45CCC(O)(CC4)CC5)ccc3c2)C[C@H]1Oc1ccc2c(c1)CN([C@H]1CCC(=O)NC1=O)C2=O ; COC[C@@H]1CN(Cc2cc(F)c3nc(C45CC(C(C)(C)O)(C4)C5)ccc3c2)C[C@H]1Oc1ccc2c(c1)CN([C@H]1CCC(=O)NC1=O)C2=O]

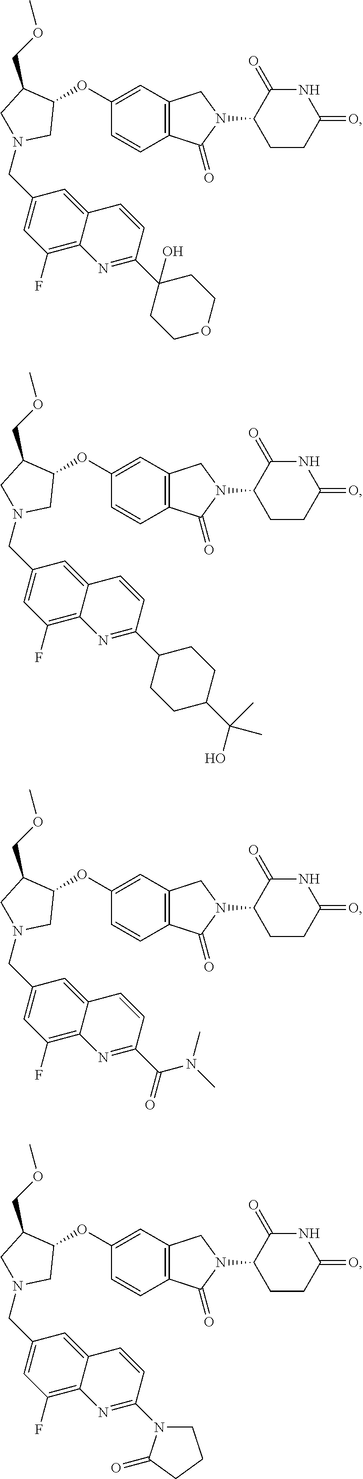 [SMILES: COC[C@@H]1CN(Cc2cc(F)c3nc(C4(O)CCOCC4)ccc3c2)C[C@H]1Oc1ccc2c(c1)CN([C@H]1CCC(=O)NC1=O)C2=O ; COC[C@@H]1CN(Cc2cc(F)c3nc(C4CCC(C(C)(C)O)CC4)ccc3c2)C[C@H]1Oc1ccc2c(c1)CN([C@H]1CCC(=O)NC1=O)C2=O ; COC[C@@H]1CN(Cc2cc(F)c3nc(C(=O)N(C)C)ccc3c2)C[C@H]1Oc1ccc2c(c1)CN([C@H]1CCC(=O)NC1=O)C2=O ; COC[C@@H]1CN(Cc2cc(F)c3nc(N4CCCC4=O)ccc3c2)C[C@H]1Oc1ccc2c(c1)CN([C@H]1CCC(=O)NC1=O)C2=O]

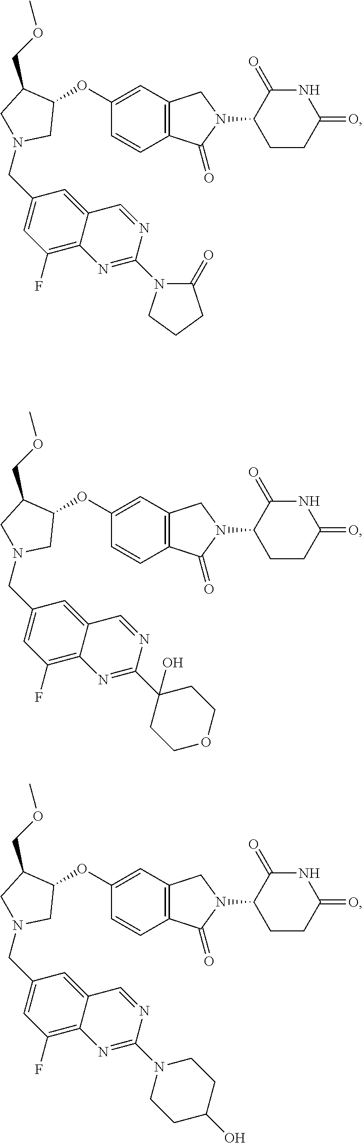 [SMILES: COC[C@@H]1CN(Cc2cc(F)c3nc(N4CCCC4=O)ncc3c2)C[C@H]1Oc1ccc2c(c1)CN([C@H]1CCC(=O)NC1=O)C2=O ; COC[C@@H]1CN(Cc2cc(F)c3nc(C4(O)CCOCC4)ncc3c2)C[C@H]1Oc1ccc2c(c1)CN([C@H]1CCC(=O)NC1=O)C2=O ; COC[C@@H]1CN(Cc2cc(F)c3nc(N4CCC(O)CC4)ncc3c2)C[C@H]1Oc1ccc2c(c1)CN([C@H]1CCC(=O)NC1=O)C2=O]

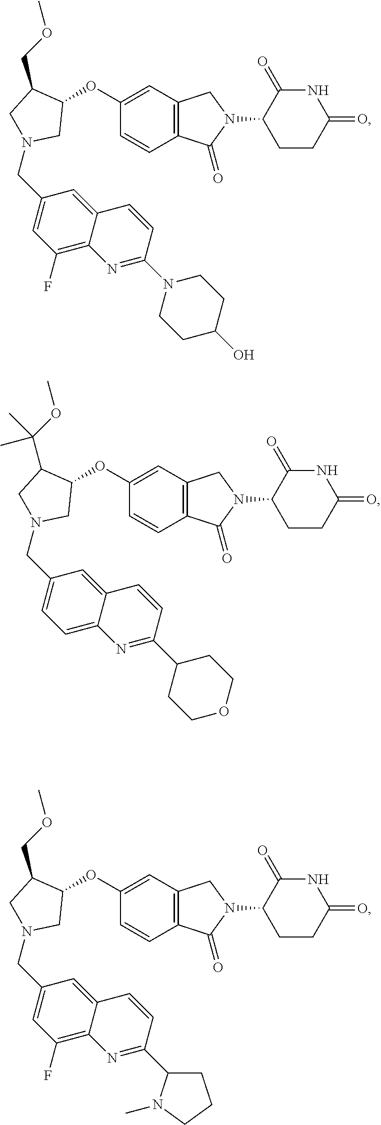 [SMILES: COC[C@@H]1CN(Cc2cc(F)c3nc(N4CCC(O)CC4)ccc3c2)C[C@H]1Oc1ccc2c(c1)CN([C@H]1CCC(=O)NC1=O)C2=O ; COC(C)(C)C1CN(Cc2ccc3nc(C4CCOCC4)ccc3c2)C[C@H]1Oc1ccc2c(c1)CN([C@H]1CCC(=O)NC1=O)C2=O ; COC[C@@H]1CN(Cc2cc(F)c3nc(C4CCCN4C)ccc3c2)C[C@H]1Oc1ccc2c(c1)CN([C@H]1CCC(=O)NC1=O)C2=O]

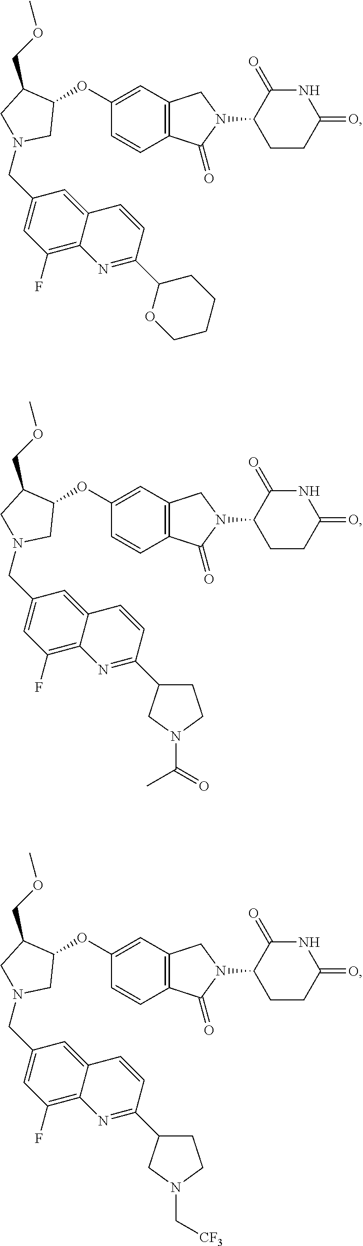 [SMILES: COC[C@@H]1CN(Cc2cc(F)c3nc(C4CCCCO4)ccc3c2)C[C@H]1Oc1ccc2c(c1)CN([C@H]1CCC(=O)NC1=O)C2=O ; COC[C@@H]1CN(Cc2cc(F)c3nc(C4CCN(C(C)=O)C4)ccc3c2)C[C@H]1Oc1ccc2c(c1)CN([C@H]1CCC(=O)NC1=O)C2=O ; COC[C@@H]1CN(Cc2cc(F)c3nc(C4CCN(CC(F)(F)F)C4)ccc3c2)C[C@H]1Oc1ccc2c(c1)CN([C@H]1CCC(=O)NC1=O)C2=O]

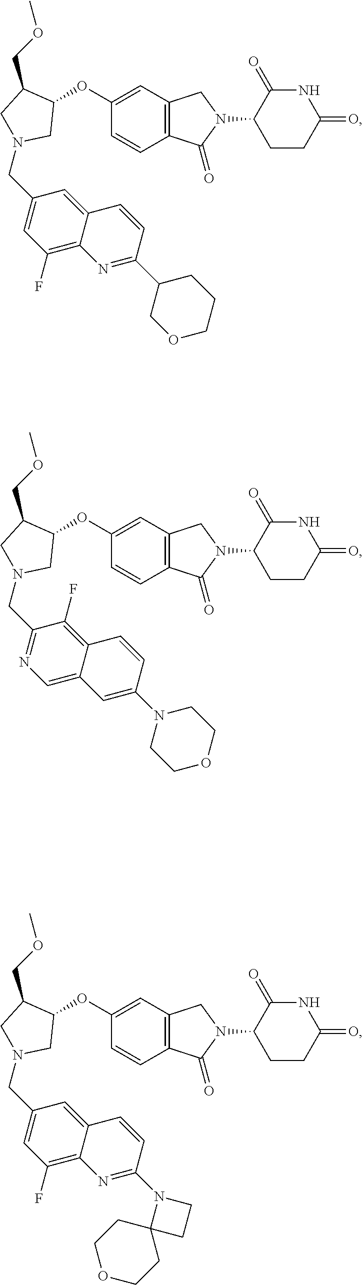 [SMILES: COC[C@@H]1CN(Cc2cc(F)c3nc(C4CCCOC4)ccc3c2)C[C@H]1Oc1ccc2c(c1)CN([C@H]1CCC(=O)NC1=O)C2=O ; COC[C@@H]1CN(Cc2ncc3cc(N4CCOCC4)ccc3c2F)C[C@H]1Oc1ccc2c(c1)CN([C@H]1CCC(=O)NC1=O)C2=O ; COC[C@@H]1CN(Cc2cc(F)c3nc(N4CCC45CCOCC5)ccc3c2)C[C@H]1Oc1ccc2c(c1)CN([C@H]1CCC(=O)NC1=O)C2=O]

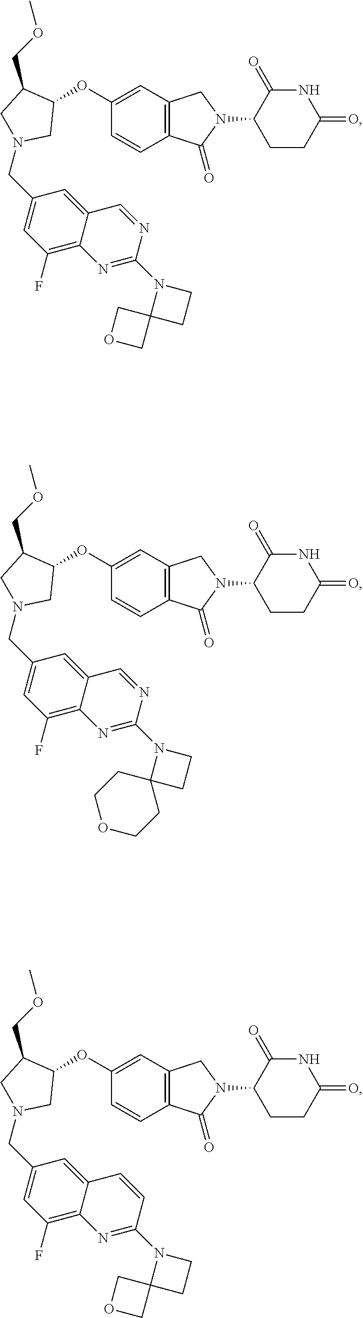 [SMILES: COC[C@@H]1CN(Cc2cc(F)c3nc(N4CCC45COC5)ncc3c2)C[C@H]1Oc1ccc2c(c1)CN([C@H]1CCC(=O)NC1=O)C2=O ; COC[C@@H]1CN(Cc2cc(F)c3nc(N4CCC45CCOCC5)ncc3c2)C[C@H]1Oc1ccc2c(c1)CN([C@H]1CCC(=O)NC1=O)C2=O ; COC[C@@H]1CN(Cc2cc(F)c3nc(N4CCC45COC5)ccc3c2)C[C@H]1Oc1ccc2c(c1)CN([C@H]1CCC(=O)NC1=O)C2=O]

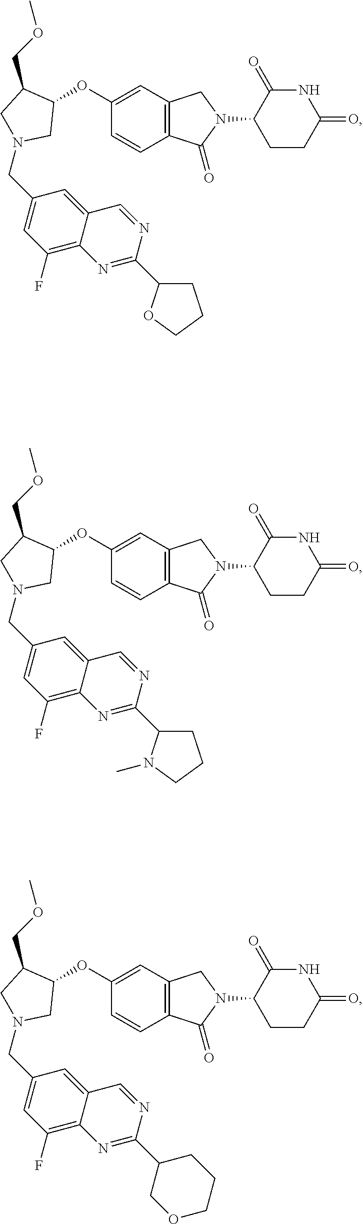 [SMILES: COC[C@@H]1CN(Cc2cc(F)c3nc(C4CCCO4)ncc3c2)C[C@H]1Oc1ccc2c(c1)CN([C@H]1CCC(=O)NC1=O)C2=O ; COC[C@@H]1CN(Cc2cc(F)c3nc(C4CCCN4C)ncc3c2)C[C@H]1Oc1ccc2c(c1)CN([C@H]1CCC(=O)NC1=O)C2=O ; COC[C@@H]1CN(Cc2cc(F)c3nc(C4CCCOC4)ncc3c2)C[C@H]1Oc1ccc2c(c1)CN([C@H]1CCC(=O)NC1=O)C2=O]

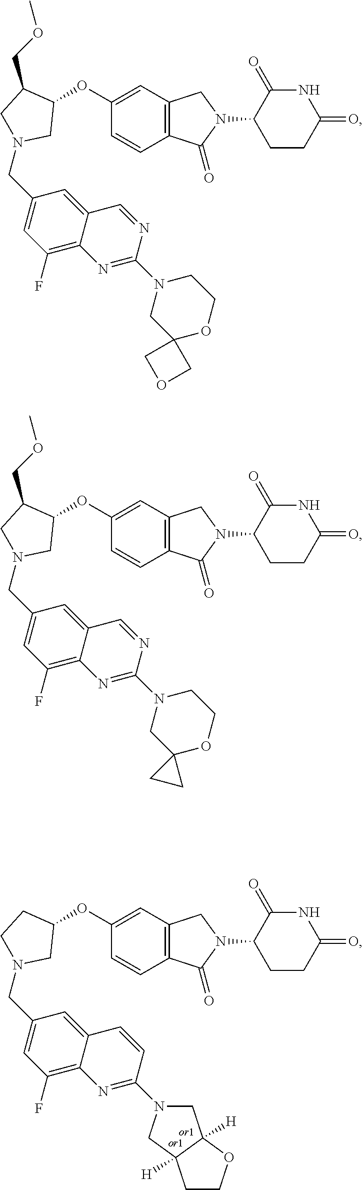 [SMILES: COC[C@@H]1CN(Cc2cc(F)c3nc(N4CCOC5(COC5)C4)ncc3c2)C[C@H]1Oc1ccc2c(c1)CN([C@H]1CCC(=O)NC1=O)C2=O ; COC[C@@H]1CN(Cc2cc(F)c3nc(N4CCOC5(CC5)C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(N7C[C@@H]8CCO[C@@H]8C7)ccc6c5)C4)ccc3C2=O)C(=O)N1]

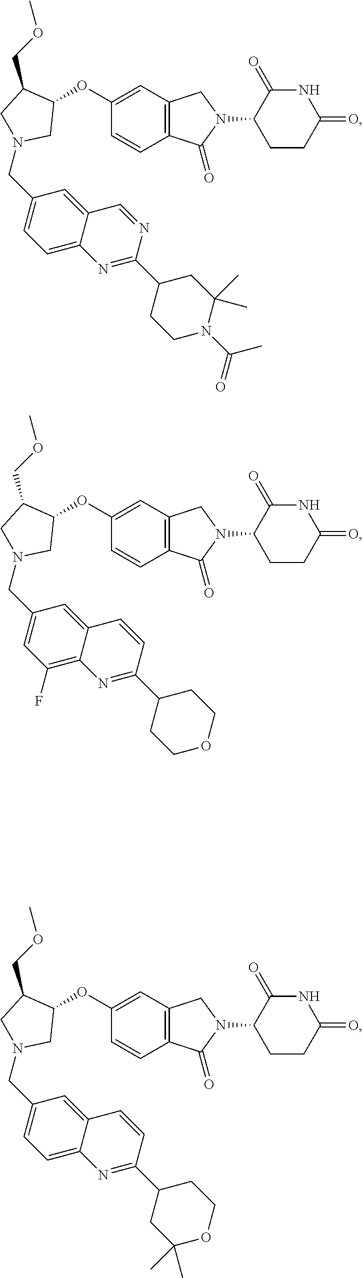 [SMILES: COC[C@@H]1CN(Cc2ccc3nc(C4CCN(C(C)=O)C(C)(C)C4)ncc3c2)C[C@H]1Oc1ccc2c(c1)CN([C@H]1CCC(=O)NC1=O)C2=O ; COC[C@H]1CN(Cc2cc(F)c3nc(C4CCOCC4)ccc3c2)C[C@H]1Oc1ccc2c(c1)CN([C@H]1CCC(=O)NC1=O)C2=O ; COC[C@@H]1CN(Cc2ccc3nc(C4CCOC(C)(C)C4)ccc3c2)C[C@H]1Oc1ccc2c(c1)CN([C@H]1CCC(=O)NC1=O)C2=O]

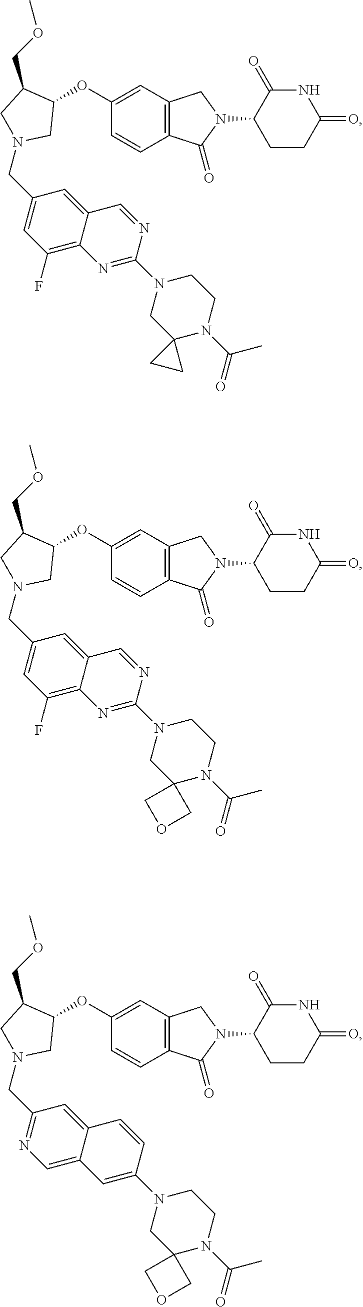 [SMILES: COC[C@@H]1CN(Cc2cc(F)c3nc(N4CCN(C(C)=O)C5(CC5)C4)ncc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(COC5)C4)ncc3c2)C[C@H]1Oc1ccc2c(c1)CN([C@H]1CCC(=O)NC1=O)C2=O ; COC[C@@H]1CN(Cc2cc3ccc(N4CCN(C(C)=O)C5(COC5)C4)cc3cn2)C[C@H]1Oc1ccc2c(c1)CN([C@H]1CCC(=O)NC1=O)C2=O]

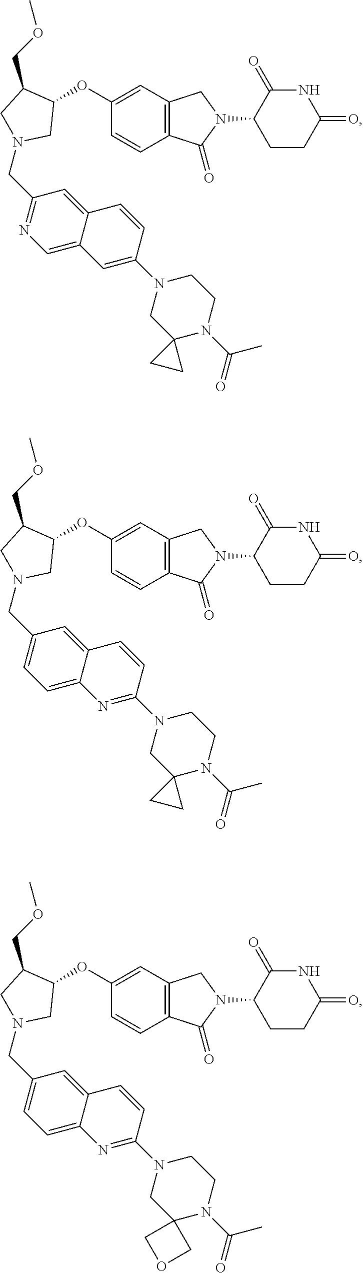 [SMILES: COC[C@@H]1CN(Cc2cc3ccc(N4CCN(C(C)=O)C5(CC5)C4)cc3cn2)C[C@H]1Oc1ccc2c(c1)CN([C@H]1CCC(=O)NC1=O)C2=O ; COC[C@@H]1CN(Cc2ccc3nc(N4CCN(C(C)=O)C5(CC5)C4)ccc3c2)C[C@H]1Oc1ccc2c(c1)CN([C@H]1CCC(=O)NC1=O)C2=O ; COC[C@@H]1CN(Cc2ccc3nc(N4CCN(C(C)=O)C5(COC5)C4)ccc3c2)C[C@H]1Oc1ccc2c(c1)CN([C@H]1CCC(=O)NC1=O)C2=O]

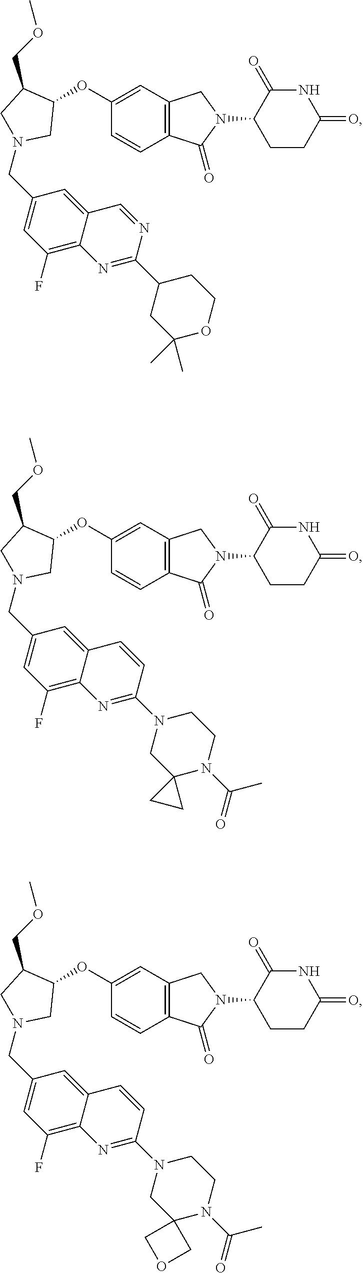 [SMILES: COC[C@@H]1CN(Cc2cc(F)c3nc(C4CCOC(C)(C)C4)ncc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(CC5)C4)ccc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(COC5)C4)ccc3c2)C[C@H]1Oc1ccc2c(c1)CN([C@H]1CCC(=O)NC1=O)C2=O]

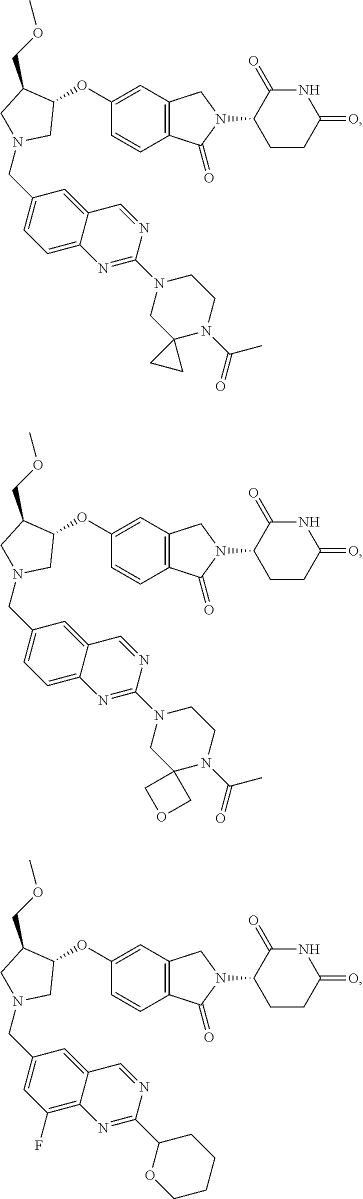 [SMILES: COC[C@@H]1CN(Cc2ccc3nc(N4CCN(C(C)=O)C5(CC5)C4)ncc3c2)C[C@H]1Oc1ccc2c(c1)CN([C@H]1CCC(=O)NC1=O)C2=O ; COC[C@@H]1CN(Cc2ccc3nc(N4CCN(C(C)=O)C5(COC5)C4)ncc3c2)C[C@H]1Oc1ccc2c(c1)CN([C@H]1CCC(=O)NC1=O)C2=O ; COC[C@@H]1CN(Cc2cc(F)c3nc(C4CCCCO4)ncc3c2)C[C@H]1Oc1ccc2c(c1)CN([C@H]1CCC(=O)NC1=O)C2=O]

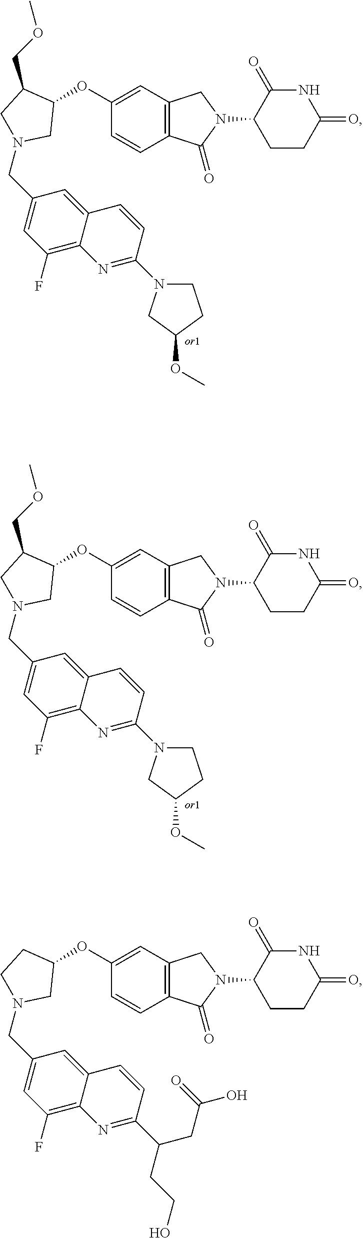 [SMILES: COC[C@@H]1CN(Cc2cc(F)c3nc(N4CC[C@@H](OC)C4)ccc3c2)C[C@H]1Oc1ccc2c(c1)CN([C@H]1CCC(=O)NC1=O)C2=O ; COC[C@@H]1CN(Cc2cc(F)c3nc(N4CC[C@H](OC)C4)ccc3c2)C[C@H]1Oc1ccc2c(c1)CN([C@H]1CCC(=O)NC1=O)C2=O ; O=C(O)CC(CCO)c1ccc2cc(CN3CC[C@H](Oc4ccc5c(c4)CN([C@H]4CCC(=O)NC4=O)C5=O)C3)cc(F)c2n1]

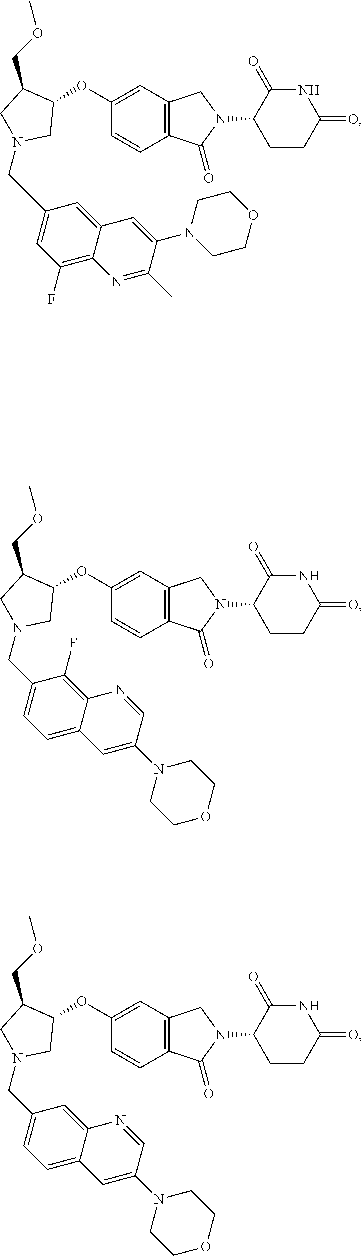 [SMILES: COC[C@@H]1CN(Cc2cc(F)c3nc(C)c(N4CCOCC4)cc3c2)C[C@H]1Oc1ccc2c(c1)CN([C@H]1CCC(=O)NC1=O)C2=O ; COC[C@@H]1CN(Cc2ccc3cc(N4CCOCC4)cnc3c2F)C[C@H]1Oc1ccc2c(c1)CN([C@H]1CCC(=O)NC1=O)C2=O ; COC[C@@H]1CN(Cc2ccc3cc(N4CCOCC4)cnc3c2)C[C@H]1Oc1ccc2c(c1)CN([C@H]1CCC(=O)NC1=O)C2=O]

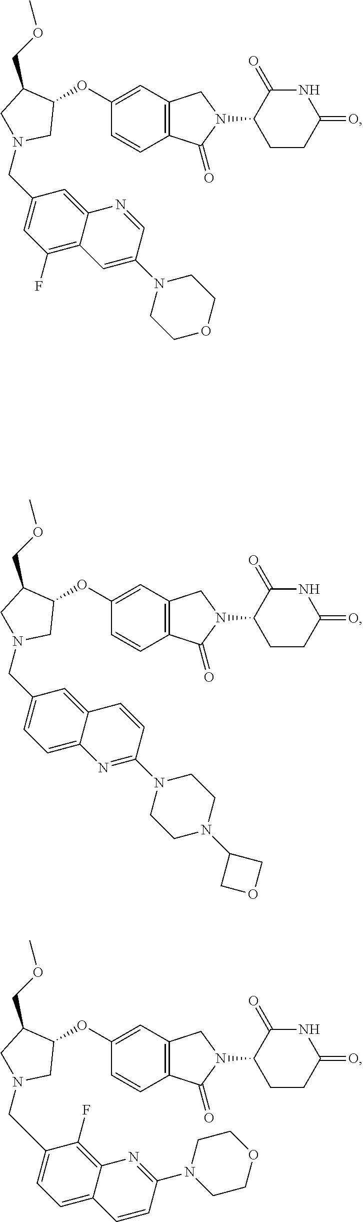 [SMILES: COC[C@@H]1CN(Cc2cc(F)c3cc(N4CCOCC4)cnc3c2)C[C@H]1Oc1ccc2c(c1)CN([C@H]1CCC(=O)NC1=O)C2=O ; COC[C@@H]1CN(Cc2ccc3nc(N4CCN(C5COC5)CC4)ccc3c2)C[C@H]1Oc1ccc2c(c1)CN([C@H]1CCC(=O)NC1=O)C2=O ; COC[C@@H]1CN(Cc2ccc3ccc(N4CCOCC4)nc3c2F)C[C@H]1Oc1ccc2c(c1)CN([C@H]1CCC(=O)NC1=O)C2=O]

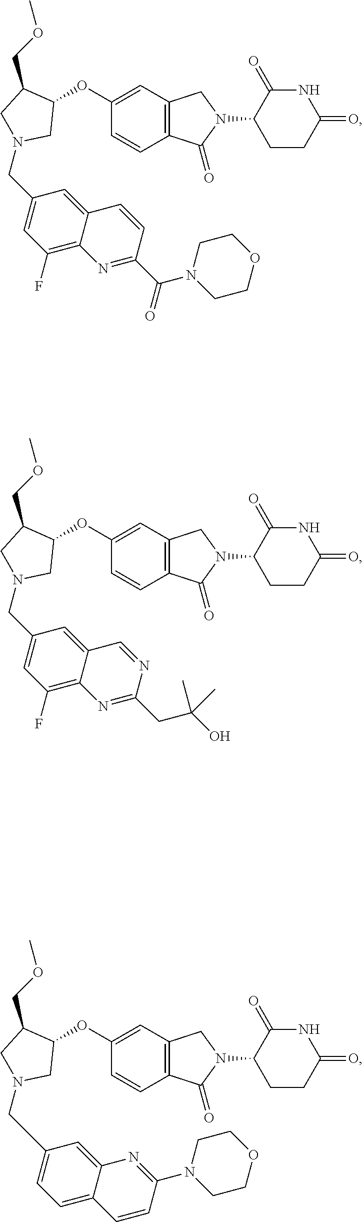 [SMILES: COC[C@@H]1CN(Cc2cc(F)c3nc(C(=O)N4CCOCC4)ccc3c2)C[C@H]1Oc1ccc2c(c1)CN([C@H]1CCC(=O)NC1=O)C2=O ; COC[C@@H]1CN(Cc2cc(F)c3nc(CC(C)(C)O)ncc3c2)C[C@H]1Oc1ccc2c(c1)CN([C@H]1CCC(=O)NC1=O)C2=O ; COC[C@@H]1CN(Cc2ccc3ccc(N4CCOCC4)nc3c2)C[C@H]1Oc1ccc2c(c1)CN([C@H]1CCC(=O)NC1=O)C2=O]

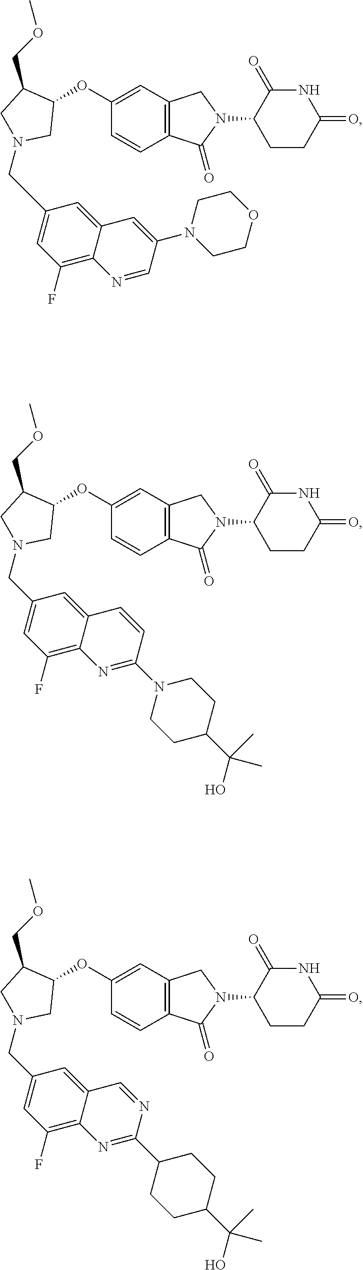 [SMILES: COC[C@@H]1CN(Cc2cc(F)c3ncc(N4CCOCC4)cc3c2)C[C@H]1Oc1ccc2c(c1)CN([C@H]1CCC(=O)NC1=O)C2=O ; COC[C@@H]1CN(Cc2cc(F)c3nc(N4CCC(C(C)(C)O)CC4)ccc3c2)C[C@H]1Oc1ccc2c(c1)CN([C@H]1CCC(=O)NC1=O)C2=O ; COC[C@@H]1CN(Cc2cc(F)c3nc(C4CCC(C(C)(C)O)CC4)ncc3c2)C[C@H]1Oc1ccc2c(c1)CN([C@H]1CCC(=O)NC1=O)C2=O]

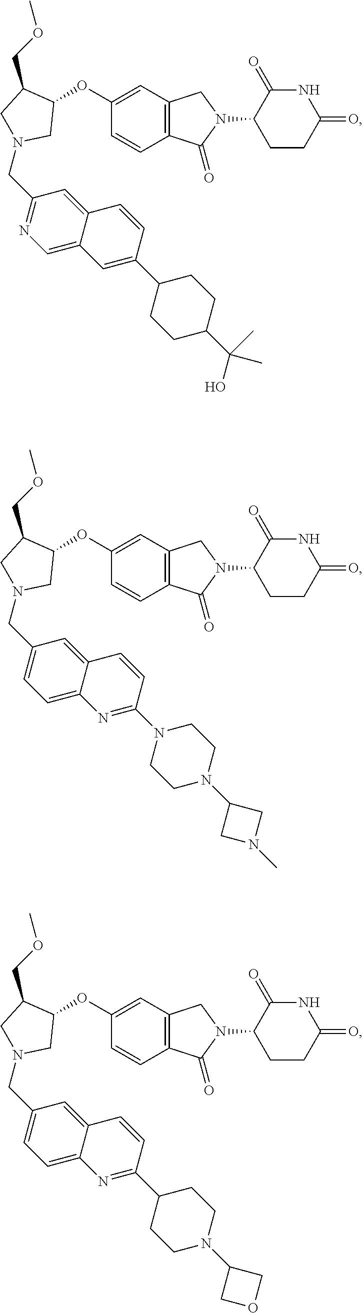 [SMILES: COC[C@@H]1CN(Cc2cc3ccc(C4CCC(C(C)(C)O)CC4)cc3cn2)C[C@H]1Oc1ccc2c(c1)CN([C@H]1CCC(=O)NC1=O)C2=O ; COC[C@@H]1CN(Cc2ccc3nc(N4CCN(C5CN(C)C5)CC4)ccc3c2)C[C@H]1Oc1ccc2c(c1)CN([C@H]1CCC(=O)NC1=O)C2=O ; COC[C@@H]1CN(Cc2ccc3nc(C4CCN(C5COC5)CC4)ccc3c2)C[C@H]1Oc1ccc2c(c1)CN([C@H]1CCC(=O)NC1=O)C2=O]

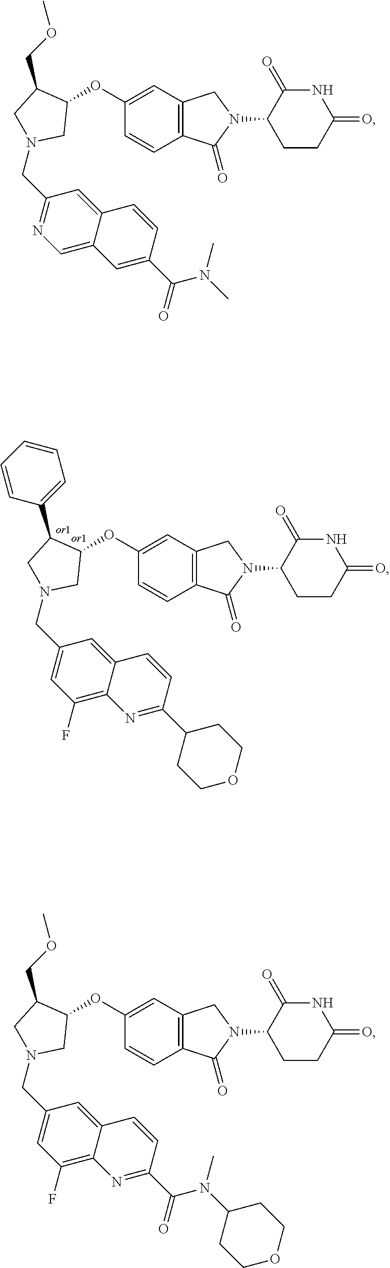 [SMILES: COC[C@@H]1CN(Cc2cc3ccc(C(=O)N(C)C)cc3cn2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4c4ccccc4)ccc3C2=O)C(=O)N1 ; COC[C@@H]1CN(Cc2cc(F)c3nc(C(=O)N(C)C4CCOCC4)ccc3c2)C[C@H]1Oc1ccc2c(c1)CN([C@H]1CCC(=O)NC1=O)C2=O]

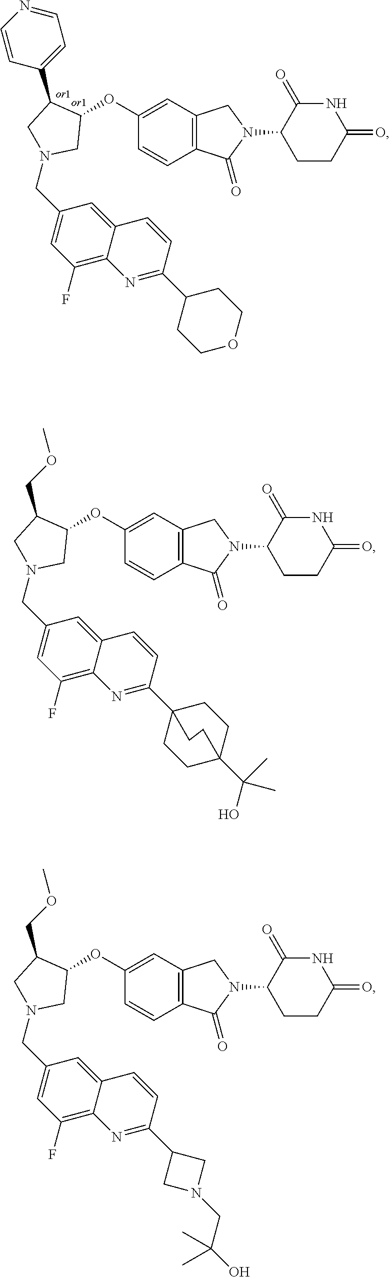 [SMILES: O=C1CC[C@H](N2Cc3cc(O[C@@H]4CN(Cc5cc(F)c6nc(C7CCOCC7)ccc6c5)C[C@H]4c4ccncc4)ccc3C2=O)C(=O)N1 ; COC[C@@H]1CN(Cc2cc(F)c3nc(C45CCC(C(C)(C)O)(CC4)CC5)ccc3c2)C[C@H]1Oc1ccc2c(c1)CN([C@H]1CCC(=O)NC1=O)C2=O ; COC[C@@H]1CN(Cc2cc(F)c3nc(C4CN(CC(C)(C)O)C4)ccc3c2)C[C@H]1Oc1ccc2c(c1)CN([C@H]1CCC(=O)NC1=O)C2=O]

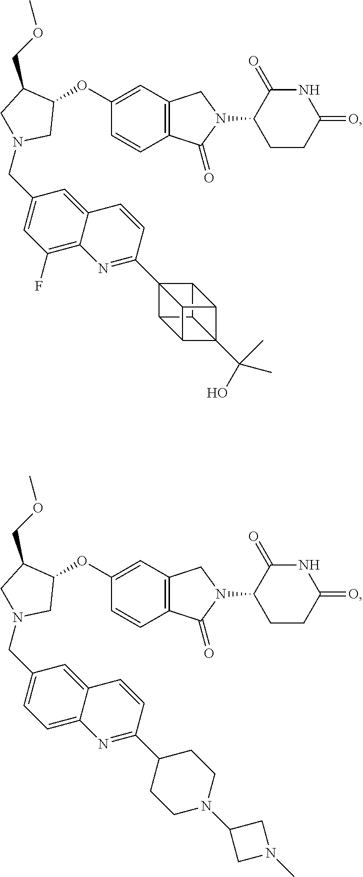 [SMILES: COC[C@@H]1CN(Cc2cc(F)c3nc(C45C6C7C4C4C5C6C74C(C)(C)O)ccc3c2)C[C@H]1Oc1ccc2c(c1)CN([C@H]1CCC(=O)NC1=O)C2=O ; COC[C@@H]1CN(Cc2ccc3nc(C4CCN(C5CN(C)C5)CC4)ccc3c2)C[C@H]1Oc1ccc2c(c1)CN([C@H]1CCC(=O)NC1=O)C2=O]

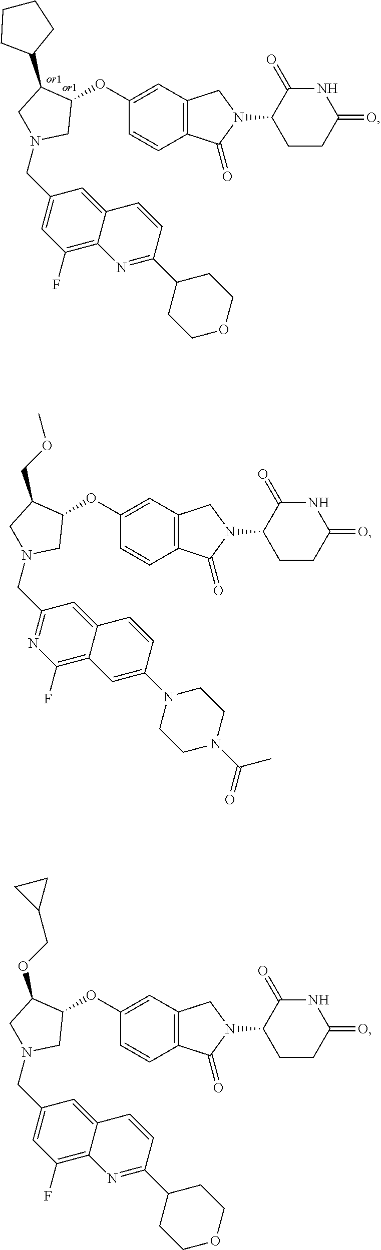 [SMILES: O=C1CC[C@H](N2Cc3cc(O[C@@H]4CN(Cc5cc(F)c6nc(C7CCOCC7)ccc6c5)C[C@H]4C4CCCC4)ccc3C2=O)C(=O)N1 ; COC[C@@H]1CN(Cc2cc3ccc(N4CCN(C(C)=O)CC4)cc3c(F)n2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4OCC4CC4)ccc3C2=O)C(=O)N1]

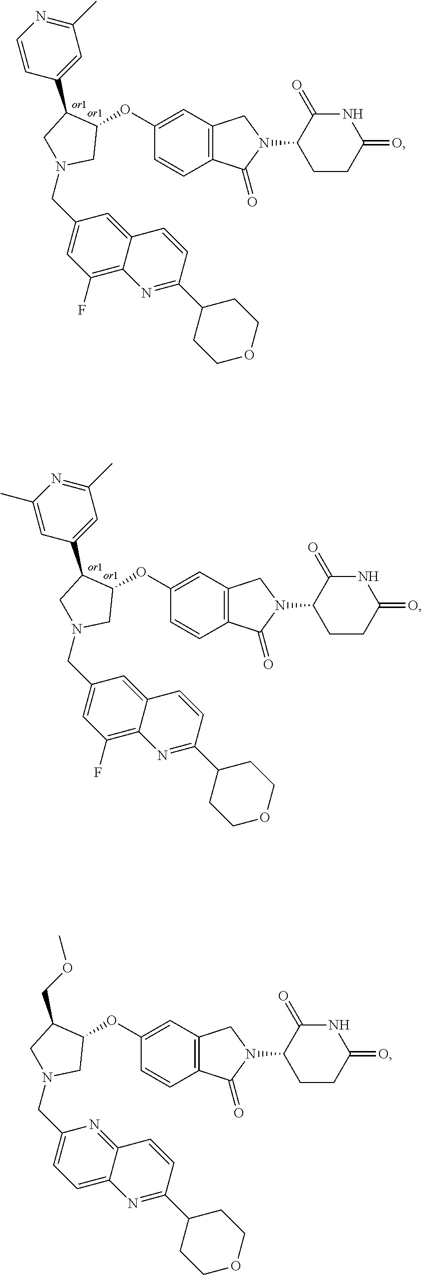 [SMILES: Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)ccn1 ; Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)cc(C)n1 ; COC[C@@H]1CN(Cc2ccc3nc(C4CCOCC4)ccc3n2)C[C@H]1Oc1ccc2c(c1)CN([C@H]1CCC(=O)NC1=O)C2=O]

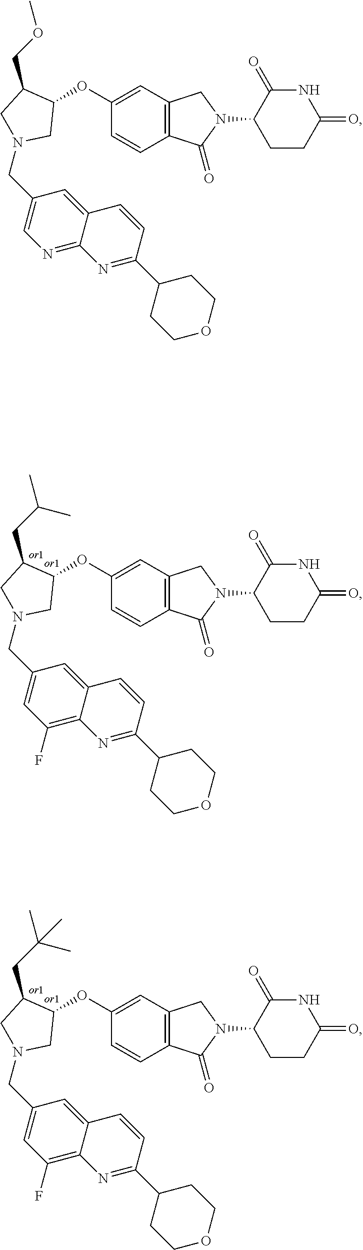 [SMILES: COC[C@@H]1CN(Cc2cnc3nc(C4CCOCC4)ccc3c2)C[C@H]1Oc1ccc2c(c1)CN([C@H]1CCC(=O)NC1=O)C2=O ; CC(C)C[C@@H]1CN(Cc2cc(F)c3nc(C4CCOCC4)ccc3c2)C[C@H]1Oc1ccc2c(c1)CN([C@H]1CCC(=O)NC1=O)C2=O ; CC(C)(C)C[C@@H]1CN(Cc2cc(F)c3nc(C4CCOCC4)ccc3c2)C[C@H]1Oc1ccc2c(c1)CN([C@H]1CCC(=O)NC1=O)C2=O]

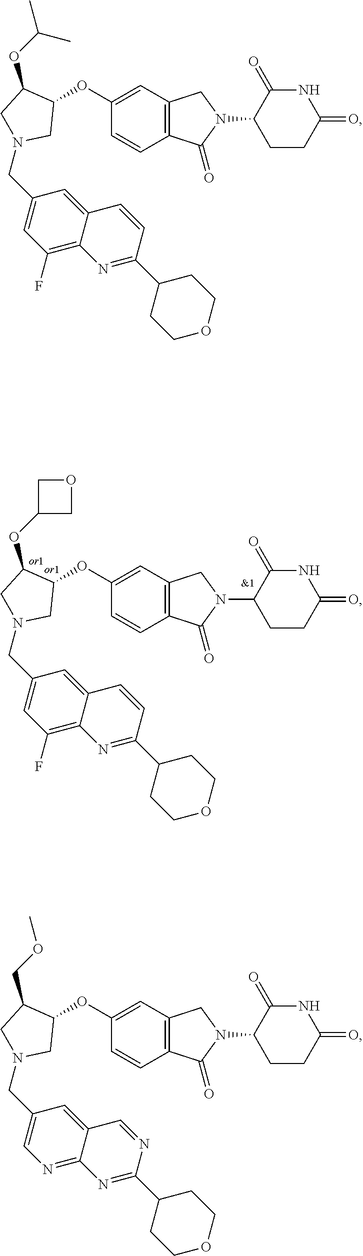 [SMILES: CC(C)O[C@@H]1CN(Cc2cc(F)c3nc(C4CCOCC4)ccc3c2)C[C@H]1Oc1ccc2c(c1)CN([C@H]1CCC(=O)NC1=O)C2=O ; O=C1CCC(N2Cc3cc(O[C@@H]4CN(Cc5cc(F)c6nc(C7CCOCC7)ccc6c5)C[C@H]4OC4COC4)ccc3C2=O)C(=O)N1 ; COC[C@@H]1CN(Cc2cnc3nc(C4CCOCC4)ncc3c2)C[C@H]1Oc1ccc2c(c1)CN([C@H]1CCC(=O)NC1=O)C2=O]

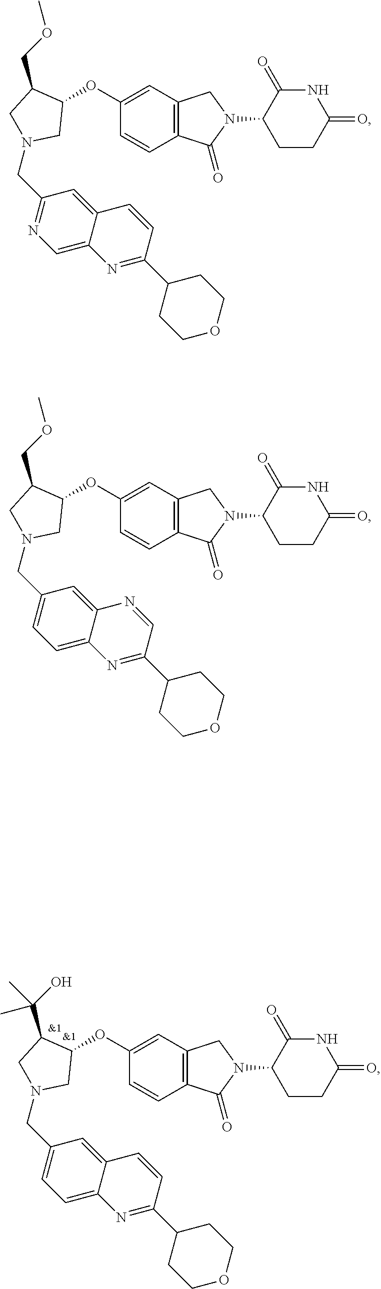 [SMILES: COC[C@@H]1CN(Cc2cc3ccc(C4CCOCC4)nc3cn2)C[C@H]1Oc1ccc2c(c1)CN([C@H]1CCC(=O)NC1=O)C2=O ; COC[C@@H]1CN(Cc2ccc3nc(C4CCOCC4)cnc3c2)C[C@H]1Oc1ccc2c(c1)CN([C@H]1CCC(=O)NC1=O)C2=O ; CC(C)(O)[C@@H]1CN(Cc2ccc3nc(C4CCOCC4)ccc3c2)C[C@H]1Oc1ccc2c(c1)CN([C@H]1CCC(=O)NC1=O)C2=O]

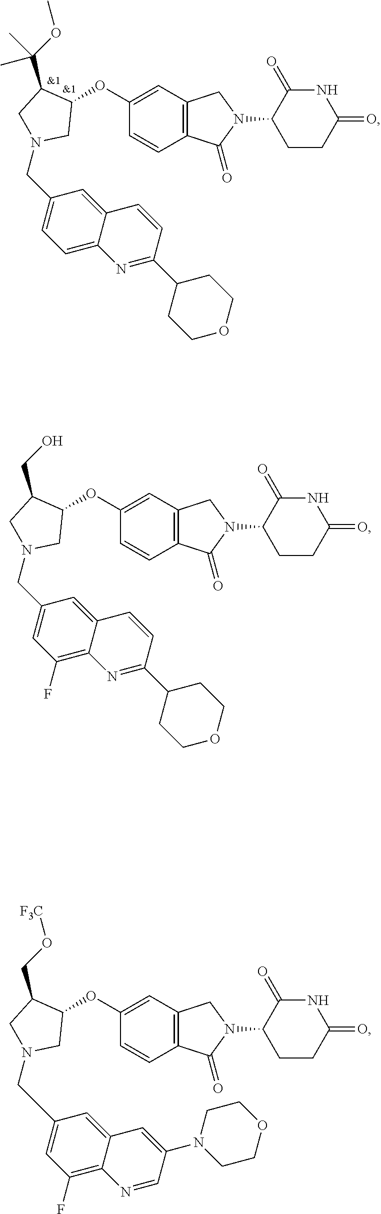 [SMILES: COC(C)(C)[C@@H]1CN(Cc2ccc3nc(C4CCOCC4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4CO)ccc3C2=O)C(=O)N1 ; O=C1CC[C@H](N2Cc3cc(O[C@@H]4CN(Cc5cc(F)c6ncc(N7CCOCC7)cc6c5)C[C@H]4COC(F)(F)F)ccc3C2=O)C(=O)N1]

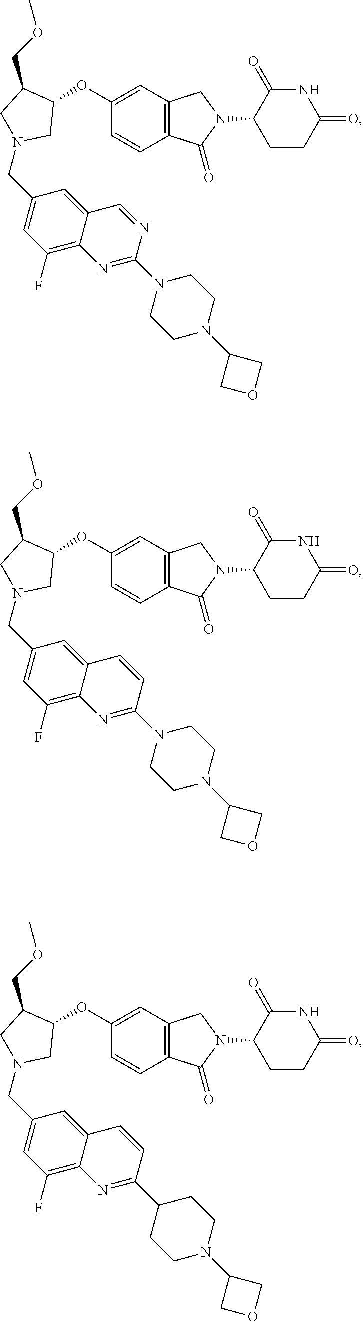 [SMILES: COC[C@@H]1CN(Cc2cc(F)c3nc(N4CCN(C5COC5)CC4)ncc3c2)C[C@H]1Oc1ccc2c(c1)CN([C@H]1CCC(=O)NC1=O)C2=O ; COC[C@@H]1CN(Cc2cc(F)c3nc(N4CCN(C5COC5)CC4)ccc3c2)C[C@H]1Oc1ccc2c(c1)CN([C@H]1CCC(=O)NC1=O)C2=O ; COC[C@@H]1CN(Cc2cc(F)c3nc(C4CCN(C5COC5)CC4)ccc3c2)C[C@H]1Oc1ccc2c(c1)CN([C@H]1CCC(=O)NC1=O)C2=O]

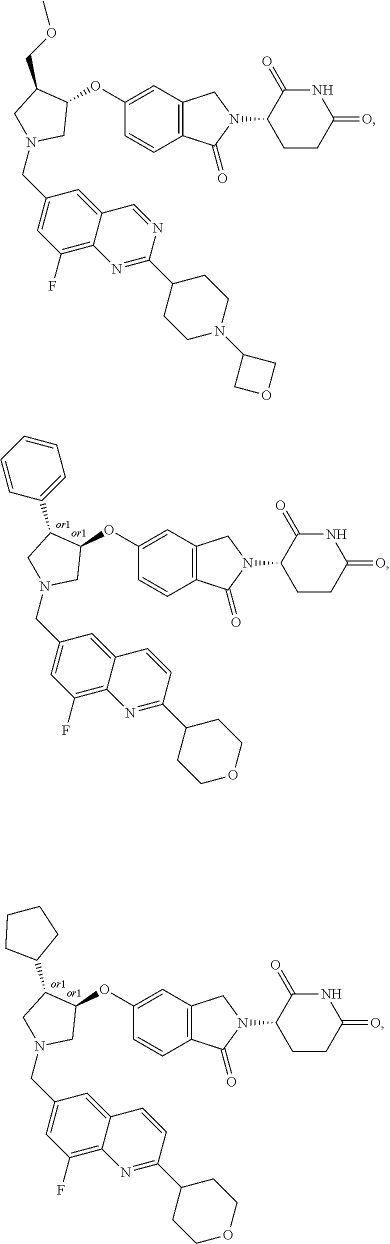 [SMILES: COC[C@@H]1CN(Cc2cc(F)c3nc(C4CCN(C5COC5)CC4)ncc3c2)C[C@H]1Oc1ccc2c(c1)CN([C@H]1CCC(=O)NC1=O)C2=O ; O=C1CC[C@H](N2Cc3cc(O[C@H]4CN(Cc5cc(F)c6nc(C7CCOCC7)ccc6c5)C[C@@H]4c4ccccc4)ccc3C2=O)C(=O)N1 ; O=C1CC[C@H](N2Cc3cc(O[C@H]4CN(Cc5cc(F)c6nc(C7CCOCC7)ccc6c5)C[C@@H]4C4CCCC4)ccc3C2=O)C(=O)N1]

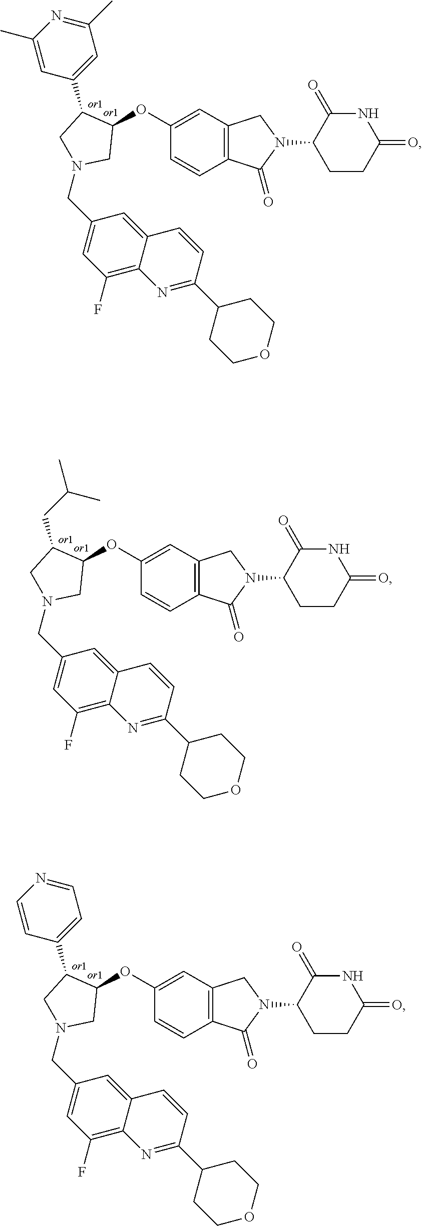 [SMILES: Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)cc(C)n1 ; CC(C)C[C@H]1CN(Cc2cc(F)c3nc(C4CCOCC4)ccc3c2)C[C@@H]1Oc1ccc2c(c1)CN([C@H]1CCC(=O)NC1=O)C2=O ; O=C1CC[C@H](N2Cc3cc(O[C@H]4CN(Cc5cc(F)c6nc(C7CCOCC7)ccc6c5)C[C@@H]4c4ccncc4)ccc3C2=O)C(=O)N1]

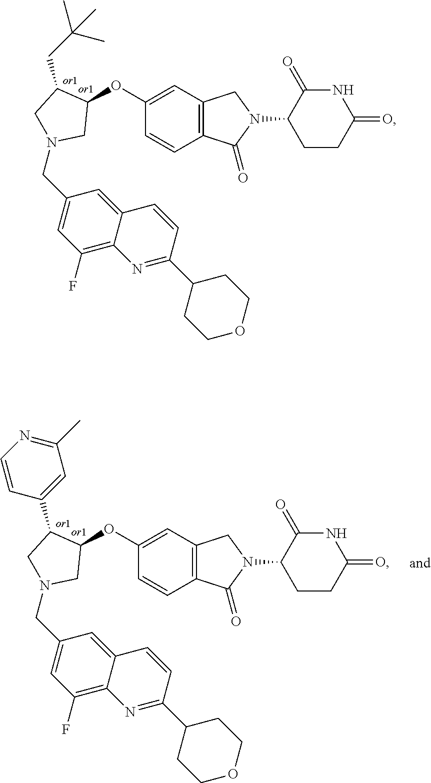 [SMILES: CC(C)(C)C[C@H]1CN(Cc2cc(F)c3nc(C4CCOCC4)ccc3c2)C[C@@H]1Oc1ccc2c(c1)CN([C@H]1CCC(=O)NC1=O)C2=O ; Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)ccn1]

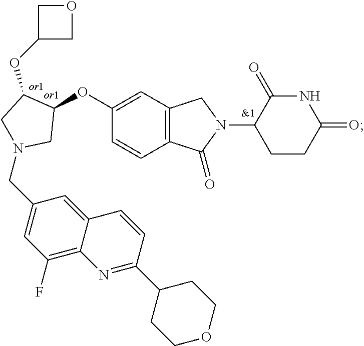 [SMILES: O=C1CCC(N2Cc3cc(O[C@H]4CN(Cc5cc(F)c6nc(C7CCOCC7)ccc6c5)C[C@@H]4OC4COC4)ccc3C2=O)C(=O)N1]

or a pharmaceutically acceptable salt, tautomer, isotopologue, or stereoisomer thereof.

In some embodiments, provided herein are methods for reducing WEE1 kinase protein levels, the method comprising contacting a cell with an effective amount of a compound of the present disclosure or a pharmaceutically acceptable salt, tautomer, isotopologue, or stereoisomer thereof. In some embodiments, provided herein are uses of a compound of the present disclosure or a pharmaceutically acceptable salt, tautomer, isotopologue, or stereoisomer thereof, in the manufacture of a medicament for reducing WEE1 kinase protein levels.

In some embodiments, provided herein are methods of preventing or treating cancer in a subject comprising administering to a subject in need thereof an effective amount of a compound of the present disclosure or a pharmaceutically acceptable salt, tautomer, isotopologue, or stereoisomer thereof. In some embodiments, provided herein are uses of a compound of the present disclosure or a pharmaceutically acceptable salt, tautomer, isotopologue, or stereoisomer thereof, in the manufacture of a medicament for the prevention or treatment of cancer.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, cycloalkylalkyloxy, arylalkyloxy, heterocyclylalkyloxy, heteroarylalkyloxy; oxo (═O); amino, alkylamino, cycloalkylamino, arylamino, heterocyclylamino, heteroarylamino, cycloalkylalkylamino, arylalkylamino, heterocyclylalkylamino, heteroarylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or —O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is —O-(alkyl), wherein alkyl is defined above.

An “oxo” group is a “═O” group bonded to a carbon.

An “amino” group is —NH₂, wherein one or both of the hydrogen atoms may be substituted with alkyl, cycloalkyl, heterocyclyl, aryl, or heteroaryl.

An “amido” group is an amide group with the formula —NHC(O)—, wherein the hydrogen atom may be substituted with alkyl, cycloalkyl, heterocyclyl, aryl, or heteroaryl.

A “heteroaryl-oxy” group is —O-(heteroaryl), wherein the heteroaryl is defined above. A “heterocyclyl-oxy” group is —O-(heterocyclyl), wherein the heterocyclyl is defined above. A “cycloalkyl-oxy” group is —O-(cycloalkyl), wherein the cycloalkyl is defined above

When the groups described herein, with the exception of alkyl group, amino group, and amido group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxo (═O); B(OH)₂, —O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl); cycloalkyloxy, aryloxy, heterocyclyloxy, heteroaryloxy, cycloalkylalkyloxy, arylalkyloxy, heterocyclylalkyloxy, and heteroarylalkyloxy.

Embodiments of the disclosure are meant to encompass pharmaceutically acceptable salts, tautomers, isotopologues, and stereoisomers of the compounds provided herein, such as the compounds of the present disclosure.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of the present disclosure include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KgaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

“Tautomers” refers to isomeric forms of a compound that are in equilibrium based on proton transfers. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

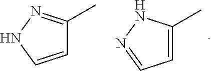

As readily understood by one skilled in the art, a wide variety of functional groups and other structures may exhibit tautomerism and all tautomers of compounds of the present disclosure are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an WEE1 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein are compounds from:

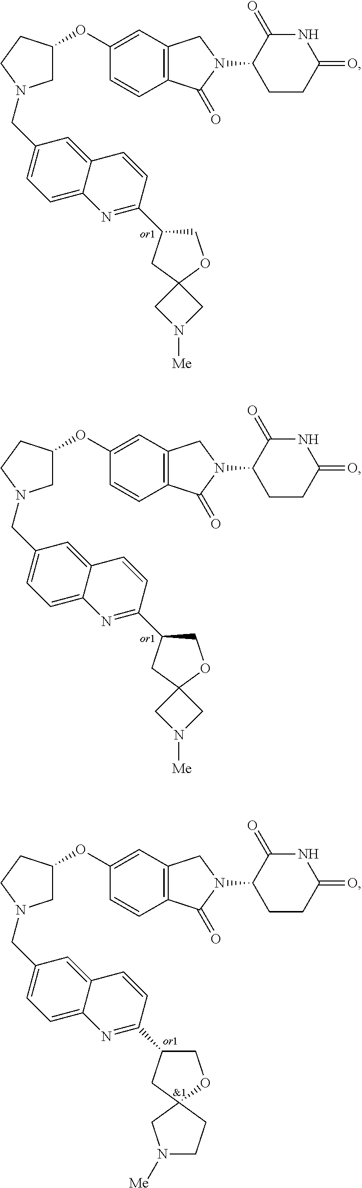 [SMILES: CN1CC2(C[C@H](c3ccc4cc(CN5CC[C@H](Oc6ccc7c(c6)CN([C@H]6CCC(=O)NC6=O)C7=O)C5)ccc4n3)CO2)C1 ; CN1CC2(C[C@@H](c3ccc4cc(CN5CC[C@H](Oc6ccc7c(c6)CN([C@H]6CCC(=O)NC6=O)C7=O)C5)ccc4n3)CO2)C1 ; CN1CC[C@]2(C[C@H](c3ccc4cc(CN5CC[C@H](Oc6ccc7c(c6)CN([C@H]6CCC(=O)NC6=O)C7=O)C5)ccc4n3)CO2)C1]

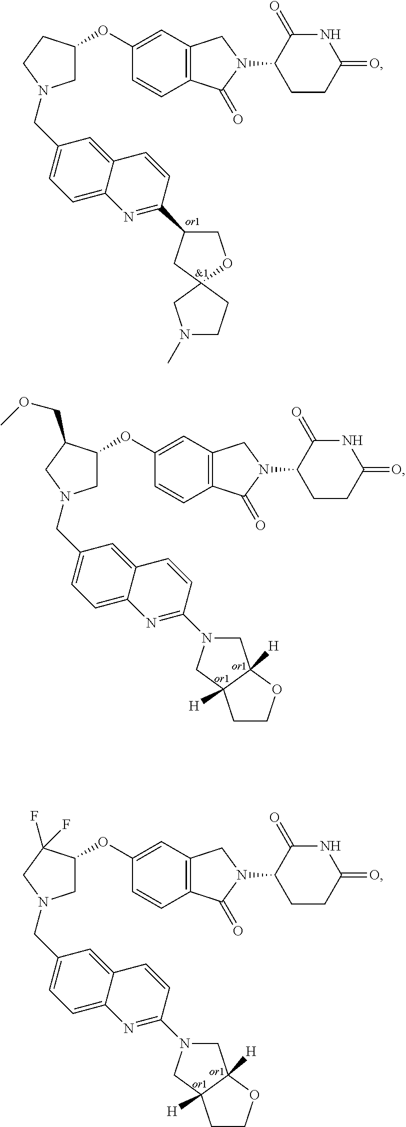 [SMILES: CN1CC[C@]2(C[C@@H](c3ccc4cc(CN5CC[C@H](Oc6ccc7c(c6)CN([C@H]6CCC(=O)NC6=O)C7=O)C5)ccc4n3)CO2)C1 ; COC[C@@H]1CN(Cc2ccc3nc(N4C[C@H]5CCO[C@H]5C4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5ccc6nc(N7C[C@H]8CCO[C@H]8C7)ccc6c5)CC4(F)F)ccc3C2=O)C(=O)N1]

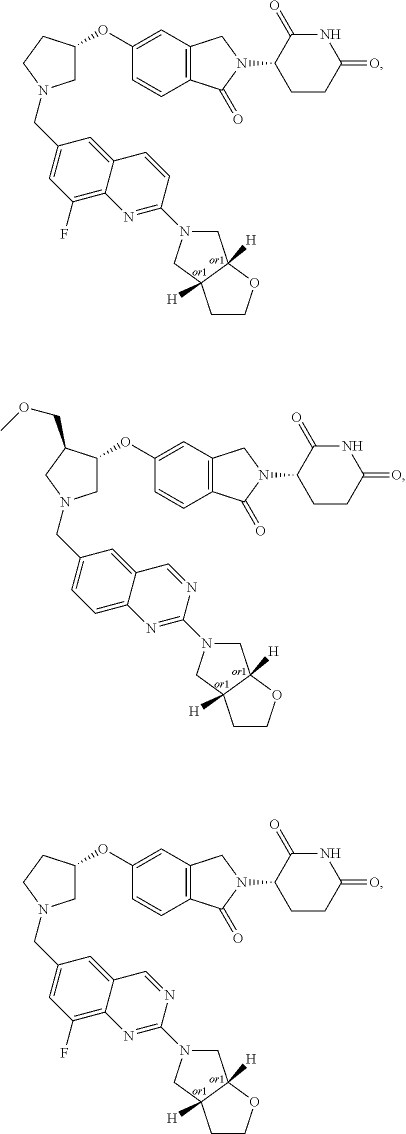 [SMILES: O=C1CC[C@H](N2Cc3cc(O[C@H]4CCN(Cc5cc(F)c6nc(N7C[C@H]8CCO[C@H]8C7)ccc6c5)C4)ccc3C2=O)C(=O)N1 ; COC[C@@H]1CN(Cc2ccc3nc(N4C[C@H]5CCO[C@H]5C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(N7C[C@H]8CCO[C@H]8C7)ncc6c5)C4)ccc3C2=O)C(=O)N1]

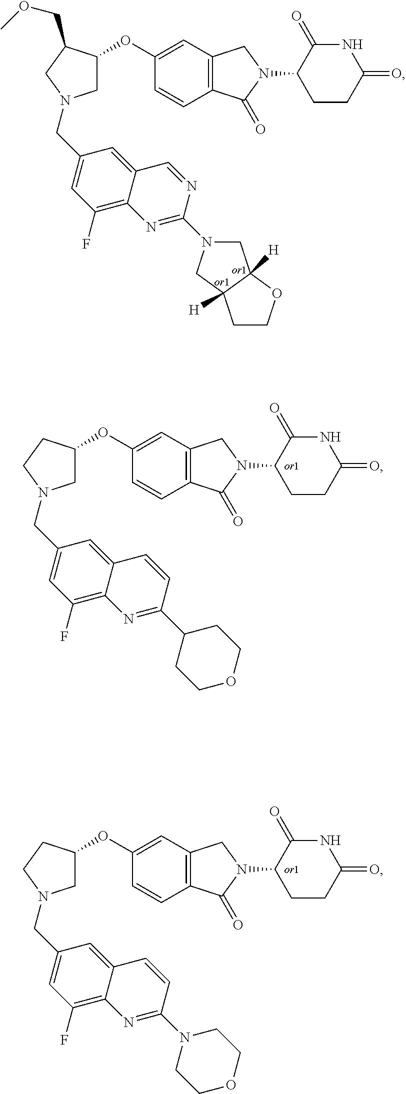 [SMILES: COC[C@@H]1CN(Cc2cc(F)c3nc(N4C[C@H]5CCO[C@H]5C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(C7CCOCC7)ccc6c5)C4)ccc3C2=O)C(=O)N1 ; O=C1CC[C@H](N2Cc3cc(O[C@H]4CCN(Cc5cc(F)c6nc(N7CCOCC7)ccc6c5)C4)ccc3C2=O)C(=O)N1]

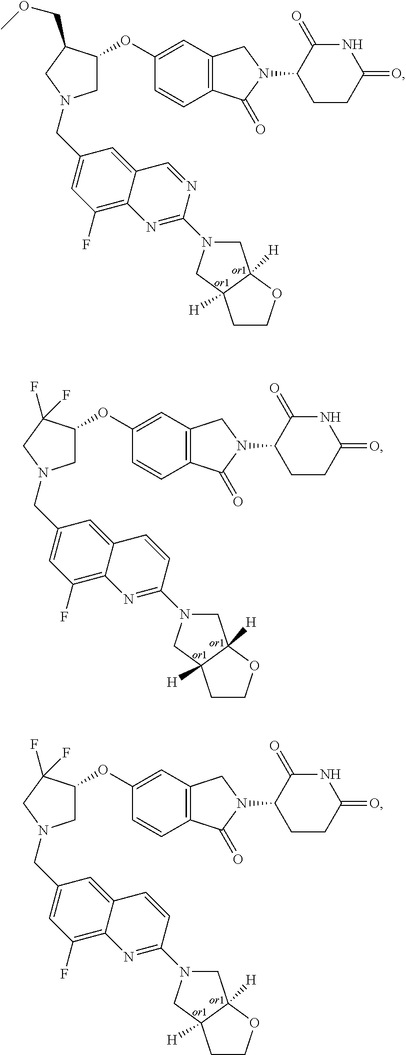 [SMILES: COC[C@@H]1CN(Cc2cc(F)c3nc(N4C[C@@H]5CCO[C@@H]5C4)ncc3c2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(N7C[C@H]8CCO[C@H]8C7)ccc6c5)CC4(F)F)ccc3C2=O)C(=O)N1 ; O=C1CC[C@H](N2Cc3cc(O[C@@H]4CN(Cc5cc(F)c6nc(N7C[C@@H]8CCO[C@@H]8C7)ccc6c5)CC4(F)F)ccc3C2=O)C(=O)N1]

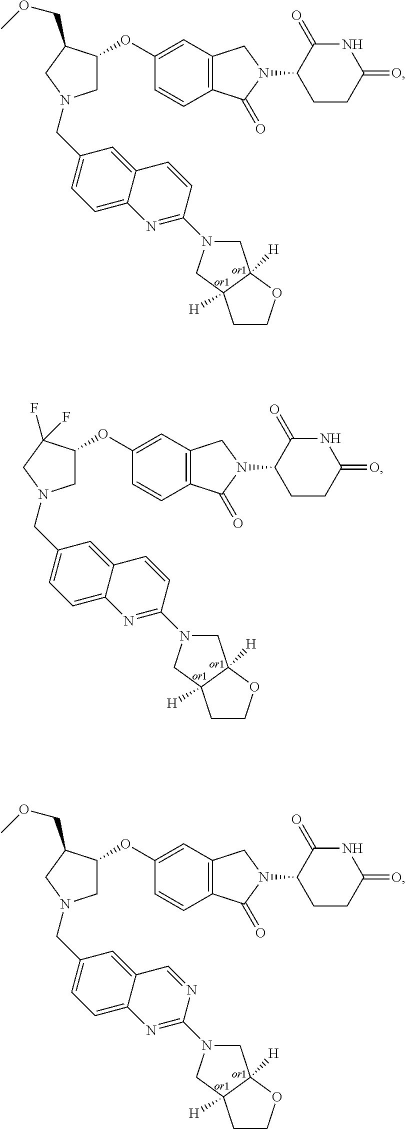 [SMILES: COC[C@@H]1CN(Cc2ccc3nc(N4C[C@@H]5CCO[C@@H]5C4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5ccc6nc(N7C[C@@H]8CCO[C@@H]8C7)ccc6c5)CC4(F)F)ccc3C2=O)C(=O)N1 ; COC[C@@H]1CN(Cc2ccc3nc(N4C[C@@H]5CCO[C@@H]5C4)ncc3c2)C[C@H]1Oc1ccc2c(c1)CN([C@H]1CCC(=O)NC1=O)C2=O]

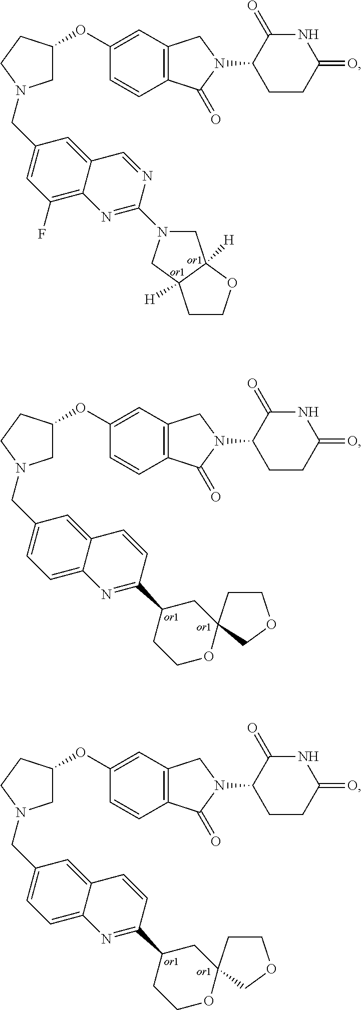 [SMILES: O=C1CC[C@H](N2Cc3cc(O[C@H]4CCN(Cc5cc(F)c6nc(N7C[C@@H]8CCO[C@@H]8C7)ncc6c5)C4)ccc3C2=O)C(=O)N1 ; O=C1CC[C@H](N2Cc3cc(O[C@H]4CCN(Cc5ccc6nc([C@@H]7CCO[C@@]8(CCOC8)C7)ccc6c5)C4)ccc3C2=O)C(=O)N1 ; O=C1CC[C@H](N2Cc3cc(O[C@H]4CCN(Cc5ccc6nc([C@@H]7CCO[C@]8(CCOC8)C7)ccc6c5)C4)ccc3C2=O)C(=O)N1]

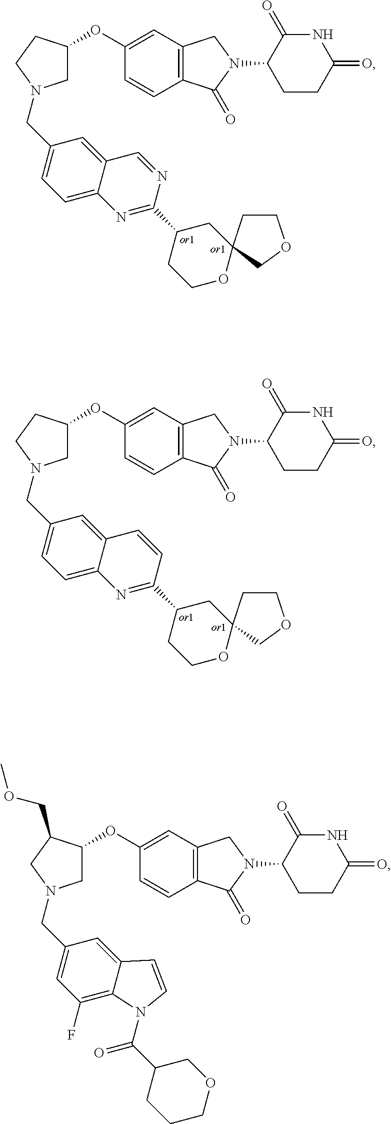 [SMILES: O=C1CC[C@H](N2Cc3cc(O[C@H]4CCN(Cc5ccc6nc([C@H]7CCO[C@@]8(CCOC8)C7)ncc6c5)C4)ccc3C2=O)C(=O)N1 ; O=C1CC[C@H](N2Cc3cc(O[C@H]4CCN(Cc5ccc6nc([C@H]7CCO[C@]8(CCOC8)C7)ccc6c5)C4)ccc3C2=O)C(=O)N1 ; COC[C@@H]1CN(Cc2cc(F)c3c(ccn3C(=O)C3CCCOC3)c2)C[C@H]1Oc1ccc2c(c1)CN([C@H]1CCC(=O)NC1=O)C2=O]

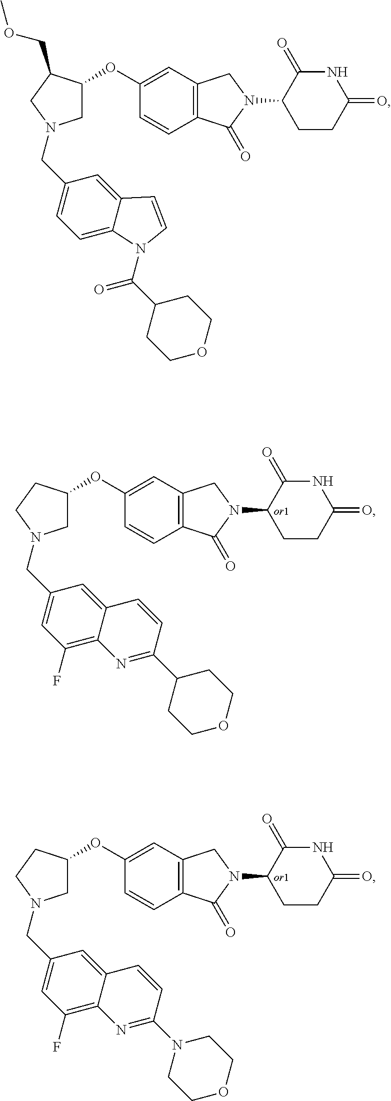 [SMILES: COC[C@@H]1CN(Cc2ccc3c(ccn3C(=O)C3CCOCC3)c2)C[C@H]1Oc1ccc2c(c1)CN([C@H]1CCC(=O)NC1=O)C2=O ; O=C1CC[C@@H](N2Cc3cc(O[C@H]4CCN(Cc5cc(F)c6nc(C7CCOCC7)ccc6c5)C4)ccc3C2=O)C(=O)N1 ; O=C1CC[C@@H](N2Cc3cc(O[C@H]4CCN(Cc5cc(F)c6nc(N7CCOCC7)ccc6c5)C4)ccc3C2=O)C(=O)N1]

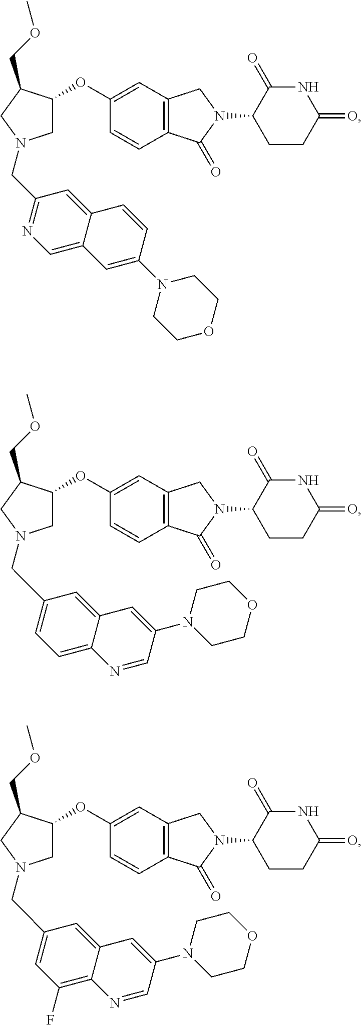 [SMILES: COC[C@@H]1CN(Cc2cc3ccc(N4CCOCC4)cc3cn2)C[C@H]1Oc1ccc2c(c1)CN([C@H]1CCC(=O)NC1=O)C2=O ; COC[C@@H]1CN(Cc2ccc3ncc(N4CCOCC4)cc3c2)C[C@H]1Oc1ccc2c(c1)CN([C@H]1CCC(=O)NC1=O)C2=O ; COC[C@@H]1CN(Cc2cc(F)c3ncc(N4CCOCC4)cc3c2)C[C@H]1Oc1ccc2c(c1)CN([C@H]1CCC(=O)NC1=O)C2=O]

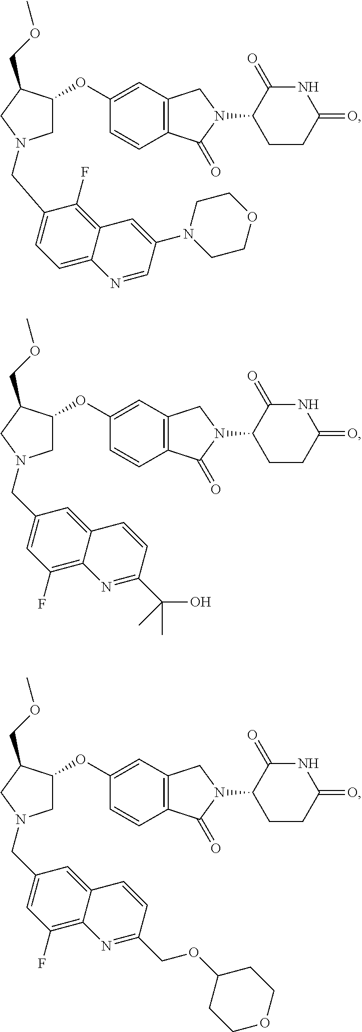 [SMILES: COC[C@@H]1CN(Cc2ccc3ncc(N4CCOCC4)cc3c2F)C[C@H]1Oc1ccc2c(c1)CN([C@H]1CCC(=O)NC1=O)C2=O ; COC[C@@H]1CN(Cc2cc(F)c3nc(C(C)(C)O)ccc3c2)C[C@H]1Oc1ccc2c(c1)CN([C@H]1CCC(=O)NC1=O)C2=O ; COC[C@@H]1CN(Cc2cc(F)c3nc(COC4CCOCC4)ccc3c2)C[C@H]1Oc1ccc2c(c1)CN([C@H]1CCC(=O)NC1=O)C2=O]

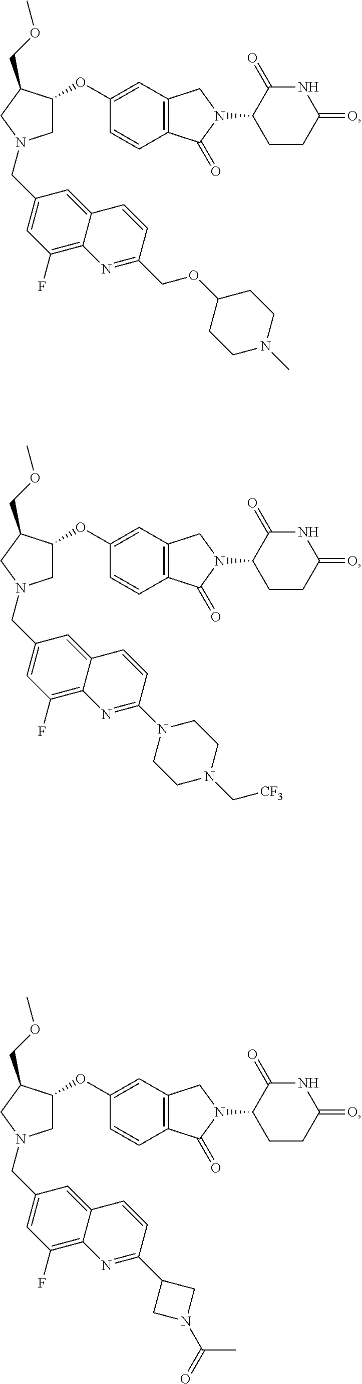 [SMILES: COC[C@@H]1CN(Cc2cc(F)c3nc(COC4CCN(C)CC4)ccc3c2)C[C@H]1Oc1ccc2c(c1)CN([C@H]1CCC(=O)NC1=O)C2=O ; COC[C@@H]1CN(Cc2cc(F)c3nc(N4CCN(CC(F)(F)F)CC4)ccc3c2)C[C@H]1Oc1ccc2c(c1)CN([C@H]1CCC(=O)NC1=O)C2=O ; COC[C@@H]1CN(Cc2cc(F)c3nc(C4CN(C(C)=O)C4)ccc3c2)C[C@H]1Oc1ccc2c(c1)CN([C@H]1CCC(=O)NC1=O)C2=O]

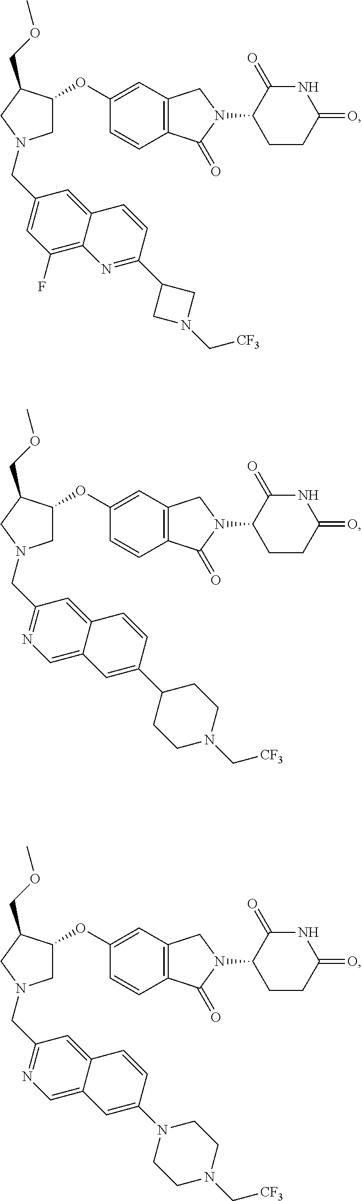 [SMILES: COC[C@@H]1CN(Cc2cc(F)c3nc(C4CN(CC(F)(F)F)C4)ccc3c2)C[C@H]1Oc1ccc2c(c1)CN([C@H]1CCC(=O)NC1=O)C2=O ; COC[C@@H]1CN(Cc2cc3ccc(C4CCN(CC(F)(F)F)CC4)cc3cn2)C[C@H]1Oc1ccc2c(c1)CN([C@H]1CCC(=O)NC1=O)C2=O ; COC[C@@H]1CN(Cc2cc3ccc(N4CCN(CC(F)(F)F)CC4)cc3cn2)C[C@H]1Oc1ccc2c(c1)CN([C@H]1CCC(=O)NC1=O)C2=O]

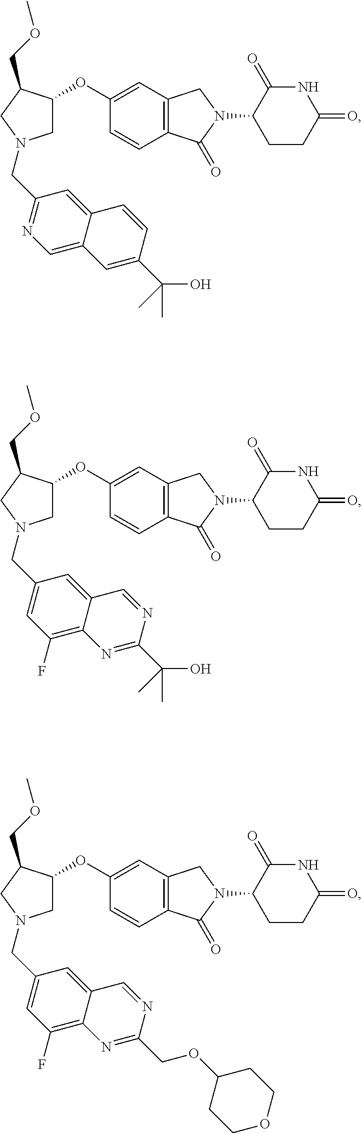 [SMILES: COC[C@@H]1CN(Cc2cc3ccc(C(C)(C)O)cc3cn2)C[C@H]1Oc1ccc2c(c1)CN([C@H]1CCC(=O)NC1=O)C2=O ; COC[C@@H]1CN(Cc2cc(F)c3nc(C(C)(C)O)ncc3c2)C[C@H]1Oc1ccc2c(c1)CN([C@H]1CCC(=O)NC1=O)C2=O ; COC[C@@H]1CN(Cc2cc(F)c3nc(COC4CCOCC4)ncc3c2)C[C@H]1Oc1ccc2c(c1)CN([C@H]1CCC(=O)NC1=O)C2=O]

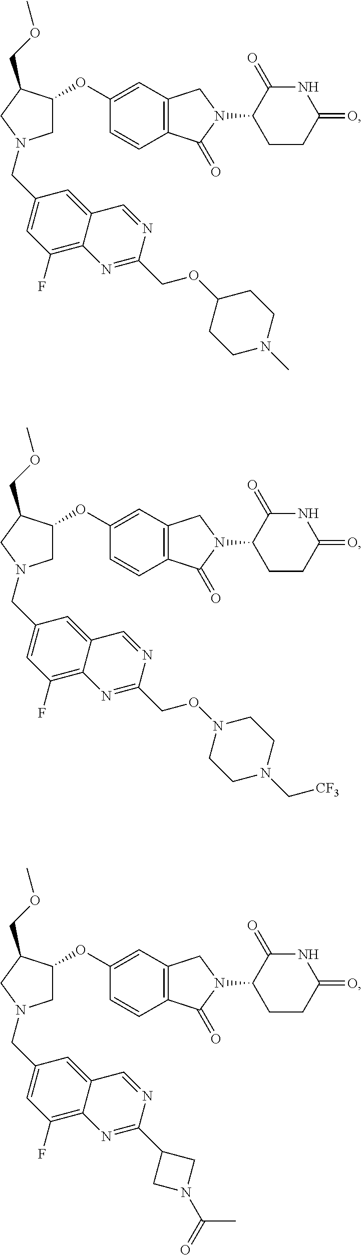 [SMILES: COC[C@@H]1CN(Cc2cc(F)c3nc(COC4CCN(C)CC4)ncc3c2)C[C@H]1Oc1ccc2c(c1)CN([C@H]1CCC(=O)NC1=O)C2=O ; COC[C@@H]1CN(Cc2cc(F)c3nc(CON4CCN(CC(F)(F)F)CC4)ncc3c2)C[C@H]1Oc1ccc2c(c1)CN([C@H]1CCC(=O)NC1=O)C2=O ; COC[C@@H]1CN(Cc2cc(F)c3nc(C4CN(C(C)=O)C4)ncc3c2)C[C@H]1Oc1ccc2c(c1)CN([C@H]1CCC(=O)NC1=O)C2=O]

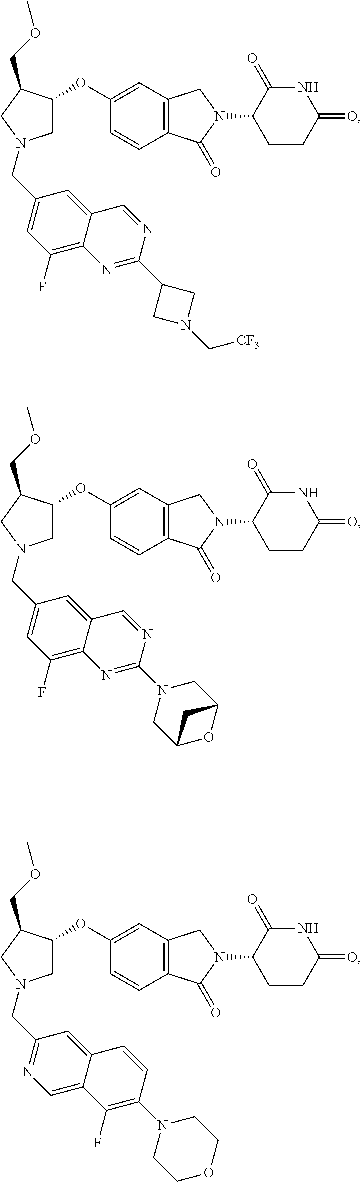 [SMILES: COC[C@@H]1CN(Cc2cc(F)c3nc(C4CN(CC(F)(F)F)C4)ncc3c2)C[C@H]1Oc1ccc2c(c1)CN([C@H]1CCC(=O)NC1=O)C2=O ; COC[C@@H]1CN(Cc2cc(F)c3nc(N4C[C@H]5C[C@@H](C4)O5)ncc3c2)C[C@H]1Oc1ccc2c(c1)CN([C@H]1CCC(=O)NC1=O)C2=O ; COC[C@@H]1CN(Cc2cc3ccc(N4CCOCC4)c(F)c3cn2)C[C@H]1Oc1ccc2c(c1)CN([C@H]1CCC(=O)NC1=O)C2=O]

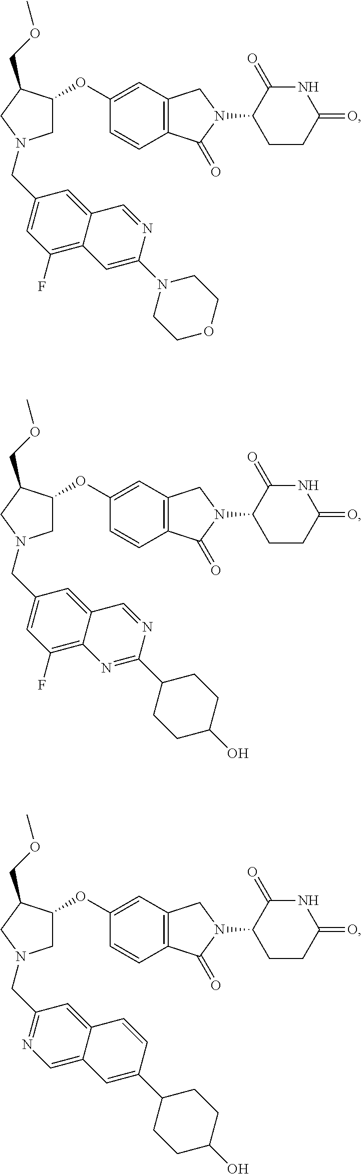 [SMILES: COC[C@@H]1CN(Cc2cc(F)c3cc(N4CCOCC4)ncc3c2)C[C@H]1Oc1ccc2c(c1)CN([C@H]1CCC(=O)NC1=O)C2=O ; COC[C@@H]1CN(Cc2cc(F)c3nc(C4CCC(O)CC4)ncc3c2)C[C@H]1Oc1ccc2c(c1)CN([C@H]1CCC(=O)NC1=O)C2=O ; COC[C@@H]1CN(Cc2cc3ccc(C4CCC(O)CC4)cc3cn2)C[C@H]1Oc1ccc2c(c1)CN([C@H]1CCC(=O)NC1=O)C2=O]

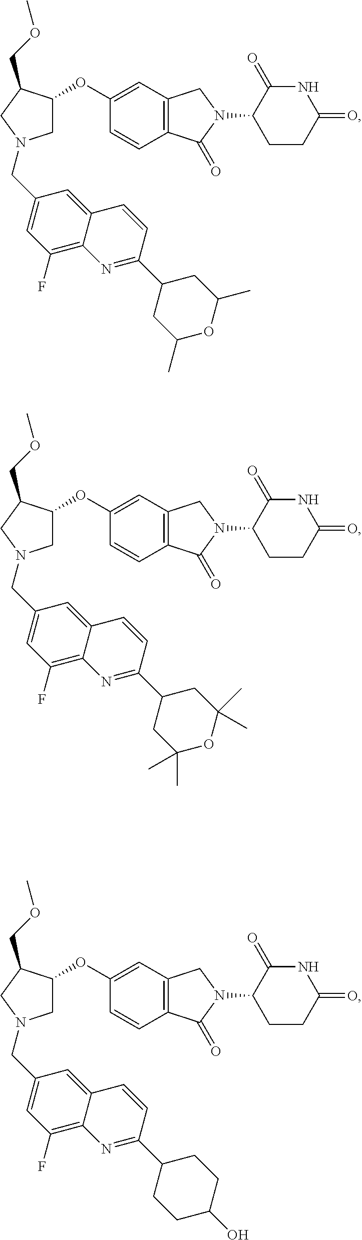 [SMILES: COC[C@@H]1CN(Cc2cc(F)c3nc(C4CC(C)OC(C)C4)ccc3c2)C[C@H]1Oc1ccc2c(c1)CN([C@H]1CCC(=O)NC1=O)C2=O ; COC[C@@H]1CN(Cc2cc(F)c3nc(C4CC(C)(C)OC(C)(C)C4)ccc3c2)C[C@H]1Oc1ccc2c(c1)CN([C@H]1CCC(=O)NC1=O)C2=O ; COC[C@@H]1CN(Cc2cc(F)c3nc(C4CCC(O)CC4)ccc3c2)C[C@H]1Oc1ccc2c(c1)CN([C@H]1CCC(=O)NC1=O)C2=O]

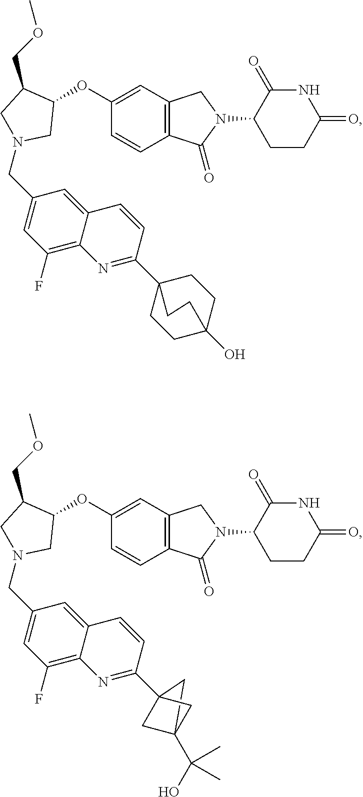 [SMILES: COC[C@@H]1CN(Cc2cc(F)c3nc(C45CCC(O)(CC4)CC5)ccc3c2)C[C@H]1Oc1ccc2c(c1)CN([C@H]1CCC(=O)NC1=O)C2=O ; COC[C@@H]1CN(Cc2cc(F)c3nc(C45CC(C(C)(C)O)(C4)C5)ccc3c2)C[C@H]1Oc1ccc2c(c1)CN([C@H]1CCC(=O)NC1=O)C2=O]

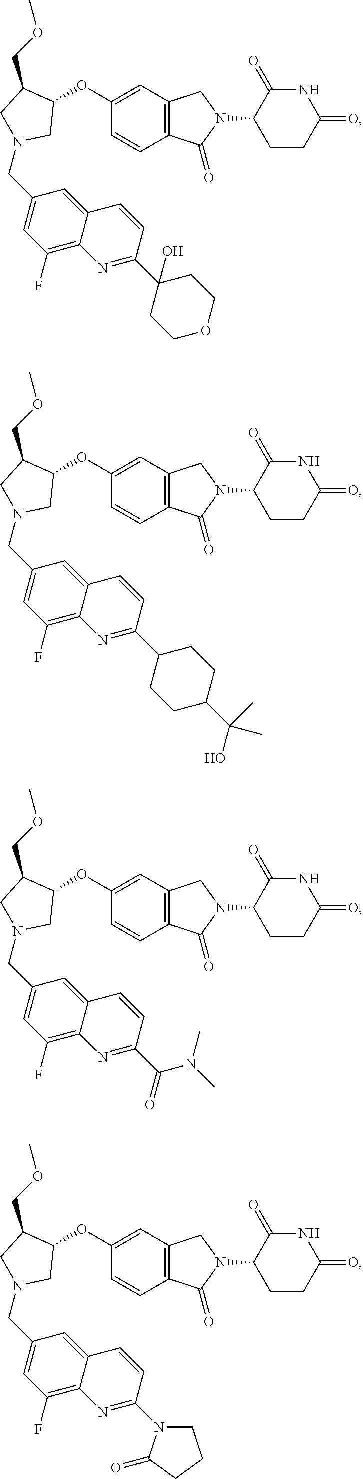 [SMILES: COC[C@@H]1CN(Cc2cc(F)c3nc(C4(O)CCOCC4)ccc3c2)C[C@H]1Oc1ccc2c(c1)CN([C@H]1CCC(=O)NC1=O)C2=O ; COC[C@@H]1CN(Cc2cc(F)c3nc(C4CCC(C(C)(C)O)CC4)ccc3c2)C[C@H]1Oc1ccc2c(c1)CN([C@H]1CCC(=O)NC1=O)C2=O ; COC[C@@H]1CN(Cc2cc(F)c3nc(C(=O)N(C)C)ccc3c2)C[C@H]1Oc1ccc2c(c1)CN([C@H]1CCC(=O)NC1=O)C2=O ; COC[C@@H]1CN(Cc2cc(F)c3nc(N4CCCC4=O)ccc3c2)C[C@H]1Oc1ccc2c(c1)CN([C@H]1CCC(=O)NC1=O)C2=O]

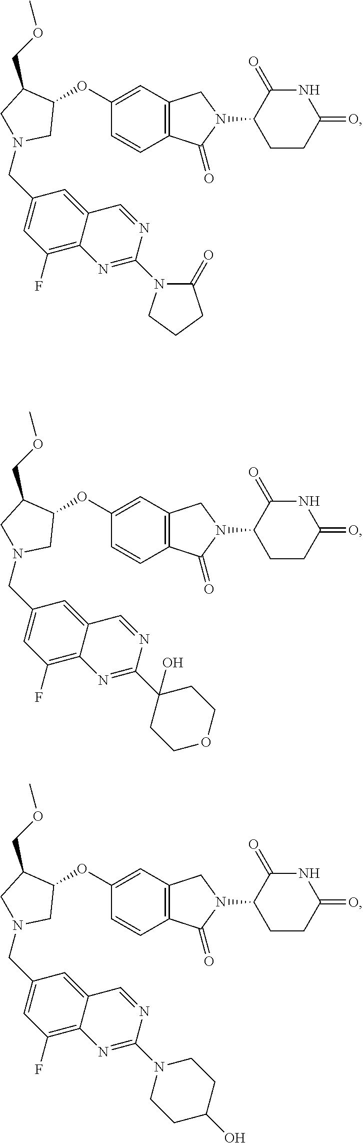 [SMILES: COC[C@@H]1CN(Cc2cc(F)c3nc(N4CCCC4=O)ncc3c2)C[C@H]1Oc1ccc2c(c1)CN([C@H]1CCC(=O)NC1=O)C2=O ; COC[C@@H]1CN(Cc2cc(F)c3nc(C4(O)CCOCC4)ncc3c2)C[C@H]1Oc1ccc2c(c1)CN([C@H]1CCC(=O)NC1=O)C2=O ; COC[C@@H]1CN(Cc2cc(F)c3nc(N4CCC(O)CC4)ncc3c2)C[C@H]1Oc1ccc2c(c1)CN([C@H]1CCC(=O)NC1=O)C2=O]

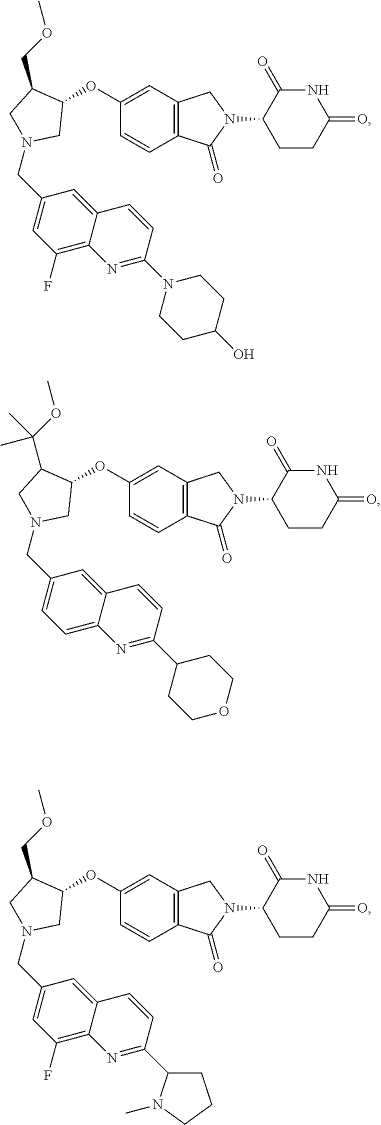 [SMILES: COC[C@@H]1CN(Cc2cc(F)c3nc(N4CCC(O)CC4)ccc3c2)C[C@H]1Oc1ccc2c(c1)CN([C@H]1CCC(=O)NC1=O)C2=O ; COC(C)(C)C1CN(Cc2ccc3nc(C4CCOCC4)ccc3c2)C[C@H]1Oc1ccc2c(c1)CN([C@H]1CCC(=O)NC1=O)C2=O ; COC[C@@H]1CN(Cc2cc(F)c3nc(C4CCCN4C)ccc3c2)C[C@H]1Oc1ccc2c(c1)CN([C@H]1CCC(=O)NC1=O)C2=O]

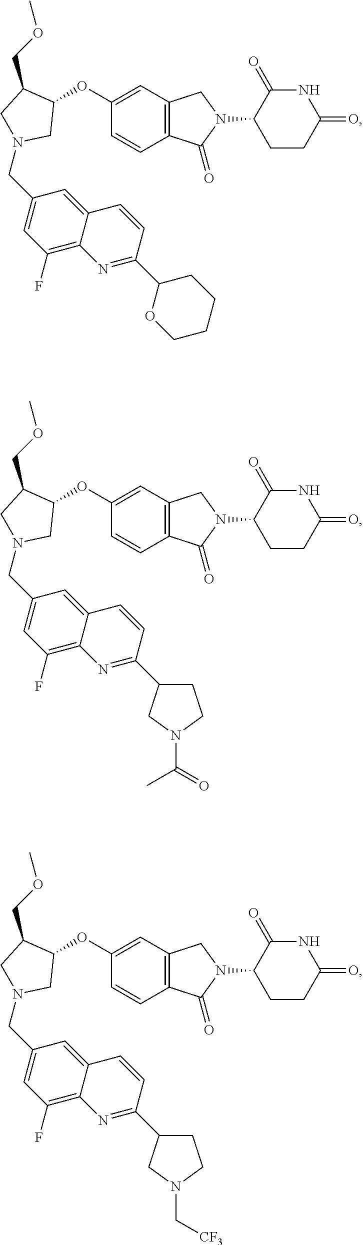 [SMILES: COC[C@@H]1CN(Cc2cc(F)c3nc(C4CCCCO4)ccc3c2)C[C@H]1Oc1ccc2c(c1)CN([C@H]1CCC(=O)NC1=O)C2=O ; COC[C@@H]1CN(Cc2cc(F)c3nc(C4CCN(C(C)=O)C4)ccc3c2)C[C@H]1Oc1ccc2c(c1)CN([C@H]1CCC(=O)NC1=O)C2=O ; COC[C@@H]1CN(Cc2cc(F)c3nc(C4CCN(CC(F)(F)F)C4)ccc3c2)C[C@H]1Oc1ccc2c(c1)CN([C@H]1CCC(=O)NC1=O)C2=O]

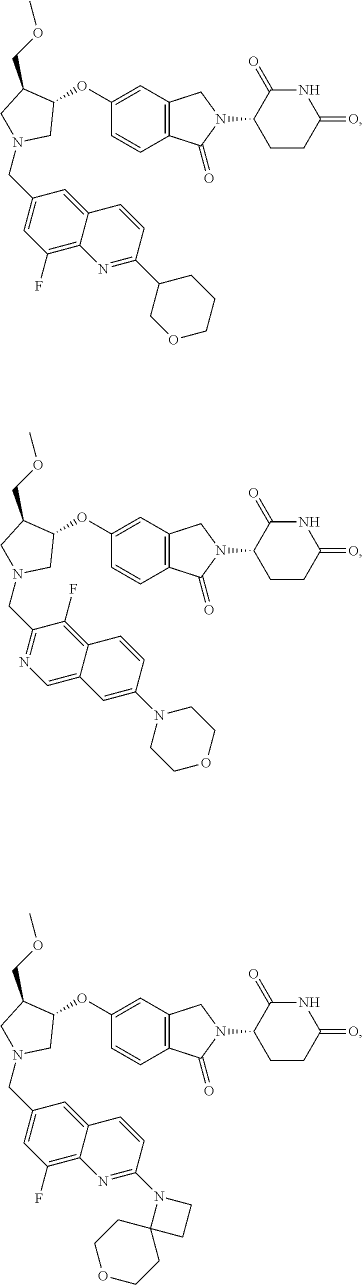 [SMILES: COC[C@@H]1CN(Cc2cc(F)c3nc(C4CCCOC4)ccc3c2)C[C@H]1Oc1ccc2c(c1)CN([C@H]1CCC(=O)NC1=O)C2=O ; COC[C@@H]1CN(Cc2ncc3cc(N4CCOCC4)ccc3c2F)C[C@H]1Oc1ccc2c(c1)CN([C@H]1CCC(=O)NC1=O)C2=O ; COC[C@@H]1CN(Cc2cc(F)c3nc(N4CCC45CCOCC5)ccc3c2)C[C@H]1Oc1ccc2c(c1)CN([C@H]1CCC(=O)NC1=O)C2=O]

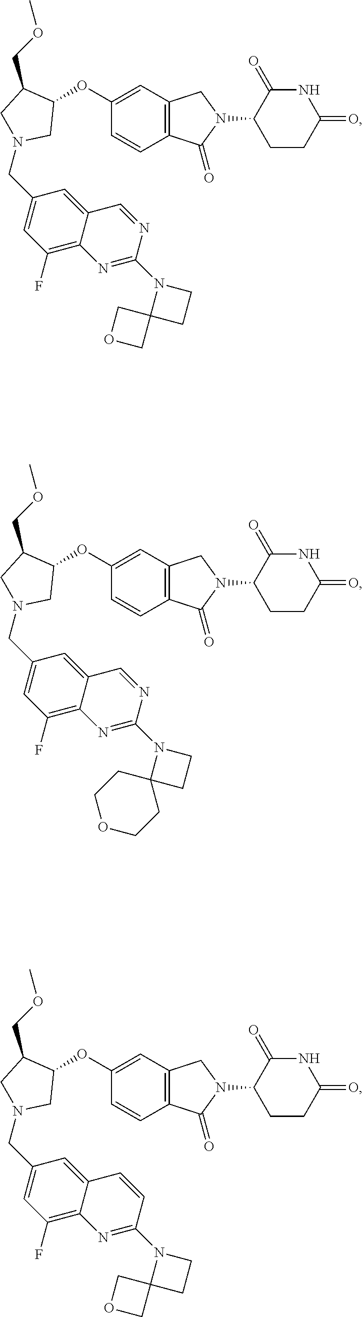 [SMILES: COC[C@@H]1CN(Cc2cc(F)c3nc(N4CCC45COC5)ncc3c2)C[C@H]1Oc1ccc2c(c1)CN([C@H]1CCC(=O)NC1=O)C2=O ; COC[C@@H]1CN(Cc2cc(F)c3nc(N4CCC45CCOCC5)ncc3c2)C[C@H]1Oc1ccc2c(c1)CN([C@H]1CCC(=O)NC1=O)C2=O ; COC[C@@H]1CN(Cc2cc(F)c3nc(N4CCC45COC5)ccc3c2)C[C@H]1Oc1ccc2c(c1)CN([C@H]1CCC(=O)NC1=O)C2=O]

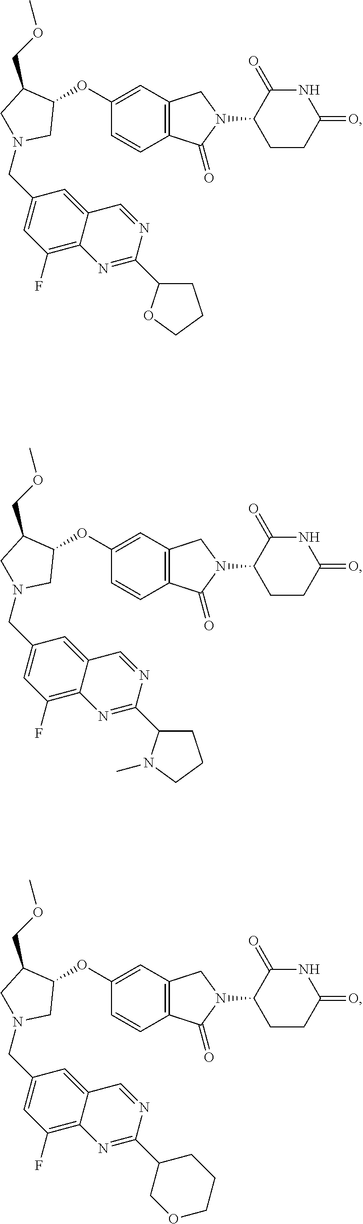 [SMILES: COC[C@@H]1CN(Cc2cc(F)c3nc(C4CCCO4)ncc3c2)C[C@H]1Oc1ccc2c(c1)CN([C@H]1CCC(=O)NC1=O)C2=O ; COC[C@@H]1CN(Cc2cc(F)c3nc(C4CCCN4C)ncc3c2)C[C@H]1Oc1ccc2c(c1)CN([C@H]1CCC(=O)NC1=O)C2=O ; COC[C@@H]1CN(Cc2cc(F)c3nc(C4CCCOC4)ncc3c2)C[C@H]1Oc1ccc2c(c1)CN([C@H]1CCC(=O)NC1=O)C2=O]

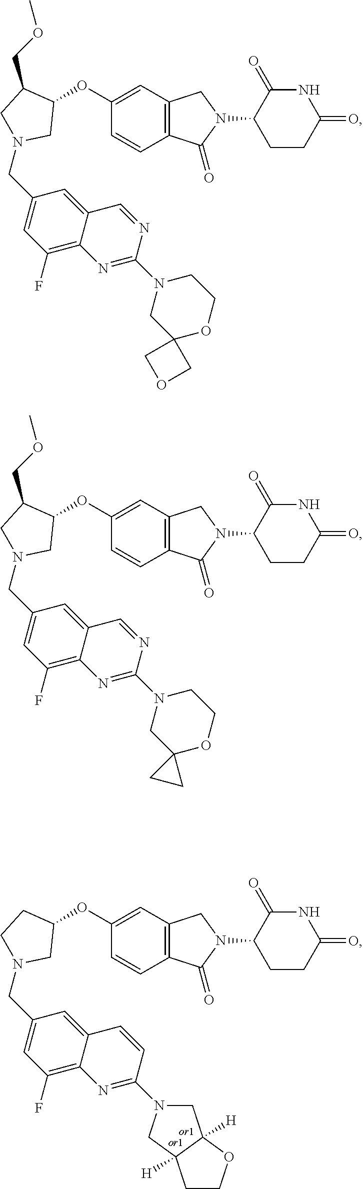 [SMILES: COC[C@@H]1CN(Cc2cc(F)c3nc(N4CCOC5(COC5)C4)ncc3c2)C[C@H]1Oc1ccc2c(c1)CN([C@H]1CCC(=O)NC1=O)C2=O ; COC[C@@H]1CN(Cc2cc(F)c3nc(N4CCOC5(CC5)C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(N7C[C@@H]8CCO[C@@H]8C7)ccc6c5)C4)ccc3C2=O)C(=O)N1]

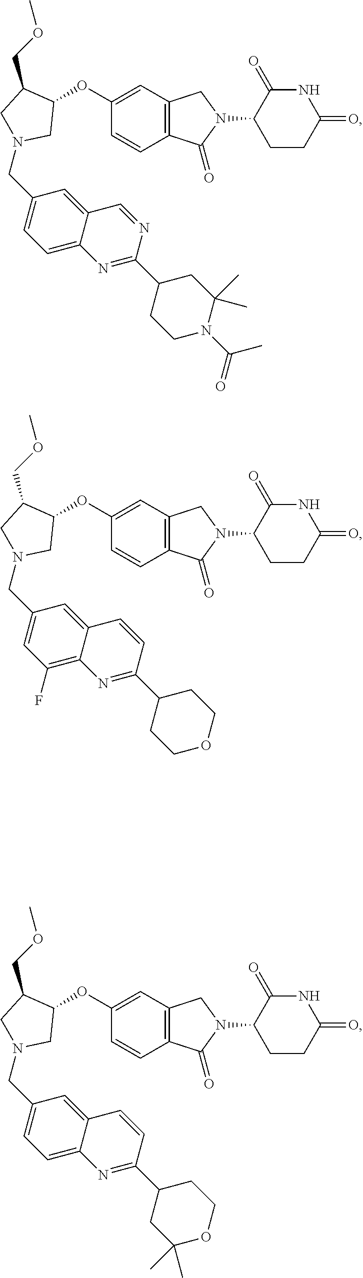 [SMILES: COC[C@@H]1CN(Cc2ccc3nc(C4CCN(C(C)=O)C(C)(C)C4)ncc3c2)C[C@H]1Oc1ccc2c(c1)CN([C@H]1CCC(=O)NC1=O)C2=O ; COC[C@H]1CN(Cc2cc(F)c3nc(C4CCOCC4)ccc3c2)C[C@H]1Oc1ccc2c(c1)CN([C@H]1CCC(=O)NC1=O)C2=O ; COC[C@@H]1CN(Cc2ccc3nc(C4CCOC(C)(C)C4)ccc3c2)C[C@H]1Oc1ccc2c(c1)CN([C@H]1CCC(=O)NC1=O)C2=O]

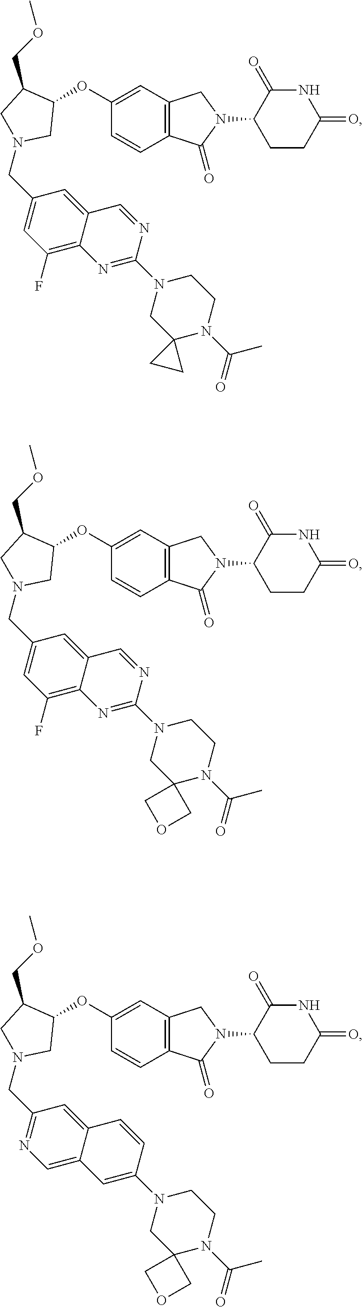 [SMILES: COC[C@@H]1CN(Cc2cc(F)c3nc(N4CCN(C(C)=O)C5(CC5)C4)ncc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(COC5)C4)ncc3c2)C[C@H]1Oc1ccc2c(c1)CN([C@H]1CCC(=O)NC1=O)C2=O ; COC[C@@H]1CN(Cc2cc3ccc(N4CCN(C(C)=O)C5(COC5)C4)cc3cn2)C[C@H]1Oc1ccc2c(c1)CN([C@H]1CCC(=O)NC1=O)C2=O]

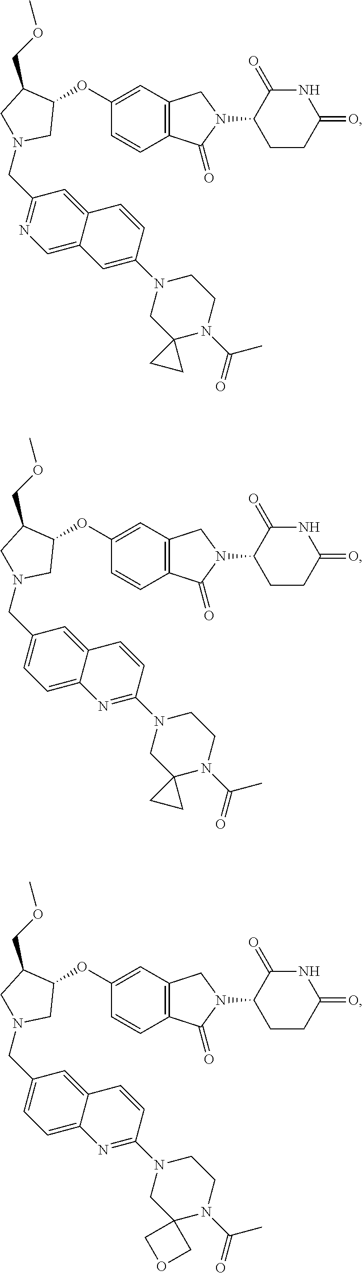 [SMILES: COC[C@@H]1CN(Cc2cc3ccc(N4CCN(C(C)=O)C5(CC5)C4)cc3cn2)C[C@H]1Oc1ccc2c(c1)CN([C@H]1CCC(=O)NC1=O)C2=O ; COC[C@@H]1CN(Cc2ccc3nc(N4CCN(C(C)=O)C5(CC5)C4)ccc3c2)C[C@H]1Oc1ccc2c(c1)CN([C@H]1CCC(=O)NC1=O)C2=O ; COC[C@@H]1CN(Cc2ccc3nc(N4CCN(C(C)=O)C5(COC5)C4)ccc3c2)C[C@H]1Oc1ccc2c(c1)CN([C@H]1CCC(=O)NC1=O)C2=O]

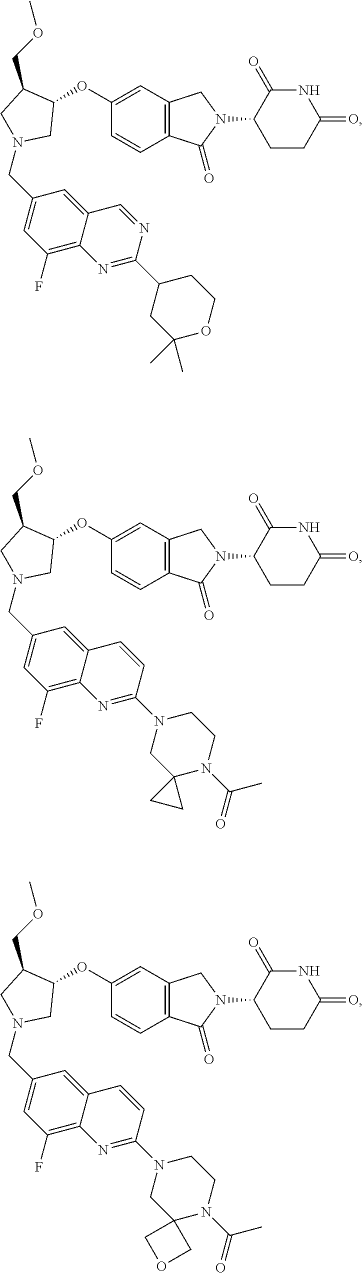 [SMILES: COC[C@@H]1CN(Cc2cc(F)c3nc(C4CCOC(C)(C)C4)ncc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(CC5)C4)ccc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(COC5)C4)ccc3c2)C[C@H]1Oc1ccc2c(c1)CN([C@H]1CCC(=O)NC1=O)C2=O]

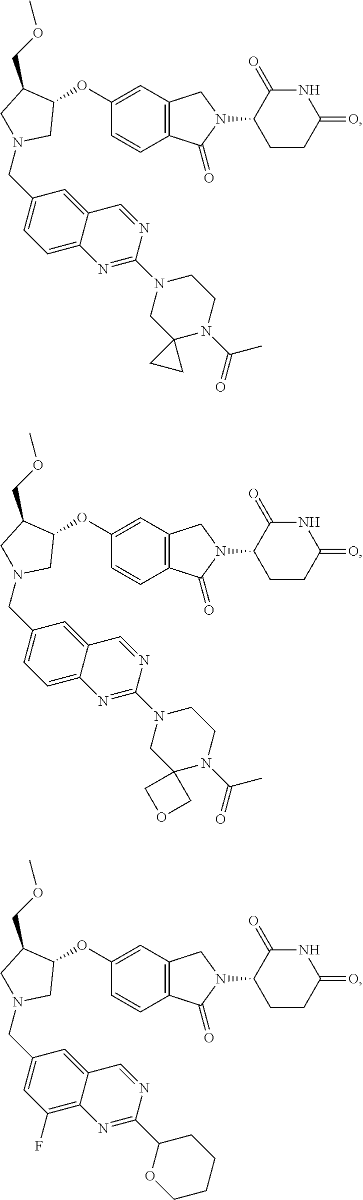 [SMILES: COC[C@@H]1CN(Cc2ccc3nc(N4CCN(C(C)=O)C5(CC5)C4)ncc3c2)C[C@H]1Oc1ccc2c(c1)CN([C@H]1CCC(=O)NC1=O)C2=O ; COC[C@@H]1CN(Cc2ccc3nc(N4CCN(C(C)=O)C5(COC5)C4)ncc3c2)C[C@H]1Oc1ccc2c(c1)CN([C@H]1CCC(=O)NC1=O)C2=O ; COC[C@@H]1CN(Cc2cc(F)c3nc(C4CCCCO4)ncc3c2)C[C@H]1Oc1ccc2c(c1)CN([C@H]1CCC(=O)NC1=O)C2=O]

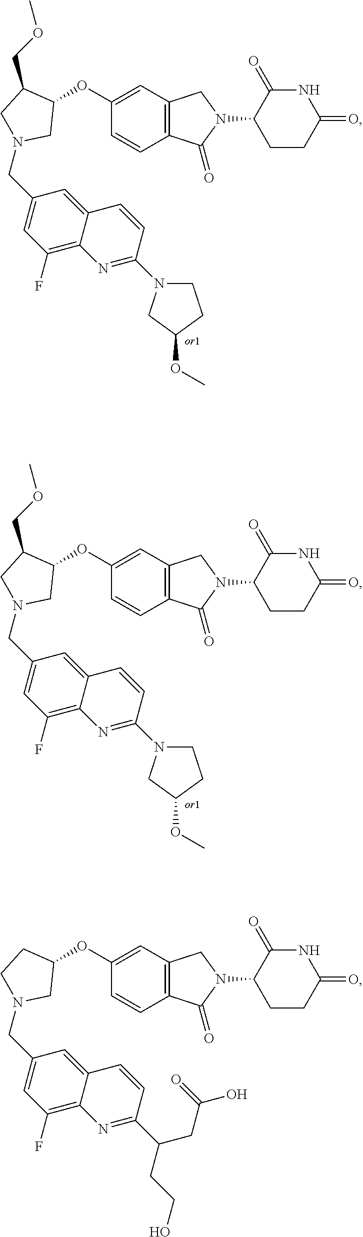 [SMILES: COC[C@@H]1CN(Cc2cc(F)c3nc(N4CC[C@@H](OC)C4)ccc3c2)C[C@H]1Oc1ccc2c(c1)CN([C@H]1CCC(=O)NC1=O)C2=O ; COC[C@@H]1CN(Cc2cc(F)c3nc(N4CC[C@H](OC)C4)ccc3c2)C[C@H]1Oc1ccc2c(c1)CN([C@H]1CCC(=O)NC1=O)C2=O ; O=C(O)CC(CCO)c1ccc2cc(CN3CC[C@H](Oc4ccc5c(c4)CN([C@H]4CCC(=O)NC4=O)C5=O)C3)cc(F)c2n1]

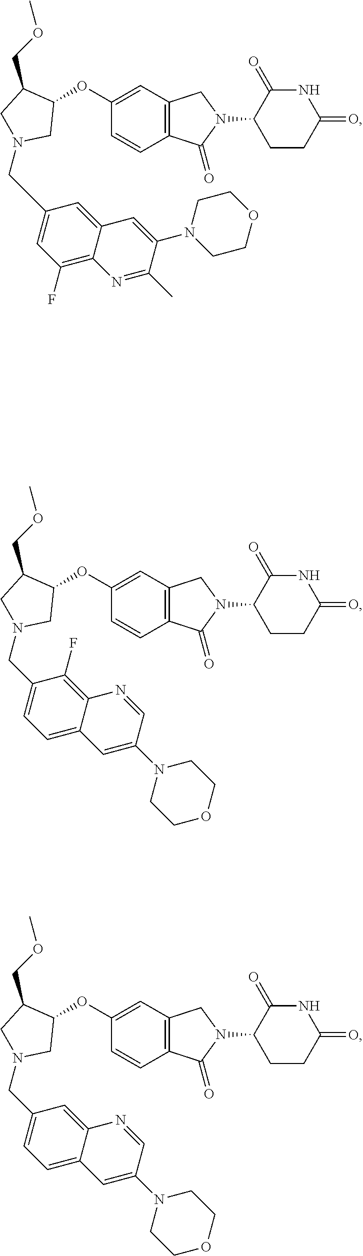 [SMILES: COC[C@@H]1CN(Cc2cc(F)c3nc(C)c(N4CCOCC4)cc3c2)C[C@H]1Oc1ccc2c(c1)CN([C@H]1CCC(=O)NC1=O)C2=O ; COC[C@@H]1CN(Cc2ccc3cc(N4CCOCC4)cnc3c2F)C[C@H]1Oc1ccc2c(c1)CN([C@H]1CCC(=O)NC1=O)C2=O ; COC[C@@H]1CN(Cc2ccc3cc(N4CCOCC4)cnc3c2)C[C@H]1Oc1ccc2c(c1)CN([C@H]1CCC(=O)NC1=O)C2=O]

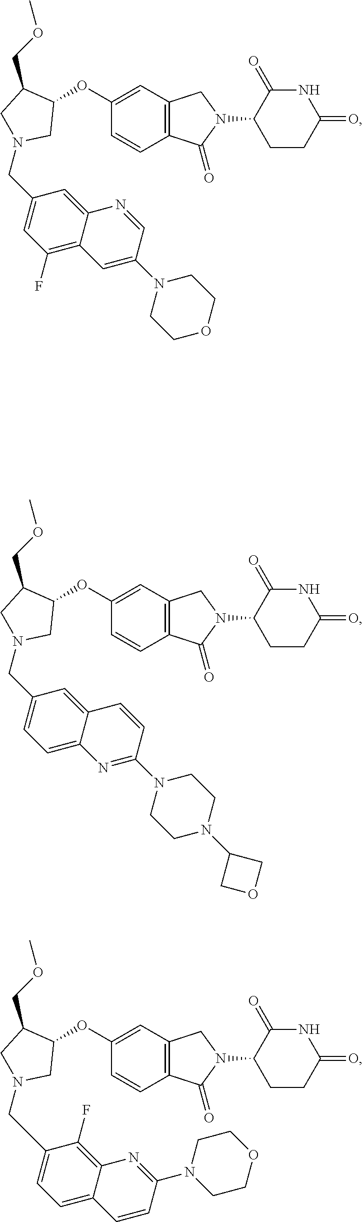 [SMILES: COC[C@@H]1CN(Cc2cc(F)c3cc(N4CCOCC4)cnc3c2)C[C@H]1Oc1ccc2c(c1)CN([C@H]1CCC(=O)NC1=O)C2=O ; COC[C@@H]1CN(Cc2ccc3nc(N4CCN(C5COC5)CC4)ccc3c2)C[C@H]1Oc1ccc2c(c1)CN([C@H]1CCC(=O)NC1=O)C2=O ; COC[C@@H]1CN(Cc2ccc3ccc(N4CCOCC4)nc3c2F)C[C@H]1Oc1ccc2c(c1)CN([C@H]1CCC(=O)NC1=O)C2=O]

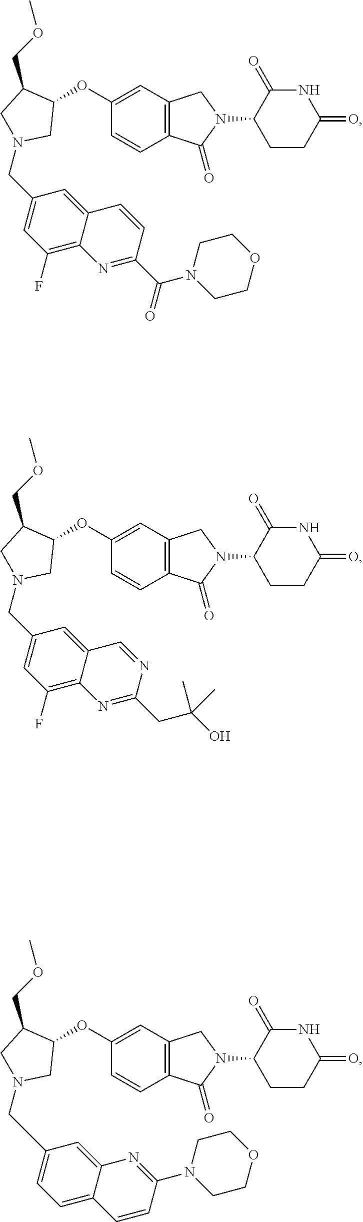 [SMILES: COC[C@@H]1CN(Cc2cc(F)c3nc(C(=O)N4CCOCC4)ccc3c2)C[C@H]1Oc1ccc2c(c1)CN([C@H]1CCC(=O)NC1=O)C2=O ; COC[C@@H]1CN(Cc2cc(F)c3nc(CC(C)(C)O)ncc3c2)C[C@H]1Oc1ccc2c(c1)CN([C@H]1CCC(=O)NC1=O)C2=O ; COC[C@@H]1CN(Cc2ccc3ccc(N4CCOCC4)nc3c2)C[C@H]1Oc1ccc2c(c1)CN([C@H]1CCC(=O)NC1=O)C2=O]

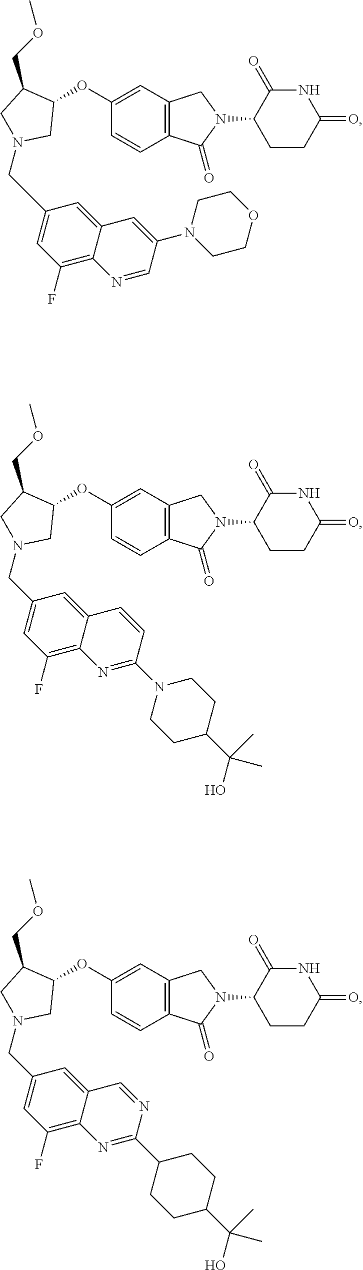 [SMILES: COC[C@@H]1CN(Cc2cc(F)c3ncc(N4CCOCC4)cc3c2)C[C@H]1Oc1ccc2c(c1)CN([C@H]1CCC(=O)NC1=O)C2=O ; COC[C@@H]1CN(Cc2cc(F)c3nc(N4CCC(C(C)(C)O)CC4)ccc3c2)C[C@H]1Oc1ccc2c(c1)CN([C@H]1CCC(=O)NC1=O)C2=O ; COC[C@@H]1CN(Cc2cc(F)c3nc(C4CCC(C(C)(C)O)CC4)ncc3c2)C[C@H]1Oc1ccc2c(c1)CN([C@H]1CCC(=O)NC1=O)C2=O]

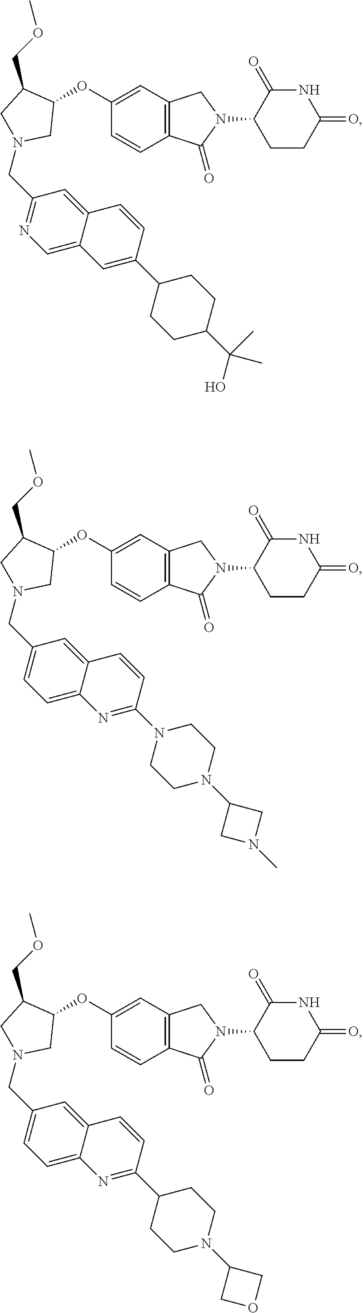 [SMILES: COC[C@@H]1CN(Cc2cc3ccc(C4CCC(C(C)(C)O)CC4)cc3cn2)C[C@H]1Oc1ccc2c(c1)CN([C@H]1CCC(=O)NC1=O)C2=O ; COC[C@@H]1CN(Cc2ccc3nc(N4CCN(C5CN(C)C5)CC4)ccc3c2)C[C@H]1Oc1ccc2c(c1)CN([C@H]1CCC(=O)NC1=O)C2=O ; COC[C@@H]1CN(Cc2ccc3nc(C4CCN(C5COC5)CC4)ccc3c2)C[C@H]1Oc1ccc2c(c1)CN([C@H]1CCC(=O)NC1=O)C2=O]

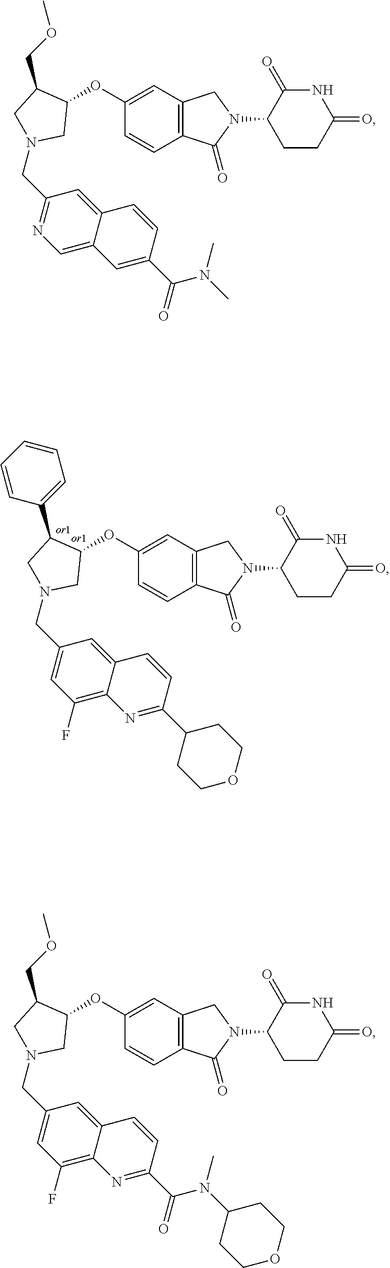 [SMILES: COC[C@@H]1CN(Cc2cc3ccc(C(=O)N(C)C)cc3cn2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4c4ccccc4)ccc3C2=O)C(=O)N1 ; COC[C@@H]1CN(Cc2cc(F)c3nc(C(=O)N(C)C4CCOCC4)ccc3c2)C[C@H]1Oc1ccc2c(c1)CN([C@H]1CCC(=O)NC1=O)C2=O]

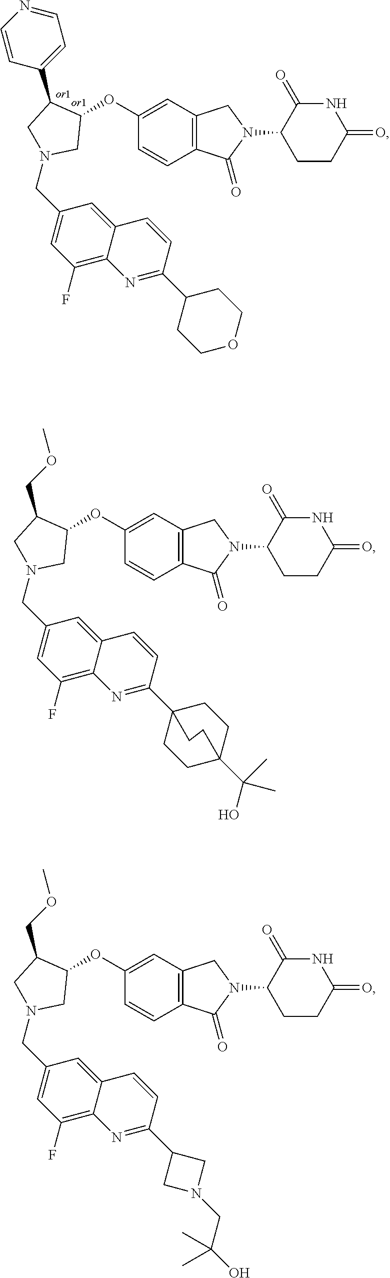 [SMILES: O=C1CC[C@H](N2Cc3cc(O[C@@H]4CN(Cc5cc(F)c6nc(C7CCOCC7)ccc6c5)C[C@H]4c4ccncc4)ccc3C2=O)C(=O)N1 ; COC[C@@H]1CN(Cc2cc(F)c3nc(C45CCC(C(C)(C)O)(CC4)CC5)ccc3c2)C[C@H]1Oc1ccc2c(c1)CN([C@H]1CCC(=O)NC1=O)C2=O ; COC[C@@H]1CN(Cc2cc(F)c3nc(C4CN(CC(C)(C)O)C4)ccc3c2)C[C@H]1Oc1ccc2c(c1)CN([C@H]1CCC(=O)NC1=O)C2=O]

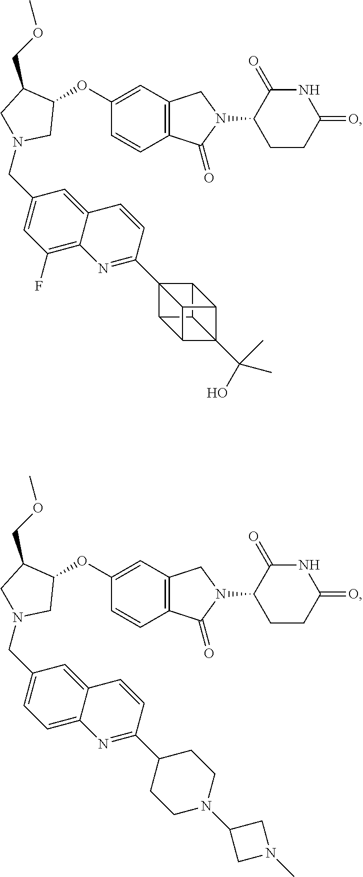 [SMILES: COC[C@@H]1CN(Cc2cc(F)c3nc(C45C6C7C4C4C5C6C74C(C)(C)O)ccc3c2)C[C@H]1Oc1ccc2c(c1)CN([C@H]1CCC(=O)NC1=O)C2=O ; COC[C@@H]1CN(Cc2ccc3nc(C4CCN(C5CN(C)C5)CC4)ccc3c2)C[C@H]1Oc1ccc2c(c1)CN([C@H]1CCC(=O)NC1=O)C2=O]

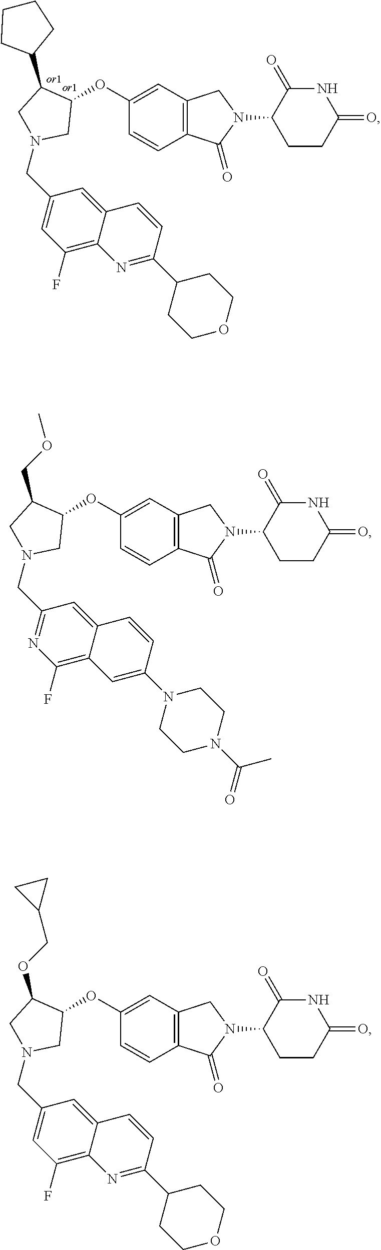 [SMILES: O=C1CC[C@H](N2Cc3cc(O[C@@H]4CN(Cc5cc(F)c6nc(C7CCOCC7)ccc6c5)C[C@H]4C4CCCC4)ccc3C2=O)C(=O)N1 ; COC[C@@H]1CN(Cc2cc3ccc(N4CCN(C(C)=O)CC4)cc3c(F)n2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4OCC4CC4)ccc3C2=O)C(=O)N1]

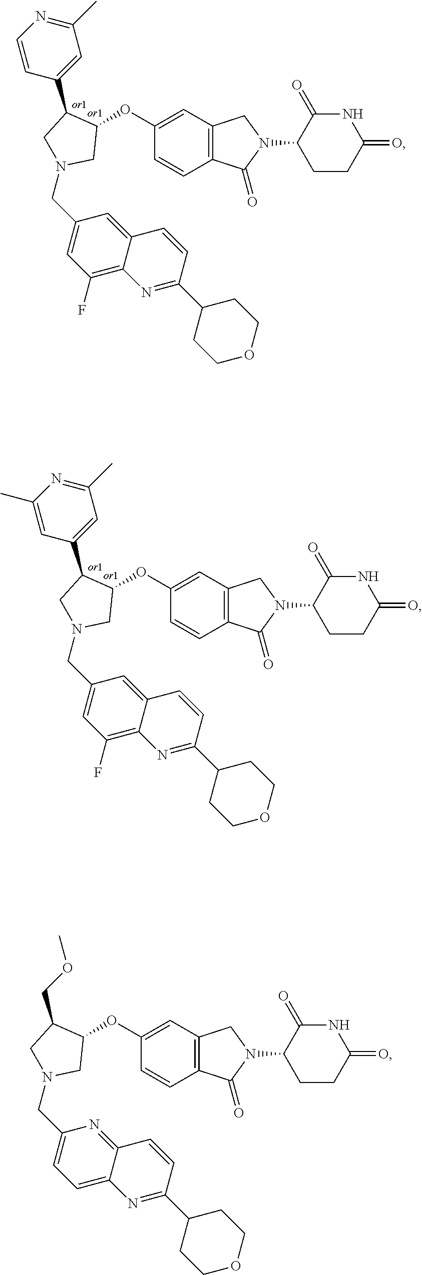 [SMILES: Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)ccn1 ; Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)cc(C)n1 ; COC[C@@H]1CN(Cc2ccc3nc(C4CCOCC4)ccc3n2)C[C@H]1Oc1ccc2c(c1)CN([C@H]1CCC(=O)NC1=O)C2=O]

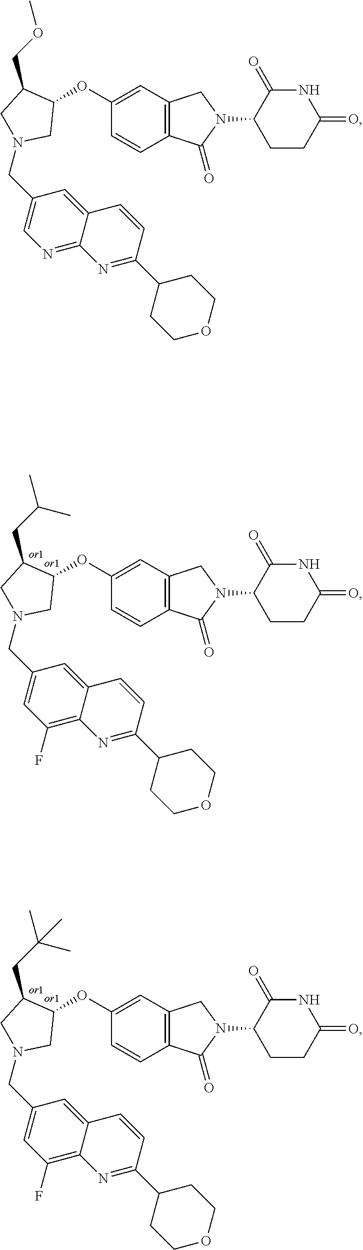 [SMILES: COC[C@@H]1CN(Cc2cnc3nc(C4CCOCC4)ccc3c2)C[C@H]1Oc1ccc2c(c1)CN([C@H]1CCC(=O)NC1=O)C2=O ; CC(C)C[C@@H]1CN(Cc2cc(F)c3nc(C4CCOCC4)ccc3c2)C[C@H]1Oc1ccc2c(c1)CN([C@H]1CCC(=O)NC1=O)C2=O ; CC(C)(C)C[C@@H]1CN(Cc2cc(F)c3nc(C4CCOCC4)ccc3c2)C[C@H]1Oc1ccc2c(c1)CN([C@H]1CCC(=O)NC1=O)C2=O]

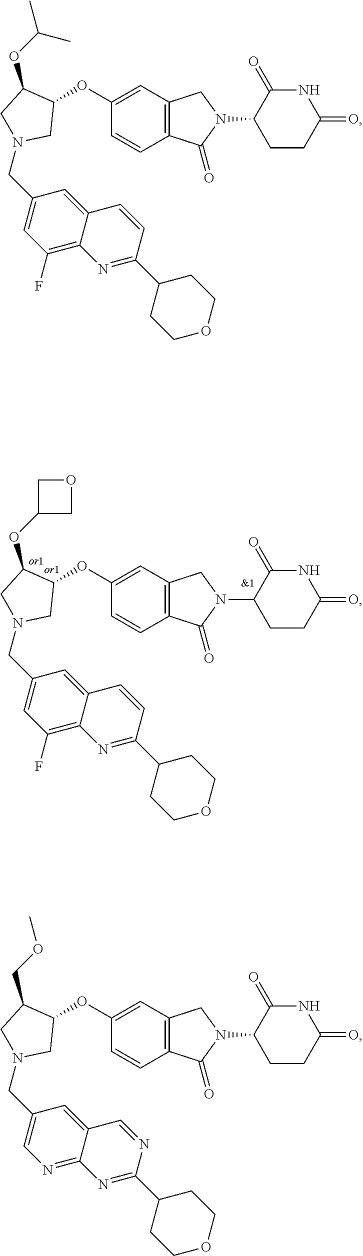 [SMILES: CC(C)O[C@@H]1CN(Cc2cc(F)c3nc(C4CCOCC4)ccc3c2)C[C@H]1Oc1ccc2c(c1)CN([C@H]1CCC(=O)NC1=O)C2=O ; O=C1CCC(N2Cc3cc(O[C@@H]4CN(Cc5cc(F)c6nc(C7CCOCC7)ccc6c5)C[C@H]4OC4COC4)ccc3C2=O)C(=O)N1 ; COC[C@@H]1CN(Cc2cnc3nc(C4CCOCC4)ncc3c2)C[C@H]1Oc1ccc2c(c1)CN([C@H]1CCC(=O)NC1=O)C2=O]

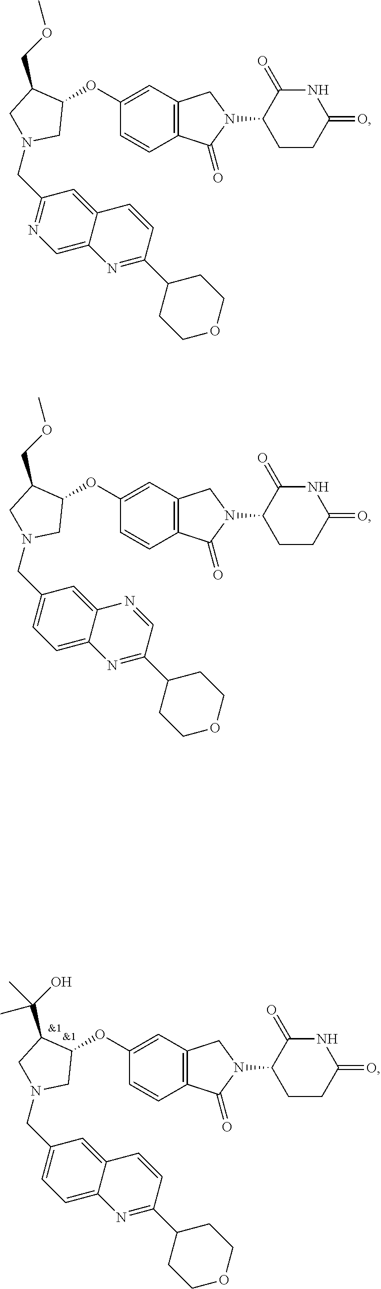 [SMILES: COC[C@@H]1CN(Cc2cc3ccc(C4CCOCC4)nc3cn2)C[C@H]1Oc1ccc2c(c1)CN([C@H]1CCC(=O)NC1=O)C2=O ; COC[C@@H]1CN(Cc2ccc3nc(C4CCOCC4)cnc3c2)C[C@H]1Oc1ccc2c(c1)CN([C@H]1CCC(=O)NC1=O)C2=O ; CC(C)(O)[C@@H]1CN(Cc2ccc3nc(C4CCOCC4)ccc3c2)C[C@H]1Oc1ccc2c(c1)CN([C@H]1CCC(=O)NC1=O)C2=O]

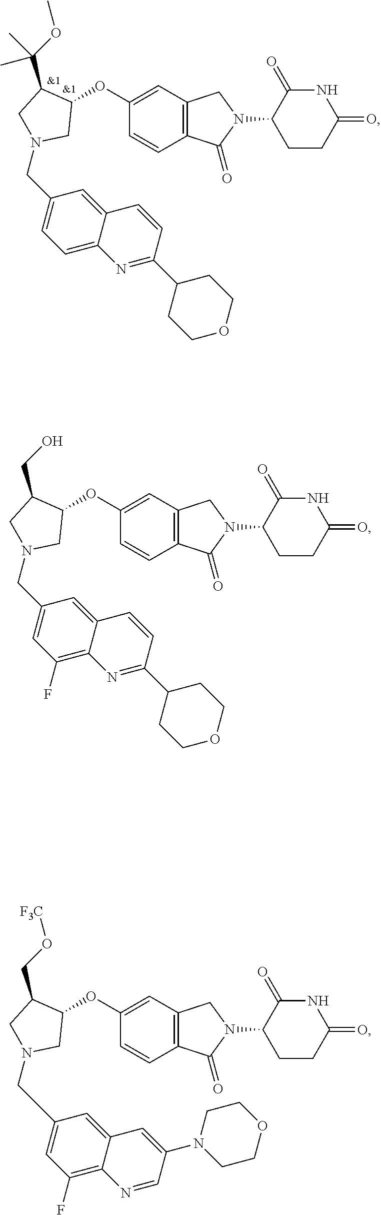 [SMILES: COC(C)(C)[C@@H]1CN(Cc2ccc3nc(C4CCOCC4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4CO)ccc3C2=O)C(=O)N1 ; O=C1CC[C@H](N2Cc3cc(O[C@@H]4CN(Cc5cc(F)c6ncc(N7CCOCC7)cc6c5)C[C@H]4COC(F)(F)F)ccc3C2=O)C(=O)N1]

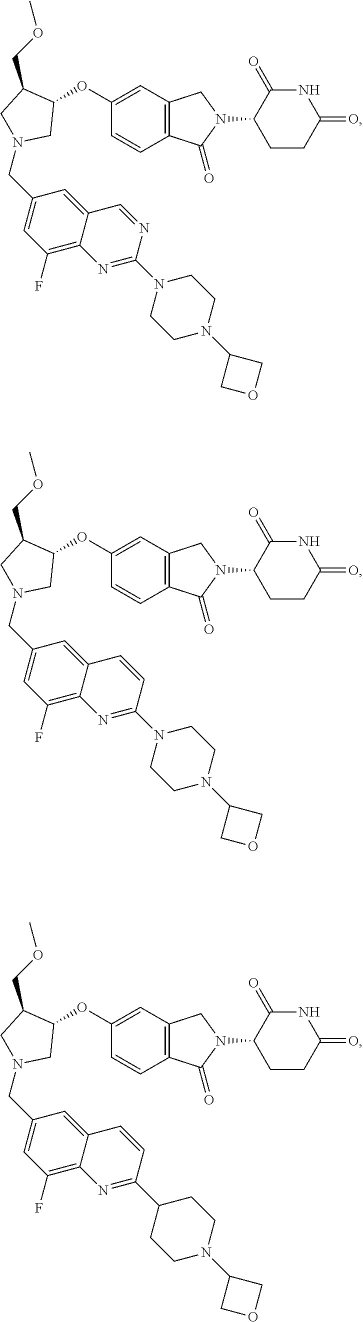 [SMILES: COC[C@@H]1CN(Cc2cc(F)c3nc(N4CCN(C5COC5)CC4)ncc3c2)C[C@H]1Oc1ccc2c(c1)CN([C@H]1CCC(=O)NC1=O)C2=O ; COC[C@@H]1CN(Cc2cc(F)c3nc(N4CCN(C5COC5)CC4)ccc3c2)C[C@H]1Oc1ccc2c(c1)CN([C@H]1CCC(=O)NC1=O)C2=O ; COC[C@@H]1CN(Cc2cc(F)c3nc(C4CCN(C5COC5)CC4)ccc3c2)C[C@H]1Oc1ccc2c(c1)CN([C@H]1CCC(=O)NC1=O)C2=O]

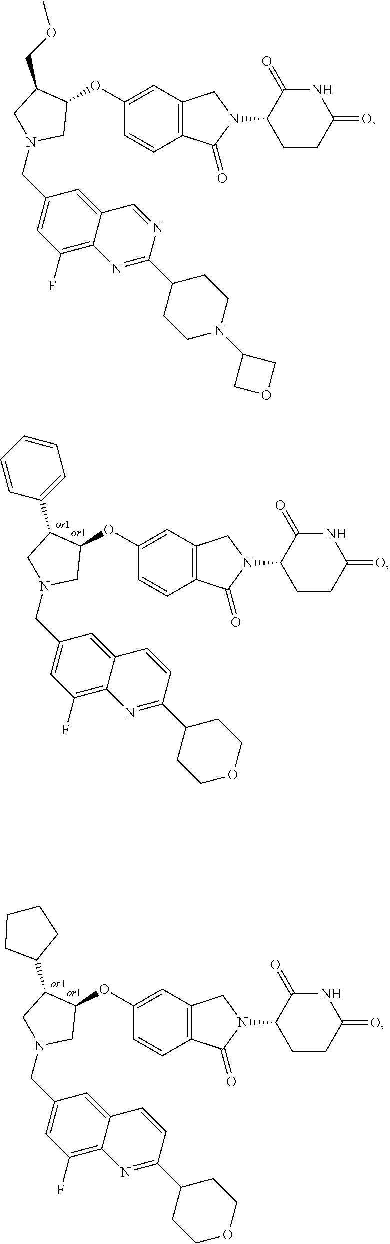 [SMILES: COC[C@@H]1CN(Cc2cc(F)c3nc(C4CCN(C5COC5)CC4)ncc3c2)C[C@H]1Oc1ccc2c(c1)CN([C@H]1CCC(=O)NC1=O)C2=O ; O=C1CC[C@H](N2Cc3cc(O[C@H]4CN(Cc5cc(F)c6nc(C7CCOCC7)ccc6c5)C[C@@H]4c4ccccc4)ccc3C2=O)C(=O)N1 ; O=C1CC[C@H](N2Cc3cc(O[C@H]4CN(Cc5cc(F)c6nc(C7CCOCC7)ccc6c5)C[C@@H]4C4CCCC4)ccc3C2=O)C(=O)N1]

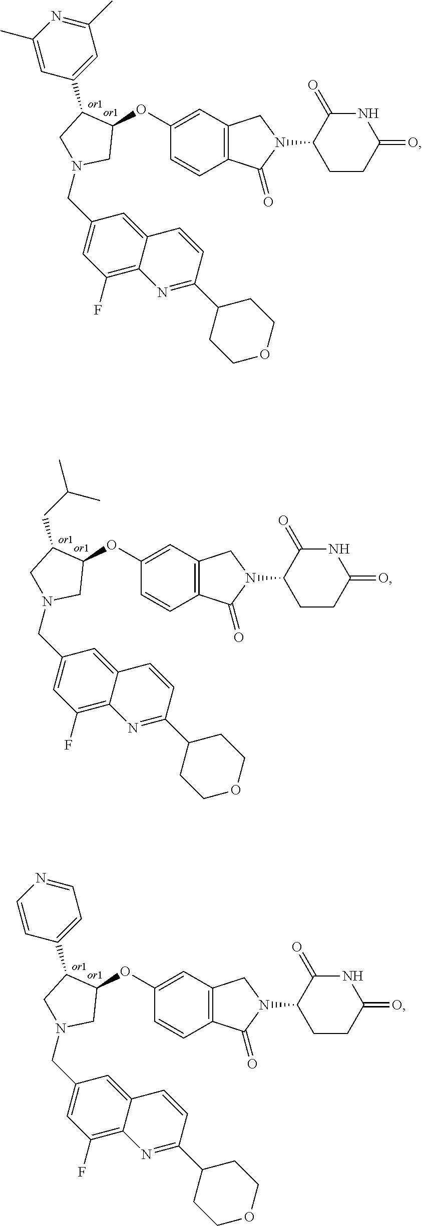 [SMILES: Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)cc(C)n1 ; CC(C)C[C@H]1CN(Cc2cc(F)c3nc(C4CCOCC4)ccc3c2)C[C@@H]1Oc1ccc2c(c1)CN([C@H]1CCC(=O)NC1=O)C2=O ; O=C1CC[C@H](N2Cc3cc(O[C@H]4CN(Cc5cc(F)c6nc(C7CCOCC7)ccc6c5)C[C@@H]4c4ccncc4)ccc3C2=O)C(=O)N1]

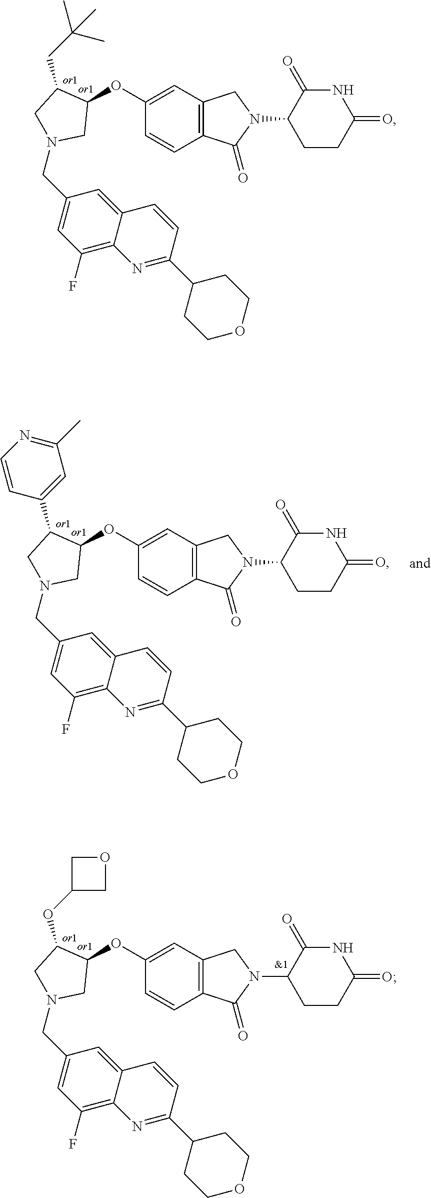 [SMILES: CC(C)(C)C[C@H]1CN(Cc2cc(F)c3nc(C4CCOCC4)ccc3c2)C[C@@H]1Oc1ccc2c(c1)CN([C@H]1CCC(=O)NC1=O)C2=O ; Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)ccn1 ; O=C1CCC(N2Cc3cc(O[C@H]4CN(Cc5cc(F)c6nc(C7CCOCC7)ccc6c5)C[C@@H]4OC4COC4)ccc3C2=O)C(=O)N1]

or a pharmaceutically acceptable salt, tautomer, isotopologue, or stereoisomer thereof, wherein

In the descriptions herein, it is understood that every description, variation, embodiment, or aspect of a moiety may be combined with every description, variation, embodiment, or aspect of other moieties the same as if each and every combination of descriptions is specifically and individually listed. It is also understood that all descriptions, variations, embodiments, or aspects of the compounds, where applicable, apply equally to other formulae detailed herein, and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, may be presented as specific stereoisomers and/or in a non-stereochemical form, it is understood that any or all stereochemical forms, including any enantiomeric or diastereomeric forms, and any tautomers or other forms of the compounds of the present disclosure, including in Table 1, are herein described.

TABLE 1

| Ex. # | Structure | Name | LCMS | NMR |
|---|---|---|---|---|
| 1 | 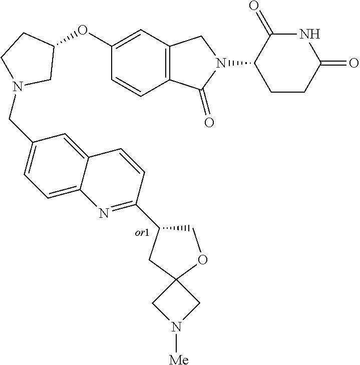 [SMILES: CN1CC2(C[C@H](c3ccc4cc(CN5CC[C@H](Oc6ccc7c(c6)CN([C@H]6CCC(=O)NC6=O)C7=O)C5)ccc4n3)CO2)C1] | (S)-3-(5-(((S)-1- ((2-((R*)-2- methyl-5-oxa-2- azaspiro[3.4]octan- 7-yl)quinolin-6- yl)methyl) pyrrolidin-3- yl)oxy)-1- oxoisoindolin-2- yl)piperidine-2,6- dione | MS (ESI) m/z 596.2 [M + 1]⁺ | ¹H NMR (400 MHz, MeOD-d₄) δ 8.38 (d, J = 8.5 Hz, 1H), 8.32 (s, 1H), 8.09 (d, J = 8.7 Hz, 1H), 8.02 (m, 1H), 7.90- 7.88 (m, 1H), 7.72-7.70 (m, 1H), 7.63 (d, J = 8.5 Hz, 1H), 7.12-7.03 (m, 2H), 5.18-5.09 (m, 2H), 4.50- 4.39 (m, 2H), 4.23 (m, 2H), 3.92 (m, 2H), 3.38-3.34 (m, 1H), 3.28-3.19 (m, 2H), 3.07- 3.00 (m, 1H), 2.96-2.87 (m, 1H), 2.84-2.75 |
| | | | | (m, 1H), 2.57- |
| | | | | 2.42 (m, 2H), |
| | | | | 2.16-2.13 (m 2H) |
| 2 | 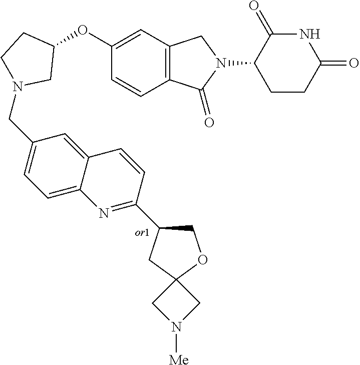 [SMILES: CN1CC2(C[C@@H](c3ccc4cc(CN5CC[C@H](Oc6ccc7c(c6)CN([C@H]6CCC(=O)NC6=O)C7=O)C5)ccc4n3)CO2)C1] | (S)-3-(5-(((S)-1- ((2-((S*)-2- methyl-5-oxa-2- azaspiro[3.4]octan- 7-yl)quinolin-6- yl)methyl) pyrrolidin-3- yl)oxy)-1- oxoisoindolin-2- yl)piperidine-2,6- dione | MS (ESI) m/z 596.2 [M + 1]⁺ | ¹H NMR (400 MHz, DMSO-d₆) δ 8.47-8.22 (m, 2H), 7.97-7.92 (d, J = 8.7 Hz, 1H), 7.82 (s, 1H), 7.74-7.70 (m, 1H), 7.61 (d, J = 8.5 Hz, 1H), 7.52-7.43 (m, 1H), 7.12 (m, 1H), 7.02-6.90 (m, 1H), 5.11- 5.01 (m, 2H), 4.45-4.34 (m, 2H), 4.40-4.29 (m, 3H), 4.19- 3.91 (m, 3H), 3.80 (m, 3H), 2.95-2.85 (m, 2H), 2.80-2.75 (m, 4H), 2.73- 2.64 (m, 1H), |
| | | | | 2.66-2.52 (m, |
| | | | | 3H), 2.50-2.38 |
| | | | | (m, 3H), 2.05- |
| | | | | 1.95 (m, 1H), |
| | | | | 1.94-1.81 (m, |
| | | | | 1H) |
| 3 | 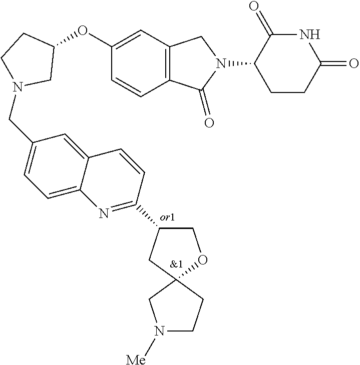 [SMILES: CN1CC[C@]2(C[C@H](c3ccc4cc(CN5CC[C@H](Oc6ccc7c(c6)CN([C@H]6CCC(=O)NC6=O)C7=O)C5)ccc4n3)CO2)C1] | rel-(3S)-3-(5- (((3S)-1-((2-(7- methyl-1-oxa-7- azaspiro[4.4]nonan- 3-yl)quinolin-6- yl)methyl) pyrrolidin-3- yl)oxy)-1- oxoisoindolin-2- yl)piperidine-2,6- dione | MS (ESI) m/z 610.4 [M + 1]⁺ | ¹H NMR (400 MHz, MeOD-d₄) δ 8.35 (s, 1H), 8.14 (d, J = 8.9 Hz, 2H), 7.91 (d, J = 8.6 Hz, 1H), 7.77-7.55 (m, 2H), 7.17 (d, J = 2.0 Hz, 1H), 7.07 (m, 1H), 5.35 (s, 1H), 5.15 (dd, J = 13.4, 5.1 Hz, 1H), 4.71 (s, 2H), 4.52-4.34 (m, 3H), 4.27-4.13 (m, 1H), 4.08- 3.95 (m, 1H), 3.93-3.42 (m, 7H), 3.19 (m, 1H), 3.08-2.94 (m, 3H), 2.92- 2.72 (m, 2H), 2.70-2.53 (m, |
| | | | | 3H), 2.51-2.38 |
| | | | | (m, 4H), 2.23- |
| | | | | 2.13 (m, 1H) |
| 4 | 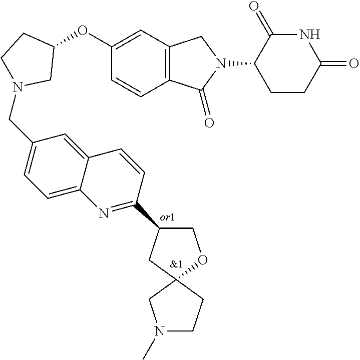 [SMILES: CN1CC[C@]2(C[C@@H](c3ccc4cc(CN5CC[C@H](Oc6ccc7c(c6)CN([C@H]6CCC(=O)NC6=O)C7=O)C5)ccc4n3)CO2)C1] | rel-(3S)-3-(5- (((3S)-1-((2-(7- methyl-1-oxa-7- azaspiro[4.4]nonan- 3-yl)quinolin-6- yl)methyl)pyrrolidin- 3-yl)oxy)-1- oxoisoindolin-2- yl)piperidine-2,6- dione | MS (ESI) m/z 610.3 [M + 1]⁺ | ¹H NMR (400 MHz, MeOD-d₄) δ 8.35 (s, 1H), 8.12 (d, J = 8.6 Hz, 2H), 7.90 (m, 1H), 7.70 (m, 1H), 7.60 (m, 1H), 7.15 (s, 1H), 7.04 (m, 1H), 5.32 (s, 1H), 5.15-5.09 (m, 1H), 4.69 (s, 2H), 4.42 (m 3H), 4.18-4.08 (m, 1H), 4.02-3.98 (m, 1H), 3.92- 3.40 (m, 7H), 3.26-3.15 (m, 2H), 3.05 (m, 1H), 2.99 (s, 3H), 2.89-2.73 (m, 1H), 2.64 (m, |
| | | | | 2H), 2.52-2.30 |
| | | | | (m, 4H), 2.15 (m, |
| | | | | 1H) |
| 5 | 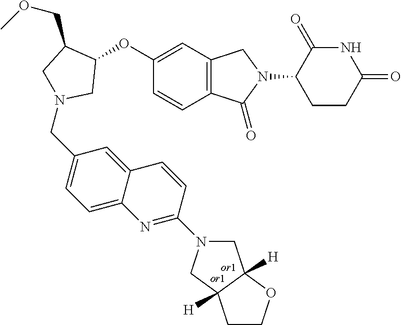 [SMILES: COC[C@@H]1CN(Cc2ccc3nc(N4C[C@H]5CCO[C@H]5C4)ccc3c2)C[C@H]1Oc1ccc2c(c1)CN([C@H]1CCC(=O)NC1=O)C2=O] | (S)-3-(5- (((3S,4S)-1-((2- ((3aR*,6aR*)- hexahydro-5H- furo[2,3-c]pyrrol- 5-yl)quinolin-6- yl)methyl)-4- (methoxymethyl) pyrrolidin-3- yl)oxy)-1- oxoisoindolin-2- yl)piperidine-2,6- dione | MS (ESI) m/z 626.4 [M + 1]+ | ¹H NMR (400 MHz, MeOD-d₄) δ 8.36 (s, 1H), 8.03 (d, J = 9.0 Hz, 1H), 7.79- 7.61 (m, 4H), 7.17-7.05 (m, 2H), 6.99 (d, J = 9.2 Hz, 1H), 5.12 (dd, J = 13.3, 4.9 Hz, 1H), 5.00 (s, 1H), 4.68 (m, 1H), 4.50-4.37 (m, 2H), 4.23- 4.09 (m, 2H), 4.04 (m, 1H), 3.92-3.88 (m, 3H), 3.76 (m, 1H), 3.63-3.45 (m, 4H), 3.40 (s, |
| | | | | 3H), 3.30 (m, |
| | | | | 3H), 3.16 (m, |
| | | | | 1H), 2.96-2.74 |
| | | | | (m, 4H), 2.54- |
| | | | | 2.39 (m, 1H), |
| | | | | 2.31-2.20 (m, |
| | | | | 1H), 2.19-2.11 |
| | | | | (m, 1H), 2.01 (m, |
| | | | | 1H) |
| 6 | 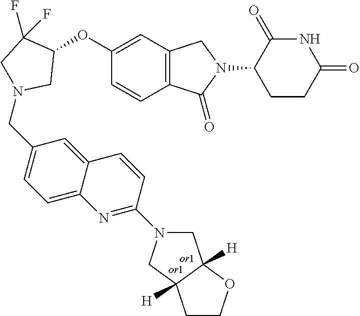 [SMILES: O=C1CC[C@H](N2Cc3cc(O[C@@H]4CN(Cc5ccc6nc(N7C[C@H]8CCO[C@H]8C7)ccc6c5)CC4(F)F)ccc3C2=O)C(=O)N1] | (S)-3-(5-(((R)- 4,4-difluoro-1- ((2-((3aR*,6aR*)- hexahydro-5H- furo[2,3-c]pyrrol- 5-yl)quinolin-6- yl)methyl)pyrrolidin- 3-yl)oxy)-1- oxoisoindolin-2- yl)piperidine-2,6- dione | MS (ESI) m/z 618.4 [M + H]⁺ | ¹H NMR (400 MHz, DMSO-d₆) δ 8.00 (d, J = 9.0 Hz, 1H), 7.66 (d, J = 8.3 Hz, 1H), 7.61 (s, 1H), 7.54 (d, J = 8.8 Hz, 1H), 7.48 (d, J = 8.3 Hz, 1H), 7.21 (s, 1H), 7.11 (d, J = 8.5 Hz, 1H), 6.91 (d, J = 9.1 Hz, 1H), 5.07 (m, 2H), 4.57 (m, 1H), 4.41 (d, J = 17.3 Hz, 1H), 4.26 (d, J = 17.3 Hz, 1H), 3.87 (m, 1H), 3.75 (m, 5H), 3.60 (m, |
| | | | | 1H), 3.35 (m, |
| | | | | 2H), 3.17 (m, |
| | | | | 1H), 3.04 (m, |
| | | | | 1H), 2.92 (m, |
| | | | | 2H), 2.69 (m, |
| | | | | 1H), 2.57 (m, |
| | | | | 1H), 2.37 (m, |
| | | | | 1H), 2.16-2.07 |
| | | | | (m, 1H), 1.98 (m, |
| | | | | 1H), 1.86 (m, |
| | | | | 1H) |
| 7 | 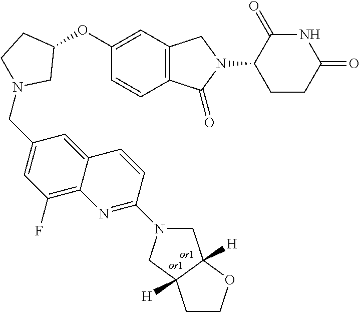 [SMILES: O=C1CC[C@H](N2Cc3cc(O[C@H]4CCN(Cc5cc(F)c6nc(N7C[C@H]8CCO[C@H]8C7)ccc6c5)C4)ccc3C2=O)C(=O)N1] | (S)-3-(5-(((S)-1- ((8-fluoro-2- ((3aR*,6aR*)- hexahydro-5H- furo[2,3-c]pyrrol- 5-yl)quinolin-6- yl)methyl)pyrrolidin- 3-yl)oxy)-1- oxoisoindolin-2- yl)piperidine-2,6- dione | MS (ESI) m/z 600.4 [M + 1]⁺ | ¹H NMR (400 MHz, MeOD-d₄) δ 8.19 (m, 1H), 7.79-7.72 (m, 3H), 7.26-7.07 (m, 3H), 5.34 (m, 1H), 5.15 (m, 1H), 4.72 (m, 1H), 4.61 (m, 2H), 4.54-4.38 (m, 2H), 4.15- 4.03 (m, 3H), 4.00-3.90 (m, 2H), 3.75 (m, 3H), 3.67 (m, 2H), 3.24 (m, 1H), 2.93 (m, 1H), 2.80 (m, 1H), 2.63 (m, |
| | | | | 1H), 2.54-2.43 |
| | | | | (m, 2H), 2.37- |
| | | | | 2.23 (m, 1H), |
| | | | | 2.18 (m, 1H), |
| | | | | 2.09-2.01 (m, |
| | | | | 1H) |
| 8 | 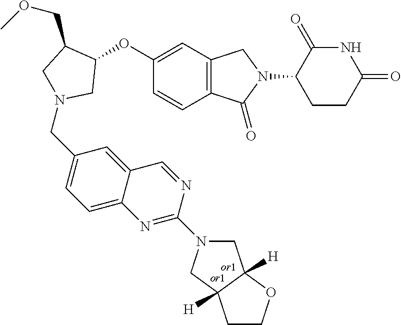 [SMILES: COC[C@@H]1CN(Cc2ccc3nc(N4C[C@H]5CCO[C@H]5C4)ncc3c2)C[C@H]1Oc1ccc2c(c1)CN([C@H]1CCC(=O)NC1=O)C2=O] | (S)-3-(5- (((3S,4S)-1-((2- ((3aR*,6aR*)- hexahydro-5H- furo[2,3-c]pyrrol- 5-yl)quinazolin- 6-yl)methyl)-4- (methoxymethyl) pyrrolidin-3- yl)oxy)-1- oxoisoindolin-2- yl)piperidine-2,6- dione | MS (ESI) m/z 627.4 [M + 1]⁺ | ¹H NMR (400 MHz, MeOD-d₄) δ 9.07 (s, 1H), 7.78 (m, 2H), 7.70 (d, J = 8.4 Hz, 1H), 7.59 (d, J = 8.6 Hz, 1H), 7.14 (d, J = 2.2 Hz, 1H), 7.08 (dd, J = 8.5, 2.2 Hz, 1H), 5.11 (dd, J = 13.3, 5.1 Hz, 1H), 4.97- 4.91 (m, 1H), 4.86-4.61 (m, 1H), 4.45 (d, J = 17.0 Hz, 1H), 4.39 (d, J = 17.0 Hz, 1H), 4.09- 3.96 (m, 4H), |
| | | | | 3.95-3.84 (m, |
| | | | | 2H), 3.74 (m, |
| | | | | 1H), 3.68-3.49 |
| | | | | (m, 3H), 3.39 (s, |
| | | | | 3H), 3.33 (m, |
| | | | | 1H), 3.20 (m, |
| | | | | 2H), 3.12 (m, |
| | | | | 1H), 2.97-2.87 |
| | | | | (m, 1H), 2.81- |
| | | | | 2.67 (m, 3H), |
| | | | | 2.54-2.38 (m, |
| | | | | 1H), 2.31-2.11 |
| | | | | (m, 2H), 2.02- |
| | | | | 1.91 (m, 1H) |
| 9 | 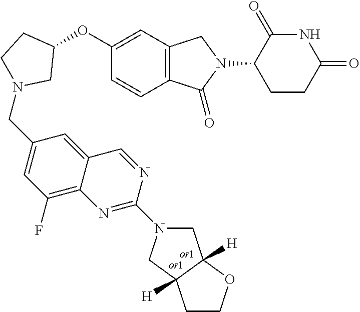 [SMILES: O=C1CC[C@H](N2Cc3cc(O[C@H]4CCN(Cc5cc(F)c6nc(N7C[C@H]8CCO[C@H]8C7)ncc6c5)C4)ccc3C2=O)C(=O)N1] | (S)-3-(5-(((S)-1- ((8-fluoro-2- ((3aR*,6aR*)- hexahydro-5H- furo[2,3-c]pyrrol- 5-yl)quinazolin- 6-yl)methyl) pyrrolidin- 3-yl)oxy)-1- oxoisoindolin-2- yl)piperidine-2,6- dione | MS (ESI) m/z 601.2 [M + 1]⁺ | ¹H NMR (400 MHz, MeOD-d₄) δ 9.11 (s, 1H), 7.72 (d, J = 8.4 Hz, 1H), 7.67- 7.62 (m, 1H), 7.60-7.54 (m, 1H), 7.11 (d, J = 2.5 Hz, 1H), 7.09-7.03 (m, 1H), 5.13 (m, 2H), 4.69-4.61 (m, 1H), 4.45 (m, 2H), 4.14-3.85 (m, 6H), 3.81- 3.56 (m, 3H), 3.32-3.10 (m, 3H), 3.08-2.70 (m, 3H), 2.60- |
| | | | | 2.40 (m, 2H), |
| | | | | 2.32-2.10 (m, |
| | | | | 3H), 2.03-1.92 |
| | | | | (m, 1H) |
| 10 | 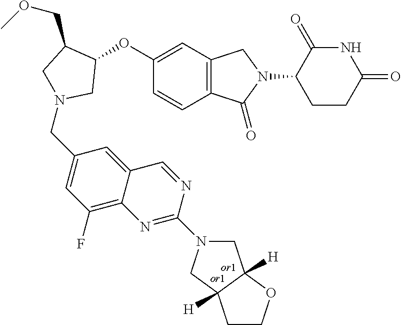 [SMILES: COC[C@@H]1CN(Cc2cc(F)c3nc(N4C[C@H]5CCO[C@H]5C4)ncc3c2)C[C@H]1Oc1ccc2c(c1)CN([C@H]1CCC(=O)NC1=O)C2=O] | (S)-3-(5- (((3S,4S)-1-((8- fluoro-2- ((3aR*,6aR*)- hexahydro-5H- furo[2,3-c]pyrrol- 5-yl)quinazolin- 6-yl)methyl)-4- (methoxymethyl) pyrrolidin-3- yl)oxy)-1- oxoisoindolin-2- yl)piperidine-2,6- dione | MS (ESI) m/z 645.2 [M + 1]⁺ | ¹H NMR (400 MHz, DMSO-d₆) δ 10.99 (s, 1H), 9.22 (s, 1H), 8.16 (d, J = 2.5 Hz, 1H), 7.60 (m, 2H), 7.52 (d, J = 12.0 Hz, 1H), 7.16 (s, 1H), 7.03 (d, J = 8.5 Hz, 1H), 5.11-5.02 (m, 1H), 4.75 (m, 1H), 4.58-4.52 (m, 1H), 4.37 (d, J = 17.2 Hz, 1H), 4.25 (d, J = 17.1 Hz, 1H), 3.92- 3.82 (m, 3H), 3.80-3.50 (m, 4H), 3.49-3.37 |
| | | | | (m, 3H), 3.25 (s, |
| | | | | 3H), 3.04 (m, |
| | | | | 1H), 2.92 (m, |
| | | | | 3H), 2.74 (m, |
| | | | | 1H), 2.63-2.53 |
| | | | | (m, 2H), 2.45- |
| | | | | 2.33 (m, 1H), |
| | | | | 2.27-2.18 (m, |
| | | | | 1H), 2.17-2.04 |
| | | | | (m, 1H), 1.98 (m, |
| | | | | 1H), 1.88 (m, |
| | | | | 1H) |
| 11 | 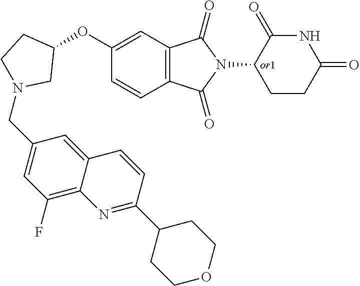 [SMILES: O=C1CC[C@H](N2C(=O)c3ccc(O[C@H]4CCN(Cc5cc(F)c6nc(C7CCOCC7)ccc6c5)C4)cc3C2=O)C(=O)N1] | 2-((S*)-2,6- dioxopiperidin-3- yl)-5-(((S)-1-((8- fluoro-2- (tetrahydro-2H- pyran-4- yl)quinolin-6- yl)methyl) pyrrolidin-3- yl)oxy)isoindoline- 1,3-dione | MS (ESI) m/z 587.2 [M + 1]⁺ | ¹H NMR (400 MHz, DMSO-d₆) δ 11.11 (s, 1H), 8.35 (d, J = 8.4 Hz, 1H), 7.82 (d, J = 8.3 Hz, 1H), 7.70 (s, 1H), 7.59 (d, J = 8.6 Hz, 1H), 7.52 (d, J = 11.9 Hz, 1H), 7.37 (d, J = 2.2 Hz, 1H), 7.32 (m, 1H), 5.18 (d, J = 6.9 Hz, 1H), 5.11 (m, 1H), 4.21- 3.95 (m, 2H), 3.86-3.73 (m, 2H), 3.62-3.45 |
| | | | | (m, 2H), 3.28- |
| | | | | 3.16 (m, 1H), |
| | | | | 2.95 (m, 1H), |
| | | | | 2.94-2.77 (m, |
| | | | | 2H), 2.76 (m, |
| | | | | 1H), 2.67-2.52 |
| | | | | (m, 3H), 2.43- |
| | | | | 2.35 (m, 1H), |
| | | | | 2.09-1.99 (m, |
| | | | | 1H), 1.92-1.80 |
| | | | | (m, 5H) |
| 12 | 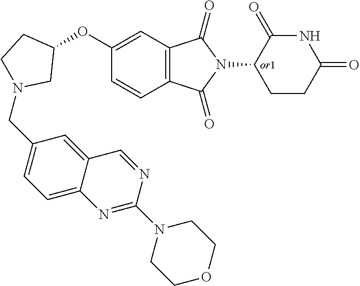 [SMILES: O=C1CC[C@H](N2C(=O)c3ccc(O[C@H]4CCN(Cc5ccc6nc(N7CCOCC7)ncc6c5)C4)cc3C2=O)C(=O)N1] | 2-((S*)-2,6- dioxopiperidin-3- yl)-5-(((S)-1-((2- morpholino- quinazolin-6- yl)methyl) pyrrolidin-3- yl)oxy)isoindoline- 1,3-dione | MS (ESI) m/z 571.3 [M + 1]⁺ | ¹H NMR (400 MHz, MeOD-d₄) δ 9.16 (s, 1H), 7.98 (d, J = 2.0 Hz, 1H), 7.89- 7.81 (m, 2H), 7.66 (d, J = 8.7 Hz, 1H), 7.45 (d, J = 2.3 Hz, 1H), 7.36 (m, 1H), 5.44 (m, 1H), 5.14 (m, 1H), 4.60 (s, 2H), 4.01-3.94 (m, 4H), 3.87-3.77 (m, 7H), 3.60 (m, 1H), 2.89 (m, 2H), 2.82-2.66 |
| | | | | (m, 2H), 2.40 (m, |
| | | | | 1H), 2.19-2.10 |
| | | | | (m, 1H) |
| 13 | 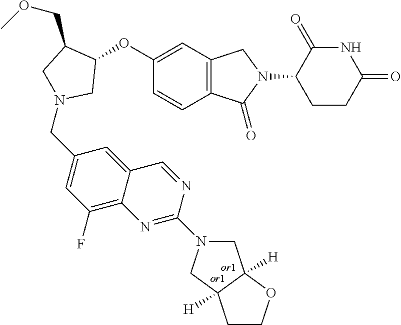 [SMILES: COC[C@@H]1CN(Cc2cc(F)c3nc(N4C[C@@H]5CCO[C@@H]5C4)ncc3c2)C[C@H]1Oc1ccc2c(c1)CN([C@H]1CCC(=O)NC1=O)C2=O] | (S)-3-(5- (((3S,4S)-1-((8- fluoro-2- ((3aS*,6aS*)- hexahydro-5H- furo[2,3-c]pyrrol- 5-yl)quinazolin- 6-yl)methyl)-4- (methoxymethyl) pyrrolidin-3- yl)oxy)-1- oxoisoindolin-2- yl)piperidine-2,6- dione | MS (ESI) m/z 645.3 [M + 1]⁺ | ¹H NMR (400 MHz, DMSO-d₆) δ 10.99 (s, 1H), 9.22 (s, 1H), 7.64-7.56 (m, 2H), 7.52 (d, J = 11.9 Hz, 1H), 7.15 (s, 1H), 7.03 (d, J = 8.5 Hz, 1H), 5.07 (dd, J = 13.6, 4.8 Hz, 1H), 4.74 (m, 1H), 4.54 (m, 1H), 4.37 (d, J = 17.3 Hz, 1H), 4.25 (d, J = 17.2 Hz, 1H), 3.91- 3.81 (m, 3H), 3.79-3.58 (m, 4H), 3.47-3.37 |
| | | | | (m, 3H), 3.26 (s, |
| | | | | 3H), 3.08-3.00 |
| | | | | (m, 1H), 2.98- |
| | | | | 2.85 (m, 3H), |
| | | | | 2.74 (m, 1H), |
| | | | | 2.63-2.53 (m, |
| | | | | 2H), 2.43-2.30 |
| | | | | (m, 1H), 2.26- |
| | | | | 2.18 (m, 1H), |
| | | | | 2.11 (m, 1H), |
| | | | | 2.01-1.93 (m, |
| | | | | 1H), 1.88 (m, |
| | | | | 1H) |
| 14 | 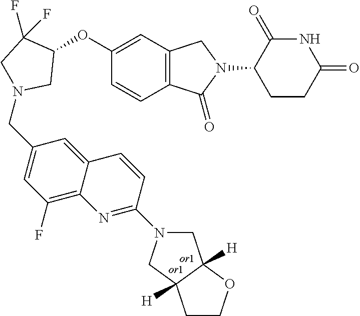 [SMILES: O=C1CC[C@H](N2Cc3cc(O[C@@H]4CN(Cc5cc(F)c6nc(N7C[C@H]8CCO[C@H]8C7)ccc6c5)CC4(F)F)ccc3C2=O)C(=O)N1] | (S)-3-(5-(((R)- 4,4-difluoro-1- ((8-fluoro-2- ((3aR*,6aR*)- hexahydro-5H- furo[2,3-c]pyrrol- 5-yl)quinolin-6- yl)methyl) pyrrolidin-3- yl)oxy)-1- oxoisoindolin-2- yl)piperidine-2,6- dione | MS (ESI) m/z 636.2 [M + 1]⁺ | ¹H NMR (400 MHz, MeOD-d₄) δ 8.00 (m, 1H), 7.74 (d, J = 8.5 Hz, 1H), 7.45 (d, J = 1.8 Hz, 1H), 7.36 (m, 1H), 7.19 (d, J = 2.2 Hz, 1H), 7.15 (m, 1H), 6.98 (d, J = 9.2 Hz, 1H), 5.14 (m, 1H), 5.04 (m, 1H), 4.69 (t, J = 5.3 Hz, 1H), 4.49 (d, J = 17.1 Hz, 1H), 4.43 (d, J = 17.1 Hz, 1H), 4.08-3.98 (m, 1H), 3.98-3.83 (m, 3H), 3.81 (m, |
| | | | | 2H), 3.73 (m, |
| | | | | 1H), 3.68-3.51 |
| | | | | (m, 1H), 3.44 (m, |
| | | | | 1H), 3.28-3.18 |
| | | | | (m, 1H), 3.16 (m, |
| | | | | 1H), 3.06-2.75 |
| | | | | (m, 3H), 2.49- |
| | | | | 2.47 (m, 1H), |
| | | | | 2.32-2.12 (m, |
| | | | | 2H), 2.06-1.94 |
| | | | | (m, 1H) |
| 15 | 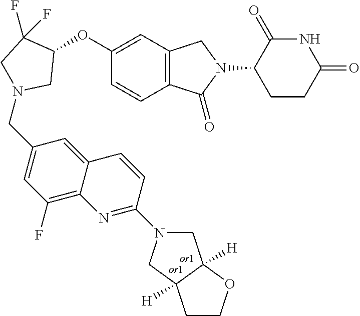 [SMILES: O=C1CC[C@H](N2Cc3cc(O[C@@H]4CN(Cc5cc(F)c6nc(N7C[C@@H]8CCO[C@@H]8C7)ccc6c5)CC4(F)F)ccc3C2=O)C(=O)N1] | (S)-3-(5-(((R)- 4,4-difluoro-1- ((8-fluoro-2- ((3aS*,6aS*)- hexahydro-5H- furo[2,3-c]pyrrol- 5-yl)quinolin-6- yl)methyl) pyrrolidin-3- yl)oxy)-1- oxoisoindolin-2- yl)piperidine-2,6- dione | MS (ESI) m/z 636.3 [M + 1]⁺ | ¹H NMR (400 MHz, DMSO-d₆) δ 10.96 (s, 1H), 8.05 (m, 1H), 7.66 (d, J = 8.4 Hz, 1H), 7.46 (d, J = 1.8 Hz, 1H), 7.32 (m, 1H), 7.22 (d, J = 2.2 Hz, 1H), 7.12 (m, 1H), 6.99 (d, J = 9.1 Hz, 1H), 5.19-5.03 (m, 2H), 4.58 (t, J = 5.5 Hz, 1H), 4.41 (d, J = 17.3 Hz, 1H), 4.27 (d, J = 17.3 Hz, 1H), 3.88 (m, 1H), 3.84-3.72 (m, |
| | | | | 4H), 3.62 (m, |
| | | | | 1H), 3.46-3.37 |
| | | | | (m, 2H), 3.19 (m, |
| | | | | 1H), 3.09-2.99 |
| | | | | (m, 1H), 2.96 (m, |
| | | | | 1H), 2.94-2.84 |
| | | | | (m, 1H), 2.71 (m, |
| | | | | 1H), 2.59 (m, |
| | | | | 2H), 2.44-2.33 |
| | | | | (m, 1H), 2.12- |
| | | | | 2.10 (m, 1H), |
| | | | | 2.02-1.95 (m, |
| | | | | 1H), 1.90-1.80 |
| | | | | (m, 1H) |
| 16 | 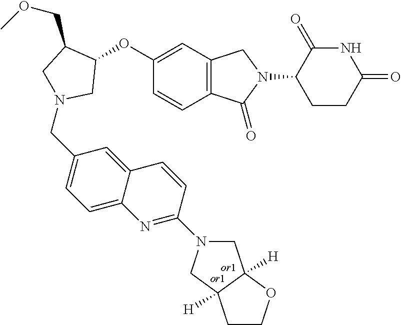 [SMILES: COC[C@@H]1CN(Cc2ccc3nc(N4C[C@@H]5CCO[C@@H]5C4)ccc3c2)C[C@H]1Oc1ccc2c(c1)CN([C@H]1CCC(=O)NC1=O)C2=O] | (S)-3-(5- (((3S,4S)-1-((2- ((3aS*,6aS*)- hexahydro-5H- furo[2,3-c]pyrrol- 5-yl)quinolin-6- yl)methyl)-4- (methoxymethyl) pyrrolidin-3- yl)oxy)-1- oxoisoindolin-2- yl)piperidine-2,6- dione | MS (ESI) m/z 626.4 [M + 1]⁺ | ¹H NMR (400 MHz, MeOD-d₄) δ 8.34 (s, 1H), 8.03 (d, J = 9.2 Hz, 1H), 7.77- 7.63 (m, 4H), 7.15-7.05 (m, 2H), 7.00 (d, J = 9.2 Hz, 1H), 5.12 (dd, J = 13.3, 5.1 Hz, 1H), 5.00- 4.98 (m, 1H), 4.69 (m, 1H), 4.48-4.37 (m, 2H), 4.20-4.13 (m, 2H), 4.04- 4.02 (m, 1H), 3.96-3.87 (m, 3H), 3.76 (m, 1H), 3.61-3.52 |
| | | | | (m, 3H), 3.48 (m, |
| | | | | 1H), 3.40 (s, 3H), |
| | | | | 3.31 (m, 2H), |
| | | | | 3.20-3.12 (m, |
| | | | | 1H), 2.97-2.75 |
| | | | | (m, 4H), 2.52- |
| | | | | 2.43 (m, 1H), |
| | | | | 2.31-2.23 (m, |
| | | | | 1H), 2.16-2.12 |
| | | | | (m, 1H), 2.00- |
| | | | | 1.95 (m, 1H) |
| 17 | 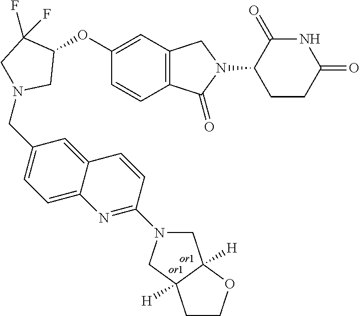 [SMILES: O=C1CC[C@H](N2Cc3cc(O[C@@H]4CN(Cc5ccc6nc(N7C[C@@H]8CCO[C@@H]8C7)ccc6c5)CC4(F)F)ccc3C2=O)C(=O)N1] | (S)-3-(5-(((R)- 4,4-difluoro-1- ((2-((3aS*,6aS*)- hexahydro-5H- furo[2,3-c]pyrrol- 5-yl)quinolin-6- yl)methyl) pyrrolidin-3- yl)oxy)-1- oxoisoindolin-2- yl)piperidine-2,6- dione | MS (ESI) m/z 618.3 [M + 1]⁺ | ¹H NMR (400 MHz, DMSO-d₆) δ 8.00 (d, J = 8.9 Hz, 1H), 7.66 (d, J = 8.4 Hz, 1H), 7.60 (d, J = 4.5 Hz, 1H), 7.54 (d, J = 8.8 Hz, 1H), 7.48 (d, J = 8.1 Hz, 1H), 7.21 (m, 1H), 7.11 (m, 1H), 6.91 (m, 1H), 5.21-5.03 (m, 2H), 4.57 (m, 1H), 4.41 (d, J = 17.1 Hz, 1H), 4.26 (m, 1H), 3.87-3.85 (m, 1H), 3.76 (m, 5H), 3.59 (m, |
| | | | | 1H), 3.33 (m, |
| | | | | 2H), 3.20 (m, |
| | | | | 1H), 3.04 (m, |
| | | | | 1H), 2.98-2.81 |
| | | | | (m, 2H), 2.69 (m, |
| | | | | 1H), 2.59 (m, |
| | | | | 1H), 2.43-2.31 |
| | | | | (m, 1H), 2.17- |
| | | | | 2.05 (m, 1H), |
| | | | | 2.02-1.93 (m, |
| | | | | 1H), 1.87 (m, |
| | | | | 1H) |
| 18 | 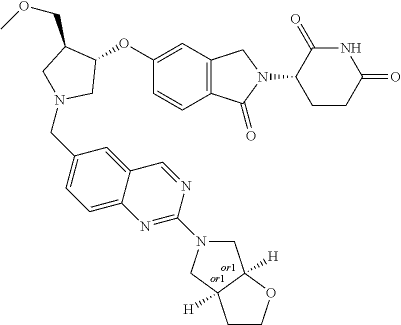 [SMILES: COC[C@@H]1CN(Cc2ccc3nc(N4C[C@@H]5CCO[C@@H]5C4)ncc3c2)C[C@H]1Oc1ccc2c(c1)CN([C@H]1CCC(=O)NC1=O)C2=O] | (S)-3-(5- (((3S,4S)-1-((2- ((3aS*,6aS*)- hexahydro-5H- furo[2,3-c]pyrrol- 5-yl)quinazolin- 6-yl)methyl)-4- (methoxymethyl) pyrrolidin-3- yl)oxy)-1- oxoisoindolin-2- yl)piperidine-2,6- dione | MS (ESI) m/z 627.2 [M + 1]⁺ | ¹H NMR (400 MHz, MeOD-d₄) δ 9.07 (s, 1H), 7.77 (d, J = 9.7 Hz, 2H), 7.71 (d, J = 8.4 Hz, 1H), 7.59 (d, J = 8.5 Hz, 1H), 7.14 (d, J = 2.0 Hz, 1H), 7.08 (dd, J = 8.5, 2.2 Hz, 1H), 5.12 (dd, J = 13.3, 5.1 Hz, 1H), 4.86 (m, 1H), 4.69-4.62 (m, 1H), 4.50- 4.36 (m, 2H), 4.07-3.97 (m, 2H), 3.95-3.82 (m, 4H), 3.74 (m, 1H), 3.59-3.50 |
| | | | | (m, 3H), 3.39 (s, |
| | | | | 3H), 3.24-3.08 |
| | | | | (m, 2H), 3.03 (m, |
| | | | | 2H), 2.98-2.84 |
| | | | | (m, 1H), 2.83- |
| | | | | 2.69 (m, 2H), |
| | | | | 2.55-2.40 (m, |
| | | | | 2H), 2.31-2.12 |
| | | | | (m, 2H), 2.01- |
| | | | | 1.93 (m, 1H) |
| 19 | 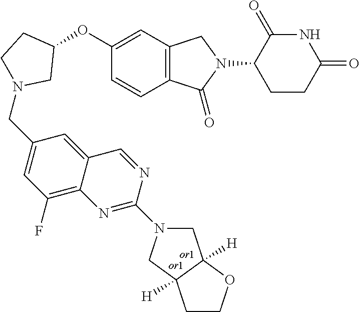 [SMILES: O=C1CC[C@H](N2Cc3cc(O[C@H]4CCN(Cc5cc(F)c6nc(N7C[C@@H]8CCO[C@@H]8C7)ncc6c5)C4)ccc3C2=O)C(=O)N1] | (S)-3-(5-(((S)-1- ((8-fluoro-2- ((3aS*,6aS*)- hexahydro-5H- furo[2,3-c]pyrrol- 5-yl)quinazolin- 6-yl)methyl) pyrrolidin-3- yl)oxy)-1- oxoisoindolin-2- yl)piperidine-2,6- dione | MS (ESI) m/z 601.2 [M + 1]⁺ | ¹H NMR (400 MHz, MeOD-d₄) δ 9.12 (s, 1H), 8.29 (s, 1H), 7.72-7.54 (m, 3H), 7.13-7.03 (m, 2H), 5.23- 5.08 (m, 2H), 4.69-4.62 (m, 1H), 4.47 (m, 2H), 4.20-3.92 (m, 6H), 3.76 (m, 1H), 3.62 (m, 2H), 3.30-3.00 (m, 4H), 2.98- 2.85 (m, 1H), 2.84-2.74 (m, 1H), 2.56-2.40 (m, 2H), 2.20 (m, |
| | | | | 3H), 2.03-1.93 |
| | | | | (m, 1H) |
| 20 | 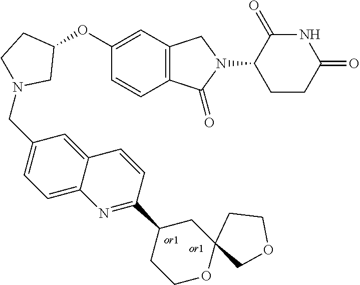 [SMILES: O=C1CC[C@H](N2Cc3cc(O[C@H]4CCN(Cc5ccc6nc([C@@H]7CCO[C@@]8(CCOC8)C7)ccc6c5)C4)ccc3C2=O)C(=O)N1] | rel-(3S)-3-(5- (((3S)-1-((2-(2,6- dioxaspiro[4.5] decan-9- yl)quinolin-6- yl)methyl) pyrrolidin-3- yl)oxy)-1- oxoisoindolin-2- yl)piperidine-2,6- dione | MS (ESI) m/z 611.3 [M + 1]⁺ | ¹H NMR (400 MHz, MeOD-d₄) δ 8.36-8.26 (m, 2H), 8.06 (d, J = 8.7 Hz, 1H), 7.95 (d, J = 2.0 Hz, 1H), 7.85-7.83 (m, 1H), 7.71 (d, J = 8.4 Hz, 1H), 7.54 (d, J = 8.6 Hz, 1H), 7.10 (d, J = 2.2 Hz, 1H), 7.07-7.05 (m, 1H), 5.15-5.11 (m, 2H), 4.45- 4.43 (m, 2H), 4.28-4.25 (m, 1H), 4.20-4.19 |
| | | | | (m, 2H), 3.95- |
| | | | | 3.82 (m, 4H), |
| | | | | 3.75 (m, 1H), |
| | | | | 3.31 (m, 1H), |
| | | | | 3.30-3.22 (m, |
| | | | | 2H), 3.19-3.10 |
| | | | | (m, 1H), 3.07- |
| | | | | 2.97 (m, 1H), |
| | | | | 2.95-2.88 (m, |
| | | | | 1H), 2.85-2.79 |
| | | | | (m, 1H), 2.53- |
| | | | | 2.46 (m, 2H), |
| | | | | 2.24-2.12 (m, |
| | | | | 3H), 2.12-1.95 |
| | | | | (m, 4H), 1.95- |
| | | | | 1.85 (m, 1H) |
| 21 | 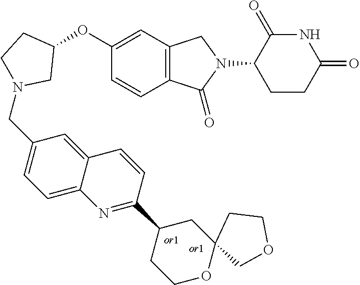 [SMILES: O=C1CC[C@H](N2Cc3cc(O[C@H]4CCN(Cc5ccc6nc([C@@H]7CCO[C@]8(CCOC8)C7)ccc6c5)C4)ccc3C2=O)C(=O)N1] | rel-(3S)-3-(5- (((3S)-1-((2-(2,6- dioxaspiro[4.5] decan-9- yl)quinolin-6- yl)methyl) pyrrolidin-3- yl)oxy)-1- oxoisoindolin-2- yl)piperidine-2,6- dione | MS (ESI) m/z 611.3 [M + 1]⁺ | ¹H NMR (400 MHz, MeOD-d₄) δ 8.31 (d, J = 8.5 Hz, 2H), 8.07 (d, J = 8.7 Hz, 1H), 7.97 (s, 1H), 7.85-7.83 (m, 1H), 7.72 (d, J = 8.4 Hz, 1H), 7.56 (d, J = 8.5 Hz, 1H), 7.11 (d, J = 2.2 Hz, 1H), 7.06-7.05 (m, 1H), 5.18-5.13 (m, 2H), 4.47- 4.41 (m, 2H), 4.25 (m, 1H), 4.18-4.11 (m, |
| | | | | 2H), 4.07-3.83 |
| | | | | (m, 4H), 3.80 (m, |
| | | | | 1H), 3.25 (m, |
| | | | | 1H), 3.15 (m, |
| | | | | 3H), 2.98-2.89 |
| | | | | (m, 2H), 2.81- |
| | | | | 2.75 (m, 1H), |
| | | | | 2.60-2.40 (m, |
| | | | | 2H), 2.24-2.14 |
| | | | | (m, 3H), 2.06- |
| | | | | 1.92 (m, 5H) |
| 22 | 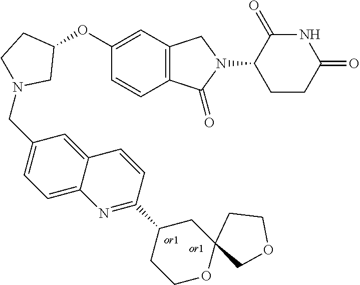 [SMILES: O=C1CC[C@H](N2Cc3cc(O[C@H]4CCN(Cc5ccc6nc([C@H]7CCO[C@@]8(CCOC8)C7)ccc6c5)C4)ccc3C2=O)C(=O)N1] | rel-(3S)-3-(5- (((3S)-1-((2-(2,6- dioxaspiro[4.5] decan-9- yl)quinolin-6- yl)methyl) pyrrolidin-3- yl)oxy)-1- oxoisoindolin-2- yl)piperidine-2,6- dione | MS (ESI) m/z 611.3 [M + 1]⁺ | ¹H NMR (400 MHz, MeOD-d₄) δ 8.34-8.27 (m, 2H), 8.06 (d, J = 8.7 Hz, 1H), 7.97 (d, J = 2.0 Hz, 1H), 7.85-7.83 (m, 1H), 7.71 (d, J = 8.4 Hz, 1H), 7.56 (d, J = 8.6 Hz, 1H), 7.11 (d, J = 2.1 Hz, 1H), 7.06-7.05 (m, 1H), 5.18-5.13 (m, 2H), 4.47- 4.41 (m, 2H), 4.34-4.22 (m, 2H), 4.07-3.83 |
| | | | | (m, 4H), 3.80 (m, |
| | | | | 1H), 3.62 (m, |
| | | | | 1H), 3.40-3.33 |
| | | | | (m, 2H), 3.29- |
| | | | | 3.26 (m, 2H), |
| | | | | 3.11 (m, 1H), |
| | | | | 2.95-2.89 (m, |
| | | | | 1H), 2.81-2.73 |
| | | | | (m, 1H), 2.60- |
| | | | | 2.40 (m, 3H), |
| | | | | 2.26-2.12 (m, |
| | | | | 2H), 2.06-1.87 |
| | | | | (m, 5H) |
| 23 | 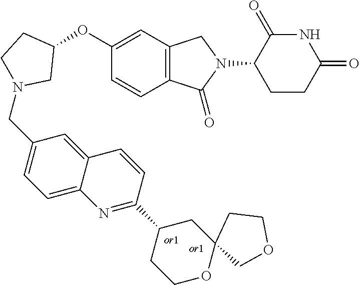 [SMILES: O=C1CC[C@H](N2Cc3cc(O[C@H]4CCN(Cc5ccc6nc([C@H]7CCO[C@]8(CCOC8)C7)ccc6c5)C4)ccc3C2=O)C(=O)N1] | rel-(3S)-3-(5- (((3S)-1-((2-(2,6- dioxaspiro[4.5] decan-9- yl)quinolin-6- yl)methyl) pyrrolidin-3- yl)oxy)-1- oxoisoindolin-2- yl)piperidine-2,6- dione | MS (ESI) m/z 611.3 [M + 1]⁺ | ¹H NMR (400 MHz, MeOD-d₄) δ 8.34-8.27 (m, 2H), 8.06 (d, J = 8.7 Hz, 1H), 7.97 (d, J = 2.0 Hz, 1H), 7.85-7.83 (m, 1H), 7.71 (d, J = 8.4 Hz, 1H), 7.56 (d, J = 8.6 Hz, 1H), 7.11 (d, J = 2.1 Hz, 1H), 7.06-7.05 (m, 1H), 5.18-5.13 (m, 2H), 4.47- 4.41 (m, 2H), 4.34-4.22 (m, 2H), 4.07-3.83 |
| | | | | (m, 4H), 3.80 (m, |
| | | | | 1H), 3.62 (m, |
| | | | | 1H), 3.40-3.33 |
| | | | | (m, 2H), 3.29- |
| | | | | 3.26 (m, 2H), |
| | | | | 3.11 (m, 1H), |
| | | | | 2.95-2.89 (m, |
| | | | | 1H), 2.81-2.73 |
| | | | | (m, 1H), 2.60- |
| | | | | 2.40 (m, 3H), |
| | | | | 2.26-2.12 (m, |
| | | | | 2H), 2.06-1.87 |
| | | | | (m, 5H) |
| 24 | 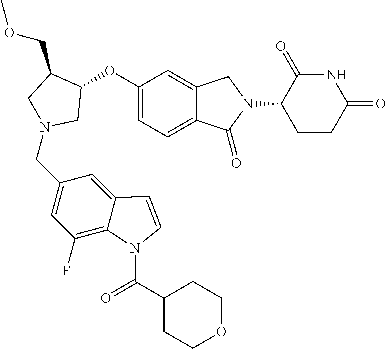 [SMILES: COC[C@@H]1CN(Cc2cc(F)c3c(ccn3C(=O)C3CCOCC3)c2)C[C@H]1Oc1ccc2c(c1)CN([C@H]1CCC(=O)NC1=O)C2=O] | (3S)-3-(5- {[(3S,4S)-1-{[7- fluoro-1-(oxane- 4-carbonyl)-1H- indol-5- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 633.4 [M + 1]⁺ | ¹H NMR (400 MHz, MeOD-d₄) δ 7.90-7.88 (m, 1H), 7.77-7.63 (m, 1H), 7.41 (m, 1H), 7.21-7.02 (m, 3H), 6.74- 6.72 (m, 1H), 5.16-5.08 (m, 1H), 4.43 (m, 2H), 4.12-3.96 (m, 2H), 3.93- 3.75 (m, 2H), 3.72-3.41 (m, 6H), 3.38 (s, 3H), 3.17 (m, 1H), 3.05-2.94 (m, 2H), 2.89 (m, 1H), 2.84-2.66 (m, 2H), 2.47 (m, 2H), 2.16 (m, 1H), 2.01-1.79 |
| | | | | (m, 4H) |
| 25 | 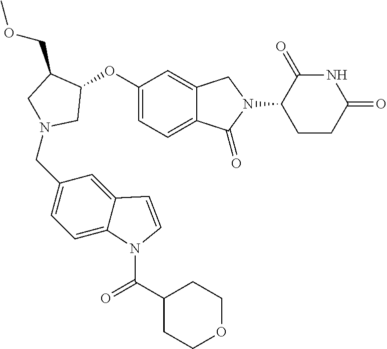 [SMILES: COC[C@@H]1CN(Cc2ccc3c(ccn3C(=O)C3CCOCC3)c2)C[C@H]1Oc1ccc2c(c1)CN([C@H]1CCC(=O)NC1=O)C2=O] | (3S)-3-(5- {[(3S,4S)-4- (methoxymethyl)- 1-{[1-(oxane-4- carbonyl)-1H- indol-5- yl]methyl} pyrrolidin-3- yl]oxy}-1- oxo-2,3-dihydro- 1H-isoindol-2- yl)piperidine-2,6- dione | MS (ESI) m/z 615.3 [M + 1]⁺ | ¹H NMR (400 MHz, MeOD-d₄) δ 8.41 (d, J = 8.5 Hz, 1H), 7.88 (d, J = 3.9 Hz, 1H), 7.70 (d, J = 8.5 Hz, 1H), 7.61 (d, J = 1.7 Hz, 1H), 7.37 (dd, J = 8.5, 1.7 Hz, 1H), 7.13 (d, J = 2.2 Hz, 1H), 7.07 (dd, J = 8.5, 2.2 Hz, 1H), 6.72 (d, J = 3.8 Hz, 1H), 5.12 (dd, J = 13.3, 5.1 Hz, 1H), 4.48- 4.36 (m, 2H), 4.08-3.88 (m, 4H), 3.63-3.60 m, 2H), 3.57-3.49 (m, 3H), 3.38 (s, |
| | | | | 3H), 3.25-3.23 |
| | | | | (m, 2H), 3.08 (m, |
| | | | | 2H), 2.95-2.85 |
| | | | | (m, 1H), 2.82- |
| | | | | 2.69 (m, 2H), |
| | | | | 2.62-2.51 (m, |
| | | | | 1H), 2.45-2.43 |
| | | | | (m, 1H), 2.19- |
| | | | | 2.13 (m, 1H), |
| | | | | 1.92-1.85 (m, |
| | | | | 4H) |
| 26 | 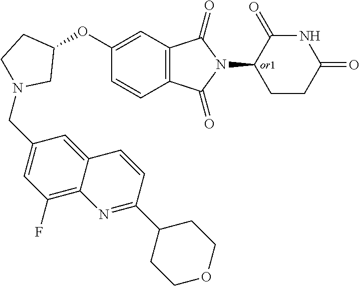 [SMILES: O=C1CC[C@@H](N2C(=O)c3ccc(O[C@H]4CCN(Cc5cc(F)c6nc(C7CCOCC7)ccc6c5)C4)cc3C2=O)C(=O)N1] | 2-((R*)-2,6- dioxopiperidin-3- yl)-5-(((S)-1-((8- fluoro-2- (tetrahydro-2H- pyran-4- yl)quinolin-6- yl)methyl) pyrrolidin-3- yl)oxy)isoindoline- 1,3-dione | MS (ESI) m/z 587.2 [M + 1]⁺ | ¹H NMR (400 MHz, MeOD-d₄) δ 8.33 (d, J = 8.6 Hz, 1H), 7.86- 7.79 (m, 2H), 7.67-7.58 (m, 2H), 7.41 (d, J = 2.3 Hz, 1H), 7.33 (m, 1H), 5.31 (m, 1H), 5.13 (m, 1H), 4.37-4.32 (m, 2H), 4.16- 4.08 (m, 2H), 3.80-3.61 (m, 2H), 3.51 (m, 1H), 3.43 (m, 2H), 3.32-3.21 (m, 1H), 3.21 (m, |
| | | | | 2H), 2.89 (m, |
| | | | | 1H), 2.82-2.73 |
| | | | | (m, 1H), 2.72 (m, |
| | | | | 1H), 2.71-2.55 |
| | | | | (m, 1H), 2.29- |
| | | | | 2.21 (m, 1H), |
| | | | | 2.19-1.98 (m, |
| | | | | 3H), 1.93 (m, |
| | | | | 2H) |
| 27 | 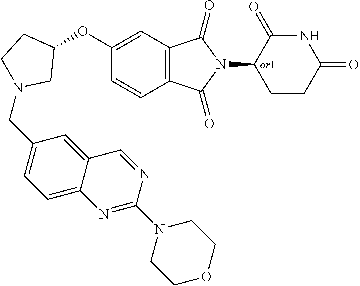 [SMILES: O=C1CC[C@@H](N2C(=O)c3ccc(O[C@H]4CCN(Cc5ccc6nc(N7CCOCC7)ncc6c5)C4)cc3C2=O)C(=O)N1] | 2-((R*)-2,6- dioxopiperidin-3- yl)-5-(((S)-1-((2- morpholino- quinazolin-6- yl)methyl) pyrrolidin-3- yl)oxy)isoindoline- 1,3-dione | MS (ESI) m/z 571.3 [M + 1]⁺ | ¹H NMR (400 MHz, MeOD-d₄) δ 9.15 (s, 1H), 7.96 (d, J = 2.1 Hz, 1H), 7.88- 7.80 (m, 2H), 7.65 (d, J = 8.8 Hz, 1H), 7.44 (d, J = 2.3 Hz, 1H), 7.35 (m, 1H), 5.41 (m, 1H), 5.13 (m, 1H), 4.54 (d, J = 2.2 Hz, 2H), 3.97 (m, 4H), 3.80 (m, 4H), 3.77-3.59 (m, 3H), 3.51 (m, 1H), 2.89 (m, |
| | | | | 1H), 2.82-2.65 |
| | | | | (m, 2H), 2.64 (m, |
| | | | | 1H), 2.40-2.32 |
| | | | | (m, 1H), 2.19- |
| | | | | 2.09 (m, 1H) |
| 28 | 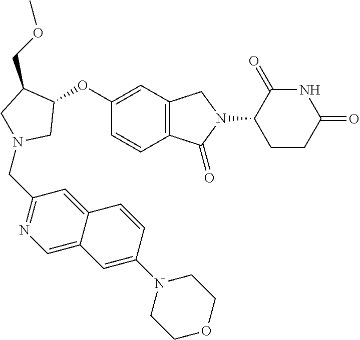 [SMILES: COC[C@@H]1CN(Cc2cc3ccc(N4CCOCC4)cc3cn2)C[C@H]1Oc1ccc2c(c1)CN([C@H]1CCC(=O)NC1=O)C2=O] | (3S)-3-(5- {[(3S,4S)-4- (methoxymethyl)- 1-{[7-(morpholin- 4-yl)isoquinolin- 3-yl]methyl} pyrrolidin-3- yl]oxy}-1- oxo-2,3-dihydro- 1H-isoindol-2- yl)piperidine-2,6- dione | MS (ESI) m/z 600.3 [M + 1]⁺ | ¹H NMR (400 MHz, DMSO-d₆) δ 9.04 (d, J = 3.8 Hz, 1H), 8.15 (d, J = 3.8 Hz, 1H), 7.86-7.74 (m, 1H), 7.61 (m, 3H), 7.31 (m, 1H), 7.14 (m, 1H), 7.03 (m, 1H), 5.05 (m, 1H), 4.73 (s, 1H), 4.41-4.25 (m, 2H), 3.87-3.75 (m, 6H), 3.66- 3.48 (m, 2H), 3.31-3.20 (m, 7H), 3.03-2.81 (m, 4H), 2.61 (m, 2H), 2.40-2.25 (m, 2H), 1.97 (m, |
| | | | | 1H) |
| 29 | 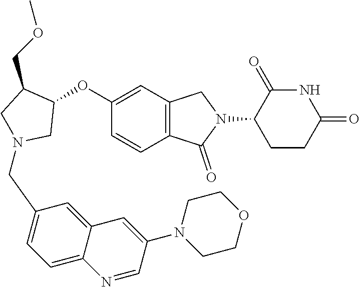 [SMILES: COC[C@@H]1CN(Cc2ccc3ncc(N4CCOCC4)cc3c2)C[C@H]1Oc1ccc2c(c1)CN([C@H]1CCC(=O)NC1=O)C2=O] | (3S)-3-(5- {[(3S,4S)-4- (methoxymethyl)- 1-{[3-(morpholin- 4-yl)quinolin-6- yl]methyl} pyrrolidin-3- yl]oxy}-1- oxo-2,3-dihydro- 1H-isoindol-2- yl)piperidine-2,6- dione | MS (ESI) m/z 600.3 [M + 1]⁺ | ¹H NMR (400 MHz, MeOD-d₄) δ 8.80 (s, 1H), 8.28 (s, 1H), 7.93 (d, J = 8.5 Hz, 1H), 7.82 (d, J = 1.9 Hz, 1H), 7.71 (d, J = 8.4 Hz, 1H), 7.61 (dd, J = 8.7, 1.8 Hz, 1H), 7.53 (d, J = 2.8 Hz, 1H), 7.13 (d, J = 2.1 Hz, 1H), 7.07 (dd, J = 8.6, 2.1 Hz, 1H), 5.12 (dd, J = 13.3, 5.2 Hz, 1H), 5.06 (m, 1H), 5.03-4.93 |
| | | | | (m, 2H), 4.49- |
| | | | | 4.35 (m, 2H), |
| | | | | 4.30-4.16 (m, |
| | | | | 2H), 3.94-3.87 |
| | | | | (m, 4H), 3.63- |
| | | | | 3.49 (m, 2H), |
| | | | | 3.48-3.40 (m, |
| | | | | 2H), 3.35 (s, 3H), |
| | | | | 3.30-3.28 (m, |
| | | | | 3H), 2.98-2.70 |
| | | | | (m, 4H), 2.46 (m, |
| | | | | 1H), 2.16 (m, |
| | | | | 1H) |
| 30 | 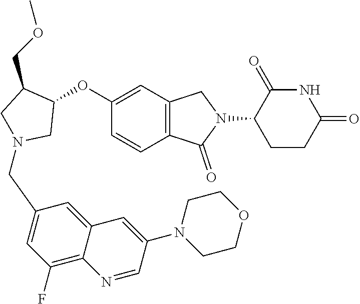 [SMILES: COC[C@@H]1CN(Cc2cc(F)c3ncc(N4CCOCC4)cc3c2)C[C@H]1Oc1ccc2c(c1)CN([C@H]1CCC(=O)NC1=O)C2=O] | (S)-3-(5- (((3S,4S)-1-((8- fluoro-3- morpholino- quinolin-6- yl)methyl)-4- (methoxymethyl) pyrrolidin-3- yl)oxy)-1- oxoisoindolin-2- yl)piperidine-2,6- dione | MS (ESI) m/z 618.3 [M + H]⁺ | ¹H NMR (400 MHz, MeOD-d₄) δ 8.79 (d, J = 2.7 Hz, 1H), 7.72 (d, J = 8.5 Hz, 1H), 7.60-7.54 (m, 2H), 7.34 (dd, J = 11.5, 1.7 Hz, 1H), 7.15 (d, J = 2.2 Hz, 1H), 7.09 (dd, J = 8.4, 2.2 Hz, 1H), 5.13 (dd, J = 13.3, 5.2 Hz, 1H), 4.90 (m, 1H), 4.51-4.36 (m, 2H), 3.97 (d, J = 2.6 Hz, 2H), 3.95-3.88 (m, 4H), 3.55 (d, J = |
| | | | | 6.3 Hz, 2H), 3.38 |
| | | | | (s, 3H), 3.25 (m, |
| | | | | 4H), 3.20 (m, |
| | | | | 1H), 3.16-3.04 |
| | | | | (m, 2H), 2.92 (m, |
| | | | | 1H), 2.84-2.75 |
| | | | | (m, 2H), 2.60 (m, |
| | | | | 1H), 2.48 (m, |
| | | | | 1H), 2.17 (m, |
| | | | | 1H) |
| 31 | 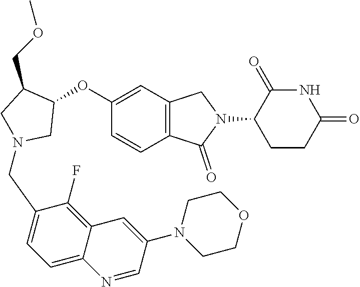 [SMILES: COC[C@@H]1CN(Cc2ccc3ncc(N4CCOCC4)cc3c2F)C[C@H]1Oc1ccc2c(c1)CN([C@H]1CCC(=O)NC1=O)C2=O] | (3S)-3-(5- {[(3S,4S)-1-{[5- fluoro-3- (morpholin-4- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.3 [M + H]⁺ | ¹H NMR (400 MHz, MeOD-d₄) δ 8.84 (d, J = 2.8 Hz, 1H), 7.76- 7.64 (m, 3H), 7.59 (t, J = 8.1 Hz, 1H), 7.13 (d, J = 2.2 Hz, 1H), 7.06 (dd, J = 8.4, 2.2 Hz, 1H), 5.12 (dd, J = 13.3, 5.2 Hz, 1H), 4.84- 4.78 (m, 1H), 4.48-4.37 (m, 2H), 3.99-3.88 (m, 6H), 3.54- 3.48 (m, 2H), 3.36 (m, 7H), |
| | | | | 3.14 (m, 1H), |
| | | | | 3.04-2.94 (m, |
| | | | | 2H), 2.93-2.86 |
| | | | | (m, 1H), 2.79- |
| | | | | 2.77 (m, 1H), |
| | | | | 2.69-2.67 (m, |
| | | | | 1H), 2.52-2.42 |
| | | | | (m, 2H), 2.16- |
| | | | | 2.14 (m, 1H) |
| 32 | 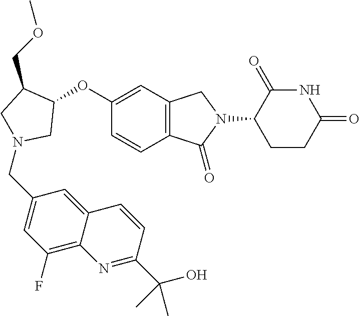 [SMILES: COC[C@@H]1CN(Cc2cc(F)c3nc(C(C)(C)O)ccc3c2)C[C@H]1Oc1ccc2c(c1)CN([C@H]1CCC(=O)NC1=O)C2=O] | (3S)-3-(5- {[(3S,4S)-1-{[8- fluoro-2-(2- hydroxypropan-2- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 591.4 [M + 1]⁺ | NMR (400 MHz, MeOD-d₄) δ 8.28-8.26 (m, 1H), 7.85-7.83 (m, 1H), 7.68- 7.65 (m, 2H), 7.52-7.51 (m, 1H), 7.08-7.05 (m, 2H), 5.10- 5.08 (m, 1H), 4.40-4.36 (m, 2H), 3.98-3.80 (m, 2H), 3.52 (m, 2H), 3.35-3.30 (m, 4H), 3.19- 2.96 (m, 3H), 2.92-2.64 (m, 3H), 2.46-2.43 (m, 2H), 2.15 (m, 1H), 1.63 (s, 6H) |
| 33 | 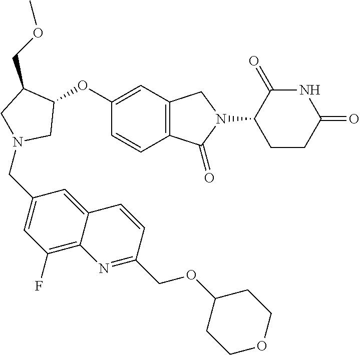 [SMILES: COC[C@@H]1CN(Cc2cc(F)c3nc(COC4CCOCC4)ccc3c2)C[C@H]1Oc1ccc2c(c1)CN([C@H]1CCC(=O)NC1=O)C2=O] | (3S)-3-(5- {[(3S,4S)-1-({8- fluoro-2-[(oxan- 4- yloxy)methyl] quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 647.2 [M + 1]⁺ | ¹H NMR (400 MHz, DMSO-d₆) δ 8.41 (dd, J = 8.6, 1.6 Hz, 1H), 7.75-7.67 (m, 2H), 7.61 (d, J = 8.4 Hz, 1H), 7.53 (dd, J = 11.8, 1.6 Hz, 1H), 7.16 (d, J = 2.1 Hz, 1H), 7.04 (dd, J = 8.4, 2.2 Hz, 1H), 5.06 (dd, J = 13.2, 5.1 Hz, 1H), 4.81- 4.72 (m, 3H), 4.37 (d, J = 17.3 Hz, 1H), 4.25 (d, J = 17.3 Hz, 1H), 3.88-3.77 (m, 3H), 3.73 (m, 1H), 3.73-3.62 (m, 1H), 3.52- |
| | | | | 3.47 (m, 1H), |
| | | | | 3.44-3.30 (m, |
| | | | | 3H), 3.26 (s, 3H), |
| | | | | 2.99-2.93 (m, |
| | | | | 1H), 2.96-2.83 |
| | | | | (m, 2H), 2.76 (m, |
| | | | | 1H), 2.64-2.52 |
| | | | | (m, 2H), 2.44- |
| | | | | 2.29 (m, 1H), |
| | | | | 2.25 (m, 1H), |
| | | | | 2.02-1.89 (m, |
| | | | | 3H), 1.58-1.44 |
| | | | | (m, 2H) |
| 34 | 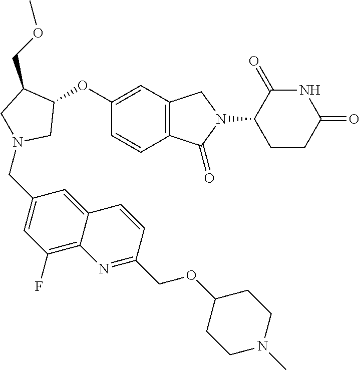 [SMILES: COC[C@@H]1CN(Cc2cc(F)c3nc(COC4CCN(C)CC4)ccc3c2)C[C@H]1Oc1ccc2c(c1)CN([C@H]1CCC(=O)NC1=O)C2=O] | (3S)-3-(5- {[(3S,4S)-1-[(8- fluoro-2-{[(1- methylpiperidin- 4- yl)oxy]methyl} quinolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 660.6 [M + 1]⁺ | ¹H NMR (400 MHz, MeOD-d₄) δ 8.49 (s, 2H, formic acid-H), 8.37 (dd, J = 8.6, 1.5 Hz, 1H), 7.77 (d, J = 8.6 Hz, 1H), 7.74-7.66 (m, 2H), 7.59 (dd, J = 11.8, 1.7 Hz, 1H), 7.14 (d, J = 2.1 Hz, 1H), 7.08 (dd, J = 8.5, 2.2 Hz, 1H), 5.11 (dd, J = 13.3, 5.1 Hz, 1H), 4.84 (m, 1H), 4.49-4.38 (m, 2H), 3.97- 3.83 (m, 3H), 3.59-3.50 (m, 2H), 3.38 (m, 6H), 3.13 (m, 3H), 3.06-2.93 |
| | | | | (m, 3H), 2.92- |
| | | | | 2.69 (m, 6H), |
| | | | | 2.45 (m, 2H), |
| | | | | 2.20-2.02 (m, |
| | | | | 5H) |
| 35 | 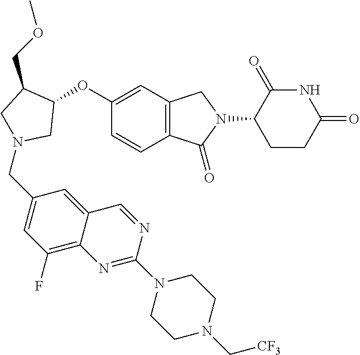 [SMILES: COC[C@@H]1CN(Cc2cc(F)c3nc(N4CCN(CC(F)(F)F)CC4)ncc3c2)C[C@H]1Oc1ccc2c(c1)CN([C@H]1CCC(=O)NC1=O)C2=O] | (3S)-3-(5- {[(3S,4S)-1-({8- fluoro-2-[4- (2,2,2- trifluoroethyl) piperazin-1- yl]quinazolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 700.4 [M + 1]⁺ | ¹H NMR (400 MHz, MeOD-d₄) δ 9.10 (d, J = 1.5 Hz, 1H), 7.71 (d, J = 8.3 Hz, 1H), 7.58-7.50 (m, 2H), 7.18-7.05 (m, 2H), 5.12 (dd, J = 13.3, 5.1 Hz, 1H), 4.85 (m, 1H), 4.50-4.34 (m, 2H), 4.01 (m, 4H), 3.82 (m, 2H), 3.53 (m, 2H), 3.39 (s, 3H), 3.30-3.22 (m, 1H), 3.20-3.10 (m, 3H), 2.99 (m, 2H), 2.95-2.85 (m, 1H), 2.79- 2.59 (m, 6H), 2.48-2.45 (m, |
| | | | | 2H), 2.21-2.12 |
| | | | | (m, 1H) |
| 36 | 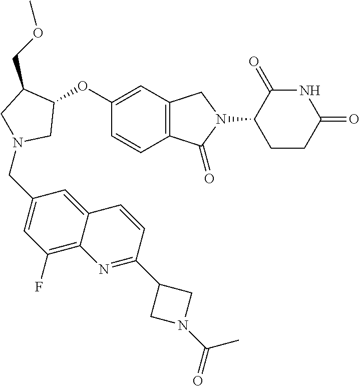 | (3S)-3-(5- {[(3S,4S)-1-{[2- (1-acetylazetidin- 3-yl)-8- fluoroquinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 680.2 [M + 1]⁺ | ¹H NMR (400 MHz, MeOD-d₄) δ 9.30 (s, 1H), 8.06-7.88 (m, 3H), 7.80 (m, 1H), 7.72 (m, 1H), 7.17 (d, J = 2.2 Hz, 1H), 7.09 (m, 1H), 5.21 (d, J = 4.7 Hz, 1H), 5.12 (m, 1H), 4.80-4.66 (m, 2H), 4.52-4.37 (m, 2H), 3.98 (m, 1H), 3.88 (m, 1H), 3.78 (m, 1H), 3.69 (m, 1H), 3.64-3.48 (m, 4H), 3.44 (m, 5H), 2.95 (m, 5H), 2.80 (m, 1H), 2.42-2.35 (m, 1H), 2.20- 2.10 (m, 1H), |
| | | | | 2.08-1.98 (m, |
| | | | | 4H) |
| 37 | 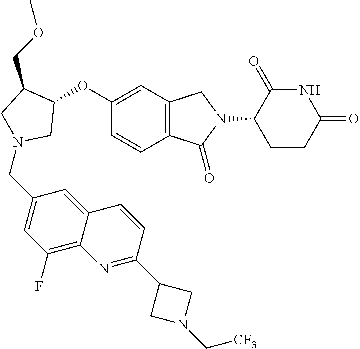 | (3S)-3-(5- {[(3S,4S)-1-({8- fluoro-2-[1- (2,2,2- trifluoroethyl) azetidin-3- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 681.2 [M + 1]⁺ | ¹H NMR (400 MHz, MeOD-d₄) δ 9.03 (s, 1H), 7.79 (d, J = 9.1 Hz, 1H), 7.66- 7.58 (m, 3H), 7.32 (d, J = 2.4 Hz, 1H), 7.15 (d, J = 2.2 Hz, 1H), 7.04 (dd, J = 8.4, 2.2 Hz, 1H), 5.06-5.02 (m, 1H), 4.75-4.73 (m, 1H), 4.38 (d, J = 17.3 Hz, 1H), 4.26 (d, J = 17.3 Hz, 1H), 3.82 (s, 2H), 3.43-3.39 (m, 2H), 3.32- 3.19 (m, 9H), 3.03-2.99 (m, 1H), 2.96-2.83 |
| | | | | (m, 2H), 2.82- |
| | | | | 2.79 (m, 5H), |
| | | | | 2.66-2.53 (m, |
| | | | | 2H), 2.45-2.38 |
| | | | | (m, 1H), 2.35- |
| | | | | 2.26 (m, 1H), |
| | | | | 2.04-1.94 (m, |
| | | | | 1H) |
| 38 | 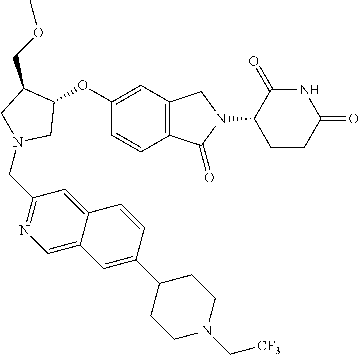 [SMILES: COC[C@@H]1CN(Cc2cc3ccc(C4CCN(CC(F)(F)F)CC4)cc3cn2)C[C@H]1Oc1ccc2c(c1)CN([C@H]1CCC(=O)NC1=O)C2=O] | (3S)-3-(5- {[(3S,4S)-4- (methoxymethyl)- 1-({7-[1-(2,2,2- trifluoroethyl) piperidin-4- yl]isoquinolin-3- yl}methyl) pyrrolidin-3- yl]oxy}-1- oxo-2,3-dihydro- 1H-isoindol-2- yl)piperidine-2,6- dione | MS (ESI) m/z 680.2 [M + 1]⁺ | ¹H NMR (400 MHz, MeOD-d₄) δ 9.30 (s, 1H), 8.06-7.88 (m, 3H), 7.80 (m, 1H), 7.72 (m, 1H), 7.17 (d, J = 2.2 Hz, 1H), 7.09 (m, 1H), 5.21 (d, J = 4.7 Hz, 1H), 5.12 (m, 1H), 4.80-4.66 (m, 2H), 4.52-4.37 (m, 2H), 3.98 (m, 1H), 3.88 (m, 1H), 3.78 (m, 1H), 3.69 (m, 1H), 3.64-3.48 (m, 4H), 3.44 (m, 5H), 2.95 (m, 5H), 2.80 (m, 1H), 2.42-2.35 |
| | | | | (m, 1H), 2.20- |
| | | | | 2.10 (m, 1H), |
| | | | | 2.08-1.98 (m, |
| | | | | 4H) |
| 39 | 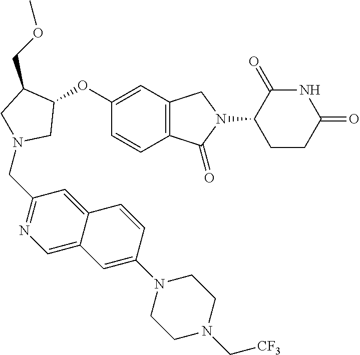 [SMILES: COC[C@@H]1CN(Cc2cc3ccc(N4CCN(CC(F)(F)F)CC4)cc3cn2)C[C@H]1Oc1ccc2c(c1)CN([C@H]1CCC(=O)NC1=O)C2=O] | (3S)-3-(5- {[(3S,4S)-4- (methoxymethyl)- 1-({7-[4-(2,2,2- trifluoroethyl) piperazin-1- yl]isoquinolin-3- yl}methyl)pyrrolidin- 3-yl]oxy}-1- oxo-2,3-dihydro- 1H-isoindol-2- yl)piperidine-2,6- dione | MS (ESI) m/z 681.2 [M + 1]⁺ | ¹H NMR (400 MHz, MeOD-d₄) δ 9.03 (s, 1H), 7.79 (d, J = 9.1 Hz, 1H), 7.66- 7.58 (m, 3H), 7.32 (d, J = 2.4 Hz, 1H), 7.15 (d, J = 2.2 Hz, 1H), 7.04 (dd, J = 8.4, 2.2 Hz, 1H), 5.06-5.02 (m, 1H), 4.75-4.73 (m, 1H), 4.38 (d, J = 17.3 Hz, 1H), 4.26 (d, J = 17.3 Hz, 1H), 3.82 (s, 2H), 3.43-3.39 (m, 2H), 3.32- 3.19 (m, 9H), 3.03-2.99 (m, 1H), 2.96-2.83 |
| | | | | (m, 2H), 2.82- |
| | | | | 2.79 (m, 5H), |
| | | | | 2.66-2.53 (m, |
| | | | | 2H), 2.45-2.38 |
| | | | | (m, 1H), 2.35- |
| | | | | 2.26 (m, 1H), |
| | | | | 2.04-1.94 (m, |
| | | | | 1H) |
| 40 | 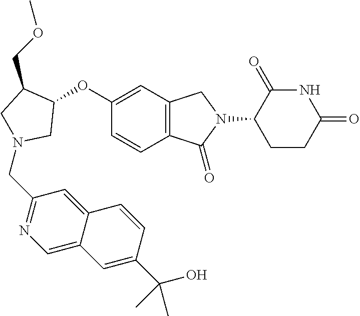 [SMILES: COC[C@@H]1CN(Cc2cc3ccc(C(C)(C)O)cc3cn2)C[C@H]1Oc1ccc2c(c1)CN([C@H]1CCC(=O)NC1=O)C2=O] | (3S)-3-(5- {[(3S,4S)-1-{[7- (2- hydroxypropan-2- yl)isoquinolin-3- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 573.2 [M + 1]⁺ | ¹H NMR (400 MHz, MeOD-d₄) δ 9.27 (s, 1H), 8.33 (s, 1H), 8.23-8.18 (m, 1H), 8.00 (dd, J = 8.7, 1.8 Hz, 1H), 7.96-7.87 (m, 2H), 7.73 (d, J = 8.4 Hz, 1H), 7.17 (d, J = 2.2 Hz, 1H), 7.11 (dd, J = 8.4, 2.2 Hz, 1H), 5.13 (dd, J = 13.3, 5.1 Hz, 1H), 5.06 (dd, J = 4.8, 2.4 Hz, 1H), 4.48 (d, J = 17.1 Hz, 1H), 4.44 (s, 1H), 4.42-4.33 |
| | | | | (m, 2H), 3.69- |
| | | | | 3.44 (m, 5H), |
| | | | | 3.42 (s, 3H), 3.06 |
| | | | | (m, 1H), 2.98- |
| | | | | 2.70 (m, 2H), |
| | | | | 2.56-2.41 (m, |
| | | | | 1H), 2.23-2.10 |
| | | | | (m, 1H), 1.65 (s, |
| | | | | 6H) |
| 41 | 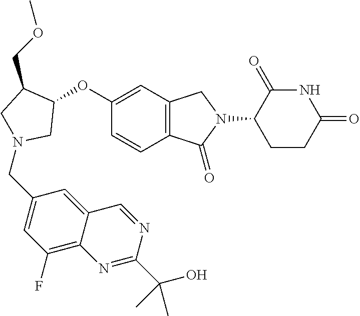 [SMILES: COC[C@@H]1CN(Cc2cc(F)c3nc(C(C)(C)O)ncc3c2)C[C@H]1Oc1ccc2c(c1)CN([C@H]1CCC(=O)NC1=O)C2=O] | (3S)-3-(5- {[(3S,4S)-1-{[8- fluoro-2-(2- hydroxypropan-2- yl)quinazolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 592.2 [M + 1]⁺ | ¹H NMR (400 MHz, MeOD-d₄) δ 9.65 (m, 1H), 7.92 (d, J = 1.6 Hz, 1H), 7.84- 7.81 (m, 1H), 7.64 (d, J = 8.4 Hz, 1H), 7.16 (d, J = 2.2 Hz, 1H), 7.06-7.04 (m, 1H), 5.05-5.02 (m, 1H), 4.76- 4.70 (m, 1H), 4.40 (d, J = 17.3 Hz, 1H), 4.27 (d, J = 17.4 Hz, 1H), 3.89-3.76 (m, 2H), 3.45-3.39 (m, 2H), 3.29 (s, 3H), 3.01-2.90 |
| | | | | (m, 2H), 2.89- |
| | | | | 2.81 (m, 1H), |
| | | | | 2.80-2.78 (m, |
| | | | | 1H), 2.68-2.54 |
| | | | | (m, 2H), 2.38- |
| | | | | 2.27 (m, 2H), |
| | | | | 2.01-1.95 (m, |
| | | | | 1H), 1.60 (s, 6H) |
| 42 | 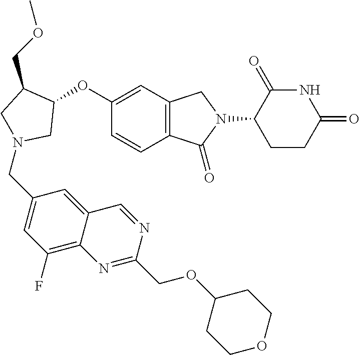 [SMILES: COC[C@@H]1CN(Cc2cc(F)c3nc(COC4CCOCC4)ncc3c2)C[C@H]1Oc1ccc2c(c1)CN([C@H]1CCC(=O)NC1=O)C2=O] | (3S)-3-(5- {[(3S,4S)-1-({8- fluoro-2-[(oxan- 4- yloxy)methyl] quinazolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 648.5 [M + 1]⁺ | ¹H NMR (400 MHz, DMSO-d₆) δ 9.64 (s, 1H), 7.93 (s, 1H), 7.84 (s, 1H), 7.62 (d, J = 8.4 Hz, 1H), 7.16 (s, 1H), 7.05 (d, J = 8.5 Hz, 1H), 5.05 (dd, J = 13.6, 5.0 Hz, 1H), 4.84 (s, 2H), 4.77 (m, 1H), 4.38 (d, J = 17.3 Hz, 1H), 4.25 (d, J = 17.2 Hz, 1H), 3.83 (m, 4H), 3.80-3.69 (m, 2H), 3.39-3.29 (m, 3H), 3.27 (s, 3H), 2.97-2.79 (m, 4H), 2.59 (m, 2H), 2.41-2.30 |
| | | | | (m, 2H), 2.02- |
| | | | | 1.89 (m, 3H), |
| | | | | 1.56-1.42 (m, |
| | | | | 2H) |
| 43 | 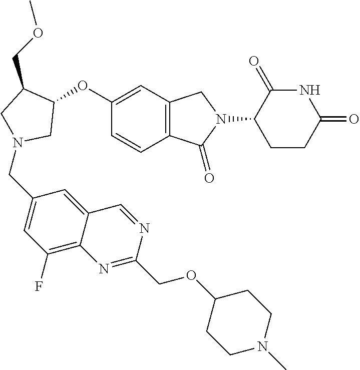 [SMILES: COC[C@@H]1CN(Cc2cc(F)c3nc(COC4CCN(C)CC4)ncc3c2)C[C@H]1Oc1ccc2c(c1)CN([C@H]1CCC(=O)NC1=O)C2=O] | (3S)-3-(5- {[(3S,4S)-1-[(8- fluoro-2-{[(1- methylpiperidin- 4- yl)oxy]methyl} quinazolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 661.4 [M + 1]⁺ | ¹H NMR (400 MHz, DMSO-d₆) δ 9.71 (s, 1H), 8.18 (s, 1H), 8.05 (d, J = 11.0 Hz, 1H), 7.67 (d, J = 8.4 Hz, 1H), 7.21 (S, 1H), 7.09 (d, J = 8.5 Hz, 1H), 5.12-5.01 (m, 2H), 4.91 (d, J = 6.2 Hz, 2H), 4.80-4.60 (m, 2H), 4.40 (d, J = 17.4 Hz, 1H), 4.27 (d, J = 17.3 Hz, 1H), 3.74- 3.68 (m, 4H), 3.49-3.42 (m, 3H), 3.30 (s, 3H), 3.25-3.14 (m, 2H), 2.98-2.70 (m, 6H), 2.65- |
| | | | | 2.56 (m, 1H), |
| | | | | 2.37-2.34 (m, |
| | | | | 1H), 2.27 (m, |
| | | | | 1H), 2.13 (m, |
| | | | | 1H), 2.03-1.95 |
| | | | | (m, 1H), 1.86 (m, |
| | | | | 1H), 1.66 (m, |
| | | | | 1H) |
| 44 | 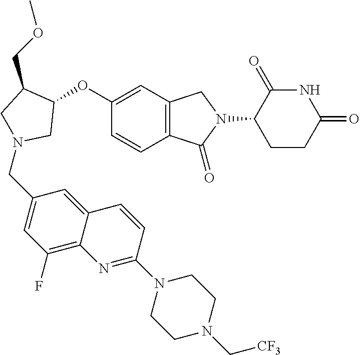 [SMILES: COC[C@@H]1CN(Cc2cc(F)c3nc(N4CCN(CC(F)(F)F)CC4)ccc3c2)C[C@H]1Oc1ccc2c(c1)CN([C@H]1CCC(=O)NC1=O)C2=O] | (3S)-3-(5- {[(3S,4S)-1-({8- fluoro-2-[4- (2,2,2- trifluoroethyl) piperazin-1- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 699.3 [M + 1]⁺ | ¹H NMR (400 MHz, MeOD-d₄) δ 8.10-7.90 (m, 1H), 7.68 (m, 1H), 7.40 (d, J = 1.8 Hz, 1H), 7.32 (m, 1H), 7.18 (m, 1H), 7.11 (d, J = 2.4 Hz, 1H), 7.16-7.00 (m, 1H), 5.09 (m, 1H), 4.82 (m, 1H), 4.48-4.33 (m, 2H), 3.85- 3.72 (m, 6H), 3.50 (m, 2H), 3.36 (s, 3H), 3.19-3.07 (m, 3H), 2.96 (d, J = 3.9 Hz, 2H), 2.92-2.82 (m, 1H), 2.82-2.72 |
| | | | | (m, 5H), 2.75- |
| | | | | 2.63 (m, 1H), |
| | | | | 2.52-2.42 (m, |
| | | | | 1H), 2.49-2.38 |
| | | | | (m, 1H), 2.19- |
| | | | | 2.10 (m, 1H) |
| 45 | 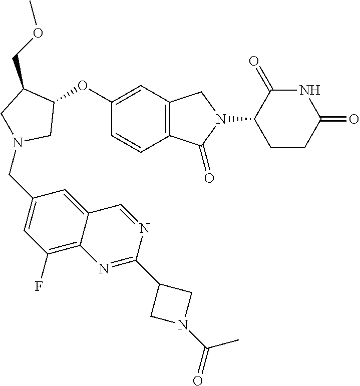 [SMILES: COC[C@@H]1CN(Cc2cc(F)c3nc(C4CN(C(C)=O)C4)ncc3c2)C[C@H]1Oc1ccc2c(c1)CN([C@H]1CCC(=O)NC1=O)C2=O] | (3S)-3-(5- {[(3S,4S)-1-{[2- (1-acetylazetidin- 3-yl)-8- fluoroquinazolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 631.2 [M + 1]⁺ | ¹H NMR (400 MHz, DMSO-d₆) δ 9.65 (s, 1H), 7.91 (s, 1H), 7.82 (d, J = 11.3 Hz, 1H), 7.65-7.58 (m, 1H), 7.14 (s, 1H), 7.03 (d, J = 8.5 Hz, 1H), 5.06-4.96 (m, 1H), 4.75 (s, 1H), 4.57 (m, 1H), 4.42 (m, 1H), 4.35-4.16 (m, 5H), 3.81 (m, 2H), 3.43 (m, 2H), 3.25 (s, 3H), 2.97-2.59 (m, 4H), 2.50 (m, 2H), 2.35-2.25 (m, 2H), 2.01- 1.93 (m, 1H), 1.80 (s, 3H) |
| 46 | 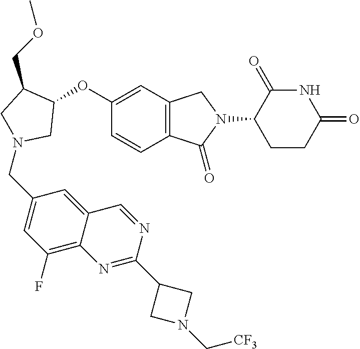 [SMILES: COC[C@@H]1CN(Cc2cc(F)c3nc(C4CN(CC(F)(F)F)C4)ncc3c2)C[C@H]1Oc1ccc2c(c1)CN([C@H]1CCC(=O)NC1=O)C2=O] | (3S)-3-(5- {[(3S,4S)-1-({8- fluoro-2-[1- (2,2,2- trifluoroethyl) azetidin-3- yl]quinazolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 671.4 [M + 1]⁺ | ¹H NMR (400 MHz, MeOD-d₄) δ 9.49 (s, 1H), 7.86 (m, 1H), 7.80-7.76 (m, 2H), 7.72-7.64 (m, 1H), 7.12- 7.08 (m, 2H), 5.09-5.05 (m, 1H), 4.96 (m, 1H) 4.48-4.41 (m, 2H), 4.20 (m, 1H), 3.98-3.90 (m, 4H), 3.85- 3.80 (m, 2H), 3.58-3.53 (m, 2H), 3.37 (s, 3H), 3.25 (m, 2H), 3.14-3.12 (m, 1H), 3.02-2.95 (m, 1H), 2.86- 2.82 (m, 2H), |
| | | | | 2.80-2.68 (m, |
| | | | | 2H), 2.56-2.38 |
| | | | | (m, 2H), 2.20- |
| | | | | 2.10 (m, 1H) |
| 47 | 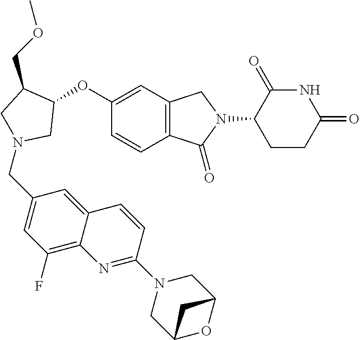 [SMILES: COC[C@@H]1CN(Cc2cc(F)c3nc(N4C[C@H]5C[C@@H](C4)O5)ccc3c2)C[C@H]1Oc1ccc2c(c1)CN([C@H]1CCC(=O)NC1=O)C2=O] | (S)-3-(5- (((3S,4S)-1-((2- ((1R,5S)-6-oxa-3- azabicyclo[3.1.1] heptan-3-yl)-8- fluoroquinolin-6- yl)methyl)-4- (methoxymethyl) pyrrolidin-3- yl)oxy)-1- oxoisoindolin-2- yl)piperidine-2,6- dione | MS (ESI) m/z 630.2 [M + 1]⁺ | ¹H NMR (400 MHz, MeOD-d₄) δ 8.33 (s, 1H), 8.04 (d, J = 8.9 Hz, 1H), 7.76- 7.68 (m, 1H), 7.53 (s, 1H), 7.40 (d, J = 11.8 Hz, 1H), 7.19-7.06 (m, 3H), 5.12 (dd, J = 13.4, 5.1 Hz, 1H), 4.99 (s, 1H), 4.83 (d, J = 6.4 Hz, 2H), 4.44 (d, J = 7.5 Hz, 2H), 4.09 (m, 2H), 4.01 (m, 2H), 3.84 (m, 2H), 3.60-3.53 (m, 2H), 3.45 (m, 5H), 3.29-3.24 |
| | | | | (m, 2H), 2.90 (m, |
| | | | | 1H), 2.81 (m, |
| | | | | 3H), 2.49 (m, |
| | | | | 1H), 2.17 (m, |
| | | | | 1H), 1.92 (m, |
| | | | | 1H) |
| 48 | 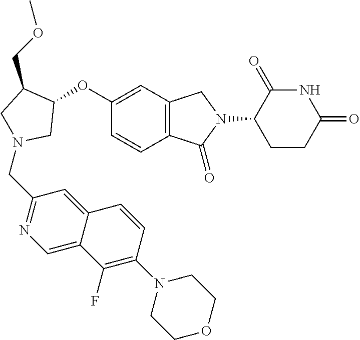 [SMILES: COC[C@@H]1CN(Cc2cc3ccc(N4CCOCC4)c(F)c3cn2)C[C@H]1Oc1ccc2c(c1)CN([C@H]1CCC(=O)NC1=O)C2=O] | (3S)-3-(5- {[(3S,4S)-1-{[8- fluoro-7- (morpholin-4- yl)isoquinolin-3- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.2 [M + 1]⁺ | ¹H NMR (400 MHz, MeOD-d₄) δ 9.35 (d, J = 3.7 Hz, 1H), 7.81 (s, 1H), 7.73-7.61 (m, 3H), 7.17- 7.04 (m, 2H), 5.11 (m, 1H), 4.95 (m, 1H), 4.42 (m, 2H), 4.29-4.13 (m, 2H), 3.89-3.86 (m, 4H), 3.62- 3.52 (m, 2H), 3.48-3.42 (m, 1H), 3.40 (s, 3H), 3.28-3.22 (m, 6H), 2.95-2.73 (m, 4H), 2.48 (m, 1H), 2.17 (m, 1H) |
| 49 | 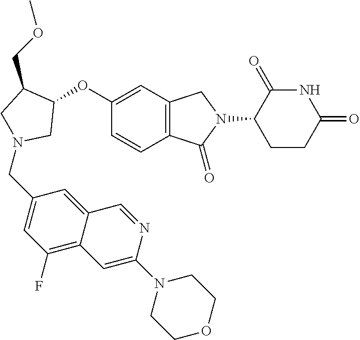 [SMILES: COC[C@@H]1CN(Cc2cc(F)c3cc(N4CCOCC4)ncc3c2)C[C@H]1Oc1ccc2c(c1)CN([C@H]1CCC(=O)NC1=O)C2=O] | (3S)-3-(5- {[(3S,4S)-1-{[5- fluoro-3- (morpholin-4- yl)isoquinolin-7- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.3 [M + 1]⁺ | ¹H NMR (400 MHz, MeOD-d₄) δ 8.96 (s, 1H), 7.71 (d, J = 8.4 Hz, 1H), 7.66 (s, 1H), 7.38 (d, J = 11.6 Hz, 1H), 7.15 (s, 1H), 7.13-7.06 (m, 1H), 6.98 (s, 1H), 5.12 (dd, J = 13.3, 5.2 Hz, 1H), 4.94-4.90 (m, 1H), 4.51- 4.36 (m, 2H), 3.91-3.78 (m, 6H), 3.60-3.50 (m, 6H), 3.39 (s, 3H), 3.13 (m, 1H), 3.04-2.92 (m, 3H), 2.90- |
| | | | | 2.75 (m, 2H), |
| | | | | 2.56-2.40 (m, |
| | | | | 2H), 2.20-2.11 |
| | | | | (m, 1H) |
| 50 | 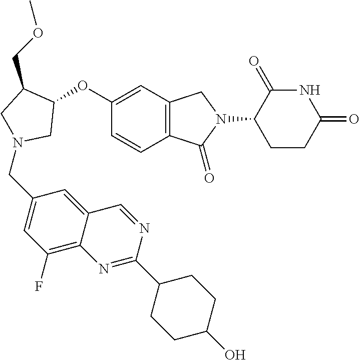 [SMILES: COC[C@@H]1CN(Cc2cc(F)c3nc(C4CCC(O)CC4)ncc3c2)C[C@H]1Oc1ccc2c(c1)CN([C@H]1CCC(=O)NC1=O)C2=O] | rel-(S)-3-(5- (((3S,4S)-1-((8- fluoro-2-(4- hydroxycyclohexyl) quinazolin-6- yl)methyl)-4- (methoxymethyl) pyrrolidin-3- yl)oxy)-1- oxoisoindolin-2- yl)piperidine-2,6- dione | MS (ESI) m/z 632.5 [M + 1]⁺ | ¹H NMR (400 MHz, MeOD-d₄) δ 9.49 (d, J = 1.4 Hz, 1H), 7.88 (s, 1H), 7.81 (dd, J = 11.2, 1.7 Hz, 1H), 7.72 (d, J = 8.4 Hz, 1H), 7.15 (d, J = 2.2 Hz, 1H), 7.10 (dd, J = 8.5, 2.2 Hz, 1H), 5.13 (dd, J = 13.3, 5.2 Hz, 1H), 4.88 (m, 1H), 4.45-4.42 (m, 2H), 4.03 (m, 2H), 3.56 (d, J = 6.3 Hz, 2H), 3.40 (s, 3H), 3.33-3.11 (m, 4H), 2.92 (m, 1H), 2.81-2.71 (m, 2H), 2.59 |
| | | | | (m, 1H), 2.48 (m, |
| | | | | 1H), 2.33-2.29 |
| | | | | (m, 2H), 2.13 (m, |
| | | | | 1H), 1.90-1.71 |
| | | | | (m, 6H) |
| 51 | 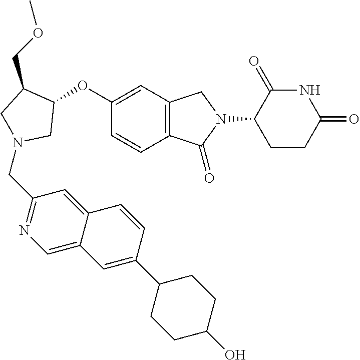 [SMILES: COC[C@@H]1CN(Cc2cc3ccc(C4CCC(O)CC4)cc3cn2)C[C@H]1Oc1ccc2c(c1)CN([C@H]1CCC(=O)NC1=O)C2=O] | rel-(S)-3-(5- (((3S,4S)-1-((7- (4- hydroxycyclohexyl) isoquinolin-3- yl)methyl)-4- (methoxymethyl) pyrrolidin-3- yl)oxy)-1- oxoisoindolin-2- yl)piperidine-2,6- dione | MS (ESI) m/z 613.2 [M + 1]⁺ | ¹H NMR (400 MHz, DMSO-d₆) δ 9.15 (s, 1H), 7.90-7.80 (m, 2H), 7.74 (s, 1H), 7.68 (d, J = 8.5 Hz, 1H), 7.62 (d, J = 8.5 Hz, 1H), 7.11 (s, 1H), 7.02 (d, J = 8.6 Hz, 1H), 4.96-4.92 (m, 1H), 4.74 (s, 1H), 4.37 (d, J = 17.6 Hz, 1H), 4.24 (d, J = 17.5 Hz, 1H), 4.01- 3.93 (m, 2H), 3.50-3.34 (m, 3H), 3.23 (s, 3H), 3.09 (m, 1H), 2.98-2.94 (m, 1H), 2.83-2.79 |
| | | | | (m, 2H), 2.71- |
| | | | | 2.68 (m, 2H), |
| | | | | 2.58-2.56 (m, |
| | | | | 2H), 2.35 (m, |
| | | | | 2H), 1.98 (m, |
| | | | | 1H), 1.91-1.81 |
| | | | | (m, 2H), 1.75 (m, |
| | | | | 2H), 1.57 (m, |
| | | | | 4H) |
| 52 | 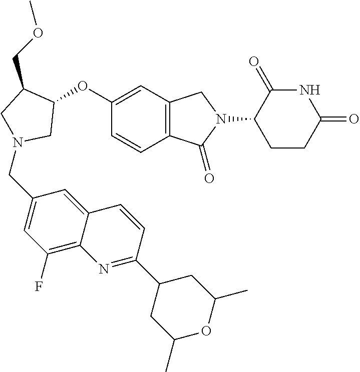 [SMILES: COC[C@@H]1CN(Cc2cc(F)c3nc(C4CC(C)OC(C)C4)ccc3c2)C[C@H]1Oc1ccc2c(c1)CN([C@H]1CCC(=O)NC1=O)C2=O] | re1-(3S)-3-(5- {[(3S,4S)-1-{[2- (2,6- dimethyloxan-4- yl)-8- fluoroquinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 645.4 [M + 1]⁺ | ¹H NMR (400 MHz, MeOD-d₄) δ 8.50-8.46 (m, 1H), 7.68-7.64 (m, 1H), 7.62 (d, J = 8.5 Hz, 1H), 7.58-7.50 (m, 2H), 7.12 (d, J = 2.2 Hz, 1H), 7.06-7.04 (m, 1H), 5.10-5.08 (m, 1H), 4.75 (m, 1H), 4.40-4.28 (m, 2H), 3.75 (m, 2H), 3.55 (m, 2H), 3.40 (m, 2H), 3.28 (s, 3H), 3.20 (m, 1H), 2.98-2.88 (m, 3H), 2.78-2.68 (m, 1H),2.62- 2.56 (m, 2H), 2.42-2.28 (m, |
| | | | | 2H),1.98-1.96 |
| | | | | (m, 1H), 1.85 (m, |
| | | | | 2H), 1.46 (m, |
| | | | | 2H), 1.06 (m, |
| | | | | 6H) |
| 53 | 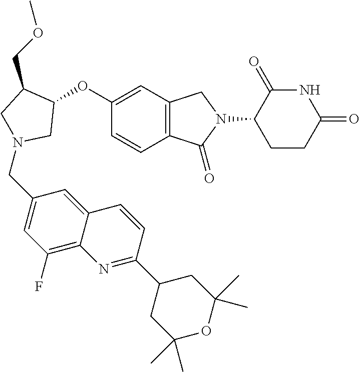 [SMILES: COC[C@@H]1CN(Cc2cc(F)c3nc(C4CC(C)(C)OC(C)(C)C4)ccc3c2)C[C@H]1Oc1ccc2c(c1)CN([C@H]1CCC(=O)NC1=O)C2=O] | (3S)-3-(5- {[(3S,4S)-1-{[8- fluoro-2-(2,2,6,6- tetramethyloxan- 4-yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 673.4 [M + 1]⁺ | ¹H NMR (400 MHz, MeOD-d₄) δ 8.29 (m, 1H), 7.75-7.65 (m, 2H), 7.63-7.51 (m, 2H), 7.15 (d, J = 2.5 Hz, 1H), 7.09-7.07 (m, 1H), 5.12-5.10 (m, 1H), 4.79 (m, 1H), 4.50-4.36 (m, 2H), 3.90 (d, J = 3.1 Hz, 2H), 3.68-3.56 (m, 1H), 3.54 (d, J = 6.5 Hz, 2H), 3.39 (s, 3H), 3.15-3.13 (m, 1H), 3.07- 2.94 (m, 2H), 2.90-2.84 (m, 1H), 2.80-2.68 (m, 2H), 2.55- 2.40 (m, 2H), |
| | | | | 2.22-2.12 (m, |
| | | | | 1H), 1.89-1.86 |
| | | | | (m, 2H), 1.75 (m, |
| | | | | 2H), 1.44 (s, 6H), |
| | | | | 1.29 (s, 6H) |
| 54 | 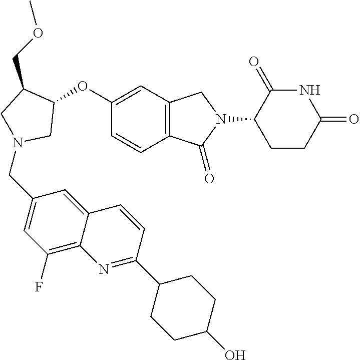 [SMILES: COC[C@@H]1CN(Cc2cc(F)c3nc(C4CCC(O)CC4)ccc3c2)C[C@H]1Oc1ccc2c(c1)CN([C@H]1CCC(=O)NC1=O)C2=O] | rel-(S)-3-(5- (((3S,4S)-1-((8- fluoro-2-(4- hydroxycyclohexyl) quinolin-6- yl)methyl)-4- (methoxymethyl) pyrrolidin-3- yl)oxy)-1- oxoisoindolin-2- yl)piperidine-2,6- dione | MS (ESI) m/z 631.2 [M + 1]⁺ | ¹H NMR (400 MHz, DMSO-d₆) δ 8.29 (m, 1H), 7.69-7.61 (m, 2H), 7.58-7.47 (m, 2H), 7.15 (d, J = 2.2 Hz, 1H), 7.05 (m, 1H), 5.02 (m, 1H), 4.72 (m, 1H), 4.40 (d, J = 17.5 Hz, 1H), 4.27 (d, J = 17.4 Hz, 1H), 3.79 (s, 2H), 3.57-3.37 (m, 3H), 3.26 (s, 3H), 3.04-2.75 (m, 5H), 2.68-2.55 (m, 2H), 2.46- 2.31 (m, 1H), 2.27 (m, 1H), 2.05-1.91 (m, |
| | | | | 5H), 1.70 (m, |
| | | | | 1H), 1.35 (m, |
| | | | | 2H) |
| 55 | 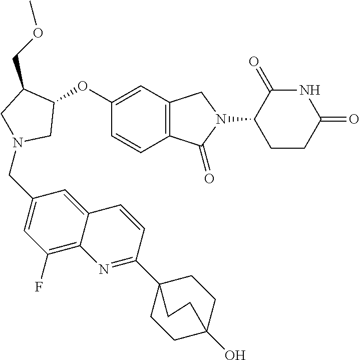 | (3S)-3-(5- {[(3S,4S)-1-[(8- fluoro-2-{4- hydroxybicyclo [2.2.2]octan-1- yl}quinolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 693.2 [M + 1]⁺ | ¹H NMR (400 MHz, DMSO-d₆) δ 8.30 (m, 1H), 7.80-7.70 (m, 3H), 7.45 (m, 1H), 7.16 (s, 1H), 7.04 (m, 1H), 5.06 (m, 1H), 4.75 (m, 1H), 4.36 (m, 1H), 4.25 (m, 1H), 3.70 (m, 2H), 3.40 (m, 2H), 3.25 (s, 3H), 2.91 (m, 3H), 2.75 (m, 1H), 2.61 (m, 1H), 2.56 (m, 2H), 2.39 (m, 1H), 2.25 (m, 1H), 1.99 (m, 7H), 1.65 (m, 6H) |
| 56 | 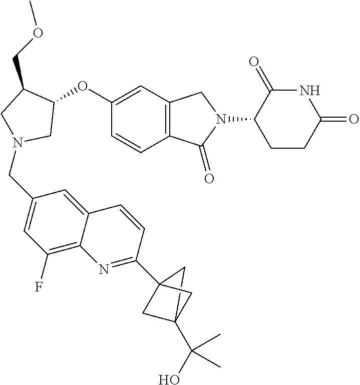 [SMILES: COC[C@@H]1CN(Cc2cc(F)c3nc(C45CC(C(C)(C)O)(C4)C5)ccc3c2)C[C@H]1Oc1ccc2c(c1)CN([C@H]1CCC(=O)NC1=O)C2=O] | (3S)-3-(5- {[(3S,4S)-1-({8- fluoro-2-[3-(2- hydroxypropan-2- yl)bicyclo[1.1.1] pentan-1- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 657.3 [M + 1]⁺ | ¹H NMR (400 MHz, DMSO-d₆) δ 10.96 (s, 1H), 8.33 (dd, J = 8.6, 1.6 Hz, 1H), 7.68 (d, J = 1.6 Hz, 1H), 7.60 (d, J = 8.4 Hz, 1H), 7.55 (d, J = 8.5 Hz, 1H), 7.50 (dd, J = 11.9, 1.7 Hz, 1H), 7.16 (d, J = 2.2 Hz, 1H), 7.04 (dd, J = 8.4, 2.2 Hz, 1H), 5.06 (dd, J = 13.3, 5.1 Hz, 1H), 4.75 (m, 1H), 4.36 (d, J = 17.2 Hz, 1H), 4.29-4.20 (m, 2H), 3.80 (d, J = 13.5 Hz, 1H), 3.72 (d, J = 13.5 Hz, 1H), 3.44 (m, |
| | | | | 2H), 3.25 (s, 3H), |
| | | | | 2.91 (m, 3H), |
| | | | | 2.75 (m, 1H), |
| | | | | 2.61 (m, 1H), |
| | | | | 2.56 (m, 1H), |
| | | | | 2.39 (m, 1H), |
| | | | | 2.25 (m, 1H), |
| | | | | 1.99 (m, 7H), |
| | | | | 1.12 (s, 6H) |
| 57 | 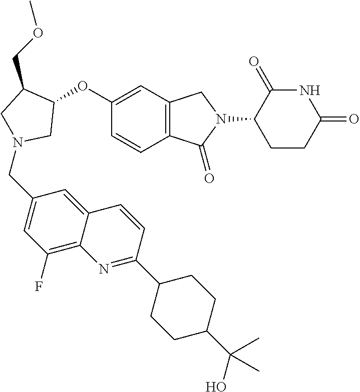 [SMILES: COC[C@@H]1CN(Cc2cc(F)c3nc(C4CCC(C(C)(C)O)CC4)ccc3c2)C[C@H]1Oc1ccc2c(c1)CN([C@H]1CCC(=O)NC1=O)C2=O] | rel-(S)-3-(5- (((3S,4S)-1-((8- fluoro-2-(4-(2- hydroxypropan-2- yl)cyclohexyl) quinolin-6- yl)methyl)-4- (methoxymethyl) pyrrolidin-3- yl)oxy)-1- oxoisoindolin-2- yl)piperidine-2,6- dione | MS (ESI) m/z 673.3 [M + 1]⁺ | ¹H NMR (400 MHz, DMSO-d₆) δ 8.29 (dd, J = 8.7, 1.6 Hz, 1H), 7.65 (d, J = 1.6 Hz, 1H), 7.60 (d, J = 8.4 Hz, 1H), 7.54 (d, J = 8.6 Hz, 1H), 7.48 (dd, J = 11.8, 1.7 Hz, 1H), 7.15 (d, J = 2.2 Hz, 1H), 7.03 (dd, J = 8.4, 2.2 Hz, 1H), 5.05 (dd, J = 13.2, 5.1 Hz, 1H), 4.74 (m, 1H), 4.36 (d, J = 17.2 Hz, 1H), 4.25 (d, J = 17.3 Hz, 1H), 3.78 (d, J = 13.4 Hz, 1H), 3.72 (d, J = 13.4 Hz, 1H), 3.45 (m, 2H), 3.26 (s, 3H), |
| | | | | 2.98-2.70 (m, |
| | | | | 5H), 2.64-2.55 |
| | | | | (m, 2H), 2.36 (m, |
| | | | | 1H), 2.25 (m, |
| | | | | 1H), 2.02-1.90 |
| | | | | (m, 5H), 1.63- |
| | | | | 1.57 (m, 2H), |
| | | | | 1.32 (m, 1H), |
| | | | | 1.18 (m, 2H), |
| | | | | 1.08 (s, 6H) |
| 58 | 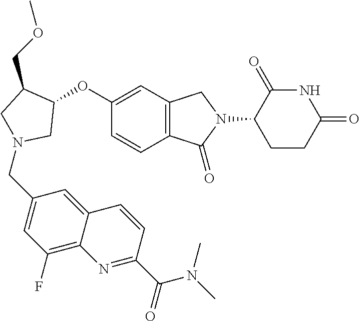 [SMILES: COC[C@@H]1CN(Cc2cc(F)c3nc(C(=O)N(C)C)ccc3c2)C[C@H]1Oc1ccc2c(c1)CN([C@H]1CCC(=O)NC1=O)C2=O] | 6-{[(3S,4S)-3- ({2-[(3S)-2,6- dioxopiperidin-3- yl]-1-oxo-2,3- dihydro-1H- isoindol-5- yl}oxy)-4- (methoxymethyl) pyrrolidin-1- yl]methyl}-8- fluoro-N,N- dimethylquinoline- 2-carboxamide | MS (ESI) m/z 604.3 [M + 1]⁺ | ¹H NMR (400 MHz, DMSO-d₆) δ 8.51 (dd, J = 8.6, 2.9 Hz, 1H), 8.15 (d, J = 3.1 Hz, 1H), 7.78 (d, J = 2.9 Hz, 1H), 7.71 (dd, J = 9.1, 3.1 Hz, 1H), 7.64-7.57 (m, 2H), 7.15 (s, 1H), 7.04 (m, 1H), 5.09-4.99 (m, 1H), 4.75 (d, J = 4.6 Hz, 1H), 4.36 (dd, J = 17.0, 3.1 Hz, 1H), 4.24 (dd, J = 17.4, 3.2 Hz, 1H), 3.86- 3.72 (m, 2H), |
| | | | | 3.44-3.37 (m, |
| | | | | 2H), 3.26 (s, 3H), |
| | | | | 3.06 (s, 3H), 2.98 |
| | | | | (m, 3H), 2.76 (m, |
| | | | | 1H), 2.63-2.54 |
| | | | | (m, 2H), 2.43- |
| | | | | 2.30 (m, 1H), |
| | | | | 2.28-2.22 (m, |
| | | | | 1H), 2.01-1.93 |
| | | | | (m, 1H) |
| 59 | 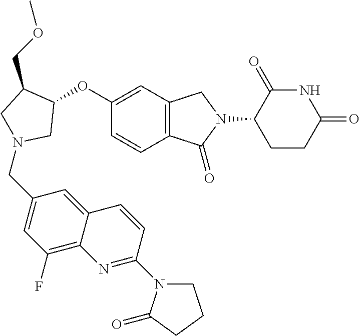 [SMILES: COC[C@@H]1CN(Cc2cc(F)c3nc(N4CCCC4=O)ccc3c2)C[C@H]1Oc1ccc2c(c1)CN([C@H]1CCC(=O)NC1=O)C2=O] | (3S)-3-(5- {[(3S,4S)-1-{[8- fluoro-2-(2- oxopyrrolidin-1- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 616.5 [M + 1]⁺ | ¹H NMR (400 MHz, DMSO-d₆) δ 8.59 (d, J = 9.2 Hz, 1H), 8.40 (dd, J = 9.3, 1.6 Hz, 1H), 7.68 (s, 1H), 7.63 (d, J = 8.4 Hz, 1H), 7.52 (dd, J = 11.8, 1.7 Hz, 1H), 7.18 (d, J = 2.1 Hz, 1H), 7.06 (dd, J = 8.5, 2.2 Hz, 1H), 5.08 (dd, J = 13.3, 5.1 Hz, 1H), 4.77- 4.75 (m, 1H), 4.39 (d, J = 17.2 Hz, 1H), 4.27 (d, J = 17.3 Hz, 1H), 4.15 (t, J = 7.1 Hz, 2H), 3.77- |
| | | | | 3.73 (m, 2H), |
| | | | | 3.52-3.44 (m, |
| | | | | 2H), 3.29 (s, 3H), |
| | | | | 3.01-2.87 (m, |
| | | | | 3H), 2.81-2.57 |
| | | | | (m, 5H), 2.39- |
| | | | | 2.27 (m, 2H), |
| | | | | 2.12 (m, 2H), |
| | | | | 1.99-1.97 (m, |
| | | | | 1H) |
| 60 | 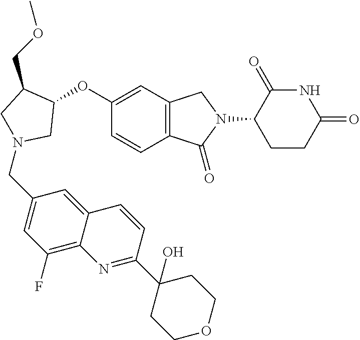 [SMILES: COC[C@@H]1CN(Cc2cc(F)c3nc(C4(O)CCOCC4)ccc3c2)C[C@H]1Oc1ccc2c(c1)CN([C@H]1CCC(=O)NC1=O)C2=O] | (3S)-3-(5- {[(3S,4S)-1-{[8- fluoro-2-(4- hydroxyoxan-4- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 634.5 [M + 1]⁺ | ¹H NMR (400 MHz, MeOD-d₄) δ 8.34-8.32 (m, 1H), 7.90 (d, J = 8.7 Hz, 1H), 7.71 (d, J = 8.8 Hz, 2H), 7.57-7.55 (m, 1H), 7.15 (d, J = 2.3 Hz, 1H), 7.09-7.07 (m, 1H), 5.12-5.10 (m, 1H), 4.95- 4.89 (m, 1H), 4.47 (d, J = 17.0 Hz, 1H), 4.40 (d, J = 16.9 Hz, 1H), 4.09-3.95 (m, 4H), 3.92-3.88 (m, 2H), 3.56 (d, J = 6.2 Hz, 2H), 3.40 (s, 3H), |
| | | | | 3.27-3.24 (m, |
| | | | | 1H), 3.20-3.11 |
| | | | | (m, 2H), 2.91- |
| | | | | 2.87 (m, 1H), |
| | | | | 2.84-2.75 (m, |
| | | | | 2H), 2.65 (m, |
| | | | | 1H), 2.48-2.42 |
| | | | | (m, 3H), 2.17- |
| | | | | 2.15 (m, 1H), |
| | | | | 1.69-1.65 (m, |
| | | | | 2H) |
| 61 | 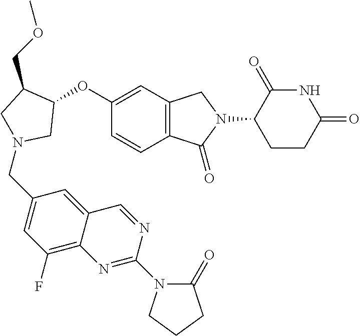 [SMILES: COC[C@@H]1CN(Cc2cc(F)c3nc(N4CCCC4=O)ncc3c2)C[C@H]1Oc1ccc2c(c1)CN([C@H]1CCC(=O)NC1=O)C2=O] | (3S)-3-(5- {[(3S,4S)-1-{[8- fluoro-2-(2- oxopyrrolidin-1- yl)quinazolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 617.2 [M + 1]⁺ | ¹H NMR (400 MHz, DMSO-d₆) δ 9.54 (d, J = 1.7 Hz, 1H), 8.15 (s, 1H), 7.83 (s, 1H), 7.75 (m, 1H), 7.62 (d, J = 8.3 Hz, 1H), 7.14 (d, J = 2.3 Hz, 1H), 7.03 (m, 1H), 5.01 (m, 1H), 4.85-4.70 (m, 1H), 4.37 (d, J = 17.4 Hz, 1H), 4.24 (d, J = 17.3 Hz, 1H), 4.07 (t, J = 7.1 Hz, 2H), 3.83-3.73 (m, 2H), 3.52-3.41 (m, 2H), 3.25 (s, 3H), 3.01-2.80 |
| | | | | (m, 3H), 2.75 (m, |
| | | | | 1H), 2.65-2.51 |
| | | | | (m, 4H), 2.45- |
| | | | | 2.32 (m, 1H), |
| | | | | 2.24 (m, 1H), |
| | | | | 2.19-2.03 (m, |
| | | | | 2H), 1.99 (m, |
| | | | | 1H) |
| 62 | 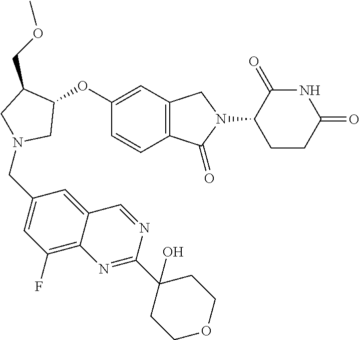 [SMILES: COC[C@@H]1CN(Cc2cc(F)c3nc(C4(O)CCOCC4)ncc3c2)C[C@H]1Oc1ccc2c(c1)CN([C@H]1CCC(=O)NC1=O)C2=O] | (3S)-3-(5- {[(3S,4S)-1-{[8- fluoro-2-(4- hydroxyoxan-4- yl)quinazolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 634.5 [M + 1]⁺ | ¹H NMR (400 MHz, MeOD-d₄) δ 9.56 (s, 1H), 7.93 (s, 1H), 7.88-7.86 (m, 1H), 7.78-7.76 (m, 1H), 7.15 (s, 1H), 7.08-7.06 (m, 1H), 5.13- 5.11 (m, 1H), 4.82-4.80 (m, 1H), 4.49-4.46 (m, 2H), 4.03- 4.01 (m, 2H), 3.90-3.88 (m, 4H), 3.58-3.56 (m, 2H), 3.36 (s, 3H), 3.19-3.17 (m, 1H), 3.05- 3.02 (m, 1H), 2.98-2.96 (m, |
| | | | | 2H), 2.88-2.85 |
| | | | | (m, 1H), 2.77- |
| | | | | 2.75 (m, 1H), |
| | | | | 2.65-2.57 (m, |
| | | | | 4H), 2.19-2.16 |
| | | | | (m, 1H), 1.89- |
| | | | | 1.86 (m, 2H) |
| 63 | 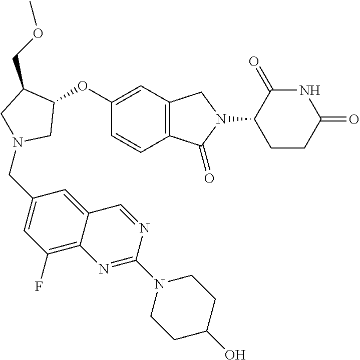 [SMILES: COC[C@@H]1CN(Cc2cc(F)c3nc(N4CCC(O)CC4)ncc3c2)C[C@H]1Oc1ccc2c(c1)CN([C@H]1CCC(=O)NC1=O)C2=O] | (3S)-3-(5- {[(3S,4S)-1-{[8- fluoro-2-(4- hydroxypiperidin- 1-yl)quinazolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 633.3 [M + 1]⁺ | ¹H NMR (400 MHz, MeOD-d₄) δ 9.06 (d, J = 1.6 Hz, 1H), 7.68 (d, J = 8.5 Hz, 1H), 7.56-7.52 (m, 2H), 7.12 (d, J = 2.3 Hz, 1H), 7.08-7.05 (m, 1H), 5.12-5.08 (m, 1H), 4.60- 4.57 (m, 2H), 4.42-4.40 (m, 2H), 3.95-3.90 (m, 3H), 3.54- 3.51 (m, 2H), 3.44-3.39 (m, 2H), 3.38 (s, 3H), 3.37-3.36 (m, 1H), 3.31-3.30 (m, 1H), 3.28- 3.08 (m, 2H), |
| | | | | 2.92-2.85 (m, |
| | | | | 1H), 2.82-2.75 |
| | | | | (m, 2H), 2.74 (m, |
| | | | | 1H), 2.52-2.40 |
| | | | | (m, 1H), 2.20- |
| | | | | 2.12 (m, 1H), |
| | | | | 1.99-1.90 (m, |
| | | | | 2H), 1.58-1.48 |
| | | | | (m, 2H) |
| 64 | 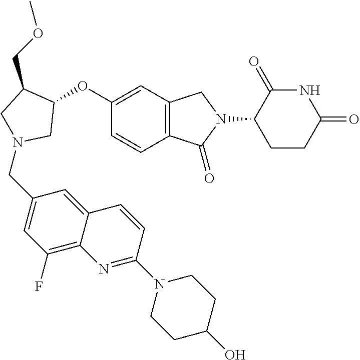 [SMILES: COC[C@@H]1CN(Cc2cc(F)c3nc(N4CCC(O)CC4)ccc3c2)C[C@H]1Oc1ccc2c(c1)CN([C@H]1CCC(=O)NC1=O)C2=O] | (S)-3-(5- (((3S,4S)-1-((8- fluoro-2-(4- hydroxypiperidin- 1-yl)quinolin-6- yl)methyl)-4- (methoxymethyl) pyrrolidin-3- yl)oxy)-1- oxoisoindolin-2- yl)piperidine-2,6- dione | MS (ESI) m/z 632.2 [M + 1]⁺ | ¹H NMR (400 MHz, DMSO-d₆) δ 8.00 (m, 1H), 7.61 (d, J = 8.4 Hz, 1H), 7.40 (d, J = 1.7 Hz, 1H), 7.32-7.24 (m, 2H), 7.13 (d, J = 2.2 Hz, 1H), 7.02 (m, 1H), 5.03 (m, 1H), 4.70 (m, 1H), 4.36 (d, J = 17.4 Hz, 1H), 4.29-4.15 (m, 3H), 3.70 (m, 1H), 3.64 (d, J = 4.0 Hz, 2H), 3.49-3.39 (m, 2H), 3.26 (s, 3H), 3.25-3.15 (m, 2H), 2.91-2.88 (m, 3H), 2.71 (m, |
| | | | | 1H), 2.65-2.52 |
| | | | | (m, 2H), 2.48- |
| | | | | 2.25 (m, 1H), |
| | | | | 2.20 (m, 1H), |
| | | | | 2.05-1.95 (m, |
| | | | | 1H), 1.86-1.82 |
| | | | | (m, 2H), 1.43- |
| | | | | 1.30 (m, 2H) |
| 65 | | rel-(3S)-3-(5- | MS | ¹H NMR (400 |
| | | (((3S,4S)-1-((8- | (ESI) | MHz, DMSO-d₆) |
| | | fluoro-2- | m/z | δ 8.39 (dd, J = |
| | | (tetrahydrofuran- | 603.2 | 8.7, 1.6 Hz, 1H), |
| | | 2-yl)quinolin-6- | [M + 1]⁺ | 7.73-7.64 (m, |
| | | yl)methyl)-4- | | 2H), 7.61 (d, J = |
| | | (methoxymethyl) | | 8.4 Hz, 1H), 7.52 |
| | | pyrrolidin-3- | | (dd, J = 11.8, 1.7 |
| | | yl)oxy)-1- | | Hz, 1H), 7.15 (d, |
| | | oxoisoindolin-2- | | J = 2.2 Hz, 1H), |
| | | yl)piperidine-2,6- | | 7.04 (dd, J = 8.4, |
| | | dione | | 2.2 Hz, 1H), |
| | | | | 5.09-5.01 (m, |
| | | | | 2H), 4.79-4.71 |
| | | | | (m, 1H), 4.37 (d, |
| | | | | J = 17.2 Hz, 1H), |
| | | | | 4.25 (d, J = 17.2 |
| | | | | Hz, 1H), 4.11- |
| | | | | 4.01 (m, 1H), |
| | | | | 3.96-3.87 (m, |
| | | | | 1H), 3.80 (dd, J = |
| | | | | 13.5, 4.0 Hz, |
| | | | | 1H), 3.74 (dd, J = |
| | | | | 13.5, 3.6 Hz, |
| | | | | 1H), 3.56-3.45 |
| | | | | (m, 2H), 3.26 (s, |
| | | | | 3H), 2.99-2.83 |
| | | | | (m, 3H), 2.79- |
| | | | | 2.71 (m, 1H), |
| | | | | 2.64-2.51 (m, |
| | | | | 2H), 2.45-2.29 |
| | | | | (m, 2H), 2.25 (m, |
| | | | | 1H), 2.04-1.87 |
| | | | | (m, 4H) |
| 66 | 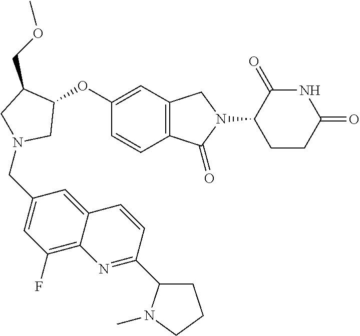 [SMILES: COC[C@@H]1CN(Cc2cc(F)c3nc(C4CCCN4C)ccc3c2)C[C@H]1Oc1ccc2c(c1)CN([C@H]1CCC(=O)NC1=O)C2=O] | rel-(3S)-3-(5- (((3S,4S)-1-((8- fluoro-2-(1- methylpyrrolidin- 2-yl)quinolin-6- yl)methyl)-4- (methoxymethyl) pyrrolidin-3- yl)oxy)-1- oxoisoindolin-2- yl)piperidine-2,6- dione | MS (ESI) m/z 616.6 [M + 1]⁺ | ¹H NMR (400 MHz, DMSO-d₆) δ 8.37 (dd, J = 8.7, 1.6 Hz, 1H), 7.74-7.66 (m, 2H), 7.60 (d, J = 8.4 Hz, 1H), 7.51 (dd, J = 11.8, 1.7 Hz, 1H), 7.04 (dd, J = 8.4, 2.2 Hz, 1H), 5.05 (dd, J = 13.3, 5.1 Hz, 1H), 4.79- 4.71 (m, 1H), 4.37 (d, J = 17.2 Hz, 1H), 4.25 (d, J = 17.3 Hz, 1H), 3.79 (d, J = 13.5 Hz, 1H), 3.74 (d, J = 13.4 Hz, 1H), 3.46-3.37 (m, |
| | | | | 3H), 3.26 (s, 3H), |
| | | | | 3.24-3.16 (m, |
| | | | | 1H), 2.99-2.81 |
| | | | | (m, 3H), 2.75 (m, |
| | | | | 1H), 2.67-2.52 |
| | | | | (m, 2H), 2.44- |
| | | | | 2.20 (m, 4H), |
| | | | | 2.16 (s, 3H), |
| | | | | 2.03-1.67 (m, |
| | | | | 4H) |
| 67 | 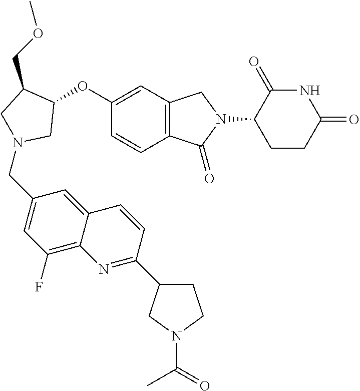 [SMILES: COC[C@@H]1CN(Cc2cc(F)c3nc(C4CCN(C(C)=O)C4)ccc3c2)C[C@H]1Oc1ccc2c(c1)CN([C@H]1CCC(=O)NC1=O)C2=O] | rel-(3S)-3-(5- (((3S,4S)-1-((2- (1- acetylpyrrolidin- 3-yl)-8- fluoroquinolin-6- yl)methyl)-4- (methoxymethyl) pyrrolidin-3- yl)oxy)-1- oxoisoindolin-2- yl)piperidine-2,6- dione | MS (ESI) m/z 644.2 [M + 1]⁺ | ¹H NMR (400 MHz, MeOD-d₄) δ 8.33-8.27 (m, 1H), 8.17 (m, 2H), 7.77-7.67 (m, 2H), 7.66- 7.54 (m, 2H), 7.15 (m, 1H), 7.13-7.06 (m, 1H), 5.17-5.08 (m, 1H), 4.95 (m, 1H), 4.51-4.36 (m, 2H), 4.18- 3.96 (m, 4H), 3.95-3.64 (m, 3H), 3.61-3.53 (m, 3H), 3.38 (s, 3H), 3.20 (m, 2H), 2.94-2.85 (m, 1H), 2.75 (m, 3H), 2.49 (m, 3H), 2.21-2.11 (m, 4H) |
| 68 | 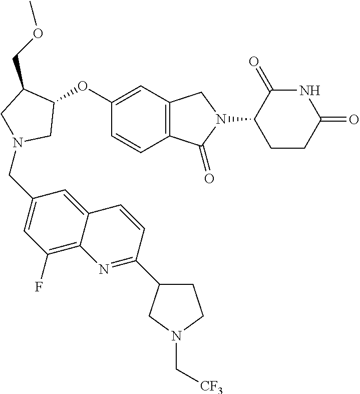 [SMILES: COC[C@@H]1CN(Cc2cc(F)c3nc(C4CCN(CC(F)(F)F)C4)ccc3c2)C[C@H]1Oc1ccc2c(c1)CN([C@H]1CCC(=O)NC1=O)C2=O] | rel-(3S)-3-(5- (((3S,4S)-1-((8- fluoro-2-(1- (2,2,2- trifluoroethyl) pyrrolidin-3- yl)quinolin-6- yl)methyl)-4- (methoxymethyl) pyrrolidin-3- yl)oxy)-1- oxoisoindolin-2- yl)piperidine-2,6- dione | MS (ESI) m/z 684.3 [M + 1]⁺ | ¹H NMR (400 MHz, MeOD-d₄) δ 8.30 (dd, J = 8.7, 1.5 Hz, 1H), 7.75-7.65 (m, 3H), 7.58 (dd, J = 11.5, 1.8 Hz, 1H), 7.16 (d, J = 2.2 Hz, 1H), 7.10 (dd, J = 8.5, 2.2 Hz, 1H), 5.12 (dd, J = 13.3, 5.2 Hz, 1H), 4.96 (m, 1H), 4.51-4.36 (m, 2H), 4.10 (s, 2H), 3.87-3.75 (m, 1H), 3.62- 3.51 (m, 2H), 3.40 (s, 3H), 3.39-3.34 (m, 2H), 3.34-3.11 (m, 5H), 3.11- 2.99 (m, 2H), 2.97-2.85 (m, |
| | | | | 2H), 2.84-2.68 |
| | | | | (m, 3H), 2.56- |
| | | | | 2.38 (m, 2H), |
| | | | | 2.23-2.10 (m, |
| | | | | 2H) |
| 69 | 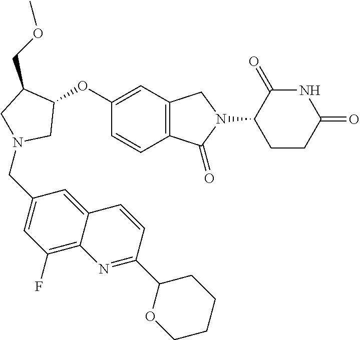 [SMILES: COC[C@@H]1CN(Cc2cc(F)c3nc(C4CCCCO4)ccc3c2)C[C@H]1Oc1ccc2c(c1)CN([C@H]1CCC(=O)NC1=O)C2=O] | rel-(3S)-3-(5- {[(3S,4S)-1-{[8- fluoro-2-(oxan-2- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 617.5 [M + 1]⁺ | ¹H NMR (400 MHz, MeOD-d₄) δ 8.35 (dd, J = 8.7, 1.5 Hz, 1H), 7.77 (d, J = 8.6 Hz, 1H), 7.71 (dd, J = 5.0, 3.4 Hz, 2H), 7.57 (dd, J = 11.8, 1.7 Hz, 1H), 7.15 (d, J = 2.2 Hz, 1H), 7.10 (dd, J = 8.5, 2.2 Hz, 1H), 5.12 (dd, J = 13.3, 5.1 Hz, 1H), 4.95 (m, 1H), 4.67 (dd, J = 11.3, 2.3 Hz, 1H), 4.51-4.36 (m, 2H), 4.23- 4.15 (m, 1H), 3.94 (s, 2H), |
| | | | | 3.78-3.67 (m, |
| | | | | 1H), 3.54 (d, J = |
| | | | | 6.4 Hz, 2H), 3.39 |
| | | | | (s, 3H), 3.18 (m, |
| | | | | 1H), 3.07-2.95 |
| | | | | (m, 2H), 2.90 (m, |
| | | | | 1H), 2.84-2.69 |
| | | | | (m, 2H), 2.56- |
| | | | | 2.40 (m, 2H), |
| | | | | 2.22-2.11 (m, |
| | | | | 1H), 2.11-1.97 |
| | | | | (m, 2H), 1.90- |
| | | | | 1.70 (m, 2H), |
| | | | | 1.70-1.53 (m, |
| | | | | 2H) |
| 70 | 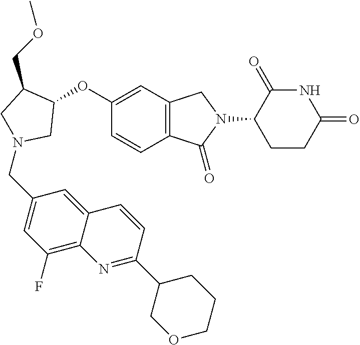 [SMILES: COC[C@@H]1CN(Cc2cc(F)c3nc(C4CCCOC4)ccc3c2)C[C@H]1Oc1ccc2c(c1)CN([C@H]1CCC(=O)NC1=O)C2=O] | rel-(3S)-3-(5- {[(3S,4S)-1-{[8- fluoro-2-(oxan-3- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 617.4 [M + 1]⁺ | ¹H NMR (400 MHz, MeOD-d₄) δ 8.25 (m, 1H), 8.21 (s, 1H), 7.72-7.70 (m, 2H), 7.59-7.55 (m, 2H), 7.15 (d, J = 2.1 Hz, 1H), 7.10 (dd, J = 8.5, 2.2 Hz, 1H), 5.10 (dd, J = 13.3, 5.1 Hz, 1H), 4.95 (m, 1H), 4.45-4.42 (m, 2H), 4.20- 3.90 (m, 4H), 3.54 (m, 3H), 3.40 (s, 3H), 3.33 (m, 1H), 3.30- 3.17 (m, 3H), 2.81 (m, 1H), 2.78 (m, 2H), |
| | | | | 2.71 (m, 1H), |
| | | | | 2.48 (m, 1H), |
| | | | | 2.17 (m, 2H), |
| | | | | 2.16-2.00 (m, |
| | | | | 1H), 1.80 (m, |
| | | | | 2H) |
| 71 | 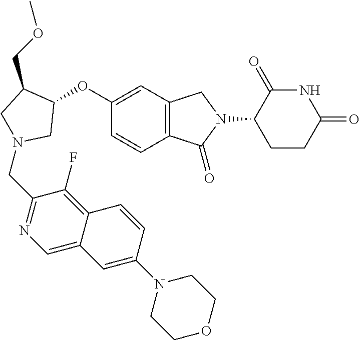 [SMILES: COC[C@@H]1CN(Cc2ncc3cc(N4CCOCC4)ccc3c2F)C[C@H]1Oc1ccc2c(c1)CN([C@H]1CCC(=O)NC1=O)C2=O] | (3S)-3-(5- {[(3S,4S)-1-{[4- fluoro-7- (morpholin-4- yl)isoquinolin-3- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.4 [M + 1]⁺ | ¹H NMR (400 MHz, DMSO-d₆) δ 8.92 (s, 1H), 7.93 (d, J = 9.2 Hz, 1H), 7.73 (dd, J = 9.2, 2.2 Hz, 1H), 7.59 (d, J = 8.4 Hz, 1H), 7.41 (s, 1H), 7.11 (s, 1H), 6.99 (d, J = 8.6 Hz, 1H), 5.04 (dd, J = 13.4, 5.1 Hz, 1H), 4.68 (m, 1H), 4.35 (d, J = 17.3 Hz, 1H), 4.24 (d, J = 17.2 Hz, 1H), 3.90- 3.70 (m, 5H), 3.40-3.20 (m, 10H), 3.01-2.82 |
| | | | | (m, 3H), 2.78 (m, |
| | | | | 1H), 2.58 (m, |
| | | | | 2H), 2.40-2.24 |
| | | | | (m, 2H), 1.97 (m, |
| | | | | 1H) |
| 72 | 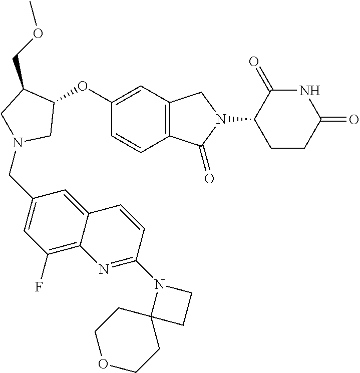 [SMILES: COC[C@@H]1CN(Cc2cc(F)c3nc(N4CCC45CCOCC5)ccc3c2)C[C@H]1Oc1ccc2c(c1)CN([C@H]1CCC(=O)NC1=O)C2=O] | (3S)-3-(5- {[(3S,4S)-1-[(8- fluoro-2-{7-oxa- 1-azaspiro [3.5]nonan- 1-yl}quinolin- 6-yl)methyl]-4- (methoxymethyl) pyrrolidin-3- yl]oxy}-1-oxo- 2,3-dihydro-1H- isoindol-2- yl)piperidine-2,6- dione | MS (ESI) m/z 658.2 [M + 1]⁺ | ¹H NMR (400 MHz, MeOD-d₄) δ 7.91 (m, 1H), 7.72 (m, 1H), 7.43 (d, J = 5.5 Hz, 1H), 7.39- 7.31 (m, 1H), 7.16 (d, J = 2.3 Hz, 1H), 7.10 (m, 1H), 6.75 (s, 1H), 5.13 (m, 1H), 4.91 (m, 1H), 4.52-4.37 (m, 2H), 4.09 (t, J = 7.5 Hz, 2H), 4.00 (m, 4H), 3.62- 3.46 (m, 5H), 3.40 (s, 3H), 3.18 (m, 2H), 2.92 (m, 2H), 2.80 (m, 3H), 2.76-2.61 (m, 1H), 2.50- 2.40 (m, 1H), |
| | | | | 2.37 (t, J = 7.5 |
| | | | | Hz, 2H), 2.20- |
| | | | | 2.10 (m, 1H), |
| | | | | 1.80 (m, 2H) |
| 73 | 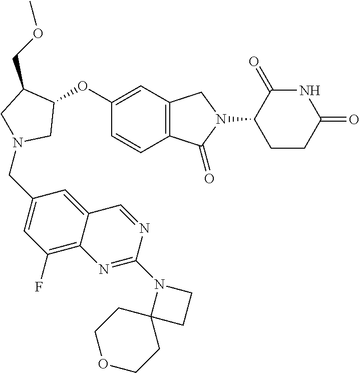 [SMILES: COC[C@@H]1CN(Cc2cc(F)c3nc(N4CCC45CCOCC5)ncc3c2)C[C@H]1Oc1ccc2c(c1)CN([C@H]1CCC(=O)NC1=O)C2=O] | (3S)-3-(5- {[(3S,4S)-1-[(8- fluoro-2-{7-oxa- 1- azaspiro[3.5] nonan-1- yl}quinazolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 659.3 [M + 1]⁺ | ¹H NMR (400 MHz, DMSO-d₆) δ 10.96 (s, 1H), 9.17 (s, 1H), 7.65-7.43 (m, 3H), 7.15 (d, J = 2.2 Hz, 1H), 7.03 (dd, J = 8.4, 2.2 Hz, 1H), 5.06 (dd, J = 13.3, 5.1 Hz, 1H), 4.74 (m, 1H), 4.45-4.19 (m, 2H), 4.10- 3.94 (m, 2H), 3.92-3.82 (m, 2H), 3.76-3.56 (m, 2H), 3.52- 3.36 (m, 3H), 3.26 (s, 3H), 3.04-2.82 (m, 3H), 2.78-2.64 (m, 2H), 2.60- 2.52 (m, 4H), |
| | | | | 2.37 (m, 1H), |
| | | | | 2.20-2.16 (m, |
| | | | | 3H), 2.05-1.91 |
| | | | | (m, 1H), 1.78- |
| | | | | 1.63 (m, 2H) |
| 74 | 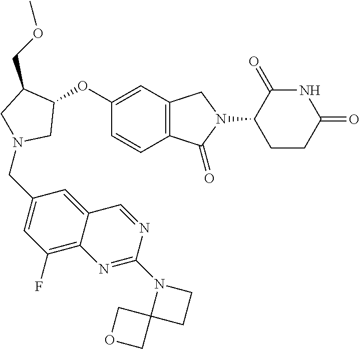 [SMILES: COC[C@@H]1CN(Cc2cc(F)c3nc(N4CCC45COC5)ncc3c2)C[C@H]1Oc1ccc2c(c1)CN([C@H]1CCC(=O)NC1=O)C2=O] | (3S)-3-(5- {[(3S,4S)-1-[(8- fluoro-2-{6-oxa- 1- azaspiro[3.3] heptan-1- yl}quinazolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 631.3 [M + 1]⁺ | ¹H NMR (400 MHz, MeOD-d₄) δ 9.13 (d, J = 1.5 Hz, 1H), 7.68 (d, J = 8.5 Hz, 1H), 7.65-7.50 (m, 2H), 7.12(d, J = 2.2 Hz, 1H), 7.06-7.04 (m, 1H), 5.60 (s, 2H), 5.10-5.08 (m, 1H), 4.83-4.81 (m, 1H), 4.70 (d, J = 6.9 Hz, 2H), 4.39 (m, 2H), 4.08-4.00 (m, 2H), 3.85-3.73 (m, 2H), 3.51 (d, J = 6.5 Hz, 2H), 3.36 (s, 3H), 3.12-3.10 (m, 1H), 3.01-2.93 |
| | | | | (m, 2H), 2.89- |
| | | | | 2.86 (m, 1H), |
| | | | | 2.81-2.72 (m, |
| | | | | 1H), 2.76-2.63 |
| | | | | (m, 3H), 2.53- |
| | | | | 2.37 (m, 2H), |
| | | | | 2.14-2.12 (m, |
| | | | | 1H) |
| 75 | 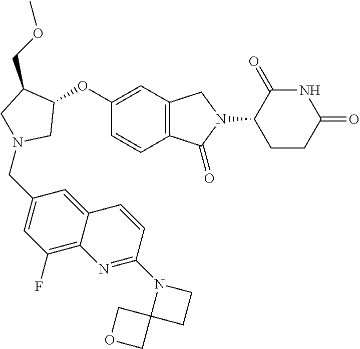 [SMILES: COC[C@@H]1CN(Cc2cc(F)c3nc(N4CCC45COC5)ccc3c2)C[C@H]1Oc1ccc2c(c1)CN([C@H]1CCC(=O)NC1=O)C2=O] | (3S)-3-(5- {[(3S,4S)-1-[(8- fluoro-2-{6-oxa- 1-azaspiro [3.3]heptan- 1-yl}quinolin- 6-yl)methyl]-4- (methoxymethyl) pyrrolidin-3- yl]oxy}-1-oxo- 2,3-dihydro-1H- isoindol-2- yl)piperidine-2,6- dione | MS (ESI) m/z 630.6 [M + 1]⁺ | ¹H NMR (400 MHz, MeOD-d₄) δ 8.02 (m, 1H), 7.71 (d, J = 8.5 Hz, 1H), 7.48 (d, J = 1.8 Hz, 1H), 7.38 (m, 1H), 7.14 (d, J = 2.1 Hz, 1H), 7.09 (m, 2H), 5.51 (d, J = 7.3 Hz, 2H), 5.12 (m, 1H), 4.85 (m, 1H), 4.80 (d, J = 7.3 Hz, 1H), 4.46 (d, J = 17.0 Hz, 2H), 4.40 (d, J = 17.0 Hz, 2H), 4.03 (t, J = 7.2 Hz, 2H), 3.99- 3.85 (m, 2H), 3.54 (d, J = 6.3 Hz, 2H), 3.39 (s, |
| | | | | 3H), 3.26 (m, |
| | | | | 1H), 3.09 (d, J = |
| | | | | 3.7 Hz, 2H), 2.91 |
| | | | | (m, 1H), 2.84- |
| | | | | 2.71 (m, 2H), |
| | | | | 2.68 (m, 2H), |
| | | | | 2.58 (m, 1H), |
| | | | | 2.51-2.42 (m, |
| | | | | 1H), 2.20-2.09 |
| | | | | (m, 1H) |
| 76 | 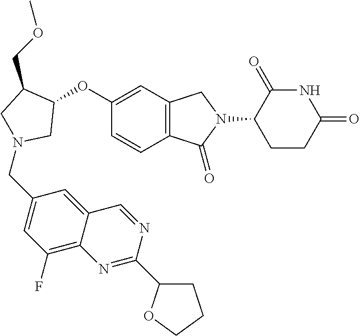 [SMILES: COC[C@@H]1CN(Cc2cc(F)c3nc(C4CCCO4)ncc3c2)C[C@H]1Oc1ccc2c(c1)CN([C@H]1CCC(=O)NC1=O)C2=O] | rel-(3S)-3-(5- {[(3S,4S)-1-{[8- fluoro-2-(oxolan- 2-yl)quinazolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 604.2 [M + 1]⁺ | ¹H NMR (400 MHz, MeOD-d₄) δ 9.63 (d, J = 1.5 Hz, 1H), 8.16 (s, 1H), 7.90 (d, J = 1.6 Hz, 1H), 7.82-7.79 (m, 1H), 7.61 (d, J = 8.4 Hz, 1H), 7.15 (d, J = 2.2 Hz, 1H), 7.04-7.02 (m, 1H), 5.13- 5.11 (m, 1H), 5.09-5.04 (m, 1H), 4.75-4.74 (m, 1H), 4.37 (d, J = 17.3 Hz, 1H), 4.24 (d, J = 17.3 Hz, 1H), 4.06 (m, 1H), 3.91-3.90 (m, 1H), 3.85 (m, |
| | | | | 1H), 3.77 (m, |
| | | | | 1H), 3.32-3.30 |
| | | | | (m, 2H), 3.26 (s, |
| | | | | 3H), 3.00-2.96 |
| | | | | (m, 2H), 2.94- |
| | | | | 2.82 (m, 1H), |
| | | | | 2.79-2.76 (m, |
| | | | | 1H), 2.64-2.52 |
| | | | | (m, 2H), 2.40- |
| | | | | 2.37 (m, 2H), |
| | | | | 2.34-2.26 (m, |
| | | | | 1H), 2.12-2.05 |
| | | | | (m, 2H), 2.00- |
| | | | | 1.96 (m, 2H) |
| 77 | 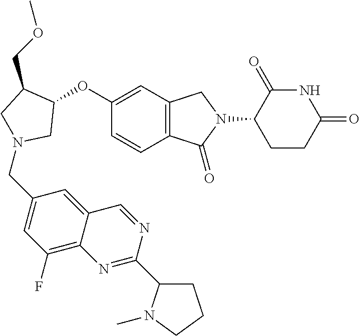 [SMILES: COC[C@@H]1CN(Cc2cc(F)c3nc(C4CCCN4C)ncc3c2)C[C@H]1Oc1ccc2c(c1)CN([C@H]1CCC(=O)NC1=O)C2=O] | rel-(3S)-3-(5- {[(3S,4S)-1-{[8- fluoro-2-(1- methylpyrrolidin- 2-yl)quinazolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 617.5 [M + 1]⁺ | ¹H NMR (400 MHz, MeOD-d₄) δ 9.65 (d, J = 1.9 Hz, 1H), 8.38 (s, 2H-formic acid), 7.98 (s, 1H), 7.96-7.88 (m, 1H), 7.69 (d, J = 8.5 Hz, 1H), 7.14 (s, 1H), 7.08 (dd, J = 8.5, 2.1 Hz, 1H), 5.12 (dd, J = 13.3, 5.1 Hz, 1H), 4.86 (m, 1H), 4.46 (d, J = 17.0 Hz, 1H), 4.39 (d, J = 17.0 Hz, 1H), 4.02- 3.93 (m, 3H), 3.71-3.49 (m, 3H), 3.48-3.37 |
| | | | | (m, 1H), 3.35 (s, |
| | | | | 3H), 3.21-3.12 |
| | | | | (m, 1H), 3.08 (s, |
| | | | | 3H), 3.07-2.86 |
| | | | | (m, 3H), 2.85- |
| | | | | 2.67 (m, 3H), |
| | | | | 2.55-2.40 (m, |
| | | | | 2H), 2.38-2.26 |
| | | | | (m, 2H), 2.25- |
| | | | | 2.11 (m, 2H) |
| 78 | 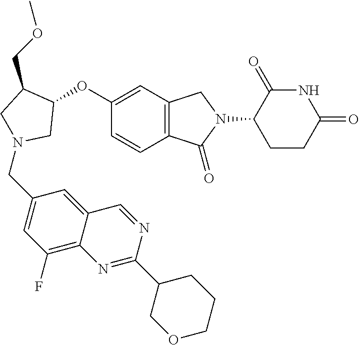 [SMILES: COC[C@@H]1CN(Cc2cc(F)c3nc(C4CCCOC4)ncc3c2)C[C@H]1Oc1ccc2c(c1)CN([C@H]1CCC(=O)NC1=O)C2=O] | rel-(3S)-3-(5- {[(3S,4S)-1-{[8- fluoro-2-(oxan-3- yl)quinazolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.5 [M + 1]⁺ | ¹H NMR (400 MHz, MeOD-d₄) δ 9.54 (d, J = 1.4 Hz, 1H), 7.90 (d, J = 1.6 Hz, 1H), 7.85 (dd, J = 11.2, 1.7 Hz, 1H), 7.71 (d, J = 8.4 Hz, 1H), 7.14 (d, J = 2.2 Hz, 1H), 7.07 (dd, J = 8.5, 2.2 Hz, 1H), 5.14 (dd, J = 13.3, 5.2 Hz, 1H), 4.87-4.76 (m, 1H), 4.43 (m, 2H), 4.18 (m, 1H), 3.95 (m, 2H), 3.74 (m, 3H), 3.50 (m, 3H), 3.35 (s, 3H), |
| | | | | 3.30 (m, 1H), |
| | | | | 3.05 (m, 1H), |
| | | | | 3.03-2.85 (m, |
| | | | | 3H), 2.71 (m, |
| | | | | 2H), 2.50 (m, |
| | | | | 2H), 2.20 (m, |
| | | | | 1H), 2.10 (m, |
| | | | | 1H), 2.00 (m, |
| | | | | 2H), 1.90-1.70 |
| | | | | (m, 2H) |
| 79 | 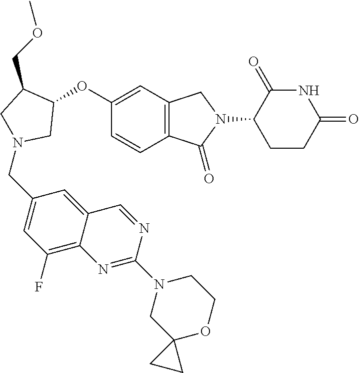 [SMILES: COC[C@@H]1CN(Cc2cc(F)c3nc(N4CCOC5(CC5)C4)ncc3c2)C[C@H]1Oc1ccc2c(c1)CN([C@H]1CCC(=O)NC1=O)C2=O] | (3S)-3-(5- {[(3S,4S)-1-[(8- fluoro-2-{4-oxa- 7- azaspiro[2.5] octan-7- yl}quinazolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 645.4 [M + 1]⁺ | ¹H NMR (400 MHz, DMSO-d₆) δ 10.96 (s, 1H), 9.25 (s, 1H), 7.61 (m, 2H), 7.54 (m, 1H), 7.16 (d, J = 4.4 Hz, 1H), 7.08-7.00 (m, 1H), 5.06 (dd, J = 13.3, 5.4 Hz, 1H), 4.74 (m, 1H), 4.43-4.32 (m, 1H), 4.31- 4.21 (m, 1H), 3.97-3.90 (m, 2H), 3.85 (m, 2H), 3.79-3.59 (m, 4H), 3.43 (m, 2H), 3.28 (s, 3H), 2.91 (m, 3H), 2.74 (m, 1H), 2.61 (m, 2H), 2.37 (m, 1H), |
| | | | | 2.27-2.19 (m, |
| | | | | 1H), 1.98 (m, |
| | | | | 1H), 0.73 (m, |
| | | | | 2H), 0.64 (m, |
| | | | | 2H) |
| 80 | 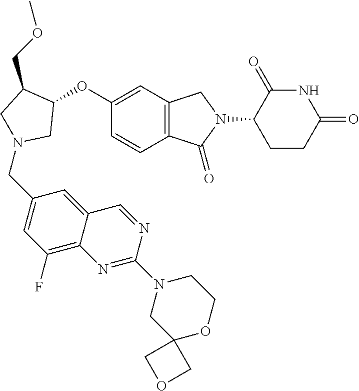 [SMILES: COC[C@@H]1CN(Cc2cc(F)c3nc(N4CCOC5(COC5)C4)ncc3c2)C[C@H]1Oc1ccc2c(c1)CN([C@H]1CCC(=O)NC1=O)C2=O] | (3S)-3-(5- {[(3S,4S)-1-[(2- {2,5-dioxa-8- azaspiro[3.5] nonan-8-yl}-8- fluoroquinazolin- 6-yl)methyl]-4- (methoxymethyl) pyrrolidin-3- yl]oxy}-1-oxo- 2,3-dihydro-1H- isoindol-2- yl)piperidine-2,6- dione | MS (ESI) m/z 661.3 [M + 1]⁺ | ¹H NMR (400 MHz, MeOD-d₄) δ 9.50-9.46 (m, 1H), 7.98-7.96 (m, 1H), 7.78 (d, J = 8.5 Hz, 1H), 7.65-7.50 (m, 1H), 7.12 (d, J = 2.2 Hz, 1H), 7.06-7.04 (m, 1H), 5.10-5.08 (m, 2H), 4.63- 4.57 (m, 4H), 4.50-4.38 (m, 3H), 4.28-4.16 (m, 1H), 4.10- 4.05 (m, 2H), 3.96-3.83 (m, 2H), 3.76-3.52 (m, 5H), 3.52- 3.49 (m, 2H), 3.30 (s, 3H), 3.12-3.08 (m, 1H), 2.98-2.88 |
| | | | | (m, 1H), 2.78- |
| | | | | 2.68 (m, 1H), |
| | | | | 2.60 (m, 2H), |
| | | | | 2.42-2.36 (m, |
| | | | | 1H), 2.02-1.88 |
| | | | | (m, 1H) |
| 81 | 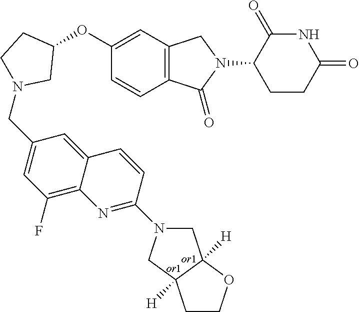 [SMILES: O=C1CC[C@H](N2Cc3cc(O[C@H]4CCN(Cc5cc(F)c6nc(N7C[C@@H]8CCO[C@@H]8C7)ccc6c5)C4)ccc3C2=O)C(=O)N1] | (S)-3-(5-(((S)-1- ((8-fluoro-2- ((3aS*,6aS*)- hexahydro-5H- furo[2,3-c]pyrrol- 5-yl)quinolin-6- yl)methyl) pyrrolidin- 3-yl)oxy)-1- oxoisoindolin-2- yl)piperidine-2,6- dione | MS (ESI) m/z 600.3 [M + 1]⁺ | ¹H NMR (400 MHz, DMSO-d₆) δ 8.03 (m, 1H), 7.61 (d, J = 8.4 Hz, 1H), 7.44 (d, J = 1.8 Hz, 1H), 7.31 (m, 1H), 7.11 (d, J = 2.1 Hz, 1H), 7.04- 6.93 (m, 2H), 5.05 (m, 1H), 5.11-4.95 (m, 1H), 4.57 (t, J = 5.4 Hz, 1H), 4.37 (d, J = 17.3 Hz, 1H), 4.24 (d, J = 17.2 Hz, 1H), 3.91-3.80 (m, 1H), 3.83-3.71 |
| | | | | (m, 3H), 3.71- |
| | | | | 3.60 (m, 3H), |
| | | | | 3.43-3.35 (m, |
| | | | | 1H), 3.11-3.01 |
| | | | | (m, 1H), 2.90 (m, |
| | | | | 2H), 2.79-2.64 |
| | | | | (m, 2H), 2.59 (m, |
| | | | | 1H), 2.54 (m, |
| | | | | 1H), 2.50-2.28 |
| | | | | (m, 2H), 2.22- |
| | | | | 2.09 (m, 1H), |
| | | | | 1.98 (m, 1H), |
| | | | | 1.86 (m, 2H) |
| 82 | 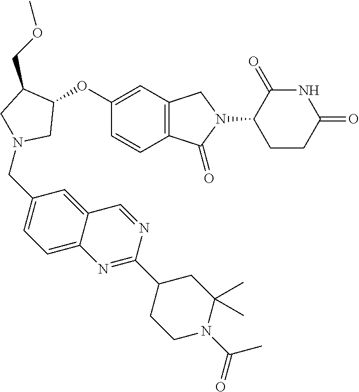 [SMILES: COC[C@@H]1CN(Cc2ccc3nc(C4CCN(C(C)=O)C(C)(C)C4)ncc3c2)C[C@H]1Oc1ccc2c(c1)CN([C@H]1CCC(=O)NC1=O)C2=O] | (3S)-3-(5- {[(3S,4S)-1-{[2- (1-acetyl-2,2- dimethylpiperidin- 4-yl)quinazolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 669.4 [M + 1]⁺ | ¹H NMR (400 MHz, MeOD-d₄) δ 9.48 (d, J = 2.2 Hz, 1H), 8.23 (s, 1H), 8.07 (d, J = 9.7 Hz, 2H), 7.98 (d, J = 9.0 Hz, 1H), 7.70 (dd, J = 8.6, 2.0 Hz, 1H), 7.22-7.02 (m, 2H), 5.12 (m, 1H), 4.54-4.36 (m, 2H), 4.08 (m, 2H), 3.82-3.80 (m, 1H), 3.64- 3.46 (m, 4H), 3.45-3.36 (m, 4H), 3.30-3.23 (m, 1H), 3.12 (m, 2H), 2.90-2.88 (m, 1H), 2.84- 2.71 (m, 2H), 2.62 (m, 1H), 2.55-2.42 (m, |
| | | | | 1H), 2.32-2.09 |
| | | | | (m, 7H), 1.92- |
| | | | | 1.83 (m, 1H), |
| | | | | 1.68-1.55 (m, |
| | | | | 6H) |
| 83 | 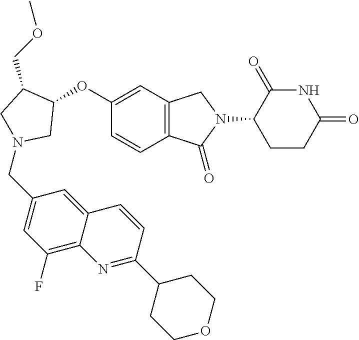 [SMILES: COC[C@H]1CN(Cc2cc(F)c3nc(C4CCOCC4)ccc3c2)C[C@H]1Oc1ccc2c(c1)CN([C@H]1CCC(=O)NC1=O)C2=O] | (3S)-3-(5- {[(3S,4R)-1-{[8- fluoro-2-(oxan-4- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 617.3 [M + 1]⁺ | ¹H NMR (400 MHz, MeOD-d₄) δ 8.28-8.20 (m, 1H), 7.66 (m, 2H), 7.54 (m, 2H), 7.12-7.02 (m, 1H), 7.03 (m, 1H), 5.09 (m, 2H), 4.48-4.32 (m, 2H), 4.14- 4.04 (m, 2H), 3.97 (s, 2H), 3.70-3.55 (m, 4H), 3.42-3.19 (m, 5H), 3.09 (m, 1H), 2.90-2.70 (m, 5H), 2.53- 2.38 (m, 1H), 2.19-2.10 (m, 1H), 2.00-1.90 (m, 4H) |
| 84 | 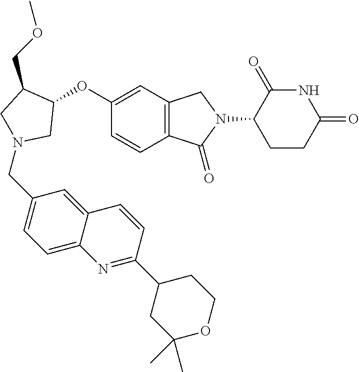 [SMILES: COC[C@@H]1CN(Cc2ccc3nc(C4CCOC(C)(C)C4)ccc3c2)C[C@H]1Oc1ccc2c(c1)CN([C@H]1CCC(=O)NC1=O)C2=O] | (3S)-3-(5- {[(3S,4S)-1-{[2- (2,2- dimethyloxan-4- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 627.6 [M + 1]⁺ | ¹H NMR (400 MHz, DMSO-d₆) δ 8.24 (d, J = 8.5 Hz, 1H), 7.90 (d, J = 8.6 Hz, 1H), 7.80 (d, J = 1.9 Hz, 1H), 7.67 (dd, J = 8.6, 1.9 Hz, 1H), 7.60 (d, J = 8.4 Hz, 1H), 7.46 (d, J = 8.5 Hz, 1H), 7.14 (d, J = 2.2 Hz, 1H), 7.03 (dd, J = 8.4, 2.2 Hz, 1H), 5.05 (dd, J = 13.3, 5.1 Hz, 1H), 4.74- 7.42 (m, 1H), 4.36 (d, J = 17.3 Hz, 1H), 4.25 (d, J = 17.3 Hz, 1H), 3.80-3.71 (m, 4H), 3.39 (m, |
| | | | | 2H), 3.29 (m, |
| | | | | 1H), 3.25 (s, 3H), |
| | | | | 2.98-2.84 (m, |
| | | | | 3H), 2.74 (m, |
| | | | | 1H), 2.65-2.53 |
| | | | | (m, 2H), 2.38- |
| | | | | 2.36 (m, 1H), |
| | | | | 2.23 (m, 1H), |
| | | | | 2.02-1.92 (m, |
| | | | | 1H), 1.76-1.72 |
| | | | | (m, 3H), 1.67 (m, |
| | | | | 1H), 1.28 (s, 3H), |
| | | | | 1.19 (s, 3H) |
| 85 | 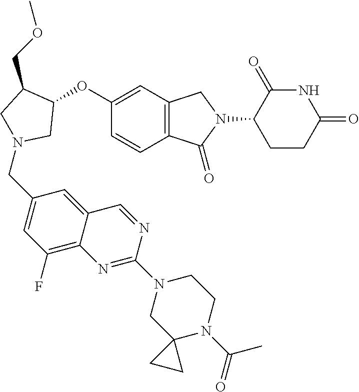 [SMILES: COC[C@@H]1CN(Cc2cc(F)c3nc(N4CCN(C(C)=O)C5(CC5)C4)ncc3c2)C[C@H]1Oc1ccc2c(c1)CN([C@H]1CCC(=O)NC1=O)C2=O] | (3S)-3-(5- {[(3S,4S)-1-[(2- {4-acetyl-4,7- diazaspiro[2.5] octan-7-yl}-8- fluoroquinazolin- 6-yl)methyl]-4- (methoxymethyl) pyrrolidin-3- yl]oxy}-1-oxo- 2,3-dihydro-1H- isoindol-2- yl)piperidine-2,6- dione | MS (ESI) m/z 686.3 [M + 1]⁺ | ¹H NMR (400 MHz, MeOD-d₄) δ 9.09 (s, 1H), 7.71 (d, J = 8.4 Hz, 1H), 7.69- 7.53 (m, 2H), 7.14 (d, J = 2.2 Hz, 1H), 7.08 (dd, J = 8.4, 2.1 Hz, 1H), 5.11 (dd, J = 13.3, 5.2 Hz, 1H), 4.87 (m, 1H), 4.47-4.41 (m, 2H), 4.04 (m, 2H), 3.96-3.70 (m, 6H), 3.54 (d, J = 6.2 Hz, 2H), 3.39 (s, 3H), 3.25 (m, 1H), 3.10 (m, 2H), 2.91 (m, 1H), 2.89-2.70 (m, 2H), 2.59 (m, 1H), 2.47 (m, 1H), 2.28 (s, 2H), |
| | | | | 2.18-2.10 (m, |
| | | | | 2H), 1.20-0.90 |
| | | | | (m, 4H) |
| 86 | 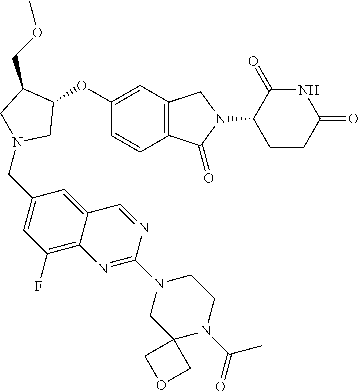 [SMILES: COC[C@@H]1CN(Cc2cc(F)c3nc(N4CCN(C(C)=O)C5(COC5)C4)ncc3c2)C[C@H]1Oc1ccc2c(c1)CN([C@H]1CCC(=O)NC1=O)C2=O] | (S)-3-(5- (((3S,4S)-1-((2- (5-acetyl-2-oxa- 5,8- diazaspiro[3.5] nonan-8-yl)-8- fluoroquinazolin- 6-yl)methyl)-4- (methoxymethyl) pyrrolidin-3- yl)oxy)-1- oxoisoindolin-2- yl)piperidine-2,6- dione | MS (ESI) m/z 702.2 [M + 1]⁺ | ¹H NMR (400 MHz, DMSO-d₆) δ 10.96 (s, 1H), 9.30 (d, J = 1.6 Hz, 1H), 7.64- 7.53 (m, 3H), 7.15 (d, J = 2.2 Hz, 1H), 7.03 (dd, J = 8.4, 2.2 Hz, 1H), 5.07 (dd, J = 13.3, 5.1 Hz, 1H), 4.75- 4.70 (m, 3H), 4.37 (d, J = 17.3 Hz, 1H), 4.31- 4.20 (m, 5H), 3.83 (m, 2H), 3.72-3.65 (m, 2H), 3.50-3.37 (m, 4H), 3.29 (s, 3H), 2.96-2.87 (m, 3H), 2.79- 2.71 (m, 1H), 2.64-2.53 (m, |
| | | | | 2H), 2.39-2.30 |
| | | | | (m, 1H), 2.24- |
| | | | | 2.21 (m, 1H), |
| | | | | 2.10 (s, 3H), |
| | | | | 1.98-1.91 (m, |
| | | | | 1H) |
| 87 | 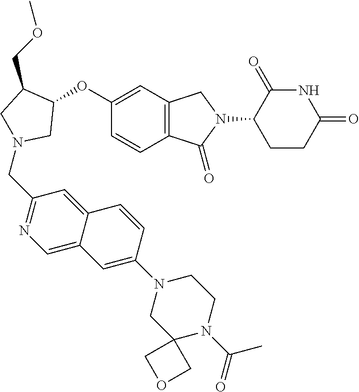 [SMILES: COC[C@@H]1CN(Cc2cc3ccc(N4CCN(C(C)=O)C5(COC5)C4)cc3cn2)C[C@H]1Oc1ccc2c(c1)CN([C@H]1CCC(=O)NC1=O)C2=O] | (3S)-3-(5- {[(3S,4S)-1-[(7- {5-acetyl-2-oxa- 5,8- diazaspiro[3.5] nonan-8- yl}isoquinolin-3- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 683.3 [M + 1]⁺ | ¹H NMR (400 MHz, MeOD-d₄) δ 9.06 (s, 1H), 7.82 (d, J = 9.0 Hz, 1H), 7.75- 7.66 (m, 3H), 7.43 (d, J = 2.3 Hz, 1H), 7.13 (d, J = 2.2 Hz, 1H), 7.07 (dd, J = 8.5, 2.2 Hz, 1H), 5.11 (dd, J = 13.3, 5.2 Hz, 1H), 4.93 (d, J = 6.8 Hz, 2H), 4.82 (m, 1H), 4.54-4.45 (m, 2H), 4.42 (d, J = 6.0 Hz, 2H), 3.95 (d, J = 13.3 Hz, 1H), 3.89 (d, J = 13.2 Hz, 1H), 3.71 (s, 2H), 3.68-3.57 (m, 2H), 3.52 (m, |
| | | | | 2H), 3.37 (s, 3H), |
| | | | | 3.28 (m, 2H), |
| | | | | 3.21-3.12 (m, |
| | | | | 1H), 3.00 (m, |
| | | | | 2H), 2.96-2.84 |
| | | | | (m, 1H), 2.83- |
| | | | | 2.74 (m, 1H), |
| | | | | 2.71 (m, 1H), |
| | | | | 2.55-2.40 (m, |
| | | | | 2H), 2.18-2.09 |
| | | | | (m, 4H) |
| 88 | 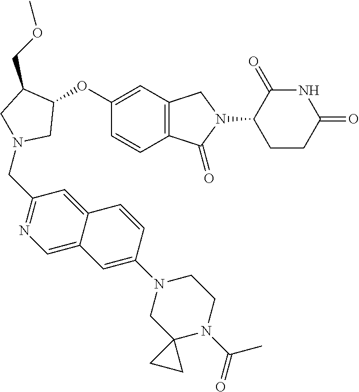 [SMILES: COC[C@@H]1CN(Cc2cc3ccc(N4CCN(C(C)=O)C5(CC5)C4)cc3cn2)C[C@H]1Oc1ccc2c(c1)CN([C@H]1CCC(=O)NC1=O)C2=O] | (3S)-3-(5- {[(3S,4S)-1-[(7- {4-acetyl-4,7- diazaspiro[2.5] octan-7- yl}isoquinolin-3- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 667.4 [M + 1]⁺ | ¹H NMR (400 MHz, DMSO-d₆) δ 9.00 (s, 1H), 7.77 (d, J = 9.1 Hz, 1H), 7.64- 7.56 (m, 3H), 7.26 (d, J = 2.4 Hz, 1H), 7.14 (d, J = 2.2 Hz, 1H), 7.03 (dd, J = 8.4, 2.2 Hz, 1H), 5.04 (dd, J = 13.3, 5.1 Hz, 1H), 4.77- 4.69 (m, 1H), 4.36 (d, J = 17.2 Hz, 1H), 4.25 (d, J = 17.3 Hz, 1H), 3.86-3.73 (m, 4H), 3.42 (s, 3H), 3.41-3.36 (m, 1H), 3.26 (m, 3H), 3.21-3.09 (m, 2H),3.05- 2.76 (m, 4H), |
| | | | | 2.64-2.53 (m, |
| | | | | 2H), 2.35 (m, |
| | | | | 2H), 2.14 (s, 2H), |
| | | | | 2.10-1.90 (m, |
| | | | | 2H), 1.25-0.81 |
| | | | | (m, 4H) |
| 89 | 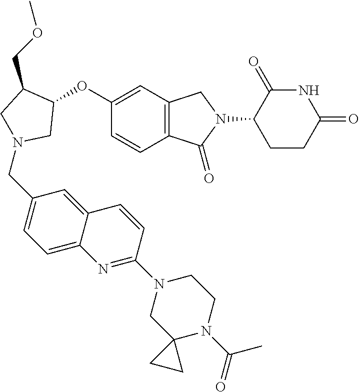 [SMILES: COC[C@@H]1CN(Cc2ccc3nc(N4CCN(C(C)=O)C5(CC5)C4)ccc3c2)C[C@H]1Oc1ccc2c(c1)CN([C@H]1CCC(=O)NC1=O)C2=O] | (3S)-3-(5- {[(3S,4S)-1-[(2- {4-acetyl-4,7- diazaspiro[2.5] octan-7-yl} quinolin- 6-yl)methyl]-4- (methoxymethyl) pyrrolidin-3- yl]oxy}-1-oxo- 2,3-dihydro-1H- isoindol-2- yl)piperidine-2,6- dione | MS (ESI) m/z 667.1 [M + 1]⁺ | ¹H NMR (400 MHz, MeOD-d₄) δ 7.97 (d, J = 9.2 Hz, 1H), 7.70 (d, J = 8.5 Hz, 1H), 7.68-7.57 (m, 3H), 7.15-7.12 (m, 2H), 7.07 (dd, J = 8.5, 2.2 Hz, 1H), 5.12 (dd, J = 13.3, 5.1 Hz, 1H), 4.87- 4.80 (m, 1H), 4.42-4.41 (m, 2H), 3.90-3.72 (m, 6H), 3.68 (m, 2H), 3.50 (m, 2H), 3.40 (s, 2H), 3.12 (m, 1H), 2.94-2.85 (m, 3H), 2.81 (m, 1H), 2.70 (m, 1H), 2.47 (m, 1H), 2.38 (m, |
| | | | | 1H), 2.28 (s, 2H), |
| | | | | 2.16 (m, 2H), |
| | | | | 1.15-1.13 (m, |
| | | | | 3H), 0.91 (m, |
| | | | | 1H) |
| 90 | 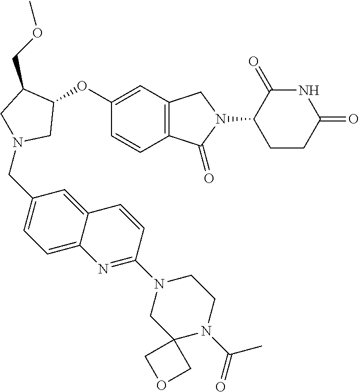 [SMILES: COC[C@@H]1CN(Cc2ccc3nc(N4CCN(C(C)=O)C5(COC5)C4)ccc3c2)C[C@H]1Oc1ccc2c(c1)CN([C@H]1CCC(=O)NC1=O)C2=O] | (3S)-3-(5- {[(3S,4S)-1-[(2- {5-acetyl-2-oxa- 5,8- diazaspiro[3.5] nonan-8- yl}quinolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 683.4 [M + 1]⁺ | ¹H NMR (400 MHz, MeOD-d₄) δ 8.03 (d, J = 8.9 Hz, 1H), 7.73- 7.57 (m, 4H), 7.23 (d, J = 9.2 Hz, 1H), 7.13 (s, 1H), 7.08 (dd, J = 8.7, 2.0 Hz, 1H), 5.11 (dd, J = 13.3, 5.1 Hz, 1H), 4.93 (m, 2H), 4.80 (m, 1H), 4.43 (m, 4H), 4.15 (s, 2H), 3.82 (m, 2H), 3.71-3.55 (m, 4H), 3.52 (m, 2H), 3.38 (s, 3H), 3.13 (m, 1H), 2.95 (m, 3H), 2.75-2.67 (m, 2H), 2.55-2.42 (m, 2H), 2.20- |
| | | | | 2.10 (m, 4H) |
| 91 | 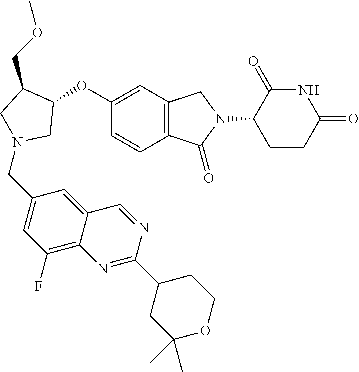 [SMILES: COC[C@@H]1CN(Cc2cc(F)c3nc(C4CCOC(C)(C)C4)ncc3c2)C[C@H]1Oc1ccc2c(c1)CN([C@H]1CCC(=O)NC1=O)C2=O] | (3S)-3-(5- {[(3S,4S)-1-{[2- (2,2- dimethyloxan-4- yl)-8- fluoroquinazolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 646.4 [M + 1]⁺ | ¹H NMR (400 MHz, DMSO-d₆) δ 9.56 (s, 1H), 7.85 (s, 1H), 7.77 (d, J = 11.5 Hz, 1H), 7.61 (d, J = 8.4 Hz, 1H), 7.13 (s, 1H), 7.06-7.00 (m, 1H), 5.01 (dd, J = 13.3, 5.1 Hz, 1H), 4.73 (d, J = 5.1 Hz, 1H), 4.37 (d, J = 17.3 Hz, 1H), 4.24 (d, J = 17.4 Hz, 1H), 3.81-3.72 (m, 4H), 3.46-3.35 (m, 3H), 3.24 (s, 3H), 3.00-2.79 (m, 3H), 2.74 (m, 1H), 2.62 (m, 1H), 2.59-2.52 (m, 1H), 2.42- |
| | | | | 2.28 (m, 1H), |
| | | | | 2.23 (m, 1H), |
| | | | | 1.98 (m, 1H), |
| | | | | 1.87 (m, 2H), |
| | | | | 1.78 (m, 1H), |
| | | | | 1.67 (m, 1H), |
| | | | | 1.27 (s, 3H), 1.18 |
| | | | | (s, 3H) |
| 92 | 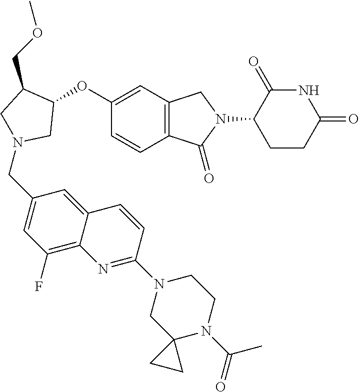 [SMILES: COC[C@@H]1CN(Cc2cc(F)c3nc(N4CCN(C(C)=O)C5(CC5)C4)ccc3c2)C[C@H]1Oc1ccc2c(c1)CN([C@H]1CCC(=O)NC1=O)C2=O] | (3S)-3-(5- {[(3S,4S)-1-[(2- {4-acetyl-4,7- diazaspiro[2.5] octan-7-yl}-8- fluoroquinolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 685.6 [M + 1]⁺ | ¹H NMR (400 MHz, MeOD-d₄) δ 7.98 (dd, J = 9.2, 1.7 Hz, 1H), 7.70 (d, J = 8.5 Hz, 1H), 7.42 (d, J = 1.8 Hz, 1H), 7.34 (dd, J = 11.9, 1.8 Hz, 1H), 7.22-7.04 (m, 3H), 5.11 (m, 1H), 4.82 (m, 1H), 4.50-4.35 (m, 2H), 3.90 (m, 2H), 3.85-3.70 (m, 5H), 3.65 (m, 1H), 3.52 (m, 2H), 3.38 (s, 3H), 3.10 (m, 1H), 2.98-2.84 (m, 3H), 2.84-2.64 (m, 2H), 2.55- 2.40 (m, 1H), 2.38 (m, 1H), |
| | | | | 2.28 (s, 2H), |
| | | | | 2.22-2.14 (m, |
| | | | | 2H), 1.14-1.07 |
| | | | | (m, 4H) |
| 93 | 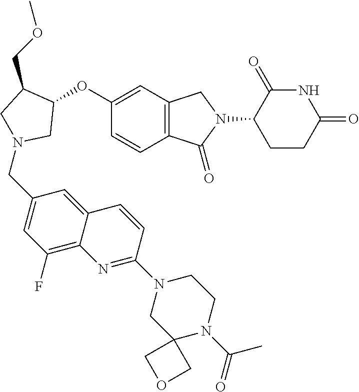 [SMILES: COC[C@@H]1CN(Cc2cc(F)c3nc(N4CCN(C(C)=O)C5(COC5)C4)ccc3c2)C[C@H]1Oc1ccc2c(c1)CN([C@H]1CCC(=O)NC1=O)C2=O] | (3S)-3-(5- {[(3S,4S)-1-[(2- {5-acetyl-2-oxa- 5,8- diazaspiro[3.5] nonan-8-yl}-8- fluoroquinolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 701.4 [M + 1]⁺ | ¹H NMR (400 MHz, MeOD-d₄) δ 8.04 (dd, J = 9.3, 1.6 Hz, 1H), 7.70 (d, J = 8.4 Hz, 1H), 7.44 (s, 1H), 7.36 (dd, J = 11.8, 1.8 Hz, 1H), 7.28 (d, J = 9.3 Hz, 1H), 7.13 (d, J = 2.1 Hz, 1H), 7.08 (dd, J = 8.5, 2.2 Hz, 1H), 5.11 (dd, J = 13.3, 5.1 Hz, 1H), 4.92 (d, J = 6.7 Hz, 2H), 4.81 (m, 1H), 4.42 (d, J = 6.6 Hz, 4H), 4.18 (s, 2H), 3.79 (m, 1H), 3.76- 3.69 (m, 3H), 3.61-3.49 (m, 4H), 3.38 (s, 3H), |
| | | | | 3.09 (m, 1H), |
| | | | | 2.98-2.84 (m, |
| | | | | 3H), 2.84-2.66 |
| | | | | (m, 2H), 2.47 (m, |
| | | | | 1H), 2.36 (m, |
| | | | | 1H), 2.19 (m, |
| | | | | 4H) |
| 94 | 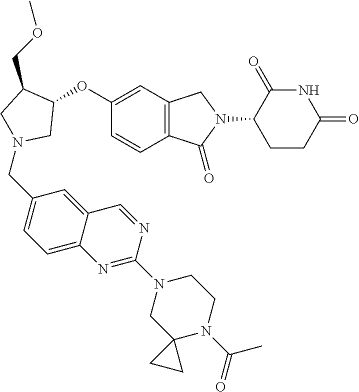 [SMILES: COC[C@@H]1CN(Cc2ccc3nc(N4CCN(C(C)=O)C5(CC5)C4)ncc3c2)C[C@H]1Oc1ccc2c(c1)CN([C@H]1CCC(=O)NC1=O)C2=O] | (3S)-3-(5- {[(3S,4S)-1-[(2- {4-acetyl-4,7- diazaspiro[2.5] octan-7- yl}quinazolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 668.2 [M + 1]⁺ | ¹H NMR (400 MHz, MeOD-d₄) δ 9.07 (s, 1H), 7.77 (dd, J = 8.7, 2.0 Hz, 1H), 7.75-7.69 (m, 2H), 7.53 (d, J = 8.6 Hz, 1H), 7.13 (d, J = 2.1 Hz, 1H), 7.08 (dd, J = 8.5, 2.2 Hz, 1H), 5.12 (dd, J = 13.3, 5.2 Hz, 1H), 4.87-4.81 (m, 1H), 4.44 (m, 2H), 4.00 (m, 2H), 3.90-3.76 (m, 6H), 3.53- 3.51 (m, 2H), 3.38 (s, 3H), 3.10 (m, 1H), 2.92- 2.90 (m, 3H), 2.81 (m, 1H), 2.70 (m, 1H), |
| | | | | 2.48 (m, 1H), |
| | | | | 2.37 (m, 1H), |
| | | | | 2.28 (s, 2H), 2.15 |
| | | | | (m, 2H), 1.10 (m, |
| | | | | 3H), 0.98-0.88 |
| | | | | (m, 1H) |
| 95 | 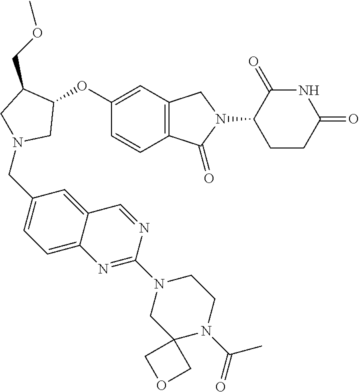 [SMILES: COC[C@@H]1CN(Cc2ccc3nc(N4CCN(C(C)=O)C5(COC5)C4)ncc3c2)C[C@H]1Oc1ccc2c(c1)CN([C@H]1CCC(=O)NC1=O)C2=O] | (3S)-3-(5- {[(3S,4S)-1-[(2- {5-acetyl-2-oxa- 5,8- diazaspiro[3.5] nonan-8- yl}quinazolin-6- yl)methyl]-4- (methoxymethyl) pyrrolidin-3- yl]oxy}-1-oxo- 2,3-dihydro-1H- isoindol-2- yl)piperidine-2,6- dione | MS (ESI) m/z 684.3 [M + 1]⁺ | ¹H NMR (400 MHz, MeOD-d₄) δ 9.12 (s, 1H), 7.82-7.73 (m, 2H), 7.70 (d, J = 8.5 Hz, 1H), 7.58 (d, J = 8.6 Hz, 1H), 7.13 (s, 1H), 7.11-7.04 (m, 1H), 5.11-5.10 (m, 1H), 4.80 (m, 2H), 4.55-4.53 (m, 1H), 4.50- 4.35 (m, 6H), 3.92 (m, 2H), 3.81 (d, J = 12.8 Hz, 1H), 3.75 (d, J = 12.8 Hz, 1H), 3.53 (m, 4H), 3.37 (s, 3H), 3.14-3.05 (m, 1H), 2.89 (m, 3H), 2.80 (m, 1H), 2.73-2.65 |
| | | | | (m, 1H), 2.54- |
| | | | | 2.40 (m, 1H), |
| | | | | 2.40-2.32 (m, |
| | | | | 1H), 2.20 (s, 3H), |
| | | | | 2.20-2.10 (m, |
| | | | | 1H) |
| 96 | 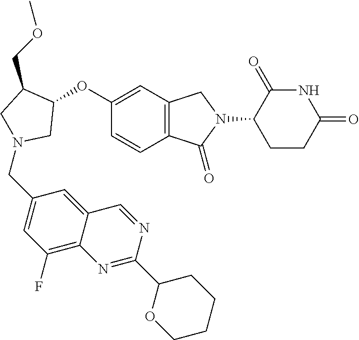 [SMILES: COC[C@@H]1CN(Cc2cc(F)c3nc(C4CCCCO4)ncc3c2)C[C@H]1Oc1ccc2c(c1)CN([C@H]1CCC(=O)NC1=O)C2=O] | rel-(3S)-3-(5- {[(3S,4S)-1-{[8- fluoro-2-(oxan-2- yl)quinazolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.3 [M + 1]⁺ | ¹H NMR (400 MHz, MeOD-d₄) δ 9.54 (d, J = 1.4 Hz, 1H), 7.90 (d, J = 1.6 Hz, 1H), 7.85 (dd, J = 11.2, 1.7 Hz, 1H), 7.71 (d, J = 8.4 Hz, 1H), 7.14 (d, J = 2.2 Hz, 1H), 7.07 (dd, J = 8.5, 2.2 Hz, 1H), 5.14 (dd, J = 13.3, 5.2 Hz, 1H), 4.87-4.76 (m, 1H), 4.43 (m, 2H), 4.18 (m, 1H), 3.95 (s, 2H), 3.74 (m, 1H), 3.55 (m, 2H), 3.39 (s, 2H), 3.15 |
| | | | | (m, 1H), 3.05 (m, |
| | | | | 1H), 3.03-2.85 |
| | | | | (m, 2H), 2.71 (m, |
| | | | | 2H), 2.50 (m, |
| | | | | 2H), 2.20 (m, |
| | | | | 1H), 2.10 (m, |
| | | | | 2H), 1.84 (m, |
| | | | | 3H), 1.68 (m, |
| | | | | 1H), 1.31 (m, |
| | | | | 1H) |
| 97 | 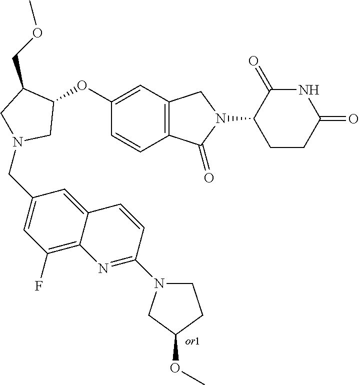 [SMILES: COC[C@@H]1CN(Cc2cc(F)c3nc(N4CC[C@@H](OC)C4)ccc3c2)C[C@H]1Oc1ccc2c(c1)CN([C@H]1CCC(=O)NC1=O)C2=O] | (S)-3-(5- (((3S,4S)-1-((8- fluoro-2-((R*)-3- methoxypyrrolidin- 1-yl)quinolin-6- yl)methyl)-4- (methoxymethyl) pyrrolidin-3- yl)oxy)-1- oxoisoindolin-2- yl)piperidine-2,6- dione | MS (ESI) m/z 632.5 [M + 1]⁺ | ¹H NMR (400 MHz, DMSO-d₆) δ 7.98 (dd, J = 9.3, 1.7 Hz, 1H), 7.61 (d, J = 8.5 Hz, 1H), 7.39 (d, J = 1.7 Hz, 1H), 7.27 (dd, J = 12.2, 1.8 Hz, 1H), 7.12 (d, J = 2.2 Hz, 1H), 7.02 (dd, J = 8.4, 2.2 Hz, 1H), 6.91 (d, J = 9.2 Hz, 1H), 4.99 (dd, J = 13.3, 5.2 Hz, 1H), 4.71-4.69 (m, 1H), 4.37 (d, J = 17.4 Hz, 1H), 4.24 (d, J = 17.5 Hz, 1H), 4.12- 4.06 (m, 1H), 3.67-3.59 (m, 4H), 3.47 (m, |
| | | | | 2H), 3.43-3.34 |
| | | | | (m, 2H), 3.25 (s, |
| | | | | 3H), 3.23 (s, 3H), |
| | | | | 2.93 (m, 1H), |
| | | | | 2.84-2.81 (m, |
| | | | | 2H), 2.72 (m, |
| | | | | 1H), 2.67-2.54 |
| | | | | (m, 2H), 2.34- |
| | | | | 2.32 (m, 1H), |
| | | | | 2.20 (m, 1H), |
| | | | | 2.06-2.03 (m, |
| | | | | 2H), 2.02-1.95 |
| | | | | (m, 1H) |
| 98 | 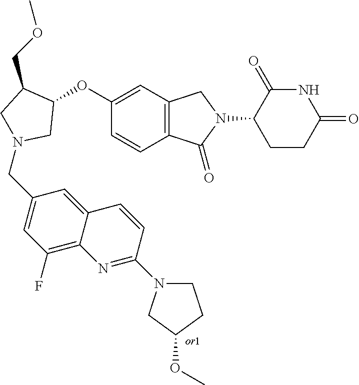 [SMILES: COC[C@@H]1CN(Cc2cc(F)c3nc(N4CC[C@H](OC)C4)ccc3c2)C[C@H]1Oc1ccc2c(c1)CN([C@H]1CCC(=O)NC1=O)C2=O] | (S)-3-(5- (((3S,4S)-1-((8- fluoro-2-((S*)-3- methoxypyrrolidin- 1-yl)quinolin-6- yl)methyl)-4- (methoxymethyl) pyrrolidin-3- yl)oxy)-1- oxoisoindolin-2- yl)piperidine-2,6- dione | MS (ESI) m/z 632.6 [M + 1]⁺ | ¹H NMR (400 MHz, DMSO-d₆) δ 7.99 (dd, J = 9.2, 1.7 Hz, 1H), 7.61 (d, J = 8.4 Hz, 1H), 7.39 (d, J = 1.7 Hz, 1H), 7.27 (dd, J = 12.1, 1.8 Hz, 1H), 7.13 (d, J = 2.2 Hz, 1H), 7.02 (dd, J = 8.5, 2.2 Hz, 1H), 6.92 (d, J = 9.2 Hz, 1H), 5.00 (dd, J = 13.2, 5.0 Hz, 1H), 4.71-4.69 (m, 1H), 4.37 (d, J = 17.4 Hz, 1H), 4.24 (d, J = 17.4 Hz, 1H), 4.09- 4.07 (m, 1H), 3.60 (m, 4H), 3.52-3.44 (m, |
| | | | | 2H), 3.43-3.35 |
| | | | | (m, 2H), 3.25 (s, |
| | | | | 3H), 3.23 (s, 3H), |
| | | | | 2.87-2.82 (m, |
| | | | | 3H), 2.72 (m, |
| | | | | 1H), 2.63-2.53 |
| | | | | (m, 2H), 2.35 (m, |
| | | | | 1H), 2.20 (m, |
| | | | | 1H), 2.08-2.04 |
| | | | | (m, 2H), 2.01- |
| | | | | 1.94 (m, 1H) |
| 99 | 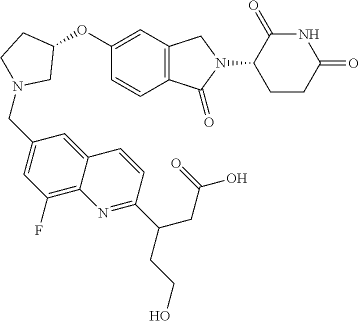 [SMILES: O=C(O)CC(CCO)c1ccc2cc(CN3CC[C@H](Oc4ccc5c(c4)CN([C@H]4CCC(=O)NC4=O)C5=O)C3)cc(F)c2n1] | rel-3-(6-{[(3S)-3- ({2-[(3S)-2,6- dioxopiperidin-3- yl]-1-oxo-2,3- dihydro-1H- isoindol-5- yl}oxy)pyrrolidin- 1-yl]methyl}-8- fluoroquinolin-2- yl)-5- hydroxypentanoic acid | MS (ESI) m/z 605.4 [M + 1]⁺ | ¹H NMR (400 MHz, DMSO-d₆) δ 11.83 (s, 1H), 10.95 (s, 1H), 8.30 (d, J = 8.6 Hz, 1H), 7.69 (s, 1H), 7.60 (d, J = 8.4 Hz, 1H), 7.52 (dd, J = 10.4, 7.0 Hz, 2H), 7.11 (s, 1H), 7.01 (dd, J = 8.4, 2.2 Hz, 1H), 5.06-5.01 (m, 2H), 4.48 (t, J = 5.1 Hz, 1H), 4.37 (d, J = 17.2 Hz, 1H), 4.24 (dd, J = 17.3, 3.9 Hz, 1H), 3.83-3.76 (m, 3H), 3.51 (m, |
| | | | | 1H), 2.98 (m, |
| | | | | 1H), 2.96-2.83 |
| | | | | (m, 2H), 2.77 (m, |
| | | | | 1H), 2.68 (m, |
| | | | | 3H), 2.60 (m, |
| | | | | 1H), 2.55 (m, |
| | | | | 1H), 2.43-2.29 |
| | | | | (m, 2H), 1.97 (m, |
| | | | | 1H), 1.83 (m, |
| | | | | 3H) |
| 100 | 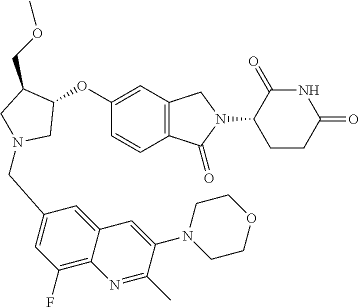 [SMILES: COC[C@@H]1CN(Cc2cc(F)c3nc(C)c(N4CCOCC4)cc3c2)C[C@H]1Oc1ccc2c(c1)CN([C@H]1CCC(=O)NC1=O)C2=O] | (3S)-3-(5- {[(3S,4S)-1-{[8- fluoro-2-methyl- 3-(morpholin-4- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 632.3 [M + 1]⁺ | ¹H NMR (400 MHz, MeOD-d₄) δ 8.39-8.12 (m, 1H), 7.82-7.74 (m, 2H), 7.69 (m, 1H), 7.61 (d, J = 11.5 Hz, 1H), 7.14 (m, 1H), 7.08 (m, 1H), 5.15-5.06 (m, 2H), 4.50-4.34 (m, 2H), 4.16- 4.01 (m, 4H), 3.78 (t, J = 4.6 Hz, 4H), 3.55 (d, J = 6.1 Hz, 2H), 3.39 (s, 3H), 3.30 (m, 2H), 3.15 (m, 2H), 2.97-2.84 |
| | | | | (m, 1H), 2.75- |
| | | | | 2.65 (m, 7H), |
| | | | | 2.54-2.39 (m, |
| | | | | 1H), 2.21-2.10 |
| | | | | (m, 1H) |
| 101 | 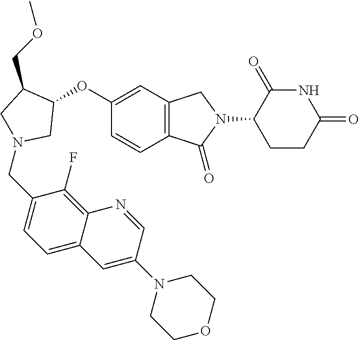 [SMILES: COC[C@@H]1CN(Cc2ccc3cc(N4CCOCC4)cnc3c2F)C[C@H]1Oc1ccc2c(c1)CN([C@H]1CCC(=O)NC1=O)C2=O] | (3S)-3-(5- {[(3S,4S)-1-{[8- fluoro-3- (morpholin-4- yl)quinolin-7- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.3 [M + 1]⁺ | ¹H NMR (400 MHz, DMSO-d₆) δ 10.96 (s, 1H), 8.90 (d, J = 2.7 Hz, 1H), 7.64- 7.54 (m, 3H), 7.50-7.40 (m, 1H), 7.14 (d, J = 2.2 Hz, 1H), 7.10-7.01 (m, 1H), 5.05 (m, 1H), 4.82-4.70 (m, 1H), 4.40- 4.22 (m, 2H), 3.82-3.62 (m, 6H), 3.52-3.37 (m, 6H), 3.22 (s, 3H), 2.99-2.84 (m, 3H), 2.76 (m, 1H), 2.68-2.54 (m, 2H), 2.43- |
| | | | | 2.31 (m, 1H), |
| | | | | 2.28-2.20 (m, |
| | | | | 1H), 2.05-1.90 |
| | | | | (m, 1H) |
| 102 | 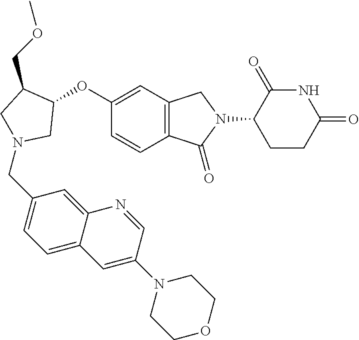 [SMILES: COC[C@@H]1CN(Cc2ccc3cc(N4CCOCC4)cnc3c2)C[C@H]1Oc1ccc2c(c1)CN([C@H]1CCC(=O)NC1=O)C2=O] | (3S)-3-(5- {[(3S,4S)-4- (methoxymethyl)- 1-{[3-(morpholin- 4-yl)quinolin-7- yl]methyl} pyrrolidin- 3-yl]oxy}-1- oxo-2,3-dihydro- 1H-isoindol-2- yl)piperidine-2,6- dione | MS (ESI) m/z 600.3 [M + 1]⁺ | ¹H NMR (400 MHz, DMSO-d₆) δ 8.83 (d, J = 2.9 Hz, 1H), 7.78- 7.70 (m, 2H), 7.60 (d, J = 8.4 Hz, 1H), 7.52 (d, J = 2.8 Hz, 1H), 7.46 (dd, J = 8.4, 1.7 Hz, 1H), 7.14 (d, J = 2.2 Hz, 1H), 7.03 (dd, J = 8.4, 2.2 Hz, 1H), 5.04 (dd, J = 13.3, 5.1 Hz, 1H), 4.75-4.66 (m, 1H), 4.36 (d, J = 17.3 Hz, 1H), 4.25 (d, J = 17.3 Hz, 1H), 3.84- 3.56 (m, 6H), |
| | | | | 3.25 (m, 7H), |
| | | | | 2.90-2.87 (m, |
| | | | | 3H), 2.77-2.72 |
| | | | | (m, 1H), 2.68- |
| | | | | 2.53 (m, 3H), |
| | | | | 2.43-2.29 (m, |
| | | | | 2H), 2.23 (m, |
| | | | | 1H), 2.00-1.93 |
| | | | | (m, 1H) |
| 103 | 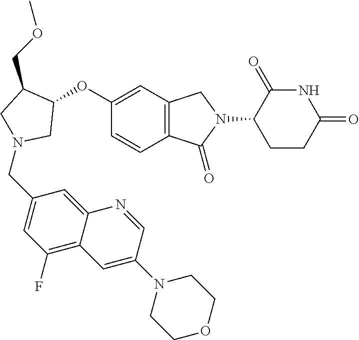 [SMILES: COC[C@@H]1CN(Cc2cc(F)c3cc(N4CCOCC4)cnc3c2)C[C@H]1Oc1ccc2c(c1)CN([C@H]1CCC(=O)NC1=O)C2=O] | (3S)-3-(5- {[(3S,4S)-1-{[5- fluoro-3- (morpholin-4- yl)quinolin-7- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.5 [M + 1]⁺ | ¹H NMR (400 MHz, MeOD-d₄) δ 8.84 (d, J = 2.8 Hz, 1H), 7.75- 7.66 (m, 2H), 7.61 (d, J = 2.8 Hz, 1H), 7.34 (d, J = 11.0 Hz, 1H), 7.13 (d, J = 2.2 Hz, 1H), 7.07 (dd, J = 8.5, 2.2 Hz, 1H), 5.11 (dd, J = 13.3, 5.1 Hz, 1H), 4.86 (m, 1H), 4.49-4.34 (m, 2H), 3.99- 3.85 (m, 6H), 3.53 (d, J = 6.5 Hz, 2H), 3.37 (s, 3H), 3.36-3.31 (m, 4H), 3.23- |
| | | | | 3.14 (m, 1H), |
| | | | | 3.06-2.99 (m, |
| | | | | 2H), 2.96-2.85 |
| | | | | (m, 1H), 2.83- |
| | | | | 2.66 (m, 2H), |
| | | | | 2.56-2.39 (m, |
| | | | | 2H), 2.17-2.11 |
| | | | | (m, 1H) |
| 104 | 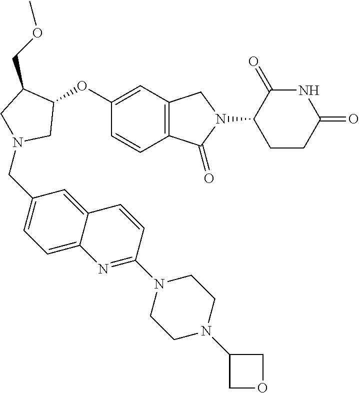 [SMILES: COC[C@@H]1CN(Cc2ccc3nc(N4CCN(C5COC5)CC4)ccc3c2)C[C@H]1Oc1ccc2c(c1)CN([C@H]1CCC(=O)NC1=O)C2=O] | (3S)-3-(5- {[(3S,4S)-4- (methoxymethyl)- 1-({2-[4-(oxetan- 3-yl)piperazin-1- yl]quinolin-6- yl}methyl) pyrrolidin- 3-yl]oxy}-1- oxo-2,3-dihydro- 1H-isoindol-2- yl)piperidine-2,6- dione | MS (ESI) m/z 655.5 [M + 1]⁺ | ¹H NMR (400 MHz, DMSO-d₆) δ 10.97 (s, 1H), 8.01 (d, J = 9.2 Hz, 1H), 7.63- 7.56 (m, 2H), 7.55-7.44 (m, 2H), 7.22 (d, J = 9.3 Hz, 1H), 7.14 (d, J = 2.2 Hz, 1H), 7.02 (dd, J = 8.4, 2.2 Hz, 1H), 5.05 (dd, J = 13.3, 5.1 Hz, 1H), 4.73-4.70 (m, 1H), 4.57- 4.49 (m, 4H), 4.35-4.24 (m, 2H), 3.73-3.59 (m, 6H), 3.45- 3.42 (m, 3H), 3.25 (s, 3H), 2.96-2.83 (m, 3H), 2.72 (m, |
| | | | | 1H), 2.58-2.52 |
| | | | | (m, 2H), 2.44- |
| | | | | 2.28 (m, 5H), |
| | | | | 2.19-2.13 (m, |
| | | | | 1H), 2.01-1.92 |
| | | | | (m, 1H) |
| 105 | 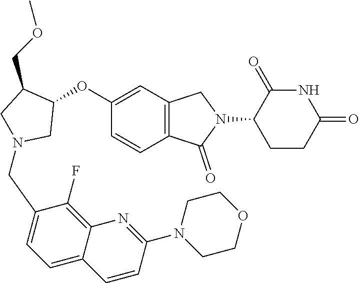 [SMILES: COC[C@@H]1CN(Cc2ccc3ccc(N4CCOCC4)nc3c2F)C[C@H]1Oc1ccc2c(c1)CN([C@H]1CCC(=O)NC1=O)C2=O] | (3S)-3-(5- {[(3S,4S)-1-{[8- fluoro-2- (morpholin-4- yl)quinolin-7- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 618.3 [M + 1]⁺ | ¹H NMR (400 MHz, DMSO-d₆) δ 10.96 (s, 1H), 8.07 (d, J = 9.0 Hz, 1H), 7.63- 7.56 (m, 1H), 7.49 (m, 1H), 7.32-7.17 (m, 2H), 7.14 (s, 1H), 7.01 (d, J = 8.5 Hz, 1H), 5.05 (m, 1H), 4.72 (s, 1H), 4.36 (d, J = 17.2 Hz, 1H), 4.25 (d, J = 17.1 Hz, 1H), 3.78 (s, 2H), 3.76-3.64 (m, 8H), 3.40 (m, |
| | | | | 2H), 3.21 (m, |
| | | | | 3H), 2.80 (m, |
| | | | | 1H), 2.60 (m, |
| | | | | 1H), 2.50 (m, |
| | | | | 1H), 2.36 (m, |
| | | | | 1H), 2.20 (m, |
| | | | | 1H), 2.01-1.93 |
| | | | | (m, 1H) |
| 106 | 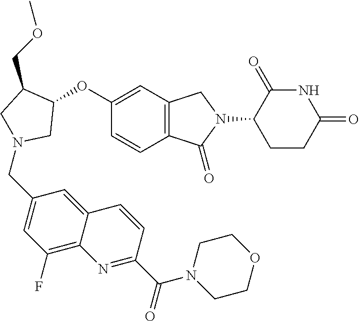 [SMILES: COC[C@@H]1CN(Cc2cc(F)c3nc(C(=O)N4CCOCC4)ccc3c2)C[C@H]1Oc1ccc2c(c1)CN([C@H]1CCC(=O)NC1=O)C2=O] | (3S)-3-(5- {[(3S,4S)-1-{[8- fluoro-2- (morpholine-4- carbonyl)quinolin- 6-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 646.3 [M + 1]⁺ | ¹H NMR (400 MHz, MeOD-d₄) δ 8.50-8.26 (m, 2H), 7.80-7.55 (m, 4H), 7.10 (d, J = 20.6 Hz, 2H), 5.14-5.07 (m, 1H), 4.80 (m, 1H), 4.40 (d, J = 7.9 Hz, 2H), 4.05-3.89 (m, 2H), 3.84 (m, 4H), 3.70-3.67 (m, 2H), 3.62 (m, 2H), 3.50 (m, 2H), 3.35 (s, 3H), 3.19 (m, 1H), 3.10-2.96 (m, 2H), 2.88 (m, 1H), 2.77 (m, |
| | | | | 2H), 2.57-2.39 |
| | | | | (m, 2H), 2.15 (m, |
| | | | | 1H) |
| 107 | 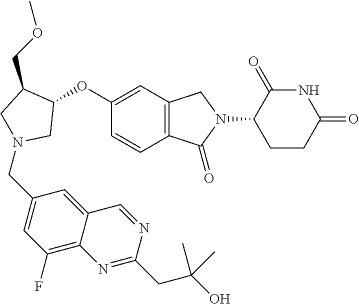 [SMILES: COC[C@@H]1CN(Cc2cc(F)c3nc(CC(C)(C)O)ncc3c2)C[C@H]1Oc1ccc2c(c1)CN([C@H]1CCC(=O)NC1=O)C2=O] | (3S)-3-(5- {[(3S,4S)-1-{[8- fluoro-2-(2- hydroxy-2- methylpropyl) quinazolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 606.5 [M + 1]⁺ | ¹H NMR (400 MHz, MeOD-d₄) δ 9.49 (d, J = 1.5 Hz, 1H), 7.86 (d, J = 1.7 Hz, 1H), 7.82-7.79 (m, 1H), 7.68 (d, J = 8.5 Hz, 1H), 7.13 (d, J = 2.1 Hz, 1H), 7.08-7.05 (m,, 1H), 5.12- 5.07 (m, 1H), 4.85-4.82 (m, 1H), 4.42-4.40 (m, 2H), 3.88 (s, 2H), 3.52-3.50 (m, 2H), 3.37 (s, 3H), 3.29 (m, 2H), 3.09-3.07 |
| | | | | (m, 1H), 2.99- |
| | | | | 2.97 (m, 1H), |
| | | | | 2.96-2.85 (m, |
| | | | | 2H), 2.75 (m, |
| | | | | 1H), 2.70 (m, |
| | | | | 1H), 2.45-2.41 |
| | | | | (m, 2H), 2.14 (m, |
| | | | | 1H), 1.30 (s, 6H) |
| 108 | 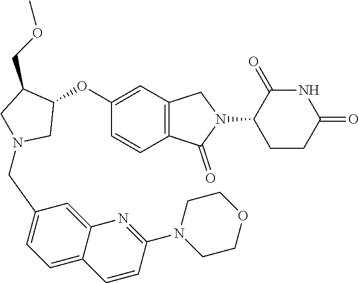 [SMILES: COC[C@@H]1CN(Cc2ccc3ccc(N4CCOCC4)nc3c2)C[C@H]1Oc1ccc2c(c1)CN([C@H]1CCC(=O)NC1=O)C2=O] | (3S)-3-(5- {[(3S,4S)-4- (methoxymethyl)- 1-{[2-(morpholin- 4-yl)quinolin-7- yl]methyl} pyrrolidin- 3-yl]oxy}-1- oxo-2,3-dihydro- 1H-isoindol-2- yl)piperidine-2,6- dione | MS (ESI) m/z 600.3 [M + 1]⁺ | ¹H NMR (400 MHz, DMSO-d₆) δ 8.16 (s, 1H- formic acid), 8.01 (d, J = 9.1 Hz, 1H), 7.64 (d, J = 8.2 Hz, 1H), 7.59 (d, J = 8.4 Hz, 1H), 7.48 (s, 1H), 7.19-7.15 (m, 2H), 7.13 (d, J = 2.2 Hz, 1H), 7.02-6.58 (m, 1H), 5.04-5.01 (m 1H), 4.72- 4.68 (m, 1H), 4.35 (d, J = 17.3 Hz, 1H), 4.24 (d, |
| | | | | J = 17.2 Hz, 1H), |
| | | | | 3.75-3.67 (m, |
| | | | | 6H), 3.61 (m, |
| | | | | 4H), 3.48-3.37 |
| | | | | (m, 2H), 3.32 (s, |
| | | | | 3H), 2.90-2.85 |
| | | | | (m, 3H), 2.73- |
| | | | | 2.70 (m, 1H), |
| | | | | 2.63-2.54 (m, |
| | | | | 2H), 2.43-2.27 |
| | | | | (m, 1H), 2.20- |
| | | | | 2.16 (m, 1H), |
| | | | | 1.96-1.92 (m, |
| | | | | 1H) |
| 109 | 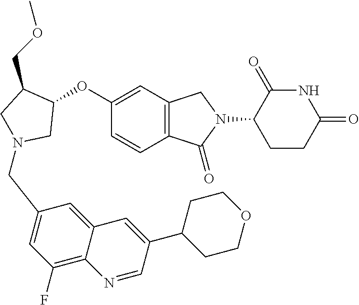 [SMILES: COC[C@@H]1CN(Cc2cc(F)c3ncc(C4CCOCC4)cc3c2)C[C@H]1Oc1ccc2c(c1)CN([C@H]1CCC(=O)NC1=O)C2=O] | (3S)-3-(5- {[(3S,4S)-1-{[8- fluoro-3-(oxan-4- yl)quinolin-6- yl]methyl}-4- (methoxymethyl) pyrrolidin-3- yl]oxy}-1-oxo- 2,3-dihydro-1H- isoindol-2- yl)piperidine-2,6- dione | MS (ESI) m/z 617.2 [M + 1]⁺ | ¹H NMR (400 MHz, DMSO-d₆) δ 8.87 (d, J = 2.2 Hz, 1H), 8.24- 8.15 (m, 1H), 7.79 (s, 1H), 7.71-7.62 (m, 1H), 7.47 (dd, J = 11.6, 1.6 Hz, 1H), 7.15 (d, J = 2.2 Hz, 1H), 7.04 (dd, J = 8.5, 2.2 Hz, 1H), 5.04 (dd, J = 13.3, 5.1 Hz, 1H), 4.75 (m, 1H), 4.37 (d, J = 17.2 Hz, 1H), 4.25 (d, J = 17.3 Hz, 2H), 4.04- |
| | | | | 3.95 (m, 2H), |
| | | | | 3.80-3.74 (m, |
| | | | | 2H), 3.52-3.42 |
| | | | | (m, 4H), 3.26 (s, |
| | | | | 3H), 3.05-2.96 |
| | | | | (m, 1H), 2.95- |
| | | | | 2.82 (m, 3H), |
| | | | | 2.76-2.65 (m, |
| | | | | 1H), 2.58 (m, |
| | | | | 2H), 2.44-2.29 |
| | | | | (m, 1H), 2.25 (m, |
| | | | | 1H), 2.03-1.90 |
| | | | | (m, 1H), 1.82 (m, |
| | | | | 4H) |
| 110 | 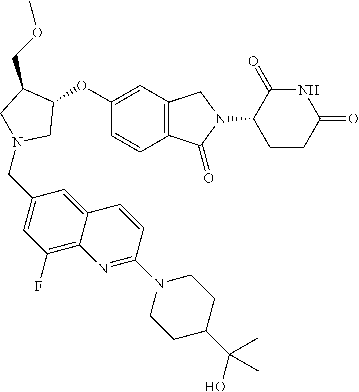 [SMILES: COC[C@@H]1CN(Cc2cc(F)c3nc(N4CCC(C(C)(C)O)CC4)ccc3c2)C[C@H]1Oc1ccc2c(c1)CN([C@H]1CCC(=O)NC1=O)C2=O] | (3S)-3-(5- {[(3S,4S)-1-({8- fluoro-2-[4-(2- hydroxypropan-2- yl)piperidin-1- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 674.5 [M + 1]⁺ | ¹H NMR (400 MHz, MeOD-d₄) δ 7.94 (dd, J = 9.3, 1.6 Hz, 1H), 7.71 (d, J = 8.4 Hz, 1H), 7.42 (s, 1H), 7.34 (dd, J = 11.8, 1.8 Hz, 1H), 7.22 (d, J = 9.3 Hz, 1H), 7.14 (d, J = 2.1 Hz, 1H), 7.09 (dd, J = 8.4, 2.2 Hz, 1H), 5.12 (dd, J = 13.3, 5.1 Hz, 1H), 4.91 (d, J = 3.4 Hz, 1H), 4.72 (m, 2H), 4.51- 4.36 (m, 2H), 3.99-3.87 (m, 2H), 3.57-3.51 (m, 2H), 3.39 (s, 3H), 3.32-3.23 (m, 1H), 3.12 (m, |
| | | | | 2H), 2.98-2.89 |
| | | | | (m, 3H), 2.84- |
| | | | | 2.71 (m, 2H), |
| | | | | 2.66-2.57 (m, |
| | | | | 1H), 2.56-2.40 |
| | | | | (m, 1H), 2.21- |
| | | | | 2.12 (m, 1H), |
| | | | | 1.92 (m, 2H), |
| | | | | 1.68-1.57 (m, |
| | | | | 1H), 1.46-1.29 |
| | | | | (m, 2H), 1.18 (s, |
| | | | | 6H) |
| 111 | 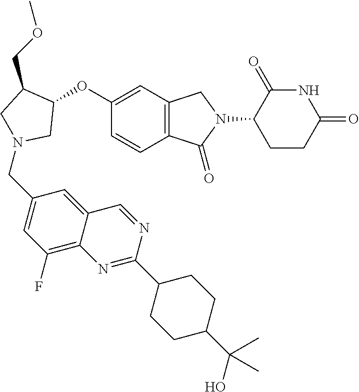 [SMILES: COC[C@@H]1CN(Cc2cc(F)c3nc(C4CCC(C(C)(C)O)CC4)ncc3c2)C[C@H]1Oc1ccc2c(c1)CN([C@H]1CCC(=O)NC1=O)C2=O] | rel-(S)-3-(5- (((3S,4S)-1-((8- fluoro-2-(4-(2- hydroxypropan-2- yl)cyclohexyl) quinazolin-6- yl)methyl)-4- (methoxymethyl) pyrrolidin-3- yl)oxy)-1- oxoisoindolin-2- yl)piperidine-2,6- dione | MS (ESI) m/z 674.6 [M + 1]⁺ | ¹H NMR (400 MHz, MeOD-d₄) δ 9.47 (d, J = 1.7 Hz, 1H), 7.85 (s, 1H), 7.80 (d, J = 11.2 Hz, 1H), 7.71 (d, J = 8.5 Hz, 1H), 7.15 (d, J = 2.2 Hz, 1H), 7.09 (dd, J = 8.5, 2.2 Hz, 1H), 5.12 (dd, J = 13.4, 5.1 Hz, 1H), 4.85- 4.75 (m, 1H), 4.51-4.36 (m, 2H), 3.92 (d, J = 2.9 Hz, 2H), 3.54 (d, J = 6.7 Hz, 2H), 3.39 (s, 3H), 3.12 (m, 1H), 3.00 (m, 2H), 2.80 (m, 2H), 2.70 (m, 2H), 2.55-2.41 (m, |
| | | | | 2H), 2.15 (m, |
| | | | | 3H), 2.07 (m, |
| | | | | 2H), 1.90-1.80 |
| | | | | (m, 2H), 1.54- |
| | | | | 1.43 (m, 1H), |
| | | | | 1.35-1.29 (m, |
| | | | | 2H), 1.22 (s, 6H) |
| 112 | 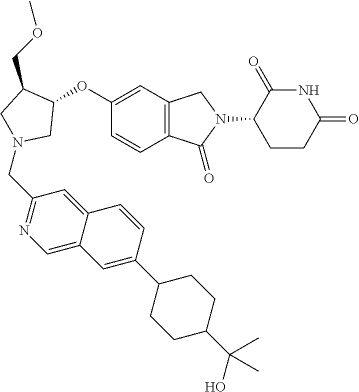 [SMILES: COC[C@@H]1CN(Cc2cc3ccc(C4CCC(C(C)(C)O)CC4)cc3cn2)C[C@H]1Oc1ccc2c(c1)CN([C@H]1CCC(=O)NC1=O)C2=O] | rel-(S)-3-(5- (((3S,4S)-1-((7- (4-(2- hydroxypropan-2- yl)cyclohexyl) isoquinolin-3- yl)methyl)-4- (methoxymethyl) pyrrolidin-3- yl)oxy)-1- oxoisoindolin-2- yl)piperidine-2,6- dione | MS (ESI) m/z 655.6 [M + 1]⁺ | ¹H NMR (400 MHz, MeOD-d₄) δ 9.18 (s, 1H), 8.06 (s, 1H), 7.90-7.79 (m, 3H), 7.69 (d, J = 8.5 Hz, 1H), 7.13 (d, J = 2.2 Hz, 1H), 7.07 (dd, J = 8.5, 2.2 Hz, 1H), 5.10 (dd, J = 13.3, 5.2 Hz, 1H), 4.90 (m, 1H), 4.49-4.34 (m, 2H), 4.11 (m, 2H), 3.53 (d, J = 6.1 Hz, 2H), 3.37 (s, 3H), 3.36-3.35 (m, 1H), 3.23- 3.15 (m, 3H), 2.89-2.83 (m, 1H), 2.82-2.63 (m, 3H), 2.54- 2.41 (m, 3H), |
| | | | | 2.19-2.10 (m, |
| | | | | 1H), 1.95-1.89 |
| | | | | (m, 2H), 1.72- |
| | | | | 1.68 (m, 2H), |
| | | | | 1.53-1.42 (m, |
| | | | | 1H), 1.38-1.24 |
| | | | | (m, 2H), 1.08 (s, |
| | | | | 6H) |
| 113 | 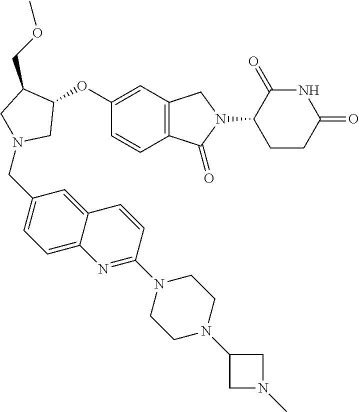 [SMILES: COC[C@@H]1CN(Cc2ccc3nc(N4CCN(C5CN(C)C5)CC4)ccc3c2)C[C@H]1Oc1ccc2c(c1)CN([C@H]1CCC(=O)NC1=O)C2=O] | (3S)-3-(5- {[(3S,4S)-4- (methoxymethyl)- 1-({2-[4-(1- methylazetidin-3- yl)piperazin-1- yl]quinolin-6-yl} methyl)pyrrolidin- 3-yl]oxy}-1- oxo-2,3-dihydro- 1H-isoindol-2- yl)piperidine-2,6- dione | MS (ESI) m/z 668.6 [M + 1]⁺ | ¹H NMR (400 MHz, MeOD-d₄) δ 8.30 (m, 1H), 8.02 (m, 1H), 7.99-7.88 (m, 2H), 7.71 (m, 1H), 7.57-7.51 (m, 1H), 7.21- 7.14 (m, 1H), 7.07 (m, 1H), 5.20 (m, 1H), 5.13-5.11 (m, 1H), 4.64 (s, 2H), 4.54-4.40 (m, 3H), 4.02-3.98 (m, 5H), 3.94- 3.78 (m, 2H), 3.76-3.65 (m, 2H), 3.58 (m, 2H), 3.44 (m, 5H), 3.27 (m, 1H), 3.00 (s, 3H), 2.96-2.85 (m, 2H), 2.84-2.75 (m, 1H), 2.68 (m, |
| | | | | 4H), 2.55-2.43 |
| | | | | (m, 1H), 2.22- |
| | | | | 2.11 (m, 1H) |
| 114 | 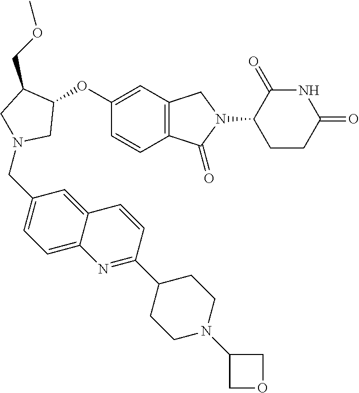 [SMILES: COC[C@@H]1CN(Cc2ccc3nc(C4CCN(C5COC5)CC4)ccc3c2)C[C@H]1Oc1ccc2c(c1)CN([C@H]1CCC(=O)NC1=O)C2=O] | (3S)-3-(5- {[(3S,4S)-4- (methoxymethyl)- 1-({2-[1-(oxetan- 3-yl)piperidin-4- yl]quinolin-6-yl} methyl)pyrrolidin- 3-yl]oxy}-1- oxo-2,3-dihydro- 1H-isoindol-2- yl)piperidine-2,6- dione | MS (ESI) m/z 654.3 [M + 1]⁺ | ¹H NMR (400 MHz, MeOD-d₄) δ 8.31 (d, J = 18.3 Hz, 1H), 8.27 (s, 1H), 8.01 (d, J = 8.7 Hz, 1H), 7.92 (d, J = 1.8 Hz, 1H), 7.81 (dd, J = 8.7, 1.9 Hz, 1H), 7.72- 7.68 (m, 1H), 7.55-7.49 (m, 1H), 7.14 (d, J = 2.4 Hz, 1H), 7.07 (dd, J = 8.5, 2.2 Hz, 1H), 5.11 (m, 1H), 4.90 (m, 1H), 4.76-4.72 (m, 5H), 4.49- 4.36 (m, 2H), 4.15 (m, 2H), 3.82 (m, 1H), 3.55 (m, 2H), 3.39 (s, 3H), |
| | | | | 3.24-3.12 (m, |
| | | | | 4H), 3.04-3.02 |
| | | | | (m, 1H), 2.94- |
| | | | | 2.85 (m, 1H), |
| | | | | 2.76 (m, 3H), |
| | | | | 2.48 (m, 1H), |
| | | | | 2.37-2.28 (m, |
| | | | | 2H), 2.11-2.00 |
| | | | | (m, 5H) |
| 115 | 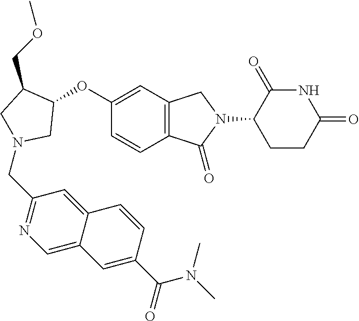 [SMILES: COC[C@@H]1CN(Cc2cc3ccc(C(=O)N(C)C)cc3cn2)C[C@H]1Oc1ccc2c(c1)CN([C@H]1CCC(=O)NC1=O)C2=O] | 3-{[(3S,4S)-3- ({2-[(3S)-2,6- dioxopiperidin-3- yl]-1-oxo-2,3- dihydro-1H- isoindol-5- yl}oxy)-4- (methoxymethyl) pyrrolidin-1- yl]methyl}-N,N- dimethyliso- quinoline-7- carboxamide | MS (ESI) m/z 586.5 [M + 1]⁺ | ¹H NMR (400 MHz, MeOD-d₄) δ 9.33 (s, 1H), 8.21 (s, 1H), 8.03 (d, J = 8.5 Hz, 1H), 7.95 (s, 1H), 7.83 (dd, J = 8.5, 1.6 Hz, 1H), 7.72 (d, J = 8.4 Hz, 1H), 7.15 (d, J = 2.2 Hz, 1H), 7.10 (dd, J = 8.5, 2.2 Hz, 1H), 5.12 (dd, J = 13.3, 5.2 Hz, 1H), 5.00- 4.94 (m, 1H), 4.51-4.23 (m, 4H), 3.68-3.52 (m, 2H), 3.49- 3.43 (m, 4H), |
| | | | | 3.28-3.25 (m, |
| | | | | 2H), 3.19 (s, 3H), |
| | | | | 3.07 (s, 3H), |
| | | | | 2.98-2.88 (m, |
| | | | | 1H), 2.85-2.71 |
| | | | | (m, 3H), 2.53- |
| | | | | 2.42 (m, 1H), |
| | | | | 2.22-2.11 (m, |
| | | | | 1H) |
| 116 | 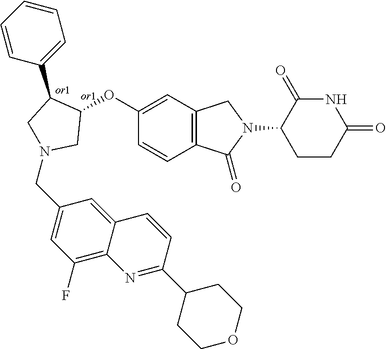 [SMILES: O=C1CC[C@H](N2Cc3cc(O[C@@H]4CN(Cc5cc(F)c6nc(C7CCOCC7)ccc6c5)C[C@H]4c4ccccc4)ccc3C2=O)C(=O)N1] | (S)-3-(5- (((3S*,4R*)-1- ((8-fluoro-2- (tetrahydro-2H- pyran-4- yl)quinolin-6- yl)methyl)-4- phenylpyrrolidin- 3-yl)oxy)-1- oxoisoindolin-2- yl)piperidine-2,6- dione | MS (ESI) m/z 649.5 [M + 1]⁺ | ¹H NMR (400 MHz, MeOD-d₄) δ 8.29-8.27 (m, 1H), 7.70 (s, 1H), 7.65 (m, 1H), 7.62-7.55 (m, 2H), 7.37-7.28 (m, 4H), 7.28- 7.26 (m, 1H), 6.97-6.94 (m,, 1H), 6.90 (d, J = 2.2 Hz, 1H), 5.10-5.08 (m, 1H), 5.03-5.04 (m, 1H), 4.35- 4.31 (m, 2H), 4.12-4.09 (m, 2H), 3.98-3.90 (m, 2H), 3.64- 3.62 (m, 2H), 3.57-3.56 (m, 1H), 3.40 (t, J = |
| | | | | 8.6 Hz, 1H), |
| | | | | 3.38-3.18 (m, |
| | | | | 2H), 3.17-3.13 |
| | | | | (m, 1H), 2.89 (m, |
| | | | | 1H), 2.78 (m, |
| | | | | 1H), 2.68 (t, J = |
| | | | | 8.9 Hz, 1H), 2.45 |
| | | | | (m, 1H), 2.05- |
| | | | | 2.04 (m, 1H), |
| | | | | 2.02-2.01 (m,, |
| | | | | 2H), 1.93-1.90 |
| | | | | (m, 2H) |
| 117 | 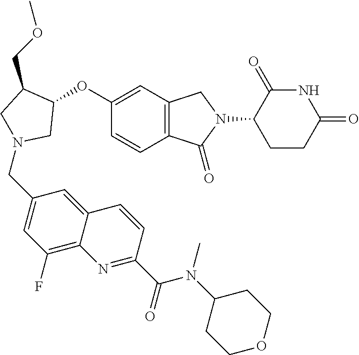 [SMILES: COC[C@@H]1CN(Cc2cc(F)c3nc(C(=O)N(C)C4CCOCC4)ccc3c2)C[C@H]1Oc1ccc2c(c1)CN([C@H]1CCC(=O)NC1=O)C2=O] | 6-{[(3S,4S)-3- ({2-[(3S)-2,6- dioxopiperidin-3- yl]-1-oxo-2,3- dihydro-1H- isoindol-5- yl}oxy)-4- (methoxymethyl) pyrrolidin-1- yl]methyl}-8- fluoro-N-methyl- N-(oxan-4- yl)quinoline-2- carboxamide | MS (ESI) m/z 674.3 [M + 1]⁺ | ¹H NMR (400 MHz, MeOD-d₄) δ 8.45 (d, J = 8.6, 2.0 Hz, 1H), 7.78-7.67 (m, 3H), 7.62-7.59 (m, 1H), 7.13 (d, J = 2.2 Hz, 1H), 7.09-7.04 (m, 1H), 5.09 (dd, J = 13.3, 5.2 Hz, 1H), 4.80-4.72 (m, 1H), 4.47- 4.34 (m, 2H), 4.11-3.83 (m, 5H), 3.62-3.48 (m, 3H), 3.37 (s, 3H), 3.19 (m, 1H), 3.14-3.06 (m, 3H), 3.00- 2.86 (m, 4H), 2.78 (m, 1H), |
| | | | | 2.75-2.67 (m, |
| | | | | 1H), 2.50-2.40 |
| | | | | (m, 2H), 2.14- |
| | | | | 2.12 (m, 1H), |
| | | | | 2.00-1.97 (m, |
| | | | | 2H), 1.89 (m, H), |
| | | | | 1.83-1.76 (m, |
| | | | | 1H) |
| 118 | 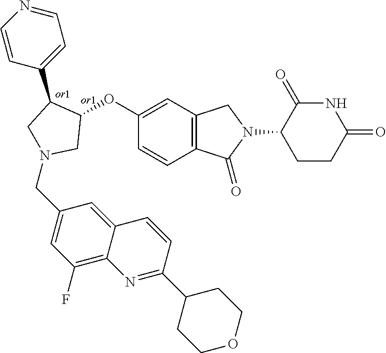 [SMILES: O=C1CC[C@H](N2Cc3cc(O[C@@H]4CN(Cc5cc(F)c6nc(C7CCOCC7)ccc6c5)C[C@H]4c4ccncc4)ccc3C2=O)C(=O)N1] | (S)-3-(5- (((3S*,4R*)-1- ((8-fluoro-2- (tetrahydro-2H- pyran-4- yl)quinolin-6- yl)methyl)-4- (pyridin-4- yl)pyrrolidin-3- yl)oxy)-1- oxoisoindolin-2- yl)piperidine-2,6- dione | MS (ESI) m/z 650.6 [M + 1]⁺ | ¹H NMR (400 MHz, MeOD-d₄) δ 8.51 (d, J = 5.3 Hz, 2H), 8.28 (m, 1H), 7.72-7.66 (m, 2H), 7.58 (dd, J = 10.0, 4.8 Hz, 2H), 7.49 (d, J = 5.4 Hz, 2H), 7.03-6.95 (m, 2H), 5.13-5.05 (m, 2H), 4.37- 4.35 (m, 2H), 4.15-4.07 (m, 2H), 3.99-3.89 (m, 2H), 3.65- 3.62 (m, 3H), 3.38 (m, 2H), 3.27-3.20 (m, 1H), 3.05 (m, 1H), 2.89-2.87 (m, 1H), 2.81- |
| | | | | 2.72 (m, 2H), |
| | | | | 2.50-2.39 (m, |
| | | | | 1H), 2.18-2.10 |
| | | | | (m, 1H), 2.02- |
| | | | | 1.99 (m, 2H), |
| | | | | 1.91 (m, 2H) |
| 119 | 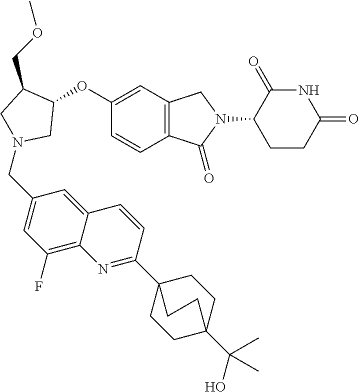 [SMILES: COC[C@@H]1CN(Cc2cc(F)c3nc(C45CCC(C(C)(C)O)(CC4)CC5)ccc3c2)C[C@H]1Oc1ccc2c(c1)CN([C@H]1CCC(=O)NC1=O)C2=O] | (S)-3-(5- (((3S,4S)-1-((8- fluoro-2-(4-(2- hydroxypropan-2- yl)bicyclo[2.2.2] octan-1- yl)quinolin-6- yl)methyl)-4- (methoxymethyl) pyrrolidin-3- yl)oxy)-1- oxoisoindolin-2- yl)piperidine-2,6- dione | MS (ESI) m/z 699.6 [M + 1]⁺ | ¹H NMR (400 MHz, MeOD-d₄) δ 8.16 (d, J = 8.6 Hz, 1H), 7.70 (d, J = 8.4 Hz, 1H), 7.61 (m, 2H), 7.47 (m, 1H), 7.14 (s, 1H), 7.08 (m, 1H), 5.11 (m, 1H), 4.84 (m, 1H), 4.50-4.35 (m, 2H), 3.85 (m, 2H), 3.53 (m, 2H), 3.38 (s, 3H), 3.12-3.09 (m, 1H), 2.97-2.85 (m, 3H), 2.78- 2.65 (m, 2H), 2.52-2.40 (m, 2H), 2.17-2.10 (m, 1H), 2.03 (m, 6H), 1.73 (m, 6H), 1.17 (s, 6H) |
| 120 | 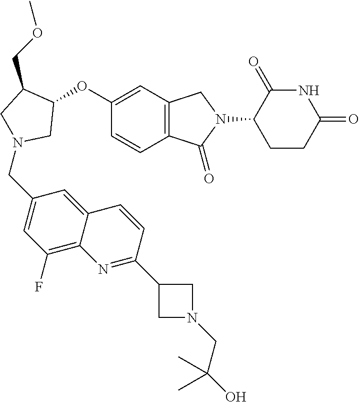 [SMILES: COC[C@@H]1CN(Cc2cc(F)c3nc(C4CN(CC(C)(C)O)C4)ccc3c2)C[C@H]1Oc1ccc2c(c1)CN([C@H]1CCC(=O)NC1=O)C2=O] | (3S)-3-(5- {[(3S,4S)-1-({8- fluoro-2-[1-(2- hydroxy-2- methylpropyl) azetidin-3- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 660.4 [M + 1]⁺ | ¹H NMR (400 MHz, DMSO-d₄) δ 8.47 (d, J = 8.8 Hz, 1H), 7.99 (s, 1H), 7.80-7.78 (m, 1H), 7.76- 7.63 (m, 2H), 7.20 (d, J = 2.3 Hz, 1H), 7.08- 7.04 (m, 1H), 5.11-5.01 (m, 2H), 4.75-4.50 (m, 4H), 4.50- 4.35 (m, 4H), 4.27 (m, 1H), 3.78-3.68 (m, 3H), 3.50-3.40 (m, 1H), 3.40- 3.20 (m, 5H), 3.19 (m, 1H),3.08-3.06 (m, 1H), 2.85 (m, 2H), 2.60 (m, 1H), 2.40 (m, 1H), 2.00 (m, |
| | | | | 1H), 1.20 (s, 6H) |
| 121 | 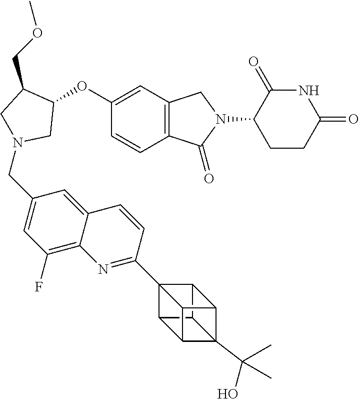 [SMILES: COC[C@@H]1CN(Cc2cc(F)c3nc(C45C6C7C4C4C5C6C74C(C)(C)O)ccc3c2)C[C@H]1Oc1ccc2c(c1)CN([C@H]1CCC(=O)NC1=O)C2=O] | (S)-3-(5- (((3S,4S)-1-((8- fluoro-2-(4-(2- hydroxypropan-2- yl)cuban-1- yl)quinolin-6- yl)methyl)-4- (methoxymethyl) pyrrolidin-3- yl)oxy)-1- oxoisoindolin-2- yl)piperidine-2,6- dione | MS (ESI) m/z 693.2 [M + 1]⁺ | ¹H NMR (400 MHz, DMSO-d₆) δ 10.96 (s, 1H), 8.34 (d, J = 8.6 Hz, 1H), 7.67 (s, 1H), 7.60 (d, J = 8.4 Hz, 1H), 7.55-7.46 (m, 2H), 7.15 (s, 1H), 7.04 (d, J = 8.5 Hz, 1H), 5.06 (dd, J = 13.3, 5.1 Hz, 1H), 4.75 (m, 1H), 4.36 (d, J = 17.2 Hz, 1H), 4.25 (d, J = 17.4 Hz, 1H), 4.12 (t, J = 4.7 Hz, 3H), 3.92 (t, J = 4.8 Hz, 3H), 3.79 (m, 2H), 3.50 (m, 2H), 3.23 (s, 3H), 2.98-2.83 (m, 3H), 2.75 (m, 1H), 2.63-2.52 |
| | | | | (m, 2H), 2.37 (m, |
| | | | | 1H), 2.25 (m, |
| | | | | 1H), 2.01-1.93 |
| | | | | (m, 1H), 1.08 (s, |
| | | | | 6H) |
| 122 | 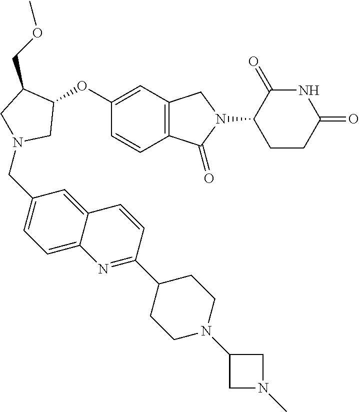 [SMILES: COC[C@@H]1CN(Cc2ccc3nc(C4CCN(C5CN(C)C5)CC4)ccc3c2)C[C@H]1Oc1ccc2c(c1)CN([C@H]1CCC(=O)NC1=O)C2=O] | (3S)-3-(5- {[(3S,4S)-4- (methoxymethyl)- 1-({2-[1-(1- methylazetidin-3- yl)piperidin-4- yl]quinolin-6-yl} methyl)pyrrolidin- 3-yl]oxy}-1- oxo-2,3-dihydro- 1H-isoindol-2- yl)piperidine-2,6- dione | MS (ESI) m/z 667.4 [M + 1]⁺ | ¹H NMR (400 MHz, MeOD-d₄) δ 8.29 (d, J = 8.7 Hz, 1H), 8.04 (dd, J = 8.7, 1.8 Hz, 1H), 8.00- 7.93 (m, 1H), 7.84 (m, 1H), 7.71 (d, J = 8.4 Hz, 1H), 7.53 (dd, J = 8.6, 1.1 Hz, 1H), 7.16 (d, J = 2.1 Hz, 1H), 7.08 (dd, J = 8.4, 2.2 Hz, 1H), 5.12 (dd, J = 13.3, 5.2 Hz, 1H), 5.00 (m, 1H), 4.66-4.37 (m, 2H), 4.32- 4.21 (m, 4H), 4.07 (m, 2H), 3.63-3.51 (m, 2H), 3.49-3.40 (s, 6H), 3.04 (m, 3H), 2.98-2.74 |
| | | | | (m, 8H), 2.48 (m, |
| | | | | 1H), 2.22-2.08 |
| | | | | (m, 3H), 2.08- |
| | | | | 1.96 (m, 4H) |
| 123 | 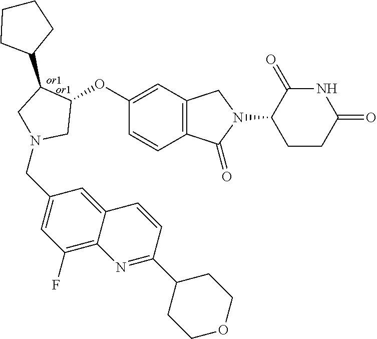 [SMILES: O=C1CC[C@H](N2Cc3cc(O[C@@H]4CN(Cc5cc(F)c6nc(C7CCOCC7)ccc6c5)C[C@H]4C4CCCC4)ccc3C2=O)C(=O)N1] | (S)-3-(5- (((3R*,4S*)-4- cyclopentyl-1- ((8-fluoro-2- (tetrahydro-2H- pyran-4- yl)quinolin-6-yl) methyl)pyrrolidin- 3-yl)oxy)-1- oxoisoindolin-2- yl)piperidine-2,6- dione | MS (ESI) m/z 641.3 [M + 1]⁺ | ¹H NMR (400 MHz, MeOD-d₄) δ 8.26 (d, J = 8.7 Hz, 1H), 7.70 (dd, J = 8.4, 1.9 Hz, 1H), 7.66 (s, 1H), 7.58-7.50 (m, 2H), 7.09- 7.01 (m, 2H), 5.11 (dd, J = 13.2, 5.1 Hz, 1H), 4.74 (t, J = 4.7 Hz, 1H), 4.48-4.35 (m, 2H), 4.14-4.04 (m, 2H), 3.97- 3.82 (m, 2H), 3.63-3.61 (m, 2H), 3.24 (m, 2H), 3.01 (m, 1H), 2.96-2.85 |
| | | | | (m, 2H), 2.77 (m, |
| | | | | 1H), 2.50-2.30 |
| | | | | (m, 3H), 2.15 (m, |
| | | | | 1H), 2.06-1.88 |
| | | | | (m, 5H), 1.84- |
| | | | | 1.76 (m, 2H), |
| | | | | 1.71-1.54 (m, |
| | | | | 4H), 1.33-1.31 |
| | | | | (m, 1H), 1.21- |
| | | | | 1.19 (m, 1H) |
| 124 | 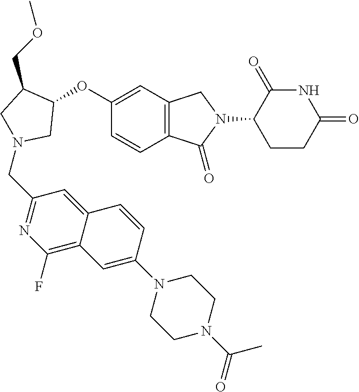 [SMILES: COC[C@@H]1CN(Cc2cc3ccc(N4CCN(C(C)=O)CC4)cc3c(F)n2)C[C@H]1Oc1ccc2c(c1)CN([C@H]1CCC(=O)NC1=O)C2=O] | (3S)-3-(5- {[(3S,4S)-1-{[7- (4- acetylpiperazin-1- yl)-1- fluoroisoquinolin- 3-yl]methyl}-4- (methoxymethyl) pyrrolidin-3- yl]oxy}-1-oxo- 2,3-dihydro-1H- isoindol-2- yl)piperidine-2,6- dione | MS (ESI) m/z 659.6 [M + 1]⁺ | ¹H NMR (400 MHz, DMSO-d₆) δ 10.96 (s, 1H), 7.92 (d, J = 9.1 Hz, 1H), 7.79- 7.71 (m, 1H), 7.61 (m, 2H), 7.28-7.12 (m, 2H), 7.11-7.01 (m, 1H), 5.10- 5.01 (m, 1H), 4.77 (m, 1H), 4.41-4.24 (m, 2H), 3.80-3.55 (m, 6H), 3.51- 3.40 (m, 2H), 3.39-3.32 (m, 2H), 3.27 (m, 4H), 3.11-2.78 (m, 4H), 2.59 (m, 2H), 2.45-2.24 (m, 3H), 2.07 (s, 3H), 2.02-1.94 (m, 1H) |
| 125 | 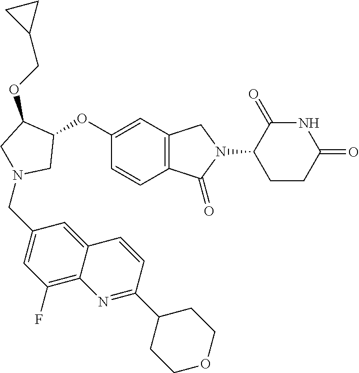 [SMILES: O=C1CC[C@H](N2Cc3cc(O[C@@H]4CN(Cc5cc(F)c6nc(C7CCOCC7)ccc6c5)C[C@H]4OCC4CC4)ccc3C2=O)C(=O)N1] | (3S)-3-(5- {[(3R,4R)-4- (cyclopropyl- methoxy)-1-{[8- fluoro-2-(oxan-4- yl)quinolin-6-yl] methyl}pyrrolidin- 3-yl]oxy}-1- oxo-2,3-dihydro- 1H-isoindol-2- yl)piperidine-2,6- dione | MS (ESI) m/z 643.3 [M + H]⁺ | ¹H NMR (400 MHz, MeOD-d₄) δ 8.28 (dd, J = 8.6, 1.5 Hz, 1H), 7.74-7.64 (m, 2H), 7.60-7.50 (m, 2H), 7.18 (d, J = 2.2 Hz, 1H), 7.12 (dd, J = 8.5, 2.2 Hz, 1H), 5.12 (dd, J = 13.3, 5.1 Hz, 1H), 4.80 (m, 1H), 4.44 (d, J = 3.8 Hz, 2H), 4.19-4.07 (m, 3H), 3.96-3.83 (m, 2H), 3.64- 3.61 (m, 2H), 3.38 (m, 1H), 3.29 (m, 1H), 3.27-3.11 (m, 3H), 2.96-2.83 (m, 2H), 2.82- |
| | | | | 2.67 (m, 2H), |
| | | | | 2.48-2.46 (m, |
| | | | | 1H), 2.17-2.15 |
| | | | | (m, 1H), 2.03- |
| | | | | 2.01 (m, 2H), |
| | | | | 1.95-1.85 (m, |
| | | | | 2H), 1.07-1.05 |
| | | | | (m, 1H), 0.58- |
| | | | | 0.48 (m, 2H), |
| | | | | 0.27-0.17 (m, |
| | | | | 2H) |
| 126 | 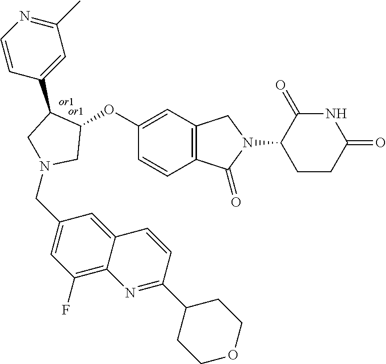 [SMILES: Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)ccn1] | (3S)-3-(5- {[(3S*,4R*)-1- {[8-fluoro-2- (oxan-4- yl)quinolin-6- yl]methyl}-4-(2- methylpyridin-4- yl)pyrrolidin-3- yl]oxy}-1-oxo- 2,3-dihydro-1H- isoindol-2- yl)piperidine-2,6- dione | MS (ESI) m/z 664.5 [M + 1]⁺ | ¹H NMR (400 MHz, DMSO-d₆) δ 10.96 (s, 1H), 8.40-8.31 (m, 2H), 7.71 (s, 1H), 7.62-7.51 (m, 3H), 7.26 (s, 1H), 7.20 (d, J = 5.0 Hz, 1H), 7.03- 6.95 (m, 2H), 5.10-5.03 (m, 2H), 4.36 (d, J = 17.2 Hz, 1H), 4.17 (d, J = 17.2 Hz, 1H), 4.03- 3.97 (m, 2H), 3.85 (s, 2H), 3.55-3.46 (m, 3H), 3.26 (m, 1H), 3.21-3.05 (m, 2H), 2.89 (m, 2H), 2.59 (m, 1H), 2.45 (s, 3H), |
| | | | | 2.31 (m, 2H), |
| | | | | 1.98-1.83 (m, |
| | | | | 5H) |
| 127 | 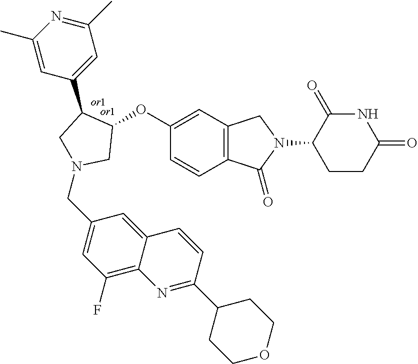 [SMILES: Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)cc(C)n1] | (3S)-3-(5- {[(3S*,4R*)-4- (2,6- dimethylpyridin- 4-yl)-1-{[8- fluoro-2-(oxan-4- yl)quinolin-6-yl] methyl}pyrrolidin- 3-yl]oxy}-1- oxo-2,3-dihydro- 1H-isoindol-2- yl)piperidine-2,6- dione | MS (ESI) m/z 678.6 [M + 1]⁺ | ¹H NMR (400 MHz, MeOD-d₄) δ 8.29 (d, J = 8.6 Hz, 1H), 8.21 (s, 1H), 7.68 (d, J = 10.0 Hz, 2H), 7.58 (dd, J = 10.3, 5.5 Hz, 2H), 7.18 (d, J = 3.7 Hz, 2H), 6.99 (d, J = 6.2 Hz, 2H), 5.15-5.03 (m, 2H), 4.38 (s, 2H), 4.11 (m, 2H), 4.01-3.88 (m, 2H), 3.65 (m, 2H), 3.56 (m, 1H), 3.31-3.20 (m, 3H), 3.09- 3.02 (m, 1H), 2.97-2.84 (m, 1H), 2.78 (m, |
| | | | | 1H), 2.74-2.66 |
| | | | | (m, 1H), 2.52 (m, |
| | | | | 6H), 2.47 (m, |
| | | | | 1H), 2.19-2.11 |
| | | | | (m, 1H), 2.10- |
| | | | | 1.96 (m, 2H), |
| | | | | 1.92 (m, 2H) |
| 128 | 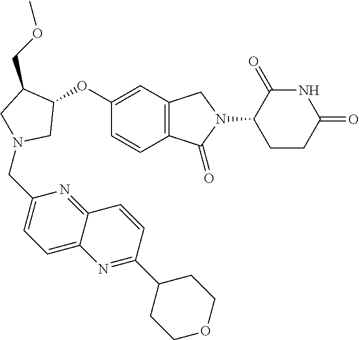 [SMILES: COC[C@@H]1CN(Cc2ccc3nc(C4CCOCC4)ccc3n2)C[C@H]1Oc1ccc2c(c1)CN([C@H]1CCC(=O)NC1=O)C2=O] | (3S)-3-(5- {[(3S,4S)-4- (methoxymethyl)- 1-{[6-(oxan-4- yl)-1,5- naphthyridin-2- yl]methyl} pyrrolidin- 3-yl]oxy}-1- oxo-2,3-dihydro- 1H-isoindol-2- yl)piperidine-2,6- dione | MS (ESI) m/z 600.3 [M + 1]⁺ | ¹H NMR (400 MHz, MeOD-d₄) δ 8.42 (d, J = 8.7 Hz, 1H), 8.33 (d, J = 8.8 Hz, 1H), 7.86 (d, J = 8.7 Hz, 1H), 7.75 (dd, J = 15.1, 8.6 Hz, 2H), 7.19 (d, J = 2.2 Hz, 1H), 7.13 (dd, J = 8.3, 2.1 Hz, 1H), 5.13 (dd, J = 13.3, 5.1 Hz, 1H), 5.03 (s, 1H), 4.53-4.40 (m, 4H), 4.12 (m, 2H), 3.65-3.51 (m, 5H), 3.43 (m, 4H), 3.30-3.19 (m, 2H), 2.99- 2.74 (m, 4H), |
| | | | | 2.49 (m, 1H), |
| | | | | 2.13-1.90 (m, |
| | | | | 5H) |
| 129 | 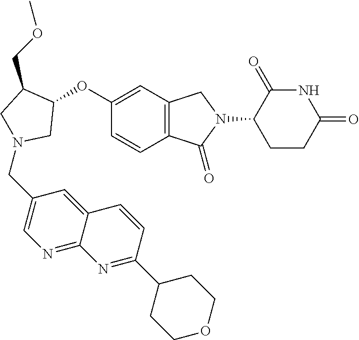 [SMILES: COC[C@@H]1CN(Cc2cnc3nc(C4CCOCC4)ccc3c2)C[C@H]1Oc1ccc2c(c1)CN([C@H]1CCC(=O)NC1=O)C2=O] | (3S)-3-(5- {[(3S,4S)-4- (methoxymethyl)- 1-{[7-(oxan-4- yl)-1,8- naphthyridin-3- yl]methyl} pyrrolidin- 3-yl]oxy}-1- oxo-2,3-dihydro- 1H-isoindol-2- yl)piperidine-2,6- dione | MS (ESI) m/z 600.5 [M + 1]⁺ | ¹H NMR (400 MHz, MeOD-d₄) δ 9.18 (s, 1H), 8.66 (s, 1H), 8.45 (d, J = 8.4 Hz, 1H), 7.74 (m, 2H), 7.18 (s, 1H), 7.10 (m, 1H), 5.24 (m, 1H), 5.14-5.12 (m, 1H), 4.79 (s, 2H), 4.47-4.45 (m, 2H), 4.14-4.11 (m, 2H), 3.92- 3.70 (m, 3H), 3.69-3.50 (m, 4H), 3.44 (m, 1H), 3.33 (s, 3H), 3.30 (m, 1H), 2.94-2.85 (m, 2H), 2.82-2.81 |
| | | | | (m, 1H), 2.55- |
| | | | | 2.48 (m, 1H), |
| | | | | 2.16 (m, 1H), |
| | | | | 2.08 (m, 2H), |
| | | | | 1.97 (m, 2H) |
| 130 | 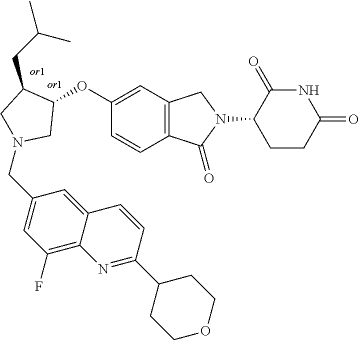 [SMILES: CC(C)C[C@@H]1CN(Cc2cc(F)c3nc(C4CCOCC4)ccc3c2)C[C@H]1Oc1ccc2c(c1)CN([C@H]1CCC(=O)NC1=O)C2=O] | (3S)-3-(5- {[(3S*,4R*)-1- {[8-fluoro-2- (oxan-4- yl)quinolin-6- yl]methyl}-4-(2- methylpropyl) pyrrolidin-3-yl] oxy}-1-oxo-2,3- dihydro-1H- isoindol-2- yl)piperidine-2,6- dione | MS (ESI) m/z 629.5 [M + 1]⁺ | ¹H NMR (400 MHz, MeOD-d₄) δ 8.31-8.24 (m, 1H), 7.71-7.66 (m, 1H), 7.56 (m, 2H), 7.09-7.01 (m, 2H), 5.12 (m, 1H), 4.66 (m, 1H), 4.46 (d, J = 17.0 Hz, 1H), 4.40 (d, J = 16.9 Hz, 1H), 4.11 (m, 2H), 3.93 (dd, J = 13.1, 5.0 Hz, 1H), 3.85 (dd, J = 13.3, 5.4 Hz, 1H), 3.70-3.59 (m, 2H), 3.29- 3.18 (m, 2H), 3.01-2.84 (m, 3H), 2.83-2.74 |
| | | | | (m, 1H), 2.60 (m, |
| | | | | 1H), 2.54-2.39 |
| | | | | (m, 1H), 2.33- |
| | | | | 2.22 (m, 1H), |
| | | | | 2.20-2.11 (m, |
| | | | | 1H), 2.11-1.96 |
| | | | | (m, 2H), 1.91 (m, |
| | | | | 2H), 1.67-1.39 |
| | | | | (m, 3H), 0.95 |
| | | | | (dd, J = 12.3, 6.5 |
| | | | | Hz, 6H) |
| 131 | 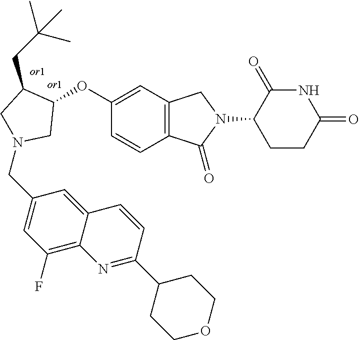 [SMILES: CC(C)(C)C[C@@H]1CN(Cc2cc(F)c3nc(C4CCOCC4)ccc3c2)C[C@H]1Oc1ccc2c(c1)CN([C@H]1CCC(=O)NC1=O)C2=O] | (3S)-3-(5- {[(3S*,4R*)-4- (2,2- dimethylpropyl)- 1-{[8-fluoro-2- (oxan-4- yl)quinolin-6-yl] methyl}pyrrolidin- 3-yl]oxy}-1- oxo-2,3-dihydro- 1H-isoindol-2- yl)piperidine-2,6- dione | MS (ESI) m/z 643.6 [M + 1]⁺ | ¹H NMR (400 MHz, MeOD-d₄) δ 8.29-8.22 (m, 1H), 7.73-7.63 (m, 2H), 7.59- 7.49 (m, 2H), 7.07-6.99 (m 2H), 5.10 (m, 1H), 4.70-4.62 (m, 1H), 4.49- 4.34 (m, 2H), 4.10 (m, 2H), 3.94 (d, J = 13.1 Hz, 1H), 3.84 (d, J = 13.1 Hz, 1H), 3.69-3.58 (m, 2H), 3.33-3.18 (m, 2H), 3.01 (m, 1H), 2.97-2.83 (m, 2H), 2.82- 2.72 (m, 1H), |
| | | | | 2.60 (m, 1H), |
| | | | | 2.52-2.47 (m, |
| | | | | 1H), 2.32-2.23 |
| | | | | (m, 1H), 2.18- |
| | | | | 2.10 (m, 1H), |
| | | | | 2.09-1.94 (m, |
| | | | | 2H), 1.94-1.86 |
| | | | | (m, 2H), 1.63 (m, |
| | | | | 1H), 1.47 (m, |
| | | | | 1H), 0.94 (s, 9H) |
| 132 | 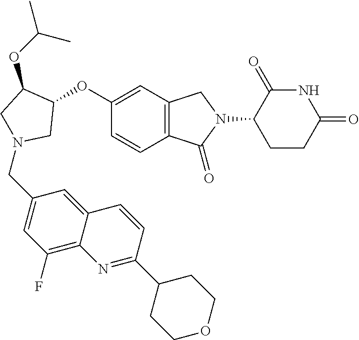 [SMILES: CC(C)O[C@@H]1CN(Cc2cc(F)c3nc(C4CCOCC4)ccc3c2)C[C@H]1Oc1ccc2c(c1)CN([C@H]1CCC(=O)NC1=O)C2=O] | (3S)-3-(5- {[(3R,4R)-1-{[8- fluoro-2-(oxan-4- yl)quinolin-6- yl]methyl}-4- (propan-2- yloxy)pyrrolidin- 3-yl]oxy}-1-oxo- 2,3-dihydro-1H- isoindol-2- yl)piperidine-2,6- dione | MS (ESI) m/z 631.4 [M + 1]⁺ | ¹H NMR (400 MHz, MeOD-d₄) δ 8.32-8.25 (m, 2H), 7.80-7.65 (m, 2H), 7.61- 7.51 (m, 2H), 7.17 (d, J = 2.1 Hz, 1H), 7.12 (dd, J = 8.4, 2.2 Hz, 1H), 5.12 (dd, J = 13.3, 5.1 Hz, 1H), 4.80 (m, 1H), 4.44 (d, J = 2.0 Hz, 1H), 4.29-4.21 (m, 1H), 4.16-4.07 (m, 2H), 3.94 (d, J = 13.2 Hz, 1H), 3.86 (d, J = 13.2 Hz, 1H), 3.71- 3.60 (m, 3H), |
| | | | | 3.29-3.11 (m, |
| | | | | 3H), 2.98-2.85 |
| | | | | (m, 2H), 2.84- |
| | | | | 2.73 (m, 1H), |
| | | | | 2.66 (m, 1H), |
| | | | | 2.57-2.41 (m, |
| | | | | 1H), 2.22-2.13 |
| | | | | (m, 1H), 2.12- |
| | | | | 1.97 (m, 2H), |
| | | | | 1.93 (m, 2H), |
| | | | | 1.18 (dd, J = 6.1, |
| | | | | 3.5 Hz, 6H) |
| 133 | 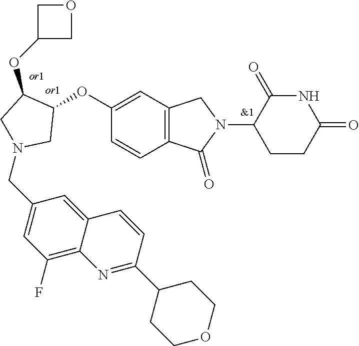 [SMILES: O=C1CCC(N2Cc3cc(O[C@@H]4CN(Cc5cc(F)c6nc(C7CCOCC7)ccc6c5)C[C@H]4OC4COC4)ccc3C2=O)C(=O)N1] | rel-3-(5- (((3R*,4R*)-1- ((8-fluoro-2- (tetrahydro-2H- pyran-4- yl)quinolin-6- yl)methyl)-4- (oxetan-3- yloxy)pyrrolidin- 3-yl)oxy)-1- oxoisoindolin-2- yl)piperidine-2,6- dione | MS (ESI) m/z 645.4 [M + 1]⁺ | ¹H NMR (400 MHz, MeOD-d₄) δ 8.44-8.26 (m, 1H), 7.87 (s, 1H), 7.76-7.58 (m, 3H), 7.24-7.00 (m, 2H), 5.35- 5.20 (m, 1H), 5.15 (dd, J = 13.6, 5.0 Hz, 1H), 4.85-4.76 (m, 3H), 4.69 (m, 1H), 4.63 (m, 2H), 4.52-4.38 (m, 3H), 4.15- 4.01 (m, 3H), 3.90-3.50 (m, 6H), 3.27 (m, 1H), 2.92-2.89 (m, 1H), 2.80 (m, 1H), 2.48-2.46 (m, 1H), 2.23- |
| | | | | 2.13 (m, 1H), |
| | | | | 2.10-2.01 (m, |
| | | | | 2H), 1.94 (m, |
| | | | | 2H) |
| 134 | 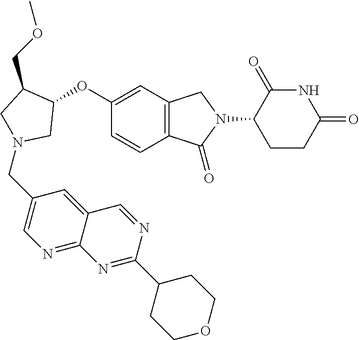 [SMILES: COC[C@@H]1CN(Cc2cnc3nc(C4CCOCC4)ncc3c2)C[C@H]1Oc1ccc2c(c1)CN([C@H]1CCC(=O)NC1=O)C2=O] | (3S)-3-(5- {[(3S,4S)-4- (methoxymethyl)- 1-{[2-(oxan-4- yl)pyrido[2,3- d]pyrimidin-6-yl] methyl}pyrrolidin- 3-yl]oxy}-1- oxo-2,3-dihydro- 1H-isoindol-2- yl)piperidine-2,6- dione | MS (ESI) m/z 601.4 [M + 1]⁺ | ¹H NMR (400 MHz, MeOD-d₄) δ 9.59 (s, 1H), 9.25 (d, J = 2.4 Hz, 1H), 8.52 (d, J = 2.5 Hz, 1H), 7.70 (d, J = 8.3 Hz, 1H), 7.14 (m, 1H), 7.10-7.04 (m, 1H), 5.11 (dd, J = 13.4, 5.1 Hz, 1H), 4.82 (m, 1H), 4.48-4.37 (m, 2H), 4.09- 4.06 (m, 2H), 4.00 (s, 2H), 3.65-3.62 (m, 2H), 3.54 (s, 3H), 3.39 (m, 3H), 3.15 (m, 1H), 3.06 (m, 1H), |
| | | | | 2.98 (m, 1H), |
| | | | | 2.92-2.84 (m, |
| | | | | 1H), 2.80 (m, |
| | | | | 1H), 2.77-2.67 |
| | | | | (m, 3H), 2.54- |
| | | | | 2.42 (m, 2H), |
| | | | | 2.20-2.00 (m, |
| | | | | 5H) |
| 135 | 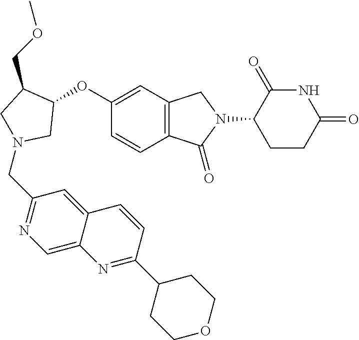 [SMILES: COC[C@@H]1CN(Cc2cc3ccc(C4CCOCC4)nc3cn2)C[C@H]1Oc1ccc2c(c1)CN([C@H]1CCC(=O)NC1=O)C2=O] | (3S)-3-(5- {[(3S,4S)-4- (methoxymethyl)- 1-{[2-(oxan-4- yl)-1,7- naphthyridin-6-yl] methyl}pyrrolidin- 3-yl]oxy}-1- oxo-2,3-dihydro- 1H-isoindol-2- yl)piperidine-2,6- dione | MS (ESI) m/z 600.5 [M + 1]⁺ | ¹H NMR (400 MHz, MeOD-d₄) δ 9.33 (s, 1H), 8.31 (d, J = 8.6 Hz, 1H), 7.90 (s, 1H), 7.79-7.67 (m, 2H), 7.15 (s, 1H), 7.09 (dd, J = 8.4, 2.2 Hz, 1H), 5.12 (dd, J = 13.3, 5.1 Hz, 1H), 4.97-4.91 (m, 1H), 4.47 (d, J = 17.0 Hz, 1H), 4.40 (d, J = 17.0 Hz, 1H), 4.26- 4.15 (m, 2H), 4.14-4.07 (m, 2H), 3.70-3.58 (m, 2H), 3.56 (d, J = 5.7 Hz, 2H), |
| | | | | 3.40 (s, 3H), 3.38 |
| | | | | (m, 1H), 3.25 (m, |
| | | | | 3H), 2.98-2.85 |
| | | | | (m, 1H), 2.84- |
| | | | | 2.72 (m, 3H), |
| | | | | 2.55-2.40 (m, |
| | | | | 1H), 2.22-1.91 |
| | | | | (m, 5H) |
| 136 | 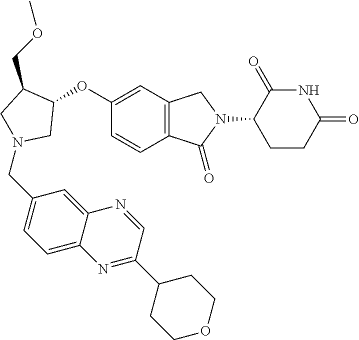 [SMILES: COC[C@@H]1CN(Cc2ccc3nc(C4CCOCC4)cnc3c2)C[C@H]1Oc1ccc2c(c1)CN([C@H]1CCC(=O)NC1=O)C2=O] | (3S)-3-(5- {[(3S,4S)-4- (methoxymethyl)- 1-{[2-(oxan-4- yl)quinoxalin-6-yl] methyl}pyrrolidin- 3-yl]oxy}-1- oxo-2,3-dihydro- 1H-isoindol-2- yl)piperidine-2,6- dione | MS (ESI) m/z 600.3 [M + 1]⁺ | ¹H NMR (400 MHz, MeOD-d₄) δ 8.85 (d, J = 2.0 Hz, 1H), 8.07- 8.02 (m, 2H), 7.88-7.86 (m, 1H), 7.70 (dd, J = 8.4, 2.0 Hz, 1H), 7.14 (s, 1H), 7.08-7.06 (m, 1H), 5.12-5.10 (m, 1H), 4.80 (m, 1H), 4.51-4.36 (m, 2H), 4.12- 4.09 (m, 2H), 3.97-3.96 (m, 2H), 3.66-3.63 (m, 2H), 3.59- 3.50 (m, 2H), 3.39 (s, 3H), 3.28 (m, 1H), 3.16 (m, |
| | | | | 1H), 3.06-2.94 |
| | | | | (m, 2H), 2.93- |
| | | | | 2.85 (m, 1H), |
| | | | | 2.83-2.68 (m, |
| | | | | 2H), 2.54-2.43 |
| | | | | (m, 2H), 2.15- |
| | | | | 2.13 (m, 1H), |
| | | | | 2.10-2.01 (m, |
| | | | | 2H), 1.96 (m, |
| | | | | 2H) |
| 137 | 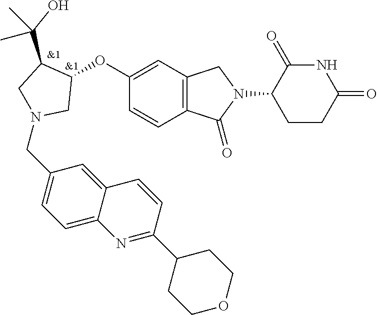 [SMILES: CC(C)(O)[C@@H]1CN(Cc2ccc3nc(C4CCOCC4)ccc3c2)C[C@H]1Oc1ccc2c(c1)CN([C@H]1CCC(=O)NC1=O)C2=O] | rel-(3S)-3-(5-((4- (2- hydroxypropan-2- yl)-1-((2- (tetrahydro-2H- pyran-4- yl)quinolin-6-yl) methyl)pyrrolidin- 3-yl)oxy)-1- oxoisoindolin-2- yl)piperidine-2,6- dione | MS (ESI) m/z 613.5 [M + 1]⁺ | ¹H NMR (400 MHz, DMSO-d₆) δ 10.95 (s, 1H), 8.24 (dd, J = 8.6, 2.4 Hz, 1H), 8.14 (s, 1H), 7.88 (d, J = 8.6 Hz, 1H), 7.81 (s, 1H), 7.68 (dd, J = 8.6, 1.9 Hz, 1H), 7.59 (dd, J = 8.4, 2.2 Hz, 1H), 7.47 (d, J = 8.5 Hz, 1H), 7.11 (dd, J = 4.1, 2.2 Hz, 1H), 7.00 (d, J = 8.5 Hz, 1H), 5.08-4.99 (m, 1H), 4.89 (t, J = 4.8 Hz, 1H), 4.45 (m, 1H), |
| | | | | 4.36 (m, 1H), |
| | | | | 4.23 (m, 1H), |
| | | | | 4.03-3.93 (m, |
| | | | | 2H), 3.79-3.67 |
| | | | | (m, 2H), 3.52- |
| | | | | 3.44 (m, 2H), |
| | | | | 3.16-3.04 (m, |
| | | | | 1H), 2.99-2.90 |
| | | | | (m, 1H), 2.89- |
| | | | | 2.79 (m, 2H), |
| | | | | 2.71-2.55 (m, |
| | | | | 2H), 2.41-2.28 |
| | | | | (m, 3H), 2.00- |
| | | | | 1.91 (m, 1H), |
| | | | | 1.89-1.78 (m, |
| | | | | 4H), 1.06 (s, 6H) |
| 138 | 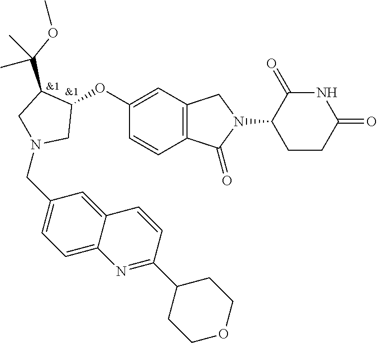 [SMILES: COC(C)(C)[C@@H]1CN(Cc2ccc3nc(C4CCOCC4)ccc3c2)C[C@H]1Oc1ccc2c(c1)CN([C@H]1CCC(=O)NC1=O)C2=O] | (3S)-3-(5-((4-(2- methoxypropan- 2-yl)-1-((2- (tetrahydro-2H- pyran-4- yl)quinolin-6-yl) methyl)pyrrolidin- 3-yl)oxy)-1- oxoisoindolin-2- yl)piperidine-2,6- dione | MS (ESI) m/z 627.6 [M + 1]⁺ | ¹H NMR (400 MHz, DMSO-d₆) δ 10.96 (s, 1H), 8.24 (dd, J = 8.5, 2.6 Hz, 1H), 8.16-8.12 (m, 1H), 7.88 (dd, J = 8.5, 1.7 Hz, 1H), 7.80 (t, J = 2.0 Hz, 1H), 7.67 (dd, J = 8.5, 1.9 Hz, 1H), 7.58 (dd, J = 8.4, 1.8 Hz, 1H), 7.46 (d, J = 8.5 Hz, 1H), 7.13 (dd, J = 4.3, 2.2 Hz, 1H), 7.05-6.97 (m, 1H), 5.11-5.01 (m, 1H), 4.83 (t, J = 4.8 Hz, 1H), |
| | | | | 4.35 (d, J = 17.2 |
| | | | | Hz, 1H), 4.23 |
| | | | | (dd, J = 17.2, 7.8 |
| | | | | Hz, 1H), 4.01- |
| | | | | 3.93 (m, 2H), |
| | | | | 3.78-3.67 (m, |
| | | | | 2H), 3.52-3.44 |
| | | | | (m, 2H), 3.09 (m, |
| | | | | 4H), 2.97-2.78 |
| | | | | (m, 3H), 2.72- |
| | | | | 2.62 (m, 1H), |
| | | | | 2.62-2.53 (m, |
| | | | | 2H), 2.40-2.25 |
| | | | | (m, 2H), 1.98- |
| | | | | 1.89 (m, 1H), |
| | | | | 1.88-1.73 (m, |
| | | | | 4H), 1.10 (s, 6H) |
| 139 | 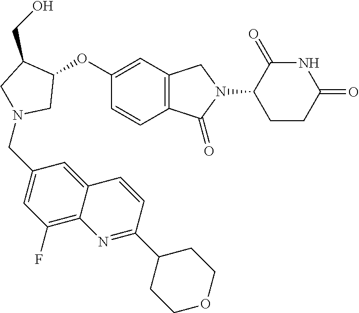 [SMILES: O=C1CC[C@H](N2Cc3cc(O[C@@H]4CN(Cc5cc(F)c6nc(C7CCOCC7)ccc6c5)C[C@H]4CO)ccc3C2=O)C(=O)N1] | (3S)-3-(5- {[(3S,4S)-1-{[8- fluoro-2-(oxan-4- yl)quinolin-6- yl]methyl}-4- (hydroxymethyl) pyrrolidin-3- yl]oxy}-1-oxo- 2,3-dihydro-1H- isoindol-2- yl)piperidine-2,6- dione | MS (ESI) m/z 603.3 [M + 1]⁺ | ¹H NMR (400 MHz, MeOD-d₄) δ 8.32-8.18 (m, 4H), 7.74-7.68 (m, 2H), 7.62- 7.53 (m, 2H), 7.16 (s, 1H), 7.10 (dd, J = 8.5, 2.2 Hz, 1H), 5.12 (dd, J = 13.3, 5.2 Hz, 1H), 4.90 (m, 1H), 4.50-4.39 (m, 2H), 4.11 (m, 2H), 4.05 (s, 2H), 3.72 (m, J2H), 3.68-3.61 (m, 2H), 3.18-3.14 (m, 5H), 2.94- 2.86 (m, 1H), 2.79 (m, 1H), |
| | | | | 2.68 (m, 2H), |
| | | | | 2.52-2.43 (m, |
| | | | | 1H), 2.21-2.13 |
| | | | | (m, 1H), 2.08- |
| | | | | 1.99 (m, 2H), |
| | | | | 1.92 (m, 2H) |
| 140 | 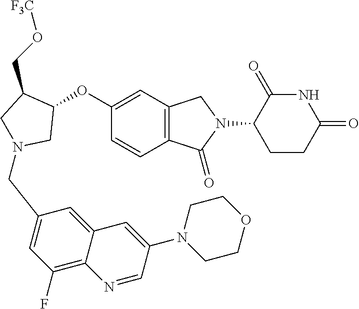 [SMILES: O=C1CC[C@H](N2Cc3cc(O[C@@H]4CN(Cc5cc(F)c6ncc(N7CCOCC7)cc6c5)C[C@H]4COC(F)(F)F)ccc3C2=O)C(=O)N1] | (3S)-3-(5- {[(3S,4S)-1-{[8- fluoro-3- (morpholin-4- yl)quinolin-6- yl]methyl}-4- [(trifluoromethoxy) methyl]pyrrolidin- 3-yl]oxy}-1- oxo-2,3-dihydro- 1H-isoindol-2- yl)piperidine-2,6- dione | MS (ESI) m/z 672.4 [M + 1]⁺ | ¹H NMR (400 MHz, MeOD-d₄) δ 8.78 (d, J = 2.7 Hz, 1H), 7.72 (dd, J = 8.5, 1.8 Hz, 1H), 7.59- 7.53 (m, 2H), 7.32 (dd, J = 11.6, 1.7 Hz, 1H), 7.15-7.05 (m, 2H), 5.16- 5.07 (m, 1H), 4.95-4.89 (m, 1H), 4.50-4.35 (m, 2H), 4.23 (dd, J = 6.6, 1.8 Hz, 2H), 3.96 (s, 2H), 3.91 (dd, J = 5.8, 3.8 Hz, 4H), |
| | | | | 3.38-3.25 (m, |
| | | | | 3H), 3.14 (m, |
| | | | | 3H), 2.91 (m, |
| | | | | 2H), 2.83-2.73 |
| | | | | (m, 1H), 2.65 (m, |
| | | | | 1H), 2.45 (m, |
| | | | | 1H), 2.16 (m, |
| | | | | 1H) |
| 141 | 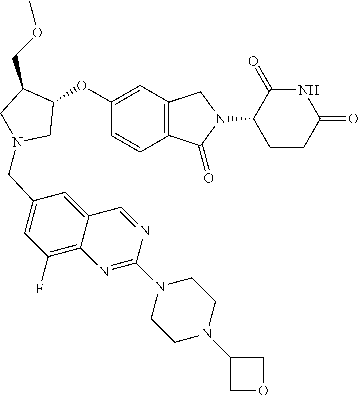 [SMILES: COC[C@@H]1CN(Cc2cc(F)c3nc(N4CCN(C5COC5)CC4)ncc3c2)C[C@H]1Oc1ccc2c(c1)CN([C@H]1CCC(=O)NC1=O)C2=O] | (3S)-3-(5- {[(3S,4S)-1-({8- fluoro-2-[4- (oxetan-3- yl)piperazin-1- yl]quinazolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 674.3 [M + 1]⁺ | ¹H NMR (400 MHz, DMSO-d₆) δ 10.96 (s, 1H), 9.24 (d, J = 1.9 Hz, 1H), 7.60 (m, 2H), 7.53 (d, J = 11.9 Hz, 1H), 7.15 (s, 1H) 7.03- 7.01 (m, 1H), 5.06-5.04 (m, 1H), 4.74 (m, 1H), 4.57 (t, J = 6.5 Hz, 2H), 4.49 (t, J = 6.0 Hz, 2H), 4.37 (d, J = 17.2 Hz, 1H), 4.25 (d, J = 17.2 Hz, 1H), 3.89 (m, 4H), 3.71-7.68 (m, 2H), 3.43- 3.32 (m, 3H), 3.29 (s, 3H), 2.91-2.87 (m, 3H), 2.74 (m, 1H), 2.63-2.52 |
| | | | | (m, 2H), 2.37 (m, |
| | | | | 5H), 2.23 (t, J = |
| | | | | 8.0 Hz, 1H), |
| | | | | 2.01-1.93 (m, |
| | | | | 1H) |
| 142 | 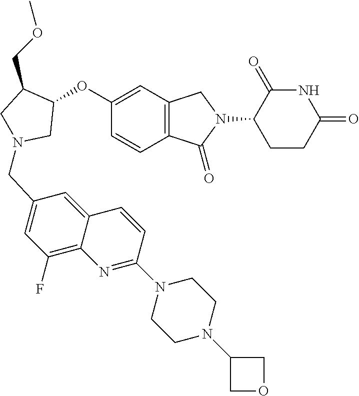 [SMILES: COC[C@@H]1CN(Cc2cc(F)c3nc(N4CCN(C5COC5)CC4)ccc3c2)C[C@H]1Oc1ccc2c(c1)CN([C@H]1CCC(=O)NC1=O)C2=O] | (3S)-3-(5- {[(3S,4S)-1-({8- fluoro-2-[4- (oxetan-3- yl)piperazin-1- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 673.5 [M + 1]⁺ | ¹H NMR (400 MHz, MeOD-d₄) δ 7.99-7.98 (m, 1H), 7.71 (d, J = 8.4 Hz, 1H), 7.42 (d, J = 1.8 Hz, 1H), 7.36-7.33 (m, 1H), 7.22 (d, J = 9.2 Hz, 1H), 7.13 (d, J = 2.2 Hz, 1H), 7.09- 7.07 (m, 1H), 5.14-5.10 (m, 1H), 4.83 (m, 1H), 4.74 (t, J = 6.6 Hz, 2H), 4.68 (t, J = 6.2 Hz, 2H), 4.50-4.35 (m, 2H), 3.84- 3.82 (m, 4H), 3.79-3.77 (m, 2H),3.56-3.51 (m, 3H), 3.38 (s, 3H), 3.12 (t, J = 8.8 Hz, 1H), |
| | | | | 2.95-2.81 (m, |
| | | | | 3H), 2.80-2.79 |
| | | | | (m, 1H), 2.70 (m, |
| | | | | 1H), 2.51-2.39 |
| | | | | (m, 6H), 2.26- |
| | | | | 2.16 (m, 1H) |
| 143 | 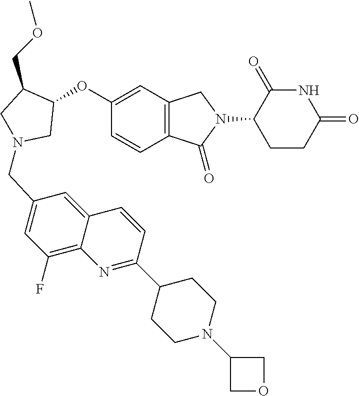 [SMILES: COC[C@@H]1CN(Cc2cc(F)c3nc(C4CCN(C5COC5)CC4)ccc3c2)C[C@H]1Oc1ccc2c(c1)CN([C@H]1CCC(=O)NC1=O)C2=O] | (3S)-3-(5- {[(3S,4S)-1-({8- fluoro-2-[1- (oxetan-3- yl)piperidin-4- yl]quinolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 672.4 [M + 1]⁺ | ¹H NMR (400 MHz, MeOD-d₄) δ 8.28 (d, J = 8.6 Hz, 1H), 7.73- 7.66 (m, 2H), 7.56 (m, 2H), 7.16-7.06 (m, 2H), 5.11 (dd, J = 13.3, 5.1 Hz, 1H), 4.80 (m, 1H), 4.77 (m, 2H), 4.70 (m, 2H), 4.49-4.36 (m, 2H), 3.91 (m, 2H), 3.73 (m, 1H), 3.54 (d, J = 6.5 Hz, 2H), 3.39 (s, 3H), 3.18-2.72 (m, 9H), 2.49- 2.47 (m, 2H), 2.30-2.03 (m, 7H) |
| 144 | 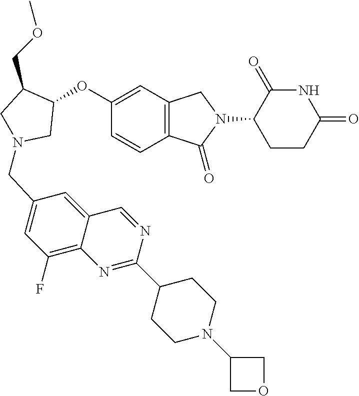 [SMILES: COC[C@@H]1CN(Cc2cc(F)c3nc(C4CCN(C5COC5)CC4)ncc3c2)C[C@H]1Oc1ccc2c(c1)CN([C@H]1CCC(=O)NC1=O)C2=O] | (3S)-3-(5- {[(3S,4S)-1-({8- fluoro-2-[1- (oxetan-3- yl)piperidin-4- yl]quinazolin-6- yl}methyl)-4- (methoxymethyl) pyrrolidin-3- yl]oxy}-1-oxo- 2,3-dihydro-1H- isoindol-2- yl)piperidine-2,6- dione | MS (ESI) m/z 673.6 [M + 1]⁺ | ¹H NMR (400 MHz, MeOD-d₄) δ 9.48 (s, 1H), 7.84-7.78 (m, 2H), 7.71 (d, J = 8.4 Hz, 1H), 7.15 (s, 1H), 7.09 (d, J = 8.6 Hz, 1H), 5.12-5.10 (m, 1H), 4.84 (m, 1H), 4.71-4.68 (m, 4H), 4.48- 4.42 (m, 2H), 3.88 (s, 2H), 3.60-3.53 (m, 3H), 3.39 (s, 3H), 3.09 (m, 2H), 2.94-2.90 (m, 5H), 2.88-2.70 (m, 2H), 2.48- 2.40 (m, 2H), 2.20-2.06 (m, 7H) |
| 145 | 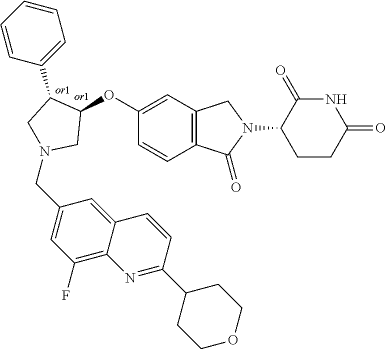 [SMILES: O=C1CC[C@H](N2Cc3cc(O[C@H]4CN(Cc5cc(F)c6nc(C7CCOCC7)ccc6c5)C[C@@H]4c4ccccc4)ccc3C2=O)C(=O)N1] | (S)-3-(5- (((3R*,4S*)-1- ((8-fluoro-2- (tetrahydro-2H- pyran-4- yl)quinolin-6- yl)methyl)-4- phenylpyrrolidin- 3-yl)oxy)-1- oxoisoindolin-2- yl)piperidine-2,6- dione | MS (ESI) m/z 649.5 [M + 1]⁺ | ¹H NMR (400 MHz, MeOD-d₄) δ 8.29-8.27 (m, 1H), 7.70 (s, 1H), 7.65 (m, 1H), 7.62-7.55 (m, 2H), 7.37-7.28 (m, 4H), 7.28- 7.26 (m, 1H), 6.97-6.94 (m,, 1H), 6.90 (d, J = 2.2 Hz, 1H), 5.10-5.08 (m, 1H), 5.03-5.04 (m, 1H), 4.35- 4.31 (m, 2H), 4.12-4.09 (m, 2H), 3.98-3.90 (m, 2H), 3.64- 3.62 (m, 2H), 3.57-3.56 (m, 1H), 3.40 (t, J = |
| | | | | 8.6 Hz, 1H), |
| | | | | 3.38-3.18 (m, |
| | | | | 2H), 3.17-3.13 |
| | | | | (m, 1H), 2.79 (m, |
| | | | | 1H), 2.78 (m, |
| | | | | 1H), 2.68 (t, J = |
| | | | | 8.9 Hz, 1H), 2.66 |
| | | | | (m, 1H), 2.05- |
| | | | | 2.04 (m, 1H), |
| | | | | 2.02-2.01 (m,, |
| | | | | 2H), 1.93-1.90 |
| | | | | (m, 2H) |
| 146 | 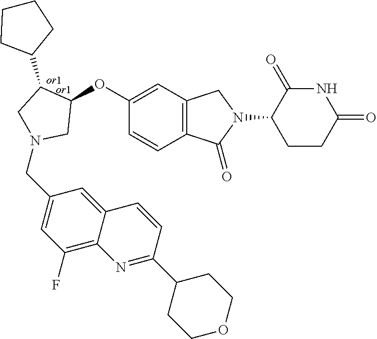 [SMILES: O=C1CC[C@H](N2Cc3cc(O[C@H]4CN(Cc5cc(F)c6nc(C7CCOCC7)ccc6c5)C[C@@H]4C4CCCC4)ccc3C2=O)C(=O)N1] | (S)-3-(5- (((3R*,4S*)-4- cyclopentyl-1- ((8-fluoro-2- (tetrahydro-2H- pyran-4- yl)quinolin-6-yl) methyl)pyrrolidin- 3-yl)oxy)-1- oxoisoindolin-2- yl)piperidine-2,6- dione | MS (ESI) m/z 641.3 [M + 1]⁺ | ¹H NMR (400 MHz, MeOD-d₄) δ 8.27 (dd, J = 8.7, 1.5 Hz, 1H), 7.76-7.65 (m, 2H), 7.59-7.50 (m, 2H), 7.10- 7.00 (m, 2H), 5.11 (dd, J = 13.3, 5.1 Hz, 1H), 4.74 (d, J = 6.3 Hz, 1H), 4.48-4.35 (m, 2H), 4.16-4.06 (m, 2H), 3.85- 3.83 (m, 2H), 3.64 (m, 2H), 3.22 (m, 2H), 3.00-2.88 (m, 3H), 2.83-2.73 (m, 1H), 2.51- |
| | | | | 2.27 (m, 3H), |
| | | | | 2.16 (m, 1H), |
| | | | | 2.09-1.87 (m, |
| | | | | 5H), 1.79 (m, |
| | | | | 2H), 1.70-1.50 |
| | | | | (m, 4H), 1.34- |
| | | | | 1.32 (m, 1H), |
| | | | | 1.25-1.15 (m, |
| | | | | 1H) |
| 147 | 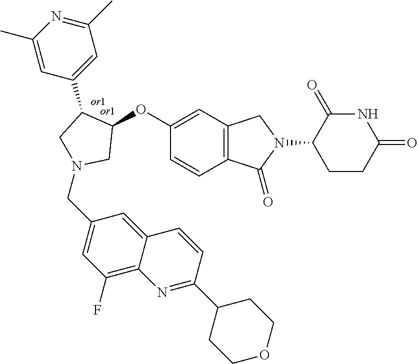 [SMILES: Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)cc(C)n1] | (S)-3-(5- (((3R*,4S*)-4- (2,6- dimethylpyridin- 4-yl)-1-((8- fluoro-2- (tetrahydro-2H- pyran-4- yl)quinolin-6-yl) methyl)pyrrolidin- 3-yl)oxy)-1- oxoisoindolin-2- yl)piperidine-2,6- dione | MS (ESI) m/z 678.5 [M + 1]⁺ | ¹H NMR (400 MHz, MeOD-d₄) δ 8.28 (d, J = 8.6 Hz, 1H), 8.17 (d, J = 6.6 Hz, 1H), 7.68 (d, J = 9.0 Hz, 2H), 7.58- 7.57 (m, 2H), 7.25 (m, 2H), 7.01-6.97 (m, 2H), 5.13-5.09 (m, 2H), 4.44 (d, J = 17.0 Hz, 1H), 4.35 (d, J = 17.0 Hz, 1H), 4.13- 4.09 (m, 2H), 4.02-3.94 (m, 2H), 3.65-3.62 (m, 2H), 3.58 (m, 1H), 3.36 (m, 2H), 3.30-3.25 (m, 1H), 3.07- 3.04 (m, 1H), |
| | | | | 2.90-2.87 (m, |
| | | | | 1H), 2.79-2.71 |
| | | | | (m, 2H), 2.56 (s, |
| | | | | 3H), 2.54 (s, 3H), |
| | | | | 2.53-2.35 (m, |
| | | | | 1H), 2.15 (m, |
| | | | | 1H), 2.04-2.01 |
| | | | | (m, 2H), 1.93 (m, |
| | | | | 2H |
| 148 | 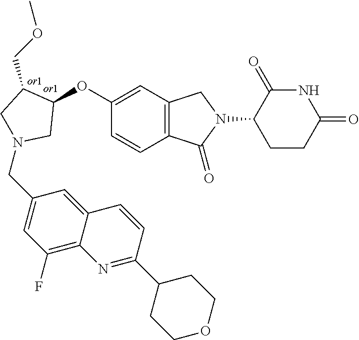 | (3S)-3-(5- {[(3R*,4S*)-1- {[8-fluoro-2- (oxan-4- yl)quinolin-6- yl]methyl}-4-(2- methylpropyl) pyrrolidin-3-yl] oxy}-1-oxo-2,3- dihydro-1H- isoindol-2- yl)piperidine-2,6- dione | MS (ESI) m/z 629.5 [M + 1]⁺ | ¹H NMR (400 MHz, MeOD-d₄) δ 8.28 (dd, J = 8.6, 1.5 Hz, 1H), 7.74-7.65 (m, 2H), 7.61-7.50 (m, 2H), 7.09- 7.00 (m, 2H), 5.12 (m, 1H), 4.67 (m, 1H), 4.50-4.36 (m, 2H), 4.15-4.07 (m, 2H), 3.98- 3.83 (m, 2H), 3.70-3.59 (m, 2H), 3.28-3.19 (m, 2H), 2.99 (m, 2H), 2.98-2.85 (m, 1H), 2.84- 2.74 (m, 1H), 2.60 (m, 1H), |
| | | | | 2.56-2.40 (m, |
| | | | | 1H), 2.30 (m, |
| | | | | 1H), 2.21-2.11 |
| | | | | (m, 1H), 2.11- |
| | | | | 1.96 (m, 2H), |
| | | | | 1.92 (m, 2H), |
| | | | | 1.67-1.38 (m, |
| | | | | 3H), 0.95 (dd, J = |
| | | | | 12.5, 6.4 Hz, 6H) |
| 149 | 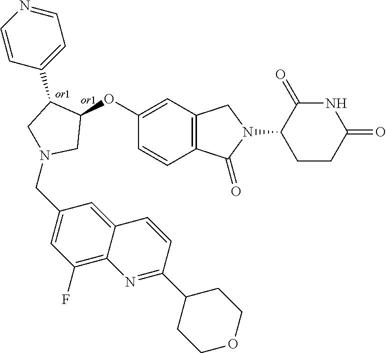 [SMILES: O=C1CC[C@H](N2Cc3cc(O[C@H]4CN(Cc5cc(F)c6nc(C7CCOCC7)ccc6c5)C[C@@H]4c4ccncc4)ccc3C2=O)C(=O)N1] | (S)-3-(5- (((3R*,4S*)-1- ((8-fluoro-2- (tetrahydro-2H- pyran-4- yl)quinolin-6- yl)methyl)-4- (pyridin-4- yl)pyrrolidin-3- yl)oxy)-1- oxoisoindolin-2- yl)piperidine-2,6- dione | MS (ESI) m/z 650.5 [M + 1]⁺ | ¹H NMR (400 MHz, MeOD-d₄) δ 8.50 (d, J = 5.2 Hz, 2H), 8.31- 8.21 (m, 2H), 7.71-7.64 (m, 2H), 7.58 (m, 2H), 7.49 (d, J = 5.2 Hz, 2H), 6.99 (m, 2H), 5.10 (m, 2H), 4.37 (s, 2H), 4.11 (m, 2H), 3.95 (m, 2H), 3.64-3.61 (m, 3H), 3.38 (m, 2H), 3.28-3.21 (m, 1H), 3.05 (m, 1H), 2.90-2.88 (m, 1H), 2.80- 2.71 (m, 2H), 2.46-2.44 (m, 1H), 2.15-2.12 |
| | | | | (m, 1H), 2.03- |
| | | | | 2.01 (m, 2H), |
| | | | | 1.96-1.87 (m, |
| | | | | 2H) |
| 150 | 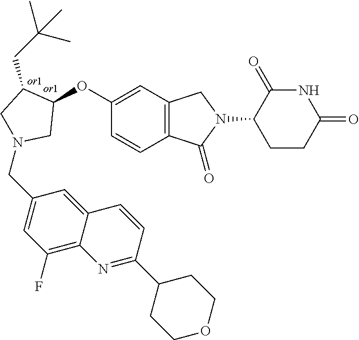 [SMILES: CC(C)(C)C[C@H]1CN(Cc2cc(F)c3nc(C4CCOCC4)ccc3c2)C[C@@H]1Oc1ccc2c(c1)CN([C@H]1CCC(=O)NC1=O)C2=O] | (3S)-3-(5- {[(3R*,4S*)-4- (2,2- dimethylpropyl)- 1-{[8-fluoro-2- (oxan-4- yl)quinolin-6-yl] methyl}pyrrolidin- 3-yl]oxy}-1- oxo-2,3-dihydro- 1H-isoindol-2- yl)piperidine-2,6- dione | MS (ESI) m/z 643.6 [M + 1]⁺ | ¹H NMR (400 MHz, MeOD-d₄) δ 8.31-8.23 (m, 1H), 7.73-7.63 (m, 2H), 7.60- 7.49 (m, 2H), 7.08-6.99 (m, 2H), 5.15-5.06 (m, 1H), 4.66 (m, 1H), 4.49-4.34 (m, 2H), 4.10 (m, 2H), 3.92 (m, 1H), 3.88-3.80 (m, 1H) 3.69- 3.58 (m, 2H), 3.32-3.18 (m, 2H), 3.01 (m, 1H), 2.97-2.83 (m, 2H), 2.83- 2.73 (m, 1H), 2.59 (m, 1H), |
| | | | | 2.44 (m, 1H), |
| | | | | 2.32-2.22 (m, |
| | | | | 1H), 2.20-2.10 |
| | | | | (m, 1H), 2.09- |
| | | | | 1.95 (m, 2H), |
| | | | | 1.90 (m, 2H), |
| | | | | 1.68-1.58 (m, |
| | | | | 1H), 1.52-1.42 |
| | | | | (m, 1H), 0.96- |
| | | | | 0.90 (s, 9H) |
| 151 | 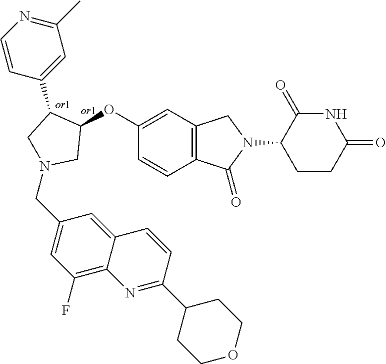 [SMILES: Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)ccn1] | (3S)-3-(5- {[(3R*,4S*)-1- {[8-fluoro-2- (oxan-4- yl)quinolin-6- yl]methyl}-4-(2- methylpyridin-4- yl)pyrrolidin-3- yl]oxy}-1-oxo- 2,3-dihydro-1H- isoindol-2- yl)piperidine-2,6- dione | MS (ESI) m/z 664.5 [M + 1]⁺ | ¹H NMR (400 MHz, DMSO-d₆) δ 10.96 (s, 1H), 8.56 (m, 2H), 7.78-7.55 (m, 4H), 7.28 (m, 2H), 7.08-6.95 (m, 2H), 5.17- 5.03 (m, 2H), 4.25 (m, 2H), 4.00 (m, 2H), 3.88 (m, 2H), 3.60-3.47 (m, 3H), 3.17 (m, 2H), 2.89 (m, 2H), 2.60 (m, 1H), 2.47 (m, 4H), 2.35 (m, 2H), 2.02-1.79 (m, 5H) |
| 152 | 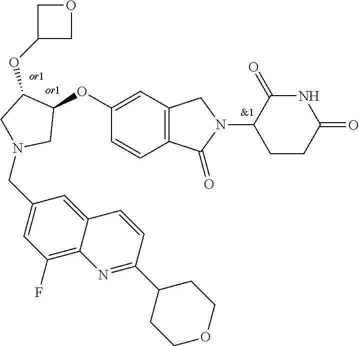 [SMILES: O=C1CCC(N2Cc3cc(O[C@H]4CN(Cc5cc(F)c6nc(C7CCOCC7)ccc6c5)C[C@@H]4OC4COC4)ccc3C2=O)C(=O)N1] | rel-3-(5- (((3S,4S)-1-((8- fluoro-2- (tetrahydro-2H- pyran-4- yl)quinolin-6- yl)methyl)-4- (oxetan-3- yloxy)pyrrolidin- 3-yl)oxy)-1- oxoisoindolin-2- yl)piperidine-2,6- dione | MS (ESI) m/z 645.4 [M + 1]⁺ | ¹H NMR (400 MHz, MeOD-d₄) δ 8.32 (d, J = 8.8 Hz, 1H), 7.91 (d, J = 19.8 Hz, 1H), 7.77-7.63 (m, 3H), 7.19-7.05 (m, 2H), 5.24 (d, J = 4.2 Hz, 1H), 5.14 (dd, J = 13.3, 5.1 Hz, 1H), 4.85-4.74 (m, 3H), 4.72- 4.59 (m, 3H), 4.55-4.40 (m, 3H), 4.17-4.02 (m, 3H), 3.93- 3.59 (m, 6H), 3.27-3.25 (m, 1H), 2.93-2.90 (m, 1H), 2.80- 2.77 (m, 1H), |
| | | | | 2.55-2.44 (m, |
| | | | | 1H), 2.19-2.18 |
| | | | | (m, 1H), 2.04- |
| | | | | 2.01 (m, 2H), |
| | | | | 1.99-1.90 (m, |
| | | | | 2H) |

or a pharmaceutically acceptable salt thereof.

It is understood that in the present description, combinations of substituents and/or variables of the depicted formulae are permissible only if such contributions result in stable compounds.

Furthermore, all compounds of the present disclosure that exist in free base or acid form can be converted to their pharmaceutically acceptable salts by treatment with the appropriate inorganic or organic base or acid by methods known to one skilled in the art. Salts of the compounds of the present disclosure can be converted to their free base or acid form by standard techniques.

### Methods of Synthesis

The compounds described herein can be made using conventional organic syntheses and commercially available starting materials, or the methods provided herein. By way of example and not limitation, compounds of the present disclosure can be prepared as outlined in Schemes 1-12, as well as in the examples set forth herein. It should be noted that one skilled in the art would know how to modify the procedures set forth in the illustrative schemes and examples to arrive at the desired products, including, for example, selecting starting materials having different stereochemistry (or racemic starting materials) to arrive at desired products having different stereochemistry.

In general, the compounds of the present disclosure can be obtained by reacting an aldehyde of interest to the amine in the pyrrolidine group of the compounds. In some embodiments, the reaction of the aldehyde is the last step in the reaction sequence. See, e.g., Scheme A-1 below. In some embodiments, the reaction of the aldehyde to the amine in the pyrrolidine group is performed in the middle of the reaction sequence. See, e.g., aldehyde synthesis is exemplified in Examples 1-12. All reactions in the schemes below are at room temperature unless noted otherwise.

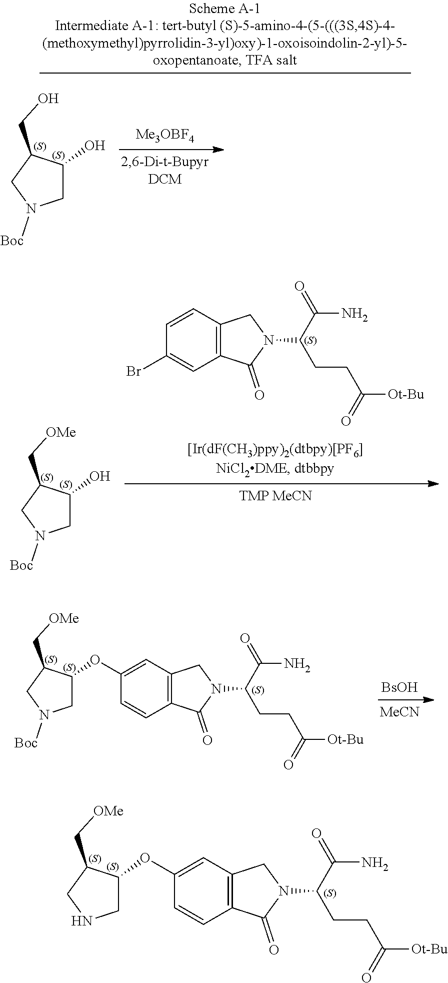

### Step 1: tert-butyl (3S,4S)-3-hydroxy-4-(methoxymethyl)pyrrolidine-1-carboxylate

Trimethyloxonium tetrafluoroborate (3000 mg, 20.27 mmol) was added to a a solution of tert-butyl (3S,4S)-3-hydroxy-4-(hydroxymethyl)pyrrolidine-1-carboxylate (3000 mg, 13.81 mmol) and 2,6-di-tert-butylpyridine (4800 mg, 25.09 mmol) in DCM (10 mL) at 0° C. The reaction was stirred at room temperature under an atmosphere of nitrogen for 16 h. The reaction was diluted with water and extracted with DCM. The combined organic layers were washed with brine, dried with Na₂SO₄, filtered, and concentrated under reduced pressure. The crude mixture was purified via reversed-phase silica gel chromatography to provide Tert-butyl (3S,4S)-3-hydroxy-4-(methoxymethyl)pyrrolidine-1-carboxylate. MS (ESI) m/z: 230.0 [M−H]⁻.

### Step 2: tert-butyl (3S,4S)-3-((2-((S)-1-amino-5-(tert-butoxy)-1,5-dioxopentan-2-yl)-1-oxoisoindolin-5-yl)oxy)-4-(methoxymethyl)pyrrolidine-1-carboxylate

A mixture of tert-butyl (4S)-5-amino-4-(5-bromo-1-oxo-isoindolin-2-yl)-5-oxopentanoate (500 mg, 1.26 mmol), tert-butyl (3S, 4S)-3-hydroxy-4-(methoxymethyl)pyrrolidine-1-carboxylate (500 mg, 2.16 mmol), Ir[dF(Me)ppy]2(dtbbpy)PF6 (15 mg, 0.01 mmol), dtbbpy (15 mg, 0.056 mmol), NiCl₂·DME (15 mg, 0.10 mmol), and TMP (300 mg, 2.12 mmol) in MeCN (12 mL) was irradiated with 100 w blue LEDs for 32 h under an atmosphere of nitrogen. The reaction was concentrated under reduced pressure, dissolved in DMSO and iteratively purified via reversed-phase silica gel column chromatography and preparative SFC [Column: (R,R)-WHELK-01-Kromasil, 250×5 mm, CO₂/(0.1% 2 M NH₃/MeOH)] to provide tert-butyl (3S,4S)-3-((2-((S)-1-amino-5-(tert-butoxy)-1,5-dioxopentan-2-yl)-1-oxoisoindolin-5-yl)oxy)-4-(methoxymethyl)pyrrolidine-1-carboxylate. MS (ESI) m/z: 548.4 [M+H]⁺.

### Step 3: Intermediate A-1, tert-butyl (S)-5-amino-4-(5-(((3S,4S)-4-(methoxymethyl)pyrrolidin-3-yl)oxy)-1-oxoisoindolin-2-yl)-5-oxopentanoate, TFA Salt

A solution of tert-butyl (3S,4S)-3-((2-((S)-1-amino-5-(tert-butoxy)-1,5-dioxopentan-2-yl)-1-oxoisoindolin-5yl)oxy)-4-(methoxymethyl)pyrrolidine-1-carboxylate (950 mg, 1.73 mmol) and BsOH (950 mg, 6.01 mmol) in MeCN (15 mL) was stirred at 60° C. for 16 h under an atmosphere of nitrogen. The reaction was carefully concentrated by bubbling nitrogen to remove the organics and purified via reversed-phase silica gel chromatography (10-40% MeCN/water (w/ 0.05% TFA) to provide tert-butyl (S)-5-amino-4-(5-(((3S,4S)-4-(methoxymethyl)pyrrolidin-3-yl)oxy)-1-oxoisoindolin-2-yl)-5-oxopentanoate, TFA salt. MS (ESI) m/z: 374.2 [M+H]⁺.

### Methods of Use

Embodiments of the present disclosure provide a method for degrading WEE1, a method for reducing WEE1 proteins levels, and a method of preventing or treating diseases such as cancer in a subject in need thereof.

In one aspect, provided herein is a method for degrading WEE1 in a subject in need thereof, the method comprising contacting a cell with an effective amount of a compound of the present disclosure. Degradation of WEE1 can be assessed and demonstrated by a wide variety of methods known in the art. Kits and commercially available assays, including cell-based assays, can be utilized for determining whether and to what degree WEE1 has been degraded. In some embodiments, the compound of the present disclosure partially degrades WEE1. In some embodiments, the compound of the present disclosure fully degrades WEE1.

In some embodiments, a compound of the present disclosure degrades WEE1 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of the present disclosure degrades WEE1 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, provided herein is a method for reducing WEE1 kinase protein levels, the method comprising contacting a cell with an effective amount of a compound of the present disclosure. Reduction of WEE1 kinase protein levels can be assessed and demonstrated by a wide variety of methods known in the art. Kits and commercially available assays, including cell-based assays, can be utilized for determining whether and to what degree kinase protein levels have been reduced.

In some embodiments, a compound of the present disclosure reduces WEE1 kinase protein levels by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of the present disclosure reduces WEE1 kinase protein levels by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, a compound of the present disclosure has an EC₅₀ value as measured in a WEE1 degradation assay of from about 0.0003 μM to about 1 μM or from about 0.0003 μM to about 0.2 μM or from about 0.0003 μM to about 0.05 μM. In some embodiments, a compound of the present disclosure has an EC₅₀ of from about 0.05 μM to about 0.2 μM. In some embodiments, a compound of the present disclosure has an EC₅₀ of from about 0.2 μM to about 1 μM. In some embodiments, a compound of the present disclosure has an EC50 of less than about 1 μM. In some embodiments, a compound of the present disclosure has an EC50 value of less than 0.2 μM, less than 0.05 μM, less than 0.001 μM, or less than about 0.0003 μM.

In another aspect, provided herein is a method for treating cancer in a subject in need thereof, comprising administering to the subject an effective amount of a compound of the present disclosure. In some embodiments, provided herein is a method for preventing cancer in a subject in need thereof, comprising administering to the subject an effective amount of a compound of the present disclosure. In some embodiments, the cancer is selected from a brain cancer, a cervicocerebral cancer, an esophageal cancer, a thyroid cancer, small cell cancer, a non-small cell cancer, a breast cancer, a lung cancer, a stomach cancer, gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

In some embodiments, the cancer is selected from gastric, lung, pancreatic, ovarian, breast, skin, colon, neuroblastoma, osteosarcoma, uterine, rectal, and kidney cancer. In some embodiments, the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).

In some embodiments, administering a compound of the present disclosure to a subject that is predisposed to cancer prevents the subject from developing any symptoms of the cancer (such as tumor growth or metastasis). In some embodiments, administering a compound of the present disclosure to a subject that does not yet display symptoms of cancer prevents the subject from developing any symptoms of the cancer. In some embodiments, administering a compound of the present disclosure to a subject in need thereof diminishes the extent of the cancer in the subject. In some embodiments, administering a compound of the present disclosure to a subject in need thereof stabilizes the cancer (prevents or delays the worsening of the cancer). In some embodiments, administering a compound of the present disclosure to a subject in need thereof delays the occurrence or recurrence of the cancer. In some embodiments, administering a compound of The present invention to a subject in need thereof slows the progression of the cancer. In some embodiments, administering a compound of The present invention to a subject in need thereof provides a partial remission of the cancer. In some embodiments, administering a compound of The present invention to a subject in need thereof provides a total remission of the cancer. In some embodiments, administering a compound of The present invention to a subject in need thereof decreases the dose of one or more other medications required to treat the cancer. In some embodiments, administering a compound of The present invention to a subject in need thereof enhances the effect of another medication used to treat the cancer. In some embodiments, administering a compound of the present disclosure to a subject in need thereof delays the progression of the cancer. In some embodiments, administering a compound of the present disclosure to a subject in need thereof increases the quality of life of the subject having cancer. In some embodiments, administering a compound of the present disclosure to a subject in need thereof prolongs survival of a subject having cancer.

In one aspect, provided herein is method of preventing a subject that is predisposed to cancer from developing cancer, the method comprising administering a compound of the present disclosure to the subject.

In some aspects, provided herein is a method of diminishing the extent of cancer in a subject, the method comprising administering a compound of the present disclosure to the subject. In some embodiments, provided herein is a method of stabilizing cancer in a subject, the method comprising administering a compound of the present disclosure to the subject. In some embodiments, the method prevents the worsening of the cancer.

In another aspect, provided herein is a method of delaying the occurrence or recurrence of cancer in a subject, the method comprising administering a compound of the present disclosure to the subject.

In some embodiments, provided herein is a method of slowing the progression of cancer in a subject, the method comprising administering a compound of the present disclosure to the subject. In some embodiments, the method provides a partial remission of the cancer. In some embodiments, the method provides a total remission of the cancer.

In further aspects, provided herein is a method of decreasing the dose of one or more other medications required to treat cancer in a subject, the method comprising administering a compound of the present disclosure to the subject. In some embodiments, provided herein is a method of enhancing the effect of another medication used to treat cancer in a subject, the method comprising administering a compound of the present disclosure to the subject.

Also provided here is a method of delaying the progression of cancer in a subject, the method comprising administering a compound of the present disclosure to the subject. In some embodiments, the method increases the quality of life of the subject having cancer. In some embodiments, the method prolongs survival of the subject having cancer.

In some embodiments, compounds of the present disclosure are useful in the manufacture of a medicament for reducing WEE1 kinase protein levels. In some embodiments, compounds of the present disclosure are useful in the manufacture of a medicament for the prevention or treatment of a disease associates with WEE1. In some embodiments, compounds of the present disclosure are useful in the manufacture of a medicament for the prevention or treatment of cancer. In some embodiments, the cancer is selected from gastric, lung, pancreatic, ovarian, breast, skin, colon, neuroblastoma, osteosarcoma, uterine, rectal, and kidney cancer. In some embodiments, the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).

The methods and uses of the present disclosure may include a compound of the present disclosure used alone or in combination with one or more additional therapies (e.g., non-drug treatments or therapeutic agents).

A compound of the present disclosure may be administered before, after, or concurrently with one or more of such additional therapies. When combined, dosages of the compound of the present disclosure and dosages of the one or more additional therapies (e.g., non-drug treatment or therapeutic agent) may provide a therapeutic effect (e.g., synergistic or additive therapeutic effect). A compound of the present disclosure and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment). For example, in some embodiments, the compounds of the present disclosure can be used in combination with a therapeutic agent that treats nausea. Examples of agents that can be used to treat nausea include, but are not limited to, dronabinol, granisetron, metoclopramide, ondansetron, prochlorperazine, and pharmaceutically acceptable salts thereof.

In some embodiments, one or more additional therapies includes a non-drug treatment (e.g., surgery or radiation therapy). In some embodiments, one or more additional therapies includes a therapeutic agent (e.g., a compound or biologic that is an antiproliferative agent). In some embodiments, one or more additional therapies includes a non-drug treatment and a therapeutic agent. In other embodiments, one or more additional therapies includes two therapeutic agents. In still other embodiments, one or more additional therapies includes three therapeutic agents. In some embodiments, one or more additional therapies includes four or more therapeutic agents.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of the present disclosure in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of the present disclosure to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of the present disclosure administered will depend on such factors as the solubility of the active component, the formulation used, and the route of administration.

In some embodiments, a compound of the present disclosure is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of the present disclosure.

In a particular embodiment, provided herein are unit dosage formulations comprising about 0.1 mg or 100 mg of a compound of the present disclosure.

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg, or 1400 mg of a compound of the present disclosure.

A compound of the present disclosure can be administered once, twice, three, four or more times daily. As a nonlimiting example, doses of 100 mg or less are administered as a once daily dose and doses of more than 100 mg are administered twice daily in an amount equal to one half of the total daily dose.

A compound of the present disclosure can be administered orally for reasons of convenience. In one embodiment, when administered orally, a compound of the present disclosure is administered with a meal and water. In another embodiment, the compound of the present disclosure is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of the present disclosure without an additional carrier, excipient or vehicle.

In another embodiment, provided herein are compositions comprising an effective amount of a compound of the present disclosure and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories, spray dried dispersions, and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of the present disclosure with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants, and disintegrators as well as the compound. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose, and waxes can also serve as binders.

A lubricant might be necessary in a tablet formulation to prevent the tablet and punches from sticking in the dye. The lubricant can be selected from such slippery solids as talc, magnesium and calcium stearate, stearic acid, and hydrogenated vegetable oils. Tablet disintegrators are substances that swell when wetted to break up the tablet and release the compound. They include starches, clays, celluloses, algins, and gums. More particularly, corn and potato starches, methylcellulose, agar, bentonite, wood cellulose, powdered natural sponge, cation-exchange resins, alginic acid, guar gum, citrus pulp, and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of the present disclosure as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of the present disclosure can be delayed or prolonged by proper formulation. For example, a slowly soluble pellet of the compound of the present disclosure can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of the present disclosure in oily or emulsified vehicles that allow it to disperse slowly in the serum.

### EXAMPLES

The following Examples are presented by way of illustration, not limitation. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

All reactions are at room temperature unless noted otherwise.

The following abbreviations may be relevant for the application.

Abbreviations

| | |
|---|---|
| AcOH | acetic Acid |
| aq. | aqueous |
| Boc | tert-butyloxycarbonyl |
| BsOH | benzenesulfonic acid |
| bpy | 2,2′-Bipyridine |
| CDI | 1,1′-Carbonyldiimidazole |
| Cp₂TiCl₂ | di(cyclopentadienyl)titanium(IV) dichloride |
| CuBr•DMS | copper(I) bromide dimethyl sulfide complex |
| Celite® | diatomaceous earth, flux-calcined |
| DCM | dichloromethane |
| DIPEA | N,N-diisopropylethylamine |
| DME | dimethyl ether |
| DMF | dimethyl formamide |
| DMPU | 1,3-Dimethyl-3,4,5,6-tetrahydro-2(1H)-pyrimidinone |
| DMSO | dimethyl sulfoxide |
| DMSO-d₆ | hexadeuterodimethyl sulfoxide |
| dtbbpy | 4,4′-di-tert-butyl-2,2′-bipyridine |
| EDCI | 1-Ethyl-3-(3-dimethylaminopropyl)carbodiimide |
| ESI | electrospray ionization |
| Et₃N | triethylamine |
| EtOAc | ethyl acetate |
| EtOH | ethanol |
| h | hour |
| HATU | O-(7-azabenzotriazol-1-yl)-N,N,N′,N′-tetramethyluronium |
| | hexafluorophosphate |
| HOBt | 1-Hydroxybenzotriazole |
| HPLC | high performance liquid chromatography |
| i-PrOH | isopropanol |
| [Ir(dF(Me)ppy)₂(dtbpy)][PF₆] | iridium(III) bis[2-(2,4-difluorophenyl)-5-methylpyridine- |
| | N,C20]-4,40-di-tert-butyl-2,20-bipyridine hexafluorophosphate |
| | 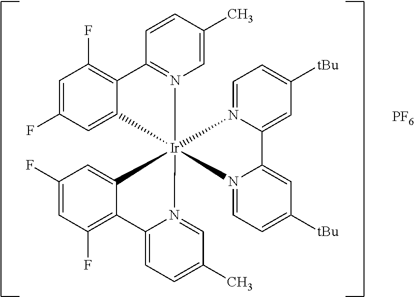 |
| LED | light-emitting diode |
| Me | methyl |
| MeCN | acetonitrile |
| MeMgBr | methylmagnesium bromide |
| MeOD-d₄ | tetradeuteromethanol |
| MeOH | methanol |
| min | minute |
| MS | mass spectrometry |
| n-BuLi | butyllithium |
| NiCl_{2•}DME | nickel(II) chloride ethylene glycol dimethyl ether complex |
| NMO | 4-methylmorpholine 4-oxide |
| NMP | N-methyl-2-pyrrolidone |
| NMR | nuclear magnetic resonance |
| Pd₂(dba)₃ | Tris(dibenzylideneacetone)dipalladium(0) |
| Pd(dppf)Cl_{2•}DCM | [1,1′-Bis(diphenylphosphino)ferrocene]dichloropalladium(II), |
| | complex with dichloromethane |
| Ph₂O | diphenyl ether |
| Py | pyridine |
| RuPhos | 2-Dicyclohexylphosphino-2′,6′-diisopropoxybiphenyl |
| sat. | saturated |
| t-BuOH | tert-butanol |
| TFA | trifluoroacetic acid |
| Tf₂O | trifluoromethanesulfonic (triflc) anhydride |
| THF | tetrahydrofuran |
| Ti(Oi-Pr)₄ | titanium(IV) isopropoxide |
| TMP | 2,2,6,6-tetramethylpiperidine |

### Synthetic Examples

As will be apparent to one skilled in the art, the compounds disclosed below and in Table 1 can exist in various stereochemical forms. Unless otherwise stated, where stereochemistry is shown for the ether linkage between the pyrrolidine and isoindolinone as well as stereochemistry shown between the piperidine-dione to the isoindolinone in the compounds in the Examples and Table 1, the stereochemistry is absolute. Other stereocenters in which stereochemistry is shown may be relative stereochemistry.

### Example 30: (S)-3-(5-(((3S,4S)-1-((8-fluoro-3-morpholinoquinolin-6-yl)methyl)-4-(methoxymethyl)pyrrolidin-3-yl)oxy)-1-oxoisoindolin-2-yl)piperidine-2,6-dione

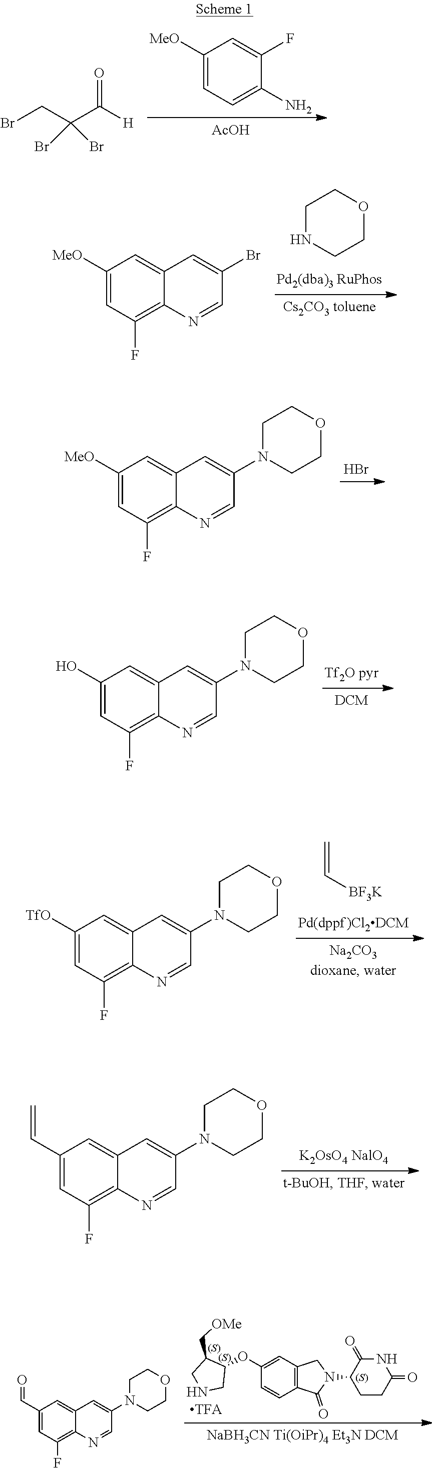

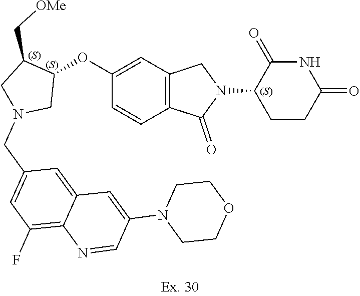

### Step 1: 3-bromo-8-fluoro-6-methoxyquinoline

2,2,3-tribromopropanal (5000 mg, 16.96 mmol) was added to a solution of 2-fluoro-4-methoxyaniline (2400 mg, 17.00 mmol) in AcOH (20 mL) at 0° C. The reaction was stirred at room temperature under an atmosphere of nitrogen. After 12 h, the reaction was concentrated under reduced pressure. The resulting residue was quenched with sat. aq. NaHCO₃ to pH 7 and extracted with EtOAc. The combined organic layers were washed with brine, dried with Na₂SO₄, filtered, and concentrated under reduced pressure. The crude mixture was purified via reversed-phase silica gel chromatography to provide 3-bromo-8-fluoro-6-methoxyquinoline. MS (ESI) m/z: 256.0 [M+H]⁺.

### Step 2: 4-(8-fluoro-6-methoxyquinolin-3-yl)morpholine

A mixture of 3-bromo-8-fluoro-6-methoxyquinoline (310 mg, 1.21 mmol), morpholine (310 mg, 3.56 mmol), Cs₂CO₃ (1180 mg, 3.62 mmol), Pd₂(dba₃) (112 mg, 0.12 mmol), and 2-RuPhos (62 mg, 0.13 mmol) in toluene (10 mL) was stirred at 100° C. for 5 h under an atmosphere of nitrogen. The reaction was cooled to room temperature, concentrated under reduced pressure, and purified via reversed-phase silica gel column chromatography to provide 4-(8-fluoro-6-methoxyquinolin-3-yl)morpholine. MS (ESI) m/z: 263.1 [M+H]⁺.

### Step 3: 8-fluoro-3-morpholinoquinolin-6-ol

A solution of 4-(8-fluoro-6-methoxyqu-inolin-3-yl)morpholine (240 mg, 0.92 mmol) in 40% HBr (10 mL) was stirred at 100° C. for 16 h under an atmosphere of nitrogen. The reaction was cooled to room temperature and poured into ice water. The mixture was adjusted to pH 5 with NaHCO₃ and extracted with DCM. The combined organic layers were washed with brine, dried with Na₂SO₄, filtered, and concentrated under reduced pressure. The crude mixture was purified via reversed-phase silica gel chromatography to provide 8-fluoro-3-morpholinoquinolin-6-ol. MS (ESI) m/z: 249.1 [M+H]⁺.

### Step 4: 8-fluoro-3-morpholinoquinolin-6-yl trifluoromethanesulfonate

Tf₂O (203 mg, 0.72 mmol) was added to a solution of 8-fluoro-3-morpholinoquinolin-6-ol (120 mg, 0.48 mmol) and pyr (119 mg, 1.50 mmol) in DCM (5 mL) at 0° C. The reaction was stirred at room temperature under an atmosphere of nitrogen. After 3 h, the reaction was carefully concentrated by bubbling nitrogen to remove the organics. The crude mixture was purified via reversed-phase silica gel chromatography to provide 8-fluoro-3-morpholinoquinolin-6-yl trifluoromethanesulfonate. MS (ESI) m/z: 381.1 [M+H]⁺.

### Step 5: 4-(8-fluoro-6-vinylquinolin-3-yl)morpholine

Pd(dppf)Cl₂·DCM (17 mg, 0.021 mmol) and Na₂CO₃ (51 mg, 0.48 mmol) were added to a solution of 8-fluoro-3-morpholinoquinolin-6-yl trifluoromethanesulfonate (93 mg, 0.24 mmol) and potassium vinyltrifluoroborate (64 mg, 0.48 mmol) in dioxane (5 mL) and water (0.5 mL). The reaction was stirred at 80° C. for 3 h under an atmosphere of nitrogen. The reaction was cooled to room temperature, diluted with water, and extracted with EtOAc. The combined organic layers were washed with brine, dried with Na₂SO₄, filtered, and concentrated under reduced pressure. The crude mixture was purified via reversed-phase silica gel chromatography to provide 4-(8-fluoro-6-vinylquinolin-3-yl)morpholine. MS (ESI) m/z: 259.2 [M+H]⁺.

### Step 6: 8-fluoro-3-morpholinoquinoline-6-carbaldehyde

K₂OsO₄ (29 mg, 0.079 mmol) and NaIO₄ (341 mg, 1.58 mmol) were added to a solution of 4-(8-fluoro-6-vinylquinolin-3-yl)morpholine (205 mg, 0.79 mmol) in 1:1:1 t-BuOH/THF/water (9 mL). The reaction was stirred at room temperature under an atmosphere of nitrogen. After 1 h, the reaction was quenched with sat. aq. NaHCO₃ to pH 7 and extracted with EtOAc. The combined organic layers were dried with Na₂SO₄, filtered, and concentrated under reduced pressure to provide, 8-fluoro-3-morpholinoquinoline-6-carbaldehyde, which was used in the subsequent step without further purification. MS (ESI) m/z: 650.6 [M+H]⁺.

### Step 7: Example 30, (S)-3-(5-(((3S,4S)-1-((8-fluoro-3-morpholinoquinolin-6-yl)methyl)-4-(methoxymethyl)pyrrolidin-3-yl)oxy)-1-oxoisoindolin-2-yl)piperidine-2,6-dione)

Ti(Oi-Pr)₄ (153 mg, 0.54 mmol) and Et₃N (61 mg, 0.60 mmol) were added to a solution of 8-fluoro-3-morpholinoquinoline-6-carbaldehyde (70 mg, 0.27 mmol) and (S)-3-(5-(((3S,4S)-4-(methoxymethyl)pyrrolidin-3-yl)oxy)-1-oxoisoindolin-2-yl)piperidine-2,6-dione, TFA salt (80 mg, 0.16 mmol) in DCM (5 mL). The reaction was stirred at room temperature under an atmosphere of nitrogen for 20 min, at which point, NaBH₃CN (14 mg, 0.22 mmol) was added. The reaction mixture was stirred for an additional 3 h at room temperature. The reaction was concentrated under reduced pressure and iteratively purified via silica gel chromatography (0-10% MeOH/DCM) and preparative chiral HPLC [Column: JW-Chiral Amylose-SA, 250×20 mm, (1:2 EtOH/DCM)/hexane] to provide (S)-3-(5-(((3S,4S)-1-((8-fluoro-3-morpholinoquinolin-6-yl)methyl)-4-(methoxymethyl)pyrrolidin-3-yl)oxy)-1-oxoisoindolin-2-yl)piperidine-2,6-dione. MS (ESI) m/z: 618.3 [M+H]⁺. ¹H NMR (400 MHz, MeOD-d₄) δ 8.79 (d, J=2.7 Hz, 1H), 7.72 (d, J=8.5 Hz, 1H), 7.60-7.54 (m, 2H), 7.34 (dd, J=11.5, 1.7 Hz, 1H), 7.15 (d, J=2.2 Hz, 1H), 7.09 (dd, J=8.4, 2.2 Hz, 1H), 5.13 (dd, J=13.3, 5.2 Hz, 1H), 4.90 (m, 1H), 4.51-4.36 (m, 2H), 3.97 (d, J=2.6 Hz, 2H), 3.95-3.88 (m, 4H), 3.55 (d, J=6.3 Hz, 2H), 3.38 (s, 3H), 3.25 (m, 4H), 3.20 (m, 1H), 3.16-3.04 (m, 2H), 2.92 (m, 1H), 2.84-2.75 (m, 2H), 2.60 (m, 1H), 2.48 (m, 1H), 2.17 (m, 1H).

### Example 50: rel-(S)-3-(5-(((3S,4S)-1-((8-fluoro-2-(4-hydroxycyclohexyl)quinazolin-6-yl)methyl)-4-(methoxymethyl)pyrrolidin-3-yl)oxy)-1-oxoisoindolin-2-yl)piperidine-2,6-dione

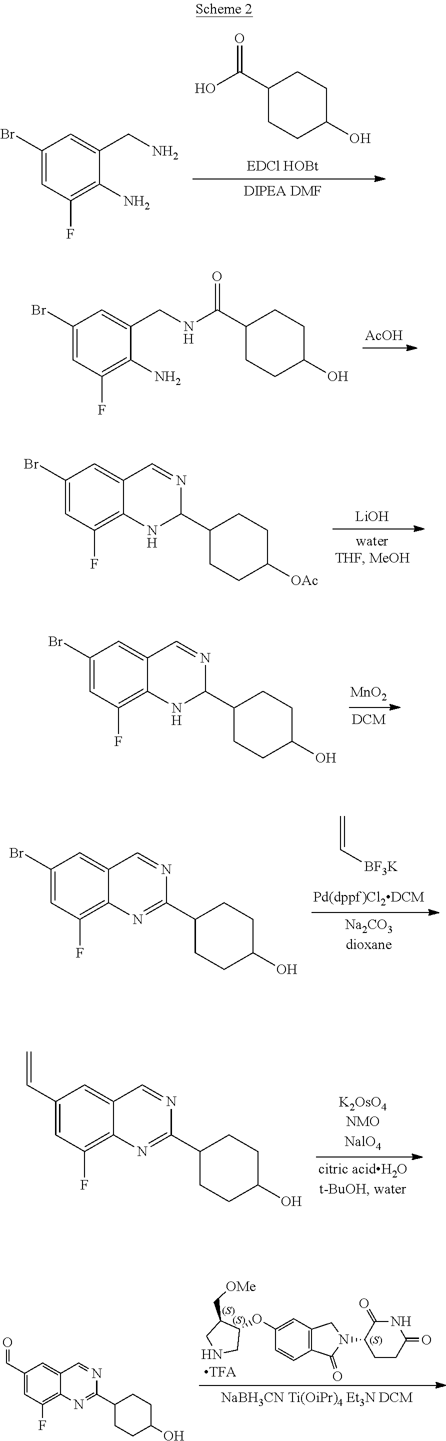

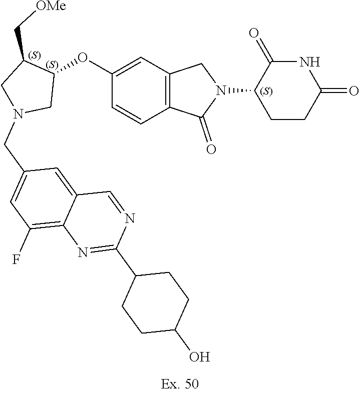

### Step 1: N-(2-amino-5-bromo-3-fluorobenzyl)-4-hydroxycyclohexane-1-carboxamide

DIPEA (1.18 g, 9.13 mmol), HOBt (925 mg, 6.85 mmol), and EDCI (1.31 g, 6.83 mmol) were added to a mixture of 2-(aminomethyl)-4-bromo-6-fluoro-aniline (1 g, 4.57 mmol) and 4-hydroxycyclohexanecarboxylic acid (658 mg, 4.57 mmol) in DMF (20 mL). The reaction was stirred at room temperature. After 1 h, the reaction was diluted with water and extracted with EtOAc. The combined organic layers were washed with brine, dried with Na₂SO₄, filtered, and concentrated under reduced pressure. The crude mixture was purified via silica gel chromatography (0-100% EtOAc/petroleum ether) to provide N-(2-amino-5-bromo-3-fluorobenzyl)-4-hydroxycyclohexane-1-carboxamide. MS (ESI) m/z: 345.0 [M+H]⁺.

### Step 2: 4-(6-bromo-8-fluoro-1,2-dihydroquinazolin-2-yl)cyclohexyl Acetate

A mixture of N-(2-amino-5-bromo-3-fluorobenzyl)-4-hydroxycyclohexane-1-carboxamide (1 g, 2.89 mmol) in AcOH (16 mL) was stirred for 16 h at 100° C. under an atmosphere of nitrogen. The reaction was concentrated under reduced pressure and purified via silica gel chromatography (0-100% EtOAc/petroleum ether) to provide 4-(6-bromo-8-fluoro-1,2-dihydroquinazolin-2-yl)cyclohexyl acetate. MS (ESI) m/z: 369.0 [M+H]⁺.

### Step 3: 4-(6-bromo-8-fluoro-1,2-dihydroquinazolin-2-yl)cyclohexan-1-ol

A solution of LiOH (65 mg, 2.71 mmol) in water (5 mL) was added to a solution of 4-(6-bromo-8-fluoro-1,2-dihydroquinazolin-2-yl)cyclohexyl acetate (500 mg, 1.36 mmol) in THF (2.5 mL) and MeOH (2.5 mL). The reaction was stirred at room temperature under an atmosphere of nitrogen. After 1 h, the reaction was diluted with water and extracted with EtOAc. The combined organic layers were washed with brine, dried with Na₂SO₄, filtered, and concentrated under reduced pressure to provide 4-(6-bromo-8-fluoro-1,2-dihydroquinazolin-2-yl)cyclohexan-1-ol, which was used in the subsequent step without further purification. MS (ESI) m/z: 327.1 [M+H]⁺.

### Step 4: 4-(6-bromo-8-fluoroquinazolin-2-yl)cyclohexan-1-ol

A mixture of 4-(6-bromo-8-fluoro-1,2-dihydroquinazolin-2-yl)cyclohexan-1-ol (700 mg, 2.14 mmol) and MnO₂ (1.86 g, 21.39 mmol) in DCM (16 mL) was stirred for 16 h at room temperature under an atmosphere of nitrogen. The reaction was filtered through Celite® and the resulting filtrate was concentrated under reduced pressure to provide 4-(6-bromo-8-fluoroquinazolin-2-yl)cyclohexan-1-ol, which was used in the subsequent step without further purification. MS (ESI) m/z: 325.0 [M+H]⁺.

### Step 5: 4-(8-fluoro-6-vinylquinazolin-2-yl)cyclohexan-1-ol

Na₂CO₃ (685 mg, 6.46 mmol) and Pd(dppf)Cl₂·DCM (176 mg, 0.21 mmol) were added to a mixture of 4-(6-bromo-8-fluoroquinazolin-2-yl)cyclohexan-1-ol (700 mg, 2.15 mmol) and potassium vinyltrifluoroborate (577 mg, 4.31 mmol) in dioxane (10 mL) and water (2 mL). The reaction was stirred at 80° C. for 2 h under an atmosphere of nitrogen. The reaction was cooled to room temperature and concentrated under reduced pressure. The crude mixture was purified via silica gel chromatography (0-100% EtOAc/petroleum ether) to provide 4-(8-fluoro-6-vinylquinazolin-2-yl)cyclohexan-1-ol. MS (ESI) m/z: 273.2 [M+H]⁺.

### Step 6: 8-fluoro-3-morpholinoquinoline-6-carbaldehyde

NMO (430 mg, 3.67 mmol), citric acid monohydrate (771 mg, 3.67 mmol), and K₂OsO₄ (68 mg, 0.18 mmol) were added to a solution of 4-(8-fluoro-6-vinylquinazolin-2-yl)cyclohexan-1-ol (500 mg, 1.83 mmol) in t-BuOH (9 mL) and water (9 mL). The reaction was stirred at room temperature for 1 h, at which, point NaIO₄ (982 mg, 4.59 mmol) was added. The reaction stirred for an additional 2 h at room temperature under an atmosphere of nitrogen. The reaction was quenched with sat. aq. NaHCO₃ to pH 7 and extracted with EtOAc. The combined organic layers were washed with brine, dried with Na₂SO₄, filtered, and concentrated under reduced pressure. The crude mixture was purified via reversed-phase silica gel chromatography to provide 8-fluoro-3-morpholinoquinoline-6-carbaldehyde. MS (ESI) m/z: 275.2 [M+H]⁺.

### Step 7: Example 50, rel-(S)-3-(5-(((3S,4S)-1-((8-fluoro-2-(4-hydroxycyclohexyl)quinazolin-6-yl)methyl)-4-(methoxymethyl)pyrrolidin-3-yl)oxy)-1-oxoisoindolin-2-yl)piperidine-2,6-dione

Et₃N (59 mg, 0.58 mmol) and Ti(Oi-Pr)₄ (165 mg, 0.58 mmol) were added to a solution of 8-fluoro-2-(4-hydroxycyclohexyl)quinazoline-6-carbaldehyde (80 mg, 0.29 mmol) and (S)-3-(5-(((3S,4S)-4-(methoxymethyl)pyrrolidin-3-yl)oxy)-1-oxoisoindolin-2-yl)piperidine-2,6-dione, TFA salt (142 mg, 0.29 mmol) in DCM (2 mL). The reaction was stirred at room temperature under an atmosphere of nitrogen for 30 min, at which point, NaBH₃CN (37 mg, 0.59 mmol) was added. The reaction mixture was stirred for an additional 2 h at room temperature. The reaction was concentrated under reduced pressure and iteratively purified via silica gel chromatography (0-10% MeOH/DCM) and preparative chiral HPLC [Column: Chiral Art Cellulose-SB, 250×20 mm, (3:1 hexanes/DCM)/EtOH] to provide rel-(S)-3-(5-(((3S,4S)-1-((8-fluoro-2-(4-hydroxycyclohexyl)quinazolin-6-yl)methyl)-4-(methoxymethyl)pyrrolidin-3-yl)oxy)-1-oxoisoindolin-2-yl)piperidine-2,6-dione as a mixture of diastereomers. MS (ESI) m/z: 632.5 [M+H]⁺. ¹H NMR (400 MHz, MeOD-d₄) δ 9.49 (d, J=1.4 Hz, 1H), 7.88 (s, 1H), 7.81 (dd, J=11.2, 1.7 Hz, 1H), 7.72 (d, J=8.4 Hz, 1H), 7.15 (d, J=2.2 Hz, 1H), 7.10 (dd, J=8.5, 2.2 Hz, 1H), 5.13 (dd, J=13.3, 5.2 Hz, 1H), 4.88 (m, 1H), 4.45-4.42 (m, 2H), 4.03 (m, 2H), 3.56 (d, J=6.3 Hz, 2H), 3.40 (s, 3H), 3.33-3.11 (m, 4H), 2.92 (m, 1H), 2.81-2.71 (m, 2H), 2.59 (m, 1H), 2.48 (m, 1H), 2.33-2.29 (m, 2H), 2.13 (m, 1H), 1.90-1.71 (m, 6H).

### Example 57: rel-(S)-3-(5-(((3S,4S)-1-((8-fluoro-2-(4-(2-hydroxypropan-2-yl)cyclohexyl)quinolin-6-yl)methyl)-4-(methoxymethyl)pyrrolidin-3-yl)oxy)-1-oxoisoindolin-2-yl)piperidine-2,6-dione

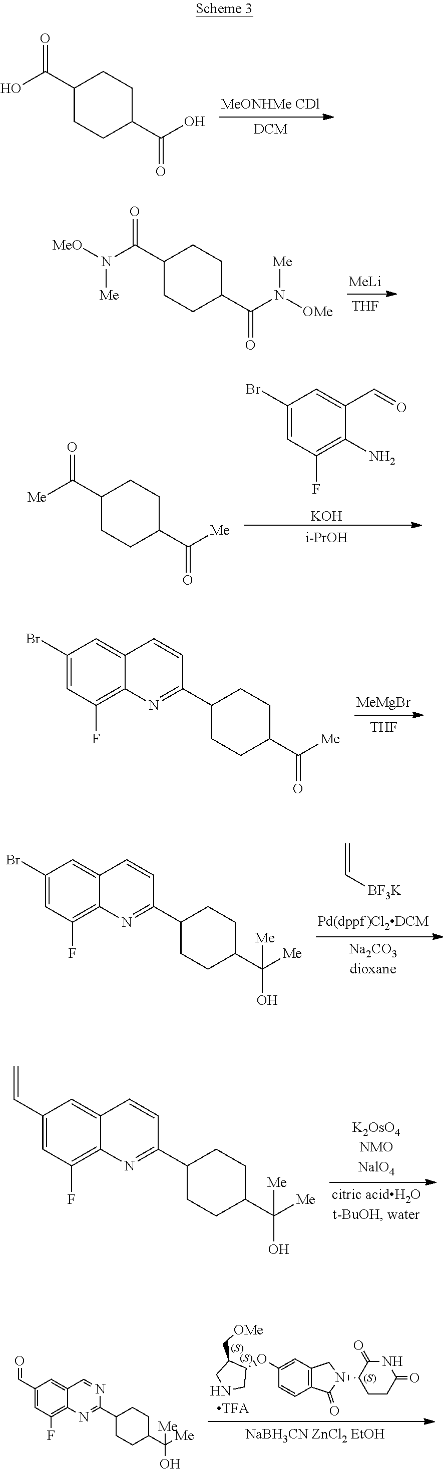

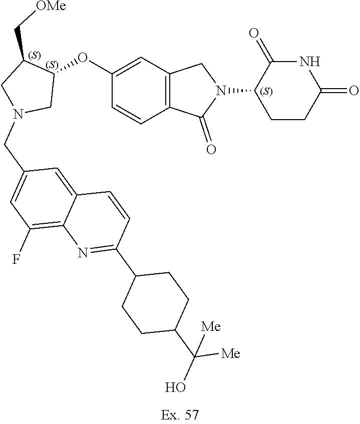

### Step 1: N1,N4-dimethoxy-N1,N4-dimethylcyclohexane-1,4-dicarboxamide

N,O-dimethylhydroxylamine (355 mg, 3.63 mmol) was added to a solution of cyclohexane-1,4-dicarboxylic acid (500 mg, 2.90 mmol) and CDI (942 mg, 5.81 mmol) in DCM (5 mL) at 0° C. The reaction was stirred at room temperature for 2 h under an atmosphere of nitrogen. The reaction was concentrated under reduced pressure and purified via reversed-phase silica gel chromatography to provide N1,N4-dimethoxy-N1,N4-dimethylcyclohexane-1,4-dicarboxamide. MS (ESI) m/z: 259.1 [M+H]⁺.

### Step 2: 1,1′-(cyclohexane-1,4-diyl)bis(ethan-1-one)

MeLi (1.6 N in THF, 4 mL, 6.40 mmol) was added to a solution of N1,N4-dimethoxycyclohexane-1,4-dicarboxamide (340 mg, 1.31 mmol) in THF (5 mL) at 0° C. The reaction was stirred at room temperature for 2 h under an atmosphere of nitrogen. The reaction was quenched with ice water, acidified to pH 7 with sat. aq. citric acid, and extracted with EtOAc. The combined organic layers were washed with brine, dried with Na₂SO₄, filtered, and concentrated under reduced pressure to provide 1,1′-(cyclohexane-1,4-diyl)bis(ethan-1-one), which was used in the subsequent step without further purification. MS (ESI) m/z: 169.2 [M+H]⁺.

### Step 3: 1-(4-(6-bromo-8-fluoroquinolin-2-yl)cyclohexyl)ethan-1-one

KOH (100 mg, 1.79 mmol) was added to a solution of 1,1′-(cyclohexane-1,4-diyl)bis(ethan-1-one) (300 mg, crude) and 2-amino-5-bromo-3-fluorobenzaldehyde (190 mg, 0.87 mmol) in i-PrOH (5 mL) at 0° C. The reaction was stirred at room temperature under an atmosphere of nitrogen. After 2 h, the reaction was diluted with water and extracted with EtOAc. The combined organic layers were washed with brine, dried with Na₂SO₄, filtered, and concentrated under reduced pressure. The crude mixture was purified via reversed-phase silica gel chromatography to provide to provide 1-(4-(6-bromo-8-fluoroquinolin-2-yl)cyclohexyl)ethan-1-one. MS (ESI) m/z: 349.9 [M+H]⁺.

### Step 4: 2-(4-(6-bromo-8-fluoroquinolin-2-yl)cyclohexyl)propan-2-ol

MeMgBr (1 M in THF, 5.1 mL, 5.1 mmol) was added dropwise to a solution of 1-(4-(6-bromo-8-fluoroquinolin-2-yl)cyclohexyl)ethan-1-one (180 mg, 0.51 mmol) in THF (3 mL) at 0° C. under an atmosphere of nitrogen. The reaction was quenched with sat. aq. NH₄Cl and extracted with EtOAc. The combined organic layers were washed with brine, dried with Na₂SO₄, filtered, and concentrated under reduced pressure. The crude mixture was purified via silica gel chromatography (0-30% EtOAc/petroleum ether) to provide 2-(4-(6-bromo-8-fluoroquinolin-2-yl)cyclohexyl)propan-2-ol. MS (ESI) m/z: 366.1 [M+H]⁺.

### Step 5: 2-(4-(8-fluoro-6-vinylquinolin-2-yl)cyclohexyl)propan-2-ol

Na₂CO₃ (122 mg, 1.15 mmol) and Pd(dppf)Cl₂·DCM (31 mg, 0.038 mmol) were added to a mixture of 2-(4-(6-bromo-8-fluoroquinolin-2-yl)cyclohexyl)propan-2-ol (140 mg, 0.38 mmol) and potassium trifluoro(vinyl)borate (103 mg, 0.77 mmol) in dioxane (3 mL) and water (0.2 mL). The reaction was stirred at 80° C. for 2 h under an atmosphere of nitrogen. The reaction was cooled to room temperature, diluted with water and extracted with EtOAc. The combined organic layers were washed with brine, dried with Na₂SO₄, filtered, and concentrated under reduced pressure. The crude mixture was purified via reversed-phase silica gel chromatography to provide 2-(4-(8-fluoro-6-vinylquinolin-2-yl)cyclohexyl)propan-2-ol. MS (ESI) m/z: 314.1 [M+H]⁺.

### Step 6: 8-fluoro-3-morpholinoquinoline-6-carbaldehyde

K₂OsO₄ (11 mg, 0.030 mmol), citric acid (123 mg, 0.64 mmol), and NMO (75 mg, 0.64 mmol) were added to a solution of 2-(4-(8-fluoro-6-vinylquinolin-2-yl)cyclohexyl)propan-2-ol (100 mg, 0.32 mmol) in t-BuOH (3 mL) and water (3 mL). The reaction was stirred at room temperature for 1 h, at which, point NaIO₄ (207 mg, 0.96 mmol) was added. The reaction stirred for an additional 1 h at room temperature under an atmosphere of nitrogen. The reaction was quenched with sat. aq. NaHCO₃ to pH 8 and extracted with EtOAc. The combined organic layers were washed with brine, dried with Na₂SO₄, filtered, and concentrated under reduced pressure to provide 8-fluoro-3-morpholinoquinoline-6-carbaldehyde, which was carried onto the next step without further purification. MS (ESI) m/z: 316.2 [M+H]⁺.

### Step 7: Example 57, rel-(S)-3-(5-(((3S,4S)-1-((8-fluoro-2-(4-(2-hydroxypropan-2-yl)cyclohexyl)quinolin-6-yl)methyl)-4-(methoxymethyl)pyrrolidin-3-yl)oxy)-1-oxoisoindolin-2-yl)piperidine-2,6-dione

NaBH₃CN (24 mg, 0.38 mmol) was added to a solution of 8-fluoro-2-(4-(2-hydroxypropan-2-yl)cyclohexy-l)quinoline-6-carbaldehyde (60 mg, 0.19 mmol), (S)-3-(5-(((3S,4S)-4-(methoxymeth-yl)pyrrolidin-3-yl)oxy)-1-oxoisoindolin-2-yl)piperidine-2,6-dione, TFA salt (80 mg, 0.16 mmol), and ZnCl₂ (0.7 M in THF, 0.3 mL, 0.21 mmol) in EtOH (3 mL). The reaction was stirred at room temperature under an atmosphere of nitrogen. After 4 h, the reaction was concentrated under reduced pressure and iteratively purified via silica gel chromatography (0-10% MeOH/DCM) and preparative chiral HPLC [Column: (R, R)-Whelk-O1, 250×21.1 mm, (1:2 i-PrOH/DCM)/hexane] to provide rel-(S)-3-(5-(((3S,4S)-1-((8-fluoro-2-(4-(2-hydroxypropan-2-yl)cyclohexyl)quinolin-6-yl)methyl)-4-(methoxymethyl)pyrrolidin-3-yl)oxy)-1-oxoisoindolin-2-yl)piperidine-2,6-dione as a single diastereomer. MS (ESI) m/z: 673.3 [M+H]⁺. ¹H NMR (400 MHz, MeOD-d₄) δ 8.29 (dd, J=8.7, 1.6 Hz, 1H), 7.65 (d, J=1.6 Hz, 1H), 7.60 (d, J=8.4 Hz, 1H), 7.54 (d, J=8.6 Hz, 1H), 7.48 (dd, J=11.8, 1.7 Hz, 1H), 7.15 (d, J=2.2 Hz, 1H), 7.03 (dd, J=8.4, 2.2 Hz, 1H), 5.05 (dd, J=13.2, 5.1 Hz, 1H), 4.74 (m, 1H), 4.36 (d, J=17.2 Hz, 1H), 4.25 (d, J=17.3 Hz, 1H), 3.78 (d, J=13.4 Hz, 1H), 3.72 (d, J=13.4 Hz, 1H), 3.45 (m, 2H), 3.26 (s, 3H), 2.98-2.70 (m, 5H), 2.64-2.55 (m, 2H), 2.36 (m, 1H), 2.25 (m, 1H), 2.02-1.90 (m, 5H), 1.63-1.57 (m, 2H), 1.32 (m, 1H), 1.18 (m, 2H), 1.08 (s, 6H).

### Example 64: (S)-3-(5-(((3S,4S)-1-((8-fluoro-2-(4-hydroxypiperidin-1-yl)quinolin-6-yl)methyl)-4-(methoxymethyl)pyrrolidin-3-yl)oxy)-1-oxoisoindolin-2-yl)piperidine-2,6-dione

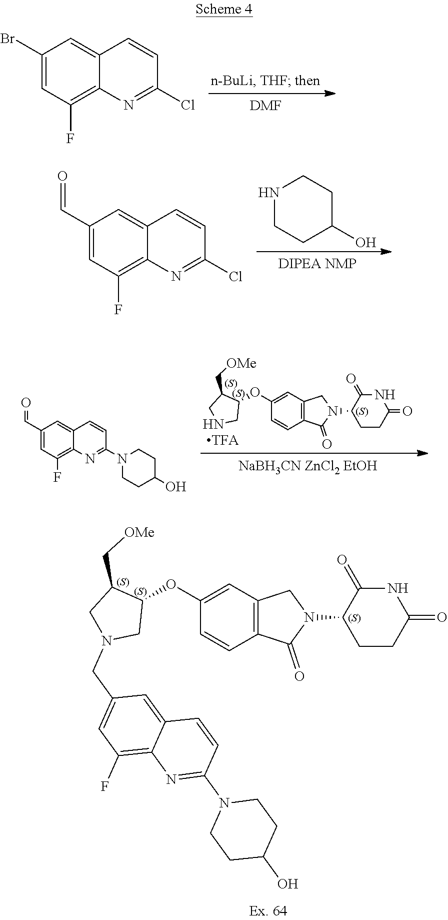

### Step 1: 2-chloro-8-fluoroquinoline-6-carbaldehyde

n-BuLi (2.5 M in THF, 0.90 mL, 2.25 mmol) was added dropwise to a mixture of 6-bromo-2-chloro-8-fluoroquinoline (500 mg, 1.92 mmol) in THF (10 mL) at −78° C. under an atmosphere of nitrogen. The reaction was stirred at −78° C. under an atmosphere of nitrogen for 40 min, at which point, a solution of DMF (182 mg, 2.49 mmol) in THF (1 mL) was added. The reaction was warmed to room temperature and stirred for an additional 1 h. The reaction was cooled to 0° C., quenched with ice water, and extracted with EtOAc. The combined organic layers were washed with brine, dried with Na₂SO₄, filtered, and concentrated under reduced pressure to provide 2-chloro-8-fluoroquinoline-6-carbaldehyde, which was used in subsequent steps without further purification. MS (ESI) m/z: 210.0 [M+H]⁺.

### Step 2: 8-fluoro-2-(4-hydroxypiperidin-1-yl)quinoline-6-carbaldehyde

A solution of 2-chloro-8-fluoroquinoline-6-carbaldehyde (200 mg, 0.95 mmol), piperidin-4-ol (193 mg, 1.91 mmol), and DIPEA (0.25 mL, 2.86 mmol) in NMP (6 mL) was stirred at 120° C. for 2 h under an atmosphere of nitrogen. The reaction was cooled to room temperature and purified via reversed-phase silica gel chromatography to provide 8-fluoro-2-(4-hydroxypiperidin-1-yl)quinoline-6-carbaldehyde. MS (ESI) m/z: 275.2 [M+H]⁺.

### Step 3: Example 64, (S)-3-(5-(((3S,4S)-1-((8-fluoro-2-(4-hydroxypiperidin-1-yl)quinolin-6-yl)methyl)-4-(methoxymethyl)pyrrolidin-3-yl)oxy)-1-oxoisoindolin-2-yl)piperidine-2,6-dione

NaBH₃CN (22 mg, 0.34 mmol) and ZnCl₂ (0.7 M in THF, 0.5 mL, 0.35 mmol) were added to a solution of 8-fluoro-2-(4-hydroxy-1-piperidyl)quinoline-6-carbaldehyde (70 mg, 0.25 mmol) and (S)-3-(5-(((3S,4S)-4-(methoxymethyl)pyrrolidin-3-yl)oxy)-1-oxoisoindolin-2-yl)piperidine-2,6-dione, TFA salt (80 mg, 0.16 mmol) in EtOH (6 mL). The reaction was stirred at room temperature for 16 h under an atmosphere of nitrogen. The reaction was iteratively purified via preparative achiral HPLC [Column: SunFire preparative C18 OBD column, 250×19 mm, water (w/ 0.1% HCO₂H)/MeCN] and preparative chiral HPLC [Column: JW-Chiral-Amylose-SB, 250×20 mm, (1:2 EtOH/DCM)/hexane] to provide (S)-3-(5-(((3S,4S)-1-((8-fluoro-2-(4-hydroxypiperidin-1-yl)quinolin-6-yl)methyl)-4-(methoxymethyl)pyrrolidin-3-yl)oxy)-1-oxoisoindolin-2-yl)piperidine-2,6-dione. MS (ESI) m/z: 632.2 [M+H]⁺. ¹H NMR (400 MHz, DMSO-d₆) δ 8.00 (m, 1H), 7.61 (d, J=8.4 Hz, 1H), 7.40 (d, J=1.7 Hz, 1H), 7.32-7.24 (m, 2H), 7.13 (d, J=2.2 Hz, 1H), 7.02 (m, 1H), 5.03 (m, 1H), 4.70 (m, 1H), 4.36 (d, J=17.4 Hz, 1H), 4.29-4.15 (m, 3H), 3.70 (m, 1H), 3.64 (d, J=4.0 Hz, 2H), 3.49-3.39 (m, 2H), 3.26 (s, 3H), 3.25-3.15 (m, 2H), 2.91-2.88 (m, 3H), 2.71 (m, 1H), 2.65-2.52 (m, 2H), 2.48-2.25 (m, 1H), 2.20 (m, 1H), 2.05-1.95 (m, 1H), 1.86-1.82 (m, 2H), 1.43-1.30 (m, 2H)

### Example 76: rel-(3S)-3-(5-{[(3S,4S)-1-{[8-fluoro-2-(oxolan-2-yl)quinazolin-6-yl]methyl}-4-(methoxymethyl)pyrrolidin-3-yl]oxy}-1-oxo-2,3-dihydro-1H-isoindol-2-yl)piperidine-2,6-dione

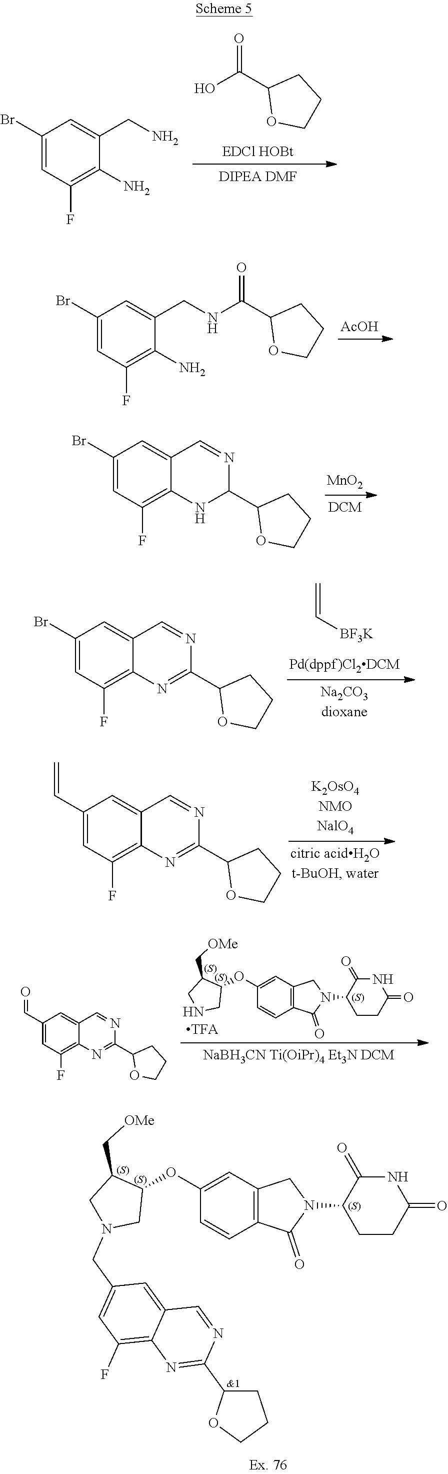

### Step 1: N-(2-amino-5-bromo-3-fluorobenzyl)tetrahydrofuran-2-carboxamide

DIPEA (425 mg, 3.29 mmol) was added to a solution of 2-(aminomethyl)-4-bromo-6-fluoroaniline (360 mg, 1.64 mmol), HOBt (332 mg, 2.46 mmol), EDCI (471 mg, 2.46 mmol), and tetrahydrofuran-2-carboxylic acid (190 mg, 1.64 mmol) in DMF (10 mL) at 0° C. The reaction was stirred at room temperature under an atmosphere of nitrogen. After 3 h, the reaction was diluted with water and extracted with EtOAc. The combined organic layers were washed with brine, dried with Na₂SO₄, filtered, and concentrated under reduced pressure. The crude mixture was purified via silica gel chromatography (0-100% EtOAc/petroleum ether) to provide N-(2-amino-5-bromo-3-fluorobenzyl)tetrahydrofuran-2-carboxamide. MS (ESI) m/z: 317.1 [M+H]⁺.

### Step 2: 6-bromo-8-fluoro-2-(tetrahydrofuran-2-yl)-1,2-dihydroquinazoline

A solution of N-(2-amino-5-bromo-3-fluorobenzyl)tetrahydrofuran-2-carboxamide 410 mg, 1.29 mmol) in AcOH (16 mL) was stirred for 12 h at 100° C. under an atmosphere of nitrogen. The reaction was concentrated under reduced pressure to provide 6-bromo-8-fluoro-2-(tetrahydrofuran-2-yl)-1,2-dihydroquinazoline, which was used in the subsequent step without further purification. MS (ESI) m/z: 298.9 [M+H]⁺.

### Step 3: 6-bromo-8-fluoro-2-(tetrahydrofuran-2-yl)quinazoline

MnO₂ (988 mg, 11.36 mmol) was added to a solution of 6-bromo-8-fluoro-2-(tetrahydrofuran-2-yl)-1,2-dihydroquinazoline (340 mg, 1.14 mmol) in DCM (16 mL). The reaction was stirred for 12 h at room temperature. The reaction was filtered through Celite® and the resulting filtrate was concentrated under reduced pressure to provide 6-bromo-8-fluoro-2-(tetrahydrofuran-2-yl)quinazoline, which was used in the subsequent step without further purification. MS (ESI) m/z: 297.1 [M+H]⁺.

### Step 4: 8-fluoro-2-(tetrahydrofuran-2-yl)-6-vinylquinazoline

Na₂CO₃ (209 mg, 1.97 mmol) and Pd(dppf)Cl₂·DCM (53 mg, 0.065 mmol) were added to a solution of 6-bromo-8-fluoro-2-(tetrahydrofuran-2-yl)quinazoline (260 mg, 0.88 mmol) and potassium vinyltrifluoroborate (176 mg, 1.31 mmol) in dioxane (5 mL) and water (1 mL). The reaction was stirred at 80° C. for 2 h under an atmosphere of nitrogen. The reaction was cooled to room temperature and concentrated under reduced pressure. The crude mixture was purified via silica gel chromatography (0-100% EtOAc/petroleum ether) to provide 8-fluoro-2-(tetrahydrofuran-2-yl)-6-vinylquinazoline. MS (ESI) m/z: 245.2 [M+H]⁺.

### Step 5: 8-fluoro-2-(tetrahydrofuran-2-yl)quinazoline-6-carbaldehyde

Citric acid monohydrate (240 mg, 1.14 mmol), NMO (134 mg, 1.14 mmol), and K₂OsO₄ (21 mg, 0.057 mmol) were added to a solution of 8-fluoro-2-(tetrahydrofuran-2-yl)-6-vinylquinazoline (140 mg, 0.57 mmol) in t-BuOH (3 mL) and water (3 mL). The reaction was stirred at room temperature for 1 h, at which, point NaIO₄ (306 mg, 1.42 mmol) was added. The reaction stirred for an additional 2 h at room temperature. The reaction was quenched with sat. aq. NaHCO₃ to pH 7 and extracted with EtOAc. The combined organic layers were washed with brine, dried with Na₂SO₄, filtered, and concentrated under reduced pressure to provide 8-fluoro-2-(tetrahydrofuran-2-yl)quinazoline-6-carbaldehyde, which was used in the subsequent step without further purification. MS (ESI) m/z: 247.2 [M+H]⁺.

### Step 6: Example 76, rel-(3S)-3-(5-{[(3S,4S)-1-{[8-fluoro-2-(oxolan-2-yl)quinazolin-6-yl]methyl}-4-(methoxymethyl)pyrrolidin-3-yl]oxy}-1-oxo-2,3-dihydro-1H-isoindol-2-yl)piperidine-2,6-dione

Et₃N (29 mg, 0.29 mmol) and Ti(Oi-Pr)₄ (163 mg, 0.57 mmol) were added to a solution of 8-fluoro-2-(tetrahydrofuran-2-yl)quinazoline-6-carbaldehyde (35 mg, 0.14 mmol) and (S)-3-(5-(((3S,4S)-4-(methoxymethyl)pyrrolidin-3-yl)oxy)-1-oxoisoindolin-2-yl)piperidine-2,6-dione, TFA salt (70 mg, 0.14 mmol) in DCM (2 mL). The reaction was stirred at room temperature under an atmosphere of nitrogen for 30 min, at which point, NaBH₃CN (18 mg, 0.29 mmol) was added. The reaction mixture was stirred for an additional 2 h at room temperature under an atmosphere of nitrogen. The reaction was concentrated under reduced pressure and iteratively purified via silica gel chromatography (0-10% MeOH/DCM) and preparative chiral HPLC [Column: JW-Chiral-Amylose-SB, 250×20 mm, (1:2 i-PrOH/DCM)/hexane] to provide rel-(3S)-3-(5-{[(3S,4S)-1-{[8-fluoro-2-(oxolan-2-yl)quinazolin-6-yl]methyl}-4-(methoxymethyl)pyrrolidin-3-yl]oxy}-1-oxo-2,3-dihydro-1H-isoindol-2-yl)piperidine-2,6-dione as a mixture of diastereomers. MS (ESI) m/z: 604.2 [M+H]⁺. ¹H NMR (400 MHz, MeOD-d₄) δ 9.63 (d, J=1.5 Hz, 1H), 8.16 (s, 1H), 7.90 (d, J=1.6 Hz, 1H), 7.82-7.79 (m, 1H), 7.61 (d, J=8.4 Hz, 1H), 7.15 (d, J=2.2 Hz, 1H), 7.04-7.02 (m, 1H), 5.13-5.11 (m, 1H), 5.09-5.04 (m, 1H), 4.75-4.74 (m, 1H), 4.37 (d, J=17.3 Hz, 1H), 4.24 (d, J=17.3 Hz, 1H), 4.06 (m, 1H), 3.91-3.90 (m, 1H), 3.85 (m, 1H), 3.77 (m, 1H), 3.32-3.30 (m, 2H), 3.26 (s, 3H), 3.00-2.96 (m, 2H), 2.94-2.82 (m, 1H), 2.79-2.76 (m, 1H), 2.64-2.52 (m, 2H), 2.40-2.37 (m, 2H), 2.34-2.26 (m, 1H), 2.12-2.05 (m, 2H), 2.00-1.96 (m, 2H).

### Example 86: (S)-3-(5-(((3S,4S)-1-((2-(5-acetyl-2-oxa-5,8-diazaspiro[3.5]nonan-8-yl)-8-fluoroquinazolin-6-yl)methyl)-4-(methoxymethyl)pyrrolidin-3-yl)oxy)-1-oxoisoindolin-2-yl)piperidine-2,6-dione

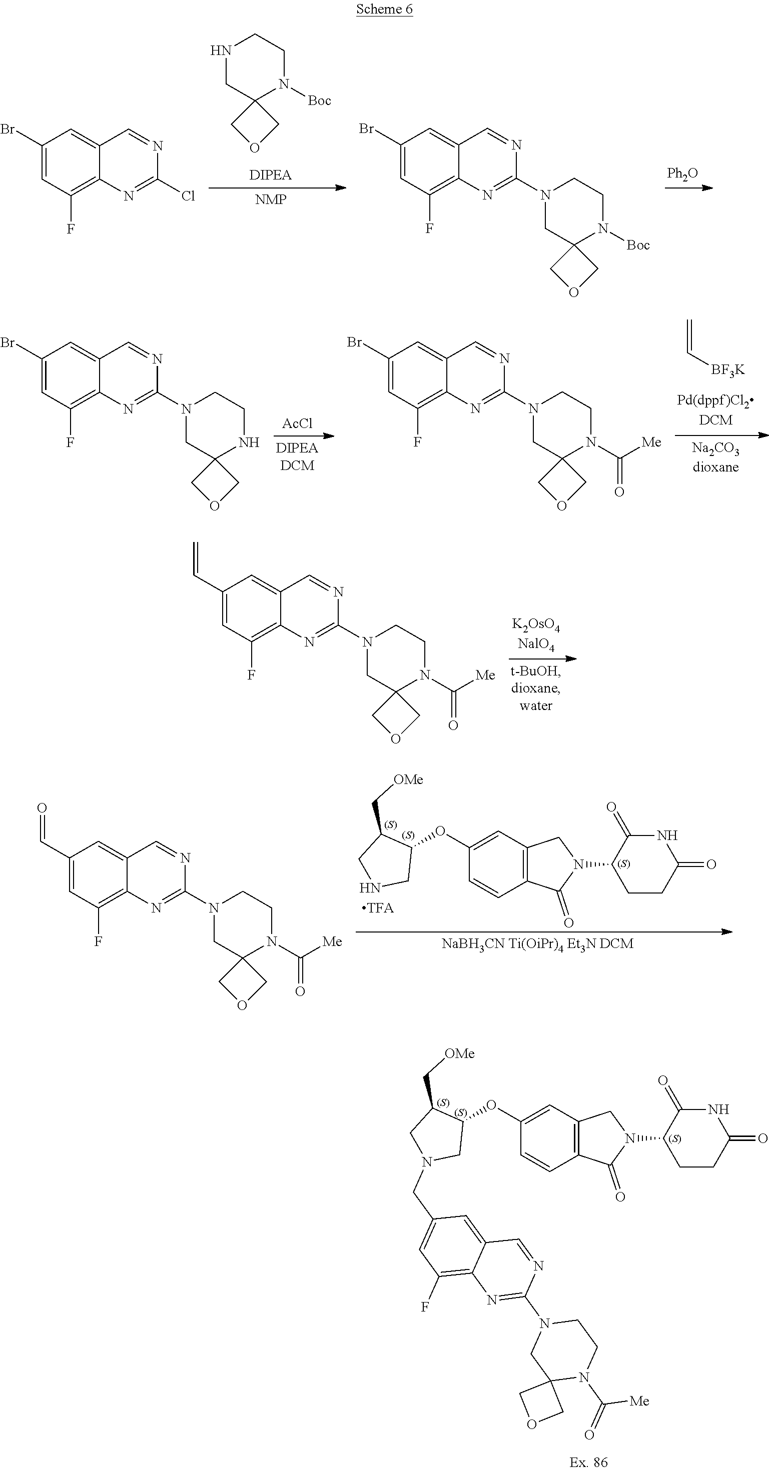

### Step 1: tert-butyl 8-(6-bromo-8-fluoroquinazolin-2-yl)-2-oxa-5,8-diazaspiro[3.5]nonane-5-carboxylate

tert-butyl 2-oxa-5,8-diazaspiro[3.5]nonane-5-carboxylate (480 mg, 2.10 mmol) and DIPEA (494 mg, 3.82 mmol) were added to a solution of 6-bromo-2-chloro-8-fluoroquinazol-ine (500 mg, 1.91 mmol) in NMP (15 mL). The reaction was stirred at 160° C. under an atmosphere of nitrogen. After 2 h, the reaction was cooled to room temperature, diluted with water and extracted with EtOAc. The combined organic layers were washed with brine, dried with Na₂SO₄, filtered, and concentrated under reduced pressure to provide tert-butyl 8-(6-bromo-8-fluoroquinazolin-2-yl)-2-oxa-5,8-diazaspiro[3.5]nonane-5-carboxylate, which was used in the subsequent step without further purification. MS (ESI) m/z: 453.1 [M+H]⁺.

### Step 2: 8-(6-bromo-8-fluoroquinazolin-2-yl)-2-oxa-5,8-diazaspiro[3.5]nonane

A solution of tert-butyl 8-(6-bromo-8-fluoroquinazolin-2-yl)-2-oxa-5,8-diazaspiro[3.5]-nonane-5-carboxylate (600 mg, 1.32 mmol) in Ph₂O (5 mL) was stirred at 180° C. under an atmosphere of nitrogen. After 24 h, the reaction was cooled to room temperature, diluted with MeCN and purified via reversed-phase silica gel chromatography to provide 8-(6-bromo-8-fluoroquinazolin-2-yl)-2-oxa-5,8-diazaspiro[3.5]nonane. MS (ESI) m/z: 353.0 [M+H]⁺.

### Step 3: 1-(8-(6-bromo-8-fluoroquinazolin-2-yl)-2-oxa-5,8-diazaspiro[3.5]nonan-5-yl)ethan-1-one

AcCl (111 mg, 1.41 mmol) was added to a solution of 8-(6-bromo-8-fluoroquinazolin-2-yl)-2-oxa-5,8-diazaspiro[3.5]nonane (250 mg, 0.70 mmol) and DIPEA (273 mg, 2.11 mmol) in DCM (6 mL) at 0° C. under an atmosphere of nitrogen. The reaction was stirred for 1 h at room temperature. The reaction was diluted with water and extracted with EtOAc. The combined organic layers were washed with brine, dried with Na₂SO₄, filtered, and concentrated under reduced pressure to provide 1-(8-(6-bromo-8-fluoroquinazolin-2-yl)-2-oxa-5,8-diazaspiro[3.5]nonan-5-yl)ethan-1-one, which was used in the subsequent step without further purification. MS (ESI) m/z: 395.0 [M+H]⁺.

### Step 4: 1-(8-(8-fluoro-6-vinylquinazolin-2-yl)-2-oxa-5,8-diazaspiro[3.5]nonan-5-yl)ethan-1-one

potassium trifluoro(vinyl)borate (155 mg, 1.16 mmol), Pd(dppf)Cl₂·DCM (47 mg, 0.058 mmol), and Na₂CO₃ (185 mg, 1.75 mmol) were added to a solution of 1-(8-(6-bromo-8-fluoroquinazolin-2-yl)-2-oxa-5,8-diazaspiro[3.5]nonan-5-yl)ethan-1-one (230 mg, 0.58 mmol) in dioxane (5 mL) and water (1 mL). The reaction was stirred at 80° C. for 2 h under an atmosphere of nitrogen. The reaction was cooled to room temperature, filtered, and rinsed with EtOAc. The resulting filtrate was concentrated under reduced pressure and purified via silica gel chromatography (0-60% EtOAc/petroleum ether) to provide 1-(8-(8-fluoro-6-vinylquinazolin-2-yl)-2-oxa-5,8-diazaspiro[3.5]nonan-5-yl)ethan-1-one. MS (ESI) m/z: 343.2 [M+H]⁺.

### Step 5: 2-(5-acetyl-2-oxa-5,8-diazaspiro[3.5]nonan-8-yl)-8-fluoroquinazoline-6-carbaldehyde

K₂OsO₄ (21 mg, 0.057 mmol) and NaIO₄ (306 mg, 1.42 mmol) were added to a solution of 1-(8-(8-fluoro-6-vinylquinazolin-2-yl)-2-oxa-5,8-diazaspiro[3.5]nonan-5-yl)ethan-1-one (130 mg, 0.38 mmol) in dioxane (4 mL), t-BuOH (2 mL), and water (2 mL). The reaction was stirred at room temperature for 1 h. The reaction was quenched with sat. aq. NaHCO₃ to pH 7 and extracted with EtOAc. The combined organic layers were washed with brine, dried with Na₂SO₄, filtered, and concentrated under reduced pressure to provide 2-(5-acetyl-2-oxa-5,8-diazaspiro[3.5]nonan-8-yl)-8-fluoroquinazoline-6-carbaldehyde, which was used in the subsequent step without further purification. MS (ESI) m/z: 345.2 [M+H]⁺.

### Step 6: Example 86, (S)-3-(5-(((3S,4S)-1-((2-(5-acetyl-2-oxa-5,8-diazaspiro[3.5]nonan-8-yl)-8-fluoroquinazolin-6-yl)methyl)-4-(methoxymethyl)pyrrolidin-3-yl)oxy)-1-oxoisoindolin-2-yl)piperidine-2,6-dione

Ti(Oi-Pr)₄ (140 mg, 0.49 mmol) was added to a solution of (S)-3-(5-(((3S,4S)-4-(methoxymethyl)pyrrolidin-3-yl)oxy)-1-oxoisoindolin-2-yl)piperidine-2,6-dione, TFA salt (80 mg, 0.16 mmol), 2-(5-acetyl-2-oxa-5,8-diazaspiro[3.5]nonan-8-yl)-8-fluoroquinazoline-6-carbaldehyde (60 mg, 0.17 mmol), and Et₃N (33 mg, 0.33 mmol) in DCM (4 mL). The reaction was stirred at room temperature under an atmosphere of nitrogen for 30 min, at which point, NaBH₃CN (16 mg, 0.25 mmol) was added. The reaction mixture was stirred for an additional 2 h at room temperature under an atmosphere of nitrogen. The reaction was carefully concentrated under reduced pressure at 25° C. and iteratively purified via silica gel chromatography (0-10% MeOH/DCM) and preparative chiral HPLC [Column: JW-Chiral-Amylose-SB, 250×20 mm, (1:2 EtOH/DCM)/hexane] to provide (S)-3-(5-(((3S,4S)-1-((2-(5-acetyl-2-oxa-5,8-diazaspiro[3.5]nonan-8-yl)-8-fluoroquinazolin-6-yl)methyl)-4-(methoxymethyl)pyrrolidin-3-yl)oxy)-1-oxoisoindolin-2-yl)piperidine-2,6-dione. MS (ESI) m/z: 702.2 [M+H]⁺. ¹H NMR (400 MHz, DMSO-d₆) δ 10.96 (s, 1H), 9.30 (d, J=1.6 Hz, 1H), 7.64-7.53 (m, 3H), 7.15 (d, J=2.2 Hz, 1H), 7.03 (dd, J=8.4, 2.2 Hz, 1H), 5.07 (dd, J=13.3, 5.1 Hz, 1H), 4.75-4.70 (m, 3H), 4.37 (d, J=17.3 Hz, 1H), 4.31-4.20 (m, 5H), 3.83 (m, 2H), 3.72-3.65 (m, 2H), 3.50-3.37 (m, 4H), 3.29 (s, 3H), 2.96-2.87 (m, 3H), 2.79-2.71 (m, 1H), 2.64-2.53 (m, 2H), 2.39-2.30 (m, 1H), 2.24-2.21 (m, 1H), 2.10 (s, 3H), 1.98-1.91 (m, 1H).

### Example 111: rel-(S)-3-(5-(((3S,4S)-1-((8-fluoro-2-(4-(2-hydroxypropan-2-yl)cyclohexyl)quinazolin-6-yl)methyl)-4-(methoxymethyl)pyrrolidin-3-yl)oxy)-1-oxoisoindolin-2-yl)piperidine-2,6-dione

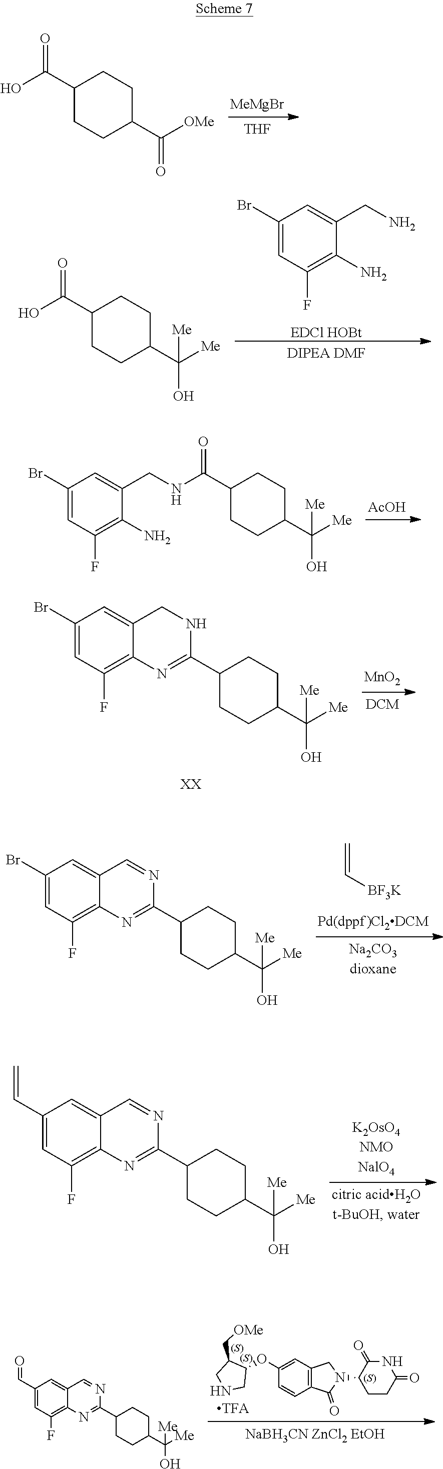

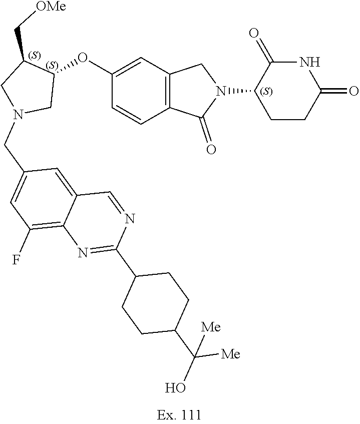

### Step 1: 4-(2-hydroxypropan-2-yl)cyclohexane-1-carboxylic Acid

MeMeBr (1 M in THF, 16 mL, 16 mmol) was added to a solution of 4-(methoxycarbonyl)cyclohexane-1-carboxylic acid (1000 mg, 5.37 mmol) in THF (20 mL) at 0° C. The reaction was stirred at room temperature for 4 h, followed by an additional 4 h at 40° C. The reaction was cooled to 0° C., quenched with sat. aq. NH₄Cl, and extracted with EtOAc. The combined organic layers were washed with brine, dried with Na₂SO₄, filtered, and concentrated under reduced pressure to provide 4-(2-hydroxypropan-2-yl)cyclohexane-1-carboxylic acid, which was used in the subsequent step without further purification. MS (ESI) m/z: 187.3 [M+H]⁺.

### Step 2: N-(2-amino-5-bromo-3-fluorobenzyl)-4-(2-hydroxypropan-2-yl)cyclohexane-1-carboxamide

DIPEA (1.18 g, 9.13 mmol) was added to a solution of 4-(2-hydroxypropan-2-yl)cyclohexane-1-carboxylic acid (1000 mg, 5.37 mmol), 2-(aminomethyl)-4-bromo-6-fluoroaniline (1176 mg, 5.37 mmol), HOBt (725 mg, 5.37 mmol), and EDCI (1026 mg, 5.37 mmol) in DMF (20 mL) at 0° C. The reaction was stirred at room temperature under an atmosphere of nitrogen. After 2 h, the reaction was diluted with water and extracted with EtOAc. The combined organic layers were washed with brine, dried with Na₂SO₄, filtered, and concentrated under reduced pressure to provide N-(2-amino-5-bromo-3-fluorobenzyl)-4-(2-hydroxypropan-2-yl)cyclohexane-1-carboxamide, which was used in the subsequent step without further purification. MS (ESI) m/z: 387.1 [M+H]⁺.

### Step 3: 2-(4-(6-bromo-8-fluoro-3,4-dihydroquinazolin-2-yl)cyclohexyl)propan-2-ol

A solution of N-(2-amino-5-bromo-3-fluorobenzyl)-4-(2-hydroxypropan-2-yl)cyclo-hexane-1-carbo-xamide (4120 mg, 10.64 mmol) in AcOH (30 mL) was stirred for 16 h at 100° C. under an atmosphere of nitrogen. The reaction was cooled to room temperature, concentrated under reduced pressure, and purified via reversed-phase silica gel chromatography to provide 2-(4-(6-bromo-8-fluoro-3,4-dihydroquinazolin-2-yl)cyclohexyl)propan-2-ol. MS (ESI) m/z: 369.0 [M+H]⁺.

### Step 4: 2-(4-(6-bromo-8-fluoroquinolin-2-yl)cyclohexyl)propan-2-ol

A mixture of 2-(4-(6-bromo-8-fluoro-3,4-dihydroquinazolin-2-yl)cyclohexyl)propan-2-ol (849 mg, 2.30 mmol) and MnO₂ (2.60 g, 29.89 mmol) in DCM (25 mL) was stirred for 16 h at room temperature under an atmosphere of nitrogen. The reaction was filtered through Celite® and the resulting filtrate was concentrated under reduced pressure to provide 4-(6-bromo-8-fluoroquinazolin-2-yl)cyclohexan-1-ol, which was used in the subsequent step without further purification. MS (ESI) m/z: 367.3 [M+H]⁺.

### Step 5: 2-(4-(8-fluoro-6-vinylquinolin-2-yl)cyclohexyl)propan-2-ol

Pd(dppf)Cl₂·DCM (1733 mg, 2.12 mmol) and Na₂CO₃ (225 mg, 2.12 mmol) were added to a solution of 2-(4-(6-bromo-8-fluoroquinazolin-2-yl)cyclohexyl)propan-2-ol (780 mg, 2.12 mmol) and potassium trifluoro(vinyl)borate (285 mg, 2.12 mmol) in dioxane (15 mL) and water (3 mL). The reaction was stirred at 80° C. for 3 h under an atmosphere of nitrogen. The reaction was cooled to room temperature, diluted with water and extracted with EtOAc. The combined organic layers were washed with brine, dried with Na₂SO₄, filtered, and concentrated under reduced pressure. The crude mixture was purified via silica gel chromatography (0-60% EtOAc/petroleum ether to provide 2-(4-(6-bromo-8-fluoro-3,4-dihydroquinazolin-2-yl)cyclohexyl)propan-2-ol. MS (ESI) m/z: 315.1 [M+H]⁺.

### Step 6: 8-fluoro-3-morpholinoquinoline-6-carbaldehyde

K₂OsO₄ (21 mg, 0.06 mmol) was added to a solution of 2-(4-(8-fluoro-6-vinylquinazolin-2-yl)cyclohexyl)propa-n-2-ol (200 mg, 0.64 mmol), NMO (149 mg, 1.27 mmol), and citric acid (267 mg, 1.27 mmol) in t-BuOH (4 mL) and water (4 mL). The reaction was stirred at room temperature for 3 h under an atmosphere of nitrogen, at which, point NaIO₄ (417 mg, 1.95 mmol) was added. The reaction stirred for an additional 1 h at room temperature under an atmosphere of nitrogen. The reaction was quenched with sat. aq. NaHCO₃ to pH 7 and extracted with EtOAc. The combined organic layers were washed with brine, dried with Na₂SO₄, filtered, and concentrated under reduced pressure to provide 8-fluoro-2-(4-(2-hydroxypropan-2-yl)cyclohexyl)quinazoline-6-carbaldehyde, which was carried onto the next step without further purification. MS (ESI) m/z: 315.2 [M+H]⁺.

### Step 7: Example 111, rel-(S)-3-(5-(((3S,4S)-1-((8-fluoro-2-(4-(2-hydroxypropan-2-yl)cyclohexyl)quinazolin-6-yl)methyl)-4-(methoxymethyl)pyrrolidin-3-yl)oxy)-1-oxoisoindolin-2-yl)piperidine-2,6-dione

Et₃N (33 mg, 0.33 mmol) and Ti(Oi-Pr)₄ (94 mg, 0.33 mmol) were added to a solution of 8-fluoro-2-(4-(2-hydroxypropan-2-yl)cyclohexyl)quinazoline-6-carbaldehyde (52 mg, 0.16 mmol) and (S)-3-(5-(((3S,4S)-4-(methoxymethyl)pyrrolidin-3-yl)oxy)-1-oxoisoindolin-2-yl)piperidine-2,6-dione, TFA salt (80 mg, 0.16 mmol) in DCM (4 mL). The reaction was stirred at room temperature under an atmosphere of nitrogen for 30 min, at which point, NaBH₃CN (6 mg, 0.10 mmol) was added. The reaction mixture was stirred for an additional 3 h at room temperature under an atmosphere of nitrogen. The reaction was concentrated under reduced pressure and iteratively purified via silica gel chromatography (0-10% MeOH/DCM) and preparative chiral HPLC [Column: Chiral Art Cullulose-SB, 250×20 mm, hexane/(1:1 EtOH/DCM)] to provide rel-(S)-3-(5-(((3S,4S)-1-((8-fluoro-2-(4-(2-hydroxypropan-2-yl)cyclohexyl)quinazolin-6-yl)methyl)-4-(methoxymethyl)pyrrolidin-3-yl)oxy)-1-oxoisoindolin-2-yl)piperidine-2,6-dione as a single diastereomer. MS (ESI) m/z: 674.6 [M+H]⁺. ¹H NMR (400 MHz, MeOD-d₄) δ 9.47 (d, J=1.7 Hz, 1H), 7.85 (s, 1H), 7.80 (d, J=11.2 Hz, 1H), 7.71 (d, J=8.5 Hz, 1H), 7.15 (d, J=2.2 Hz, 1H), 7.09 (dd, J=8.5, 2.2 Hz, 1H), 5.12 (dd, J=13.4, 5.1 Hz, 1H), 4.85-4.75 (m, 1H), 4.51-4.36 (m, 2H), 3.92 (d, J=2.9 Hz, 2H), 3.54 (d, J=6.7 Hz, 2H), 3.39 (s, 3H), 3.12 (m, 1H), 3.00 (m, 2H), 2.80 (m, 2H), 2.70 (m, 2H), 2.55-2.41 (m, 2H), 2.15 (m, 3H), 2.07 (m, 2H), 1.90-1.80 (m, 2H), 1.54-1.43 (m, 1H), 1.35-1.29 (m, 2H), 1.22 (s, 6H).

### Example 119: (S)-3-(5-(((3S,4S)-1-((8-fluoro-2-(4-(2-hydroxypropan-2-yl)bicyclo[2.2.2]octan-1-yl)quinolin-6-yl)methyl)-4-(methoxymethyl)pyrrolidin-3-yl)oxy)-1-oxoisoindolin-2-yl)piperidine-2,6-dione

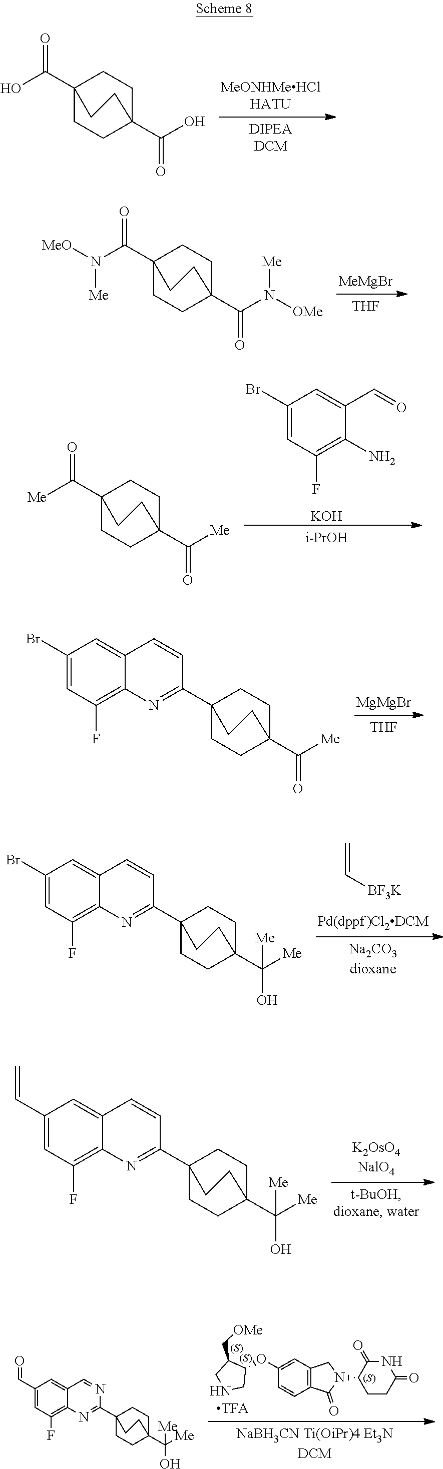

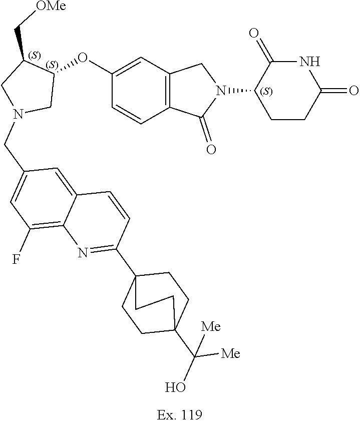

### Step 1: N1,N4-dimethoxy-N1,N4-dimethylbicyclo[2.2.2]octane-1,4-dicarboxamide

DIPEA (2.6 g, 20.12 mmol) was added to a solution of bicyclo[2.2.2]octane-1,4-dicarboxylic acid (1 g, 5.04 mmol), N,O-dimethylhydroxylamine hydrochloride (984 mg, 10.09 mmol) and HATU (4.22 g, 11.10 mmol) in DMF (20 mL) at 0° C. The reaction was stirred at room temperature under an atmosphere of nitrogen. After 12 h, the reaction was diluted with water and extracted with EtOAc. The combined organic layers were washed with brine, dried with Na₂SO₄, filtered, and concentrated under reduced pressure. The crude mixture was purified via reversed-phase silica gel chromatography to provide N1,N4-dimethoxy-N1,N4-dimethylbicyclo[2.2.2]octane-1,4-dicarboxamide. MS (ESI) m/z: 285.2 [M+H]⁺.

### Step 2: 1,1′-(bicyclo[2.2.2]octane-1,4-diyl)bis(ethan-1-one)

MeMgBr (1.0 M in THF, 16 mL, 16 mmol) was added dropwise to a solution of N¹,N⁴-dimethoxy-N¹,N⁴-dimethylbicyclo[2.2.2]octane-1,4-dicarboxamide (900 mg, 3.17 mmol) in THF (20 mL) at 0° C. The reaction was stirred at room temperature under an atmosphere of nitrogen. After 12 h, the reaction was quenched with sat. aq. NH₄Cl and extracted with DCM. The combined organic layers were dried with Na₂SO₄, filtered, and concentrated under reduced pressure to provide 1,1′-(bicyclo[2.2.2]octane-1,4-diyl)bis(ethan-1-one), which was used in the subsequent step without further purification. ¹H NMR (400 MHz, DMSO-d₆) δ 2.08 (s, 6H), 1.65 (s, 12H).

### Step 3: 1-(4-(6-bromo-8-fluoroquinolin-2-yl)bicyclo[2.2.2]octan-1-yl)ethan-1-one

KOH (187 mg, 3.33 mmol) was added to a solution of 1,1′-(bicyclo[2.2.2]octane-1,4-diyl)bis(ethan-1-one) (650 mg, 3.35 mmol) and 2-amino-5-bromo-3-fluorobenzaldehyde (729 mg, 3.34 mmol) in i-PrOH (10 mL). The reaction was stirred at room temperature under an atmosphere of nitrogen. After 1 h, the reaction was purified via reversed-phase silica gel chromatography to provide to provide 1-(4-(6-bromo-8-fluoroquinolin-2-yl)bicyclo[2.2.2]octan-1-yl)ethan-1-one. MS (ESI) m/z: 376.2 [M+H]⁺.

### Step 4: 2-(4-(6-bromo-8-fluoroquinolin-2-yl)bicyclo[2.2.2]octan-1-yl)propan-2-ol

MeMgBr (1 M in THF, 13 mL, 13 mmol) was added dropwise to a solution of 1-(4-(6-bromo-8-fluoroquinolin-2-yl)bicyclo[2.2.2]octan-1-yl)ethan-1-one (500 mg, 1.33 mmol) in THF (30 mL) at 0° C. The reaction was stirred at room temperature under an atmosphere of nitrogen. After 12 h, the reaction was quenched with sat. aq. NH₄Cl and extracted with DCM. The combined organic layers were dried with Na₂SO₄, filtered, and concentrated under reduced pressure to provide 2-(4-(6-bromo-8-fluoroquinolin-2-yl)bicyclo[2.2.2]octan-1-yl)propan-2-ol, which was used in the subsequent step without further purification. MS (ESI) m/z: 392.2 [M+H]⁺.

### Step 5: 2-(4-(8-fluoro-6-vinylquinolin-2-yl)bicyclo[2.2.2]octan-1-yl)propan-2-ol

Na₂CO₃ (380 mg, 3.59 mmol), Pd(dppf)Cl₂·DCM (97 mg, 0.12 mmol), and potassium trifluoro(vinyl)borate (321 mg, 2.40 mmol) were added to a solution of 2-(4-(6-bromo-8-fluoroquinolin-2-yl)bicyclo[2.2.2]octan-1-yl)propa-n-2-ol (470 mg, 1.20 mmol) in dioxane (12.5 mL) and water (2.5 mL). The reaction was stirred at 80° C. for 2 h under an atmosphere of nitrogen. The reaction was cooled to room temperature, filtered, and rinsed with EtOAc. The resulting filtrate was concentrated under reduced pressure and purified via silica gel chromatography (40-90% EtOAc/petroleum ether) to provide 2-(4-(8-fluoro-6-vinylquinolin-2-yl)bicyclo[2.2.2]octan-1-yl)propan-2-ol. MS (ESI) m/z: 340.3 [M+H]⁺.

### Step 6: 8-fluoro-2-(4-(2-hydroxypropan-2-yl)bicyclo[2.2.2]octan-1-yl)quinoline-6-carbaldehyde

NaIO₄ (529 mg, 2.45 mmol) and K₂OsO₄ (30 mg, 0.081 mmol) were added to a solution of 2-(4-(8-fluoro-6-vinylquinolin-2-yl)bicycle-[2.2.2]octan-1-yl)propan-2-ol (280 mg, 0.82 mmol) in t-BuOH (2 mL), dioxane (4 mL), and water (2 mL). The reaction was stirred at room temperature under an atmosphere of nitrogen. After 1 h, the reaction was quenched with sat. aq. NaHCO₃ to pH 7 and extracted with EtOAc. The combined organic layers were washed with brine, dried with Na₂SO₄, filtered, and concentrated under reduced pressure. The crude mixture was purified via reversed-phase silica gel chromatography to provide 8-fluoro-2-(4-(2-hydroxypropan-2-yl)bicyclo[2.2.2]octan-1-yl)quinoline-6-carbaldehyde. MS (ESI) m/z: 342.1 [M+H]⁺.

### Step 7: Example 119, (S)-3-(5-(((3S,4S)-1-((8-fluoro-2-(4-(2-hydroxypropan-2-yl)bicyclo[2.2.2]octan-1-yl)quinolin-6-yl)methyl)-4-(methoxymethyl)pyrrolidin-3-yl)oxy)-1-oxoisoindolin-2-yl)piperidine-2,6-dione

Ti(Oi-Pr)₄ (93 mg, 0.33 mmol) was added to a solution of (S)-3-(5-(((3S,4S)-4-(methoxymethyl)pyrrolidin-3-yl)oxy)-1-oxoisoindolin-2-yl)piperidine-2,6-dione, TFA salt (80 mg, 0.16 mmol), 8-fluoro-2-(4-(2-hydroxypropan-2-yl)-bicyclo[2.2.2]octan-1-yl)quinoline-6-carbaldehyde (56 mg, 0.16 mmol), and Et₃N (33 mg, 0.33 mmol) in DCM (4 mL). The reaction was stirred at room temperature under an atmosphere of nitrogen for 30 min, at which point, NaBH₃CN (10 mg, 0.16 mmol) was added. The reaction mixture was stirred for an additional 2 h at room temperature under an atmosphere of nitrogen. The reaction was carefully concentrated under reduced pressure at 25° C. and iteratively purified via silica gel chromatography (0-10% MeOH/DCM) and preparative chiral HPLC [Column: Chiral Art Amylose-SA, 250×20 mm, (1:2 hexane/DCM)/EtOH] to provide (S)-3-(5-(((3S,4S)-1-((8-fluoro-2-(4-(2-hydroxypropan-2-yl)bicyclo[2.2.2]octan-1-yl)quinolin-6-yl)methyl)-4-(methoxymethyl)pyrrolidin-3-yl)oxy)-1-oxoisoindolin-2-yl)piperidine-2,6-dione. MS (ESI) m/z: 699.6 [M+H]⁺. ¹H NMR (400 MHz, MeOD-d₄) δ 8.16 (d, J=8.6 Hz, 1H), 7.70 (d, J=8.4 Hz, 1H), 7.61 (m, 2H), 7.47 (m, 1H), 7.14 (s, 1H), 7.08 (m, 1H), 5.11 (m, 1H), 4.84 (m, 1H), 4.50-4.35 (m, 2H), 3.85 (m, 2H), 3.53 (m, 2H), 3.38 (s, 3H), 3.12-3.09 (m, 1H), 2.97-2.85 (m, 3H), 2.78-2.65 (m, 2H), 2.52-2.40 (m, 2H), 2.17-2.10 (m, 1H), 2.03 (m, 6H), 1.73 (m, 6H), 1.17 (s, 6H).

### Example 121: (S)-3-(5-(((3S,4S)-1-((8-fluoro-2-(4-(2-hydroxypropan-2-yl)cuban-1-yl)quinolin-6-yl)methyl)-4-(methoxymethyl)pyrrolidin-3-yl)oxy)-1-oxoisoindolin-2-yl)piperidine-2,6-dione

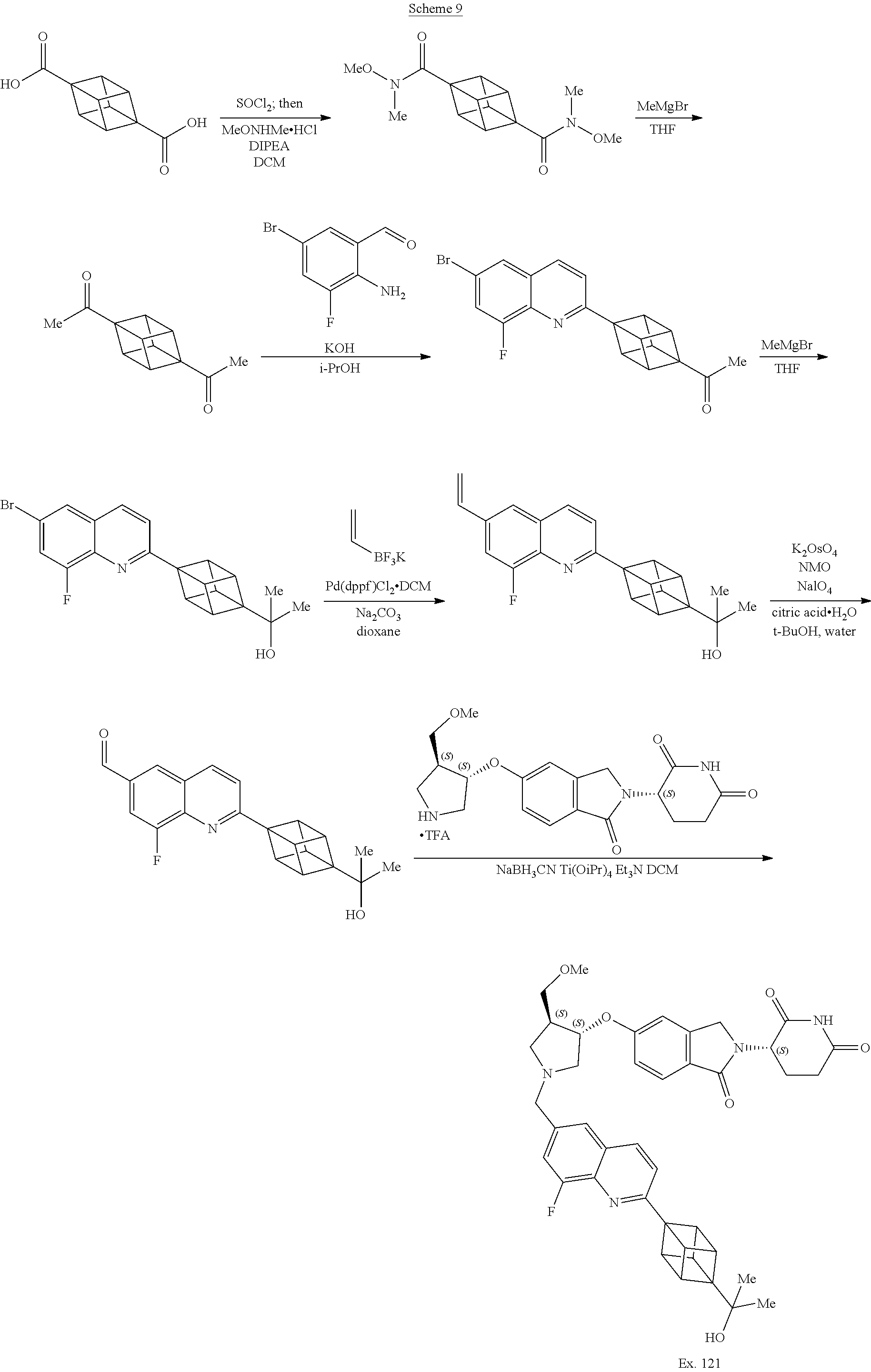

### Step 1: N1,N4-Dimethoxy-N1,N4-dimethylcubane-1,4-dicarboxamide

A solution of cubane-1,4-dicarboxylic acid (800 mg, 4.16 mmol) in SOCl₂ (10 mL) was stirred at 80° C. under an atmosphere of nitrogen. After 2 h, the reaction was cooled to room temperature and concentrated under reduced pressure. The concentrated mixture was re-dissolved in DCM (8 mL) and stirred at 0° C., at which point, a solution of DIPEA (1078 mg, 8.34 mmol) and N,O-dimethylhydroxylamine hydrochloride (800 mg, 8.20 mmol) in DCM (2 mL) was added. The reaction was stirred at room temperature under an atmosphere of nitrogen After 2 h, the reaction was carefully concentrated by bubbling nitrogen to remove the organics. The crude mixture was purified via reversed-phase silica gel chromatography to provide N1,N4-Dimethoxy-N1,N4-dimethylcubane-1,4-dicarboxamide. MS (ESI) m/z: 279.2 [M+H]⁺.

### Step 2: 1,1′-(Cubane-1,4-diyl)bis(ethan-1-one)

MeMgBr (2.0 M in THF, 5 mL, 10 mmol) was added dropwise to a solution of N¹,N⁴-dimethoxy-N¹,N⁴-dimethylcubane-1,4-dicarboxamide (750 mg, 2.69 mmol) in THF (10 mL) at 0° C. The reaction was stirred at room temperature under an atmosphere of nitrogen. After 3 h, the reaction was diluted with water and extracted with EtOAc. The combined organic layers were washed with brine, dried with Na₂SO₄, filtered, and concentrated under reduced pressure. The crude mixture was purified via reversed-phase silica gel chromatography to provide 1,1′-(Cubane-1,4-diyl)bis(ethan-1-one). MS (ESI) m/z: 189.2 [M+H]⁺.

### Step 3: 1-(4-(6-Bromo-8-fluoroquinolin-2-yl)cuban-1-yl)ethan-1-one

KOH (100 mg, 1.78 mmol) was added to a solution of 1,1′-(cubane-1,4-diyl)bis(ethan-1-one) (350 mg, 1.86 mmol) and 2-amino-5-bromo-3-fluorobenzaldehyde (406 mg, 1.86 mmol) in i-PrOH (8 mL). The reaction was stirred at room temperature under an atmosphere of nitrogen. After 2 h, the reaction was diluted with water and extracted with EtOAc. The combined organic layers were washed with brine, dried with Na₂SO₄, filtered, and concentrated under reduced pressure. The crude mixture was purified via reversed-phase silica gel chromatography 1-(4-(6-Bromo-8-fluoroquinolin-2-yl)cuban-1-yl)ethan-1-one. MS (ESI) m/z: 370.2 [M+H]⁺.

### Step 4: 1-(4-(6-Bromo-8-fluoroquinolin-2-yl)cuban-1-yl)ethan-1-one

MeMgBr (1 M in THF, 1.5 mL, 1.5 mmol) was added dropwise to a solution of 1-(4-(6-bromo-8-fluoroquinolin-2-yl)cuban-1-yl)ethan-1-one (190 mg, 0.51 mmol) in THF (3 mL) at 0° C. The reaction was stirred at room temperature under an atmosphere of nitrogen. The reaction was quenched with sat. aq. NH₄Cl and extracted with EtOAc. The combined organic layers were dried with Na₂SO₄, filtered, and concentrated under reduced pressure to provide 1-(4-(6-Bromo-8-fluoroquinolin-2-yl)cuban-1-yl)ethan-1-one, which was used in the subsequent step without further purification. MS (ESI) m/z: 392.2 [M+H]⁺.

### Step 5: 2-(4-(6-Bromo-8-fluoroquinolin-2-yl)cuban-1-yl)propan-2-ol

Pd(dppf)Cl₂·DCM (15 mg, 0.019 mmol) and Na₂CO₃ (164 mg, 1.62 mmol) were added to a solution of 1-(4-(6-bromo-8-fluoroquinolin-2-yl)cuban-1-yl)ethan-1-one (300 mg, crude) and potassium vinyltrifluoroborate (145 mg, 1.08 mmol) in dioxane (10 mL) and water (6 mL). The reaction was stirred at 80° C. for 3 h under an atmosphere of nitrogen. The reaction was cooled to room temperature, diluted with water, and extracted with EtOAc. The combined organic layers were washed with brine, dried with Na₂SO₄, filtered, and concentrated under reduced pressure. The crude mixture was purified via silica gel chromatography (0-50% EtOAc/petroleum ether) to provide 2-(4-(6-Bromo-8-fluoroquinolin-2-yl)cuban-1-yl)propan-2-ol. MS (ESI) m/z: 386.0 [M+H]⁺.

### Step 6: 8-Fluoro-2-(4-(2-hydroxypropan-2-yl)cuban-1-yl)quinoline-6-carbaldehyde

K₂OsO₄ (4 mg, 0.011 mmol) was added to a solution of 2-(4-(6-bromo-8-fluoroquinolin-2-yl)cuban-1-yl)propan-2-ol (40 mg, 0.10 mmol), NMO (23 mg, 0.20 mmol), and citric acid monohydrate (42 mg, 0.20 mmol) in t-BuOH (2 mL) and water (2 mL). The reaction was stirred for 3 h at room temperature under an atmosphere of nitrogen, at which point, NaIO₄ (43 mg, 0.20 mmol) was added. The reaction stirred for an additional 1 h at room temperature under an atmosphere of nitrogen. The reaction was quenched with sat. aq. NaHCO₃ to pH 7 and extracted with EtOAc. The combined organic layers were washed with brine, dried with Na₂SO₄, filtered, and concentrated under reduced pressure to provide 8-Fluoro-2-(4-(2-hydroxypropan-2-yl)cuban-1-yl)quinoline-6-carbaldehyde, which was used in the subsequent step without further purification. MS (ESI) m/z: 336.0 [M+H]⁺.

### Step 7: Example 121, (S)-3-(5-(((3S,4S)-1-((8-fluoro-2-(4-(2-hydroxypropan-2-yl)cuban-1-yl)quinolin-6-yl)methyl)-4-(methoxymethyl)pyrrolidin-3-yl)oxy)-1-oxoisoindolin-2-yl)piperidine-2,6-dione

Et₃N (43 mg, 0.33 mmol) and Ti(Oi-Pr)₄ (63 mg, 0.22 mmol) were added to a solution of (8-fluoro-2-(4-(2-hydroxypropan-2-yl)cuban-1-yl)quinoline-6-carbaldehyde (40 mg, crude) and (S)-3-(5-(((3S,4S)-4-(methoxymethyl)pyrrolidin-3-yl)oxy)-1-oxoisoindolin-2-yl)piperidine-2,6-dione, TFA salt (80 mg, 0.16 mmol) in DCM (4 mL). The reaction was stirred at room temperature under an atmosphere of nitrogen for 30 min, at which point, NaBH₃CN (14 mg, 0.22 mmol) was added. The reaction mixture was stirred for an additional 3 h at room temperature under an atmosphere of nitrogen. The reaction was carefully concentrated under reduced pressure at 25° C. and iteratively purified via silica gel chromatography (0-10% MeOH/DCM) and preparative chiral HPLC [Column: Chiralpak IF, 250×20 mm, hexane/(1:2 EtOH/DCM)] to provide (S)-3-(5-(((3S,4S)-1-((8-fluoro-2-(4-(2-hydroxypropan-2-yl)cuban-1-yl)quinolin-6-yl)methyl)-4-(methoxymethyl)pyrrolidin-3-yl)oxy)-1-oxoisoindolin-2-yl)piperidine-2,6-dione. MS (ESI) m/z: 693.2 [M+H]⁺. ¹H NMR (400 MHz, DMSO-d₆) δ 10.96 (s, 1H), 8.34 (d, J=8.6 Hz, 1H), 7.67 (s, 1H), 7.60 (d, J=8.4 Hz, 1H), 7.55-7.46 (m, 2H), 7.15 (s, 1H), 7.04 (d, J=8.5 Hz, 1H), 5.06 (dd, J=13.3, 5.1 Hz, 1H), 4.75 (m, 1H), 4.36 (d, J=17.2 Hz, 1H), 4.25 (d, J=17.4 Hz, 1H), 4.12 (t, J=4.7 Hz, 3H), 3.92 (t, J=4.8 Hz, 3H), 3.79 (m, 2H), 3.50 (m, 2H), 3.23 (s, 3H), 2.98-2.83 (m, 3H), 2.75 (m, 1H), 2.63-2.52 (m, 2H), 2.37 (m, 1H), 2.25 (m, 1H), 2.01-1.93 (m, 1H), 1.08 (s, 6H).

### Examples 146 and 123 (S)-3-(5-(((3R*,4S*)-4-Cyclopentyl-1-((8-fluoro-2-(tetrahydro-2H-pyran-4-yl)-quinolin-6-yl)methyl)pyrrolidin-3-yl)oxy)-1-oxoisoindolin-2-yl)piperidine-2,6-dione and (S)-3-(5-(((3S*,4R*)-4-Cyclopentyl-1-((8-fluoro-2-(tetrahydro-2H-pyran-4-yl)-quinolin-6-yl)methyl)pyrrolidin-3-yl)oxy)-1-oxoisoindolin-2-yl)piperidine-2,6-dione

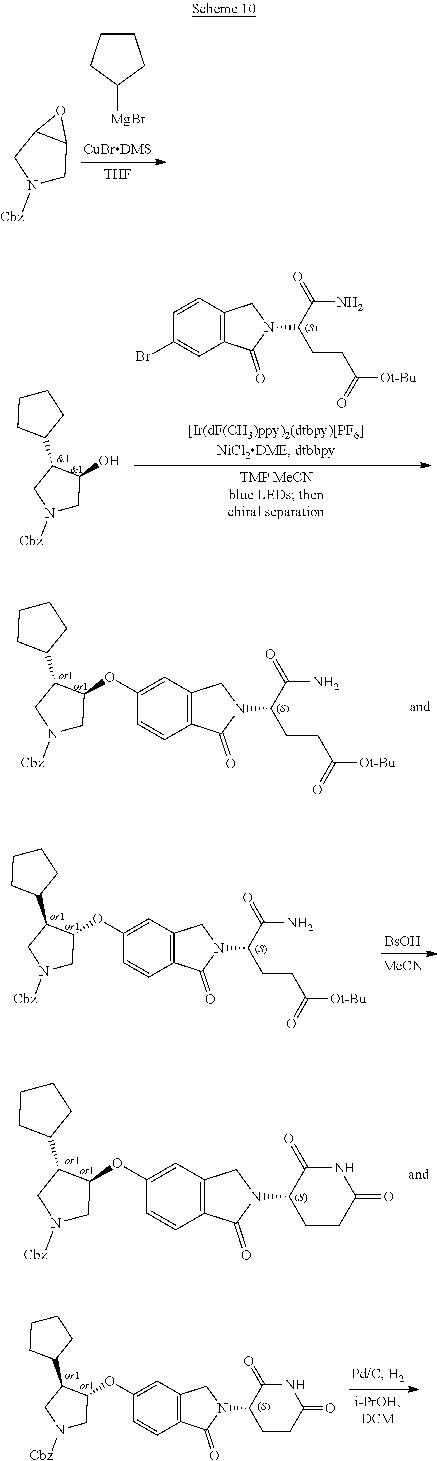

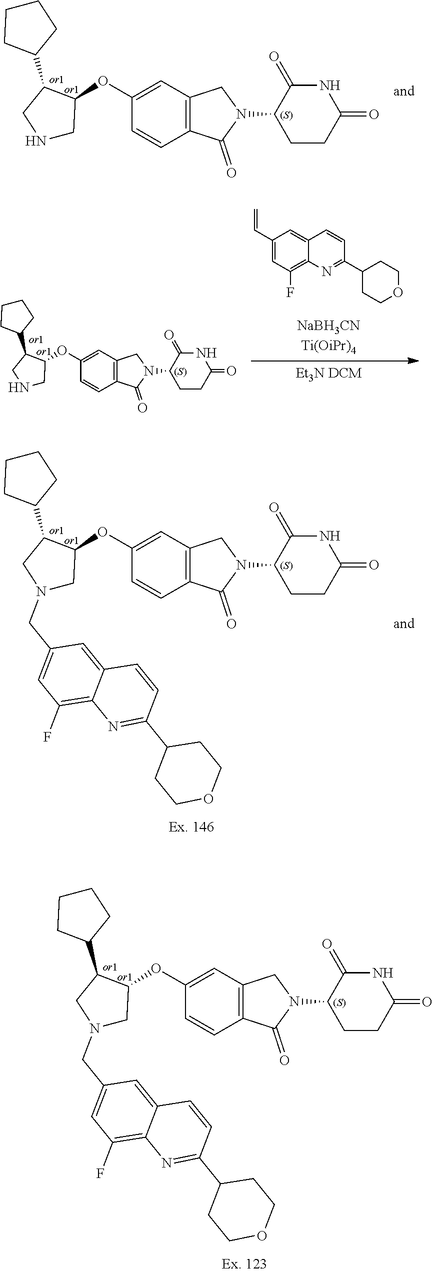

### Step 1: rac-benzyl 3-cyclopentyl-4-hydroxypyrrolidine-1-carboxylate

cyclopentylmagnesium bromide (1 M in THF, 20 mL, 20 mmol) was added dropwise to a solution of benzyl 6-oxa-3-azabicyclo(3.1.0)hexane-3-carboxylate (1 g, 4.56 mmol) and CuBr-DMS (282 mg, 1.37 mmol) in THF (20 mL) at −30° C. The reaction was stirred at −30° C. under an atmosphere of nitrogen. After 1 h, the reaction was diluted with sat. aq. NH₄Cl and extracted. The combined organic lavers were washed with brine, dried with Na₂SO₄, filtered, and concentrated under reduced pressure. The crude mixture was purified via reversed-phase silica gel chromatography to provide rac-benzyl 3-cyclopentyl-4-hydroxypyrrolidine-1-carboxylate as a mixture of trans isomers. MS (ESI) m/z: 290.5 [M+H]⁺.

### Step 2: benzyl (3R*,4S*)-3-((2-((S)-1-amino-5-(tert-butoxy)-1,5-dioxopentan-2-yl)-1-oxoisoindolin-5-yl)oxy)-4-cyclopentylpyrrolidine-1-carboxylate and benzyl (3S*,4R*)-3-((2-((S)-1-amino-5-(tert-butoxy)-1,5-dioxopentan-2-yl)-1-oxoisoindolin-5-yl)oxy)-4-cyclopentylpyrrolidine-1-carboxylate

A solution of rac-benzyl 3-cyclopentyl-4-hydroxypyrrolidine-1-carboxylate (600 mg, 2.07 mmol), TMP (396 mg, 2.80 mmol), tert-butyl (4S)-5-amino-4-(5-bromo-1-oxo-isoindolin-2-yl)-5-oxopentanoate (988 mg, 2.49 mmol), [Ir(dF(Me)ppy)₂(dtbpy)][PF₆] (46 mg, 0.045 mmol), NiCl₂·DME (23 mg, 0.10 mmol), and dtbbpy (28 mg, 0.10 mmol) in MeCN (10 mL) was irradiated with 100 w blue LEDs for 16 h under an atmosphere of nitrogen. The reaction was purified via reversed-phase silica gel column chromatography and the resulting diastereomeric mixture was further separated by preparative chiral HPLC [Column: JW-Chiral Art Cellulose-SB, 250×20 mm, i-PrOH/(2:1 hexane/DCM)] to provide each diastereomer, which was independently carried onto the subsequent steps. benzyl (3R*,4S*)-3-((2-((S)-1-amino-5-(tert-butoxy)-1,5-dioxopentan-2-yl)-1-oxoisoindolin-5-yl)oxy)-4-cyclopentylpyrrolidine-1-carboxylate (faster eluting peak): MS (ESI) m/z: 606.7 [M+H]⁺. benzyl (3S*,4R*)-3-((2-((S)-1-amino-5-(tert-butoxy)-1,5-dioxopentan-2-yl)-1-oxoisoindolin-5-yl)oxy)-4-cyclopentylpyrrolidine-1-carboxylate (slower eluting peak): MS (ESI) m/z: 606.7 [M+H]⁺.

### Step 3A: Benzyl (3S*,4R*)-3-cyclopentyl-4-((2-((S)-2,6-dioxopiperidin-3-yl)-1-oxoisoindolin-5-yl)oxy)pyrrolidine-1-carboxylate (from Faster Eluting Intermediate in Step 2)

A solution of benzyl (3R*,4S*)-3-((2-((S)-1-amino-5-(tert-butoxy)-1,5-dioxopentan-2-yl)-1-oxoisoindolin-5-yl)oxy)-4-cyclopentylpyrrolidine-1-carboxylate (200 mg, 0.38 mmol) and BsOH (208 mg, 1.31 mmol) in MeCN (7 mL) was stirred at 60° C. for 16 h under an atmosphere of nitrogen. The reaction was cooled to room temperature, concentrated under reduced pressure, and purified via reversed-phase silica gel chromatography to provide Benzyl (3S*,4R*)-3-cyclopentyl-4-((2-((S)-2,6-dioxopiperidin-3-yl)-1-oxoisoindolin-5-yl)oxy)pyrrolidine-1-carboxylate. MS (ESI) m/z: 532.3 [M+H]⁺.

### Step 3B: Benzyl (3R*,4S*)-3-cyclopentyl-4-((2-((S)-2,6-dioxopiperidin-3-yl)-1-oxoisoindolin-5-yl)oxy)pyrrolidine-1-carboxylate (from Slower Eluting Intermediate in Step 2)

This intermediate was prepared according to Step 3A, using the slower eluting intermediate from Step 2. MS (ESI) m/z: 532.4 [M+H]⁺.

### Step 4A: (S)-3-(5-(((3R*,4S*)-4-cyclopentylpyrrolidin-3-yl)oxy)-1-oxoisoindolin-2-yl)-piperidine-2,6-dione (from Intermediate in Step 3A)

A mixture of benzyl (3S*,4R*)-3-cyclopentyl-4-((2-((S)-2,6-dioxopiperidin-3-yl)-1-oxoisoindolin-5-yl)oxy)-pyrrolidine-1-carboxylate (150 mg, 0.28 mmol) and 10% Pd/C (60 mg, wetted with ca. 50% water) in EtOH (6 mL) and DCM (2 mL) was stirred at room temperature under an atmosphere of hydrogen (˜2 bar) for 5 hours. The reaction was filtered through Celite®, and the resulting filtrate was concentrated under reduced pressure. The crude mixture was purified via reversed-phase silica gel chromatography to provide (S)-3-(5-(((3R*,4S*)-4-Cyclopentylpyrrolidin-3-yl)oxy)-1-oxoisoindolin-2-yl)-piperidine-2,6-dione. MS (ESI) m/z: 398.4 [M+H]⁺.

### Step 4B: (S)-3-(5-(((3S*,4R*)-4-cyclopentylpyrrolidin-3-yl)oxy)-1-oxoisoindolin-2-yl)-piperidine-2,6-dione (from (from Intermediate in Step 4B)

This intermediate was prepared according to Step 4A, using the intermediate from Step 3B. MS (ESI) m/z: 398.4 [M+H]⁺.

### Step 5A: Example 146, (S)-3-(5-(((3R*,4S*)-4-Cyclopentyl-1-((8-fluoro-2-(tetrahydro-2H-pyran-4-yl)-quinolin-6-yl)methyl)pyrrolidin-3-yl)oxy)-1-oxoisoindolin-2-yl)piperidine-2,6-dione (from Intermediate in Step 4A)

Ti(Oi-Pr)₄ (60 mg, 0.21 mmol), Et₃N (62 mg, 0.62 mmol), and NaBH₃CN (25 mg, 0.40 mmol) were sequentially added to a solution of (S)-3-(5-(((3R*,4S*)-4-cyclopentylpyrrolidin-3-yl)oxy)-1-oxoiso-indolin-2-yl)piperidine-2,6-dione (80 mg, 0.20 mmol) and 8-fluoro-2-(tetrahydro-2H-pyran-4-yl)quinoline-6-carbaldehyde (50 mg, 0.19 mmol) in DCM (5 mL). The reaction mixture was stirred for 12 h at room temperature under an atmosphere of nitrogen. The reaction was concentrated under reduced pressure and iteratively purified via silica gel chromatography (0-10% MeOH/DCM) and preparative chiral HPLC [Column: Chiral Art Amylose-SA, 250×20 mm, EtOH/DCM] to provide (S)-3-(5-(((3R*,4S*)-4-Cyclopentyl-1-((8-fluoro-2-(tetrahydro-2H-pyran-4-yl)-quinolin-6-yl)methyl)pyrrolidin-3-yl)oxy)-1-oxoisoindolin-2-yl)piperidine-2,6-dione. MS (ESI) m/z: 641.3 [M+H]⁺. ¹H NMR (400 MHz, MeOD-d₄) δ 8.27 (dd, J=8.7, 1.5 Hz, 1H), 7.76-7.65 (m, 2H), 7.59-7.50 (m, 2H), 7.10-7.00 (m, 2H), 5.11 (dd, J=13.3, 5.1 Hz, 1H), 4.74 (d, J=6.3 Hz, 1H), 4.48-4.35 (m, 2H), 4.16-4.06 (m, 2H), 3.85-3.83 (m, 2H), 3.64 (m, 2H), 3.22 (m, 2H), 3.00-2.88 (m, 3H), 2.83-2.73 (m, 1H), 2.51-2.27 (m, 3H), 2.16 (m, 1H), 2.09-1.87 (m, 5H), 1.79 (m, 2H), 1.70-1.50 (m, 4H), 1.34-1.32 (m, 1H), 1.25-1.15 (m, 1H).

### Step 5B: Example 123, (S)-3-(5-(((3S*,4R*)-4-Cyclopentyl-1-((8-fluoro-2-(tetrahydro-2H-pyran-4-yl)-quinolin-6-yl)methyl)pyrrolidin-3-yl)oxy)-1-oxoisoindolin-2-yl)piperidine-2,6-dione (from Intermediate in Step 4B)

This example was prepared according to Step 5A, using the intermediate from Step 4B. MS (ESI) m/z: 641.3 [M+H]⁺. ¹H NMR (400 MHz, MeOD-d₄) δ 8.26 (d, J=8.7 Hz, 1H), 7.70 (dd, J=8.4, 1.9 Hz, 1H), 7.66 (s, 1H), 7.58-7.50 (m, 2H), 7.09-7.01 (m, 2H), 5.11 (dd, J=13.2, 5.1 Hz, 1H), 4.74 (t, J=4.7 Hz, 1H), 4.48-4.35 (m, 2H), 4.14-4.04 (m, 2H), 3.97-3.82 (m, 2H), 3.63-3.61 (m, 2H), 3.24 (m, 2H), 3.01 (m, 1H), 2.96-2.85 (m, 2H), 2.77 (m, 1H), 2.50-2.30 (m, 3H), 2.15 (m, 1H), 2.06-1.88 (m, 5H), 1.84-1.76 (m, 2H), 1.71-1.54 (m, 4H), 1.33-1.31 (m, 1H), 1.21-1.19 (m, 1H).

### Examples 149 and 118: ((S)-3-(5-(((3R*,4S*)-1-((8-fluoro-2-(tetrahydro-2H-pyran-4-yl)quinolin-6-yl)methyl)-4-(pyridin-4-yl)pyrrolidin-3-yl)oxy)-1-oxoisoindolin-2-yl)piperidine-2,6-dione and (S)-3-(5-(((3S*,4R*)-1-((8-fluoro-2-(tetrahydro-2H-pyran-4-yl)quinolin-6-yl)methyl)-4-(pyridin-4-yl)pyrrolidin-3-yl)oxy)-1-oxoisoindolin-2-yl)piperidine-2,6-dione

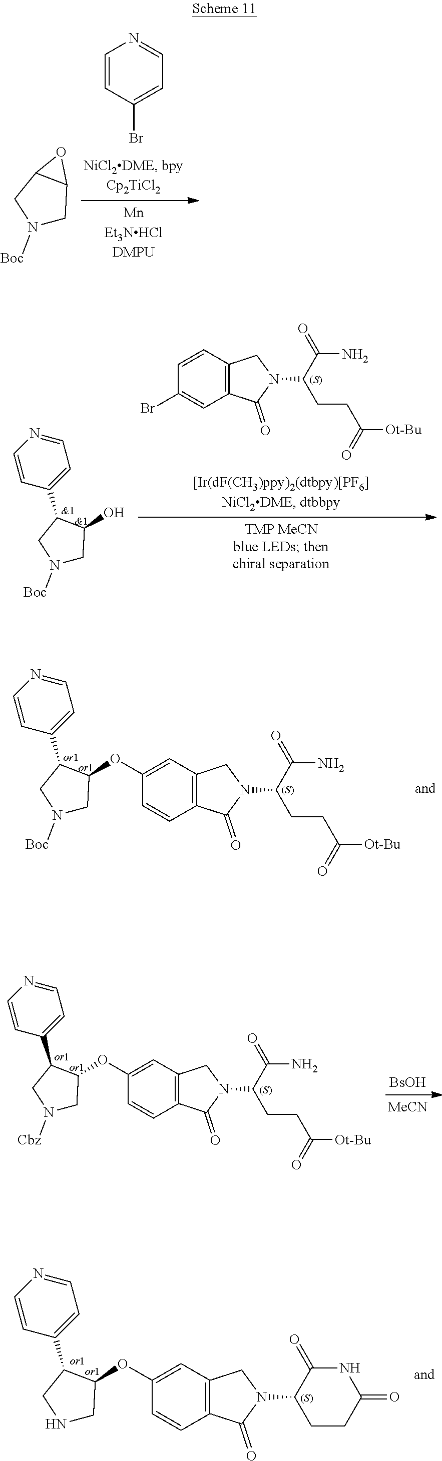

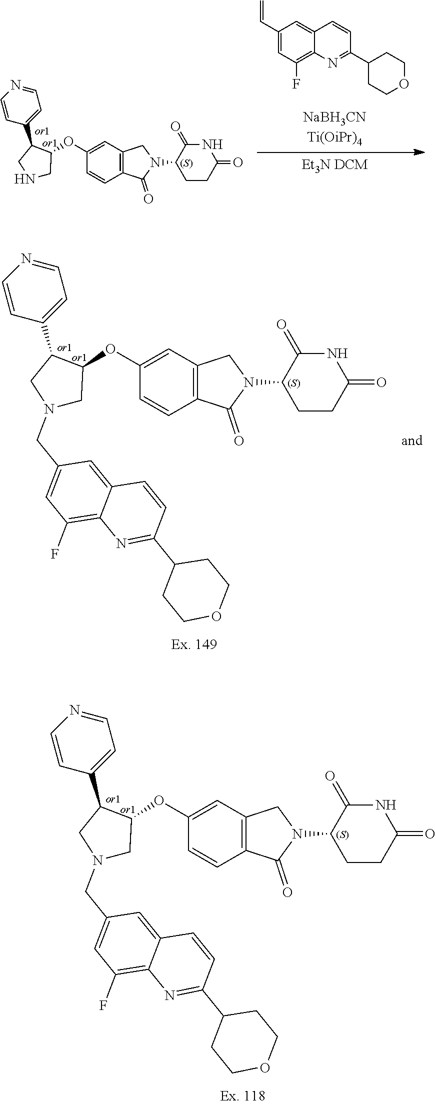

### Step 1: rac-tert-butyl 3-hydroxy-4-(pyridin-4-yl)pyrrolidine-1-carboxylate

NiCl₂·DME (59 mg, 0.27 mmol) and Mn (296 mg, 5.40 mmol) were added to a mixture of tert-butyl 6-oxa-3-azabicyclo[3.1.0]hexane-3-carboxylate (500 mg, 2.70 mmol), 4-bromopyridine (426 mg, 2.70 mmol), Cp₂TiCl₂ (67 mg, 0.27 mmol), bpy (42 mg, 0.27 mmol), and Et₃N·HCl (371 mg, 2.70 mmol) in DMPU (6 mL). The reaction was stirred at room temperature under an atmosphere of nitrogen. After 16 h, the reaction was filtered and the resulting filtrate was purified via reversed-phase silica gel chromatography to provide rac-tert-butyl 3-hydroxy-4-(pyridin-4-yl)pyrrolidine-1-carboxylate as a mixture of trans isomers. MS (ESI) m/z: 265.2 [M+H]⁺.

### Step 2: tert-butyl (3R*,4S*)-3-((2-((S)-1-amino-5-(tert-butoxy)-1,5-dioxopentan-2-yl)-1-oxoisoindolin-5-yl)oxy)-4-(pyridin-4-yl)pyrrolidine-1-carboxylate and tert-butyl (3S*,4R*)-3-((2-((S)-1-amino-5-(tert-butoxy)-1,5-dioxopentan-2-yl)-1-oxoisoindolin-5-yl)oxy)-4-(pyridin-4-yl)pyrrolidine-1-carboxylate

A mixture of rac-tert-butyl 3-hydroxy-4-(pyridin-4-yl)pyrrolidine-1-carboxylate (360 mg, 1.36 mmol), TMP (300 mg, 2.12 mmol), tert-butyl (4S)-5-amino-4-(5-bromo-1-oxo-isoindolin-2-yl)-5-oxopentanoate (600 mg, 1.51 mmol), [Ir(dF(Me)ppy)₂(dtbpy)][PF₆] (30 mg, 0.03 mmol), NiCl₂·DME (20 mg, 0.091 mmol), and dtbbpy (20 mg, 0.075 mmol) in MeCN (20 mL) was irradiated with 100 w blue LEDs for 2 days under an atmosphere of nitrogen. The reaction was purified via reversed-phase silica gel column chromatography and the resulting diastereomeric mixture was further separated by preparative chiral HPLC [Column: Chiralpak ID, 250×20 mm, hexane (w/ 0.5% 2M NH₃ in MeOH)/(1:1 EtOH/DCM)] to provide each diastereomer, which was independently carried onto the subsequent steps. tert-butyl (3R*,4S*)-3-((2-((S)-1-amino-5-(tert-butoxy)-1,5-dioxopentan-2-yl)-1-oxoisoindolin-5-yl)oxy)-4-(pyridin-4-yl)pyrrolidine-1-carboxylate (faster eluting peak): MS (ESI) m/z: 581.3 [M+Htert-butyl (3S*,4R*)-3-((2-((S)-1-amino-5-(tert-butoxy)-1,5-dioxopentan-2-yl)-1-oxoisoindolin-5-yl)oxy)-4-(pyridin-4-yl)pyrrolidine-1-carboxylate (slower eluting peak): MS (ESI) m/z: 581.3 [M+H]⁺.

### Step 3A: (S)-3-(1-Oxo-5-(((3R*,4S*)-4-(pyridin-4-yl)pyrrolidin-3-yl)oxy)isoindolin-2-yl)-piperidine-2,6-dione, TFA Salt (from Faster Eluting Intermediate in Step 2)

A solution of tert-butyl (3R*,4S*)-3-((2-((S)-1-amino-5-(tert-butoxy)-1,5-dioxopentan-2-yl)-1-oxoisoindolin-5-yl)oxy)-4-(pyridin-4-yl)pyrrolidine-1-carboxylate (70 mg, 0.12 mmol), and BsOH (76 mg, 0.48 mmol) in MeCN (5 mL) was stirred at 60° C. for 12 h under an atmosphere of nitrogen. The reaction was cooled to room temperature and purified via reversed-phase silica gel chromatography to provide (S)-3-(1-Oxo-5-(((3R*,4S*)-4-(pyridin-4-yl)pyrrolidin-3-yl)oxy)isoindolin-2-yl)-piperidine-2,6-dione, TFA salt. MS (ESI) m/z: 407.2 [M+H]⁺.

### Step 3B: (S)-3-(1-Oxo-5-(((3S*,4R*)-4-(pyridin-4-yl)pyrrolidin-3-yl)oxy)isoindolin-2-yl)-piperidine-2,6-dione, TFA salt (from slower eluting intermediate in Step 2)

This intermediate was prepared according to Step 3A, using the slower eluting intermediate from Step 2. MS (ESI) m/z: 407.2 [M+H]⁺.

### Step 4A: Example 149, ((S)-3-(5-(((3R*,4S*)-1-((8-fluoro-2-(tetrahydro-2H-pyran-4-yl)quinolin-6-yl)methyl)-4-(pyridin-4-yl)pyrrolidin-3-yl)oxy)-1-oxoisoindolin-2-yl)piperidine-2,6-dione (from Intermediate in Step 3A)

Ti(Oi-Pr)₄ (31 mg, 0.11 mmol) and Et₃N (71 mg, 0.70 mmol) were added to a solution of 8-fluoro-2-(tetrahydro-2H-pyran-4-yl)quinoline-6-carbalde-hyde (30 mg, 0.12 mmol) and (S)-3-(1-oxo-5-(((3R*,4S*)-4-(pyridin-4-yl)pyrrolidin-3-yl)oxy)isoindolin-2-yl)piperidine-2,6-dione, TFA salt (60 mg, 0.12 mmol) in DCM (5 mL). The reaction was stirred at room temperature for 20 min, at which point, NaBH₃CN (14 mg, 0.22 mmol) was added. The reaction mixture was stirred for an additional 3 h at room temperature under an atmosphere of nitrogen. The reaction was iteratively purified via silica gel chromatography (0-10% MeOH/DCM), reversed-phased silica gel chromatography, preparative achiral preparative HPLC [Column: Torus Diol OBD, 250×30 mm, CO₂/i-PrOH], and preparative chiral HPLC [Column: Chiral Art Amylose-SA, 250×20 mm, hexane/EtOH] to provide ((S)-3-(5-(((3R*,4S*)-1-((8-fluoro-2-(tetrahydro-2H-pyran-4-yl)quinolin-6-yl)methyl)-4-(pyridin-4-yl)pyrrolidin-3-yl)oxy)-1-oxoisoindolin-2-yl)piperidine-2,6-dione. MS (ESI) m/z: 650.5 [M+H]⁺. ¹H NMR (400 MHz, MeOD-d₄) δ 8.50 (d, J=5.2 Hz, 2H), 8.31-8.21 (m, 2H), 7.71-7.64 (m, 2H), 7.58 (m, 2H), 7.49 (d, J=5.2 Hz, 2H), 6.99 (m, 2H), 5.10 (m, 2H), 4.37 (s, 2H), 4.11 (m, 2H), 3.95 (m, 2H), 3.64-3.61 (m, 3H), 3.38 (m, 2H), 3.28-3.21 (m, 1H), 3.05 (m, 1H), 2.90-2.88 (m, 1H), 2.80-2.71 (m, 2H), 2.46-2.44 (m, 1H), 2.15-2.12 (m, 1H), 2.03-2.01 (m, 2H), 1.96-1.87 (m, 2H).

### Step 4B: Example 118, ((S)-3-(5-(((3S*,4R*)-1-((8-fluoro-2-(tetrahydro-2H-pyran-4-yl)quinolin-6-yl)methyl)-4-(pyridin-4-yl)pyrrolidin-3-yl)oxy)-1-oxoisoindolin-2-yl)piperidine-2,6-dione (from Intermediate in Step 3B)

This example was prepared according to Step 4A, using the intermediate from Step 3B. MS (ESI) m/z: 650.6 [M+H]⁺. ¹H NMR (400 MHz, MeOD-d₄) δ 8.51 (d, J=5.3 Hz, 2H), 8.28 (m, 1H), 7.72-7.66 (m, 2H), 7.58 (dd, J=10.0, 4.8 Hz, 2H), 7.49 (d, J=5.4 Hz, 2H), 7.03-6.95 (m, 2H), 5.13-5.05 (m, 2H), 4.37-4.35 (m, 2H), 4.15-4.07 (m, 2H), 3.99-3.89 (m, 2H), 3.65-3.62 (m, 3H), 3.38 (m, 2H), 3.27-3.20 (m, 1H), 3.05 (m, 1H), 2.89-2.87 (m, 1H), 2.81-2.72 (m, 2H), 2.50-2.39 (m, 1H), 2.18-2.10 (m, 1H), 2.02-1.99 (m, 2H), 1.91 (m, 2H).

### Examples 133 and 152: rel-3-(5-(((3R*,4R*)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)piperidine-2,6-dione and rel-3-(5-(((3S*,4S*)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)piperidine-2,6-dione

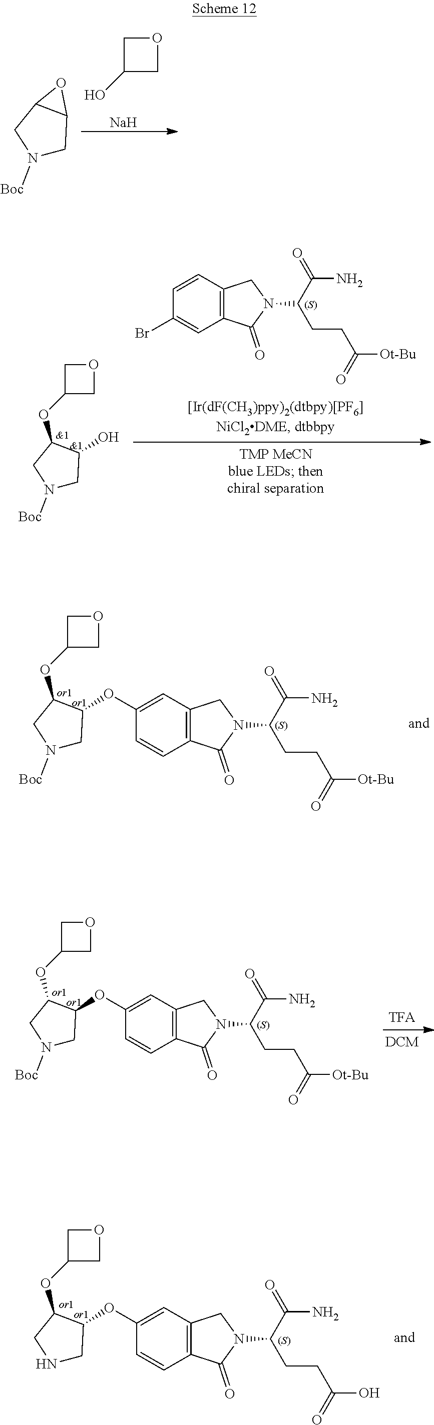

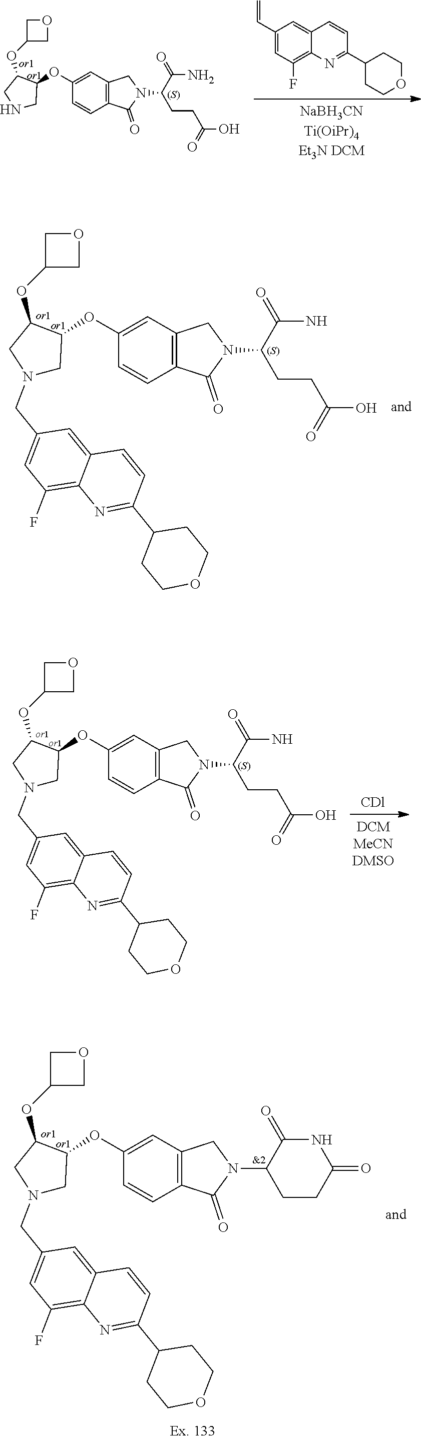

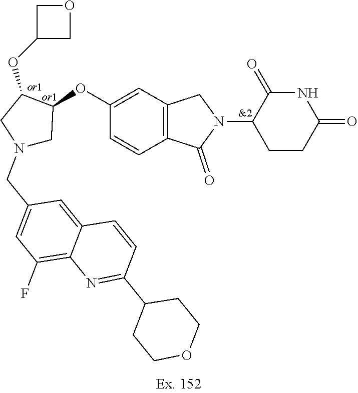

### Step 1: rac-tert-butyl 3-hydroxy-4-(oxetan-3-yloxy)pyrrolidine-1-carboxylate

NaH (60% dispersion in mineral oil, 173 mg, 4.32 mmol) was added to a solution of tert-butyl 6-oxa-3-azabicyclo[13.1.0]hexane-3-carboxylate (800 mg, 4.32 mmol) in oxetan-3-ol (2 mL) at 0° C. under an atmosphere of nitrogen. The reaction was heated to 50° C. After stirring for 16 h, the reaction was quenched with minimal amounts of water and the resulting mixture was purified via reversed-phase silica gel chromatography to provide rac-tert-butyl 3-hydroxy-4-(oxetan-3-yloxy)pyrrolidine-1-carboxylate as a mixture of trans isomers. MS (ESI) m/z: 260.0 [M+H]⁺.

### Step 2: tert-butyl (3R*,4R*)-3-((2-((S)-1-amino-5-(tert-butoxy)-1,5-dioxopentan-2-yl)-1-oxoisoindolin-5-yl)oxy)-4-(oxetan-3-yloxy)pyrrolidine-1-carboxylate and tert-butyl (3S*,4S*)-3-((2-((S)-1-amino-5-(tert-butoxy)-1,5-dioxopentan-2-yl)-1-oxoisoindolin-5-yl)oxy)-4-(oxetan-3-yloxy)pyrrolidine-1-carboxylate

NiCl₂·DME (13 mg, 0.06 mmol), dtbbpy (14 mg, 0.05 mmol), TMP (268 mg, 1.90 mmol), and [Ir(dF(Me)ppy)₂(dtbpy)][PF₆] (20 mg, 0.020 mmol) were added to a solution of rac-tert-butyl 3-hydroxy-4-(oxetan-3-yloxy)pyrrolidine-1-carboxylate (600 mg, 2.31 mmol) and tert-butyl (4S)-5-amino-4-(5-bromo-1-oxo-isoindolin-2-yl)-5-oxopentanoate (400 mg, 1.01 mmol) in MeCN (10 mL) under an atmosphere of nitrogen. The reaction was irradiated with 100 w blue LEDs for 12 h under an atmosphere of nitrogen. The reaction was purified via reversed-phase silica gel column chromatography and the resulting diastereomeric mixture was further separated by preparative chiral HPLC [Column: Chiral Art Cellulose-SB, 250×20 mm, hexane (w/ 0.5% NH₃/MeOH)/EtOH] to provide each diastereomer, which was independently carried onto the subsequent steps. (3R*,4R*)-3-((2-((S)-1-amino-5-(tert-butoxy)-1,5-dioxopentan-2-yl)-1-oxoisoindolin-5-yl)oxy)-4-(oxetan-3-yloxy)pyrrolidine-1-carboxylate (faster eluting peak): MS (ESI) m/z: 576.3 [M+H]⁺. tert-butyl (3S*,4S*)-3-((2-((S)-1-amino-5-(tert-butoxy)-1,5-dioxopentan-2-yl)-1-oxoisoindolin-5-yl)oxy)-4-(oxetan-3-yloxy)pyrrolidine-1-carboxylate (slower eluting peak): MS (ESI) m/z: 576.3 [M+H]⁺.

### Step 3A: (S)-5-amino-4-(5-(((3R*,4R*)-4-(oxetan-3-yloxy)pyrrolidin-3-yl)oxy)-1-oxoisoindolin-2-yl)-5-oxopentanoic Acid, TFA Salt (from Faster Eluting Intermediate from Step 2)

TFA (0.5 mL) was added to a solution of (3R*,4R*)-3-((2-((S)-1-amino-5-(tert-butoxy)-1,5-dioxopentan-2-yl)-1-oxoisoindolin-5-yl)oxy)-4-(oxetan-3-yloxy)pyrrolidine-1-carboxylate in DCM (5 mL) at 0° C. The reaction was stirred for 16 h at room temperature under an atmosphere of nitrogen. The reaction was concentrated under reduced pressure and purified via semi-preparative reversed-phase-HPLC to provide (S)-5-amino-4-(5-(((3R*,4R*)-4-(oxetan-3-yloxy)pyrrolidin-3-yl)oxy)-1-oxoisoindolin-2-yl)-5-oxopentanoic acid, TFA salt. MS (ESI) m/z: 420.3 [M+H]⁺.

### Step 3B: (S)-5-amino-4-(5-(((3S*,4S*)-4-(oxetan-3-yloxy)pyrrolidin-3-yl)oxy)-1-oxoisoindolin-2-yl)-5-oxopentanoic Acid, TFA Salt (from Slower Eluting Intermediate from Step 2)

This intermediate was prepared according to Step 3A, using the slower eluting intermediate from Step 2. MS (ESI) m/z: 420.3 [M+H]⁺.

### Step 4A: (S)-5-amino-4-(5-(((3R*,4R*)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)-5-oxopentanoic Acid (from Intermediate in Step 3A)

Ti(Oi-Pr)₄ (43 mg, 0.15 mmol) and Et₃N (85 mg, 0.84 mmol) were added to a solution of 8-fluoro-2-(tetrahydro-2H-pyran-4-yl)quinoline-6-carbalde-hyde (30 mg, 0.12 mmol) and (S)-5-amino-4-(5-(((3R*,4R*1-4-(oxetan-3-yloxy)pyrrolidin-3-yl)oxy)-1-oxoisoindolin-2-yl)-5-oxopentanoic acid, TFA salt (60 mg, 0.11 mmol) in DCM (5 mL). The reaction was stirred at room temperature for 20 min, at which point, NaBH₃CN (15 mg, 0.24 mmol) was added. The reaction mixture was stirred for an additional 3 h at room temperature under an atmosphere of nitrogen. The reaction was concentrated under reduced pressure and purified via silica gel chromatography (0-8% MeOH/DCM to provide (S)-5-amino-4-(5-(((3R*,4R*)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)-5-oxopentanoic acid. MS (ESI) m/z: 663.4 [M+H]⁺.

### Step 4B: (S)-5-amino-4-(5-(((3S*,4S*)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)-5-oxopentanoic Acid (from Intermediate in Step 3B)

This example was prepared according to Step 4A, using the intermediate from Step 3B. MS (ESI) m/z: 663.4 [M+H]⁺.

### Step 5A: Example 133, rel-3-(5-(((3R,4R)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)piperidine-2,6-dione (from Intermediate in Step 4A)

CDI (37 mg, 0.23 mmol) was added to a solution of (S)-5-amino-4-(5-(((3R*,4R*)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)-5-oxopentanoic acid (30 mg, 0.045 mmol) in DCM (0.5 mL), MeCN (0.5 mL), and DMSO (0.5 mL). The reaction was stirred at room temperature for 16 h under an atmosphere of nitrogen. The reaction was concentrated under reduced pressure and purified via preparative HPLC to provide rel-3-(5-(((3R,4R)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)piperidine-2,6-dione as a mixture of diastereomers. MS (ESI) m/z: 645.4 [M+H]⁺. ¹H NMR (400 MHz, MeOD-d₄) δ 8.44-8.26 (m, 1H), 7.87 (s, 1H), 7.76-7.58 (m, 3H), 7.24-7.00 (m, 2H), 5.35-5.20 (m, 1H), 5.15 (dd, J=13.6, 5.0 Hz, 1H), 4.85-4.76 (m, 3H), 4.69 (m, 1H), 4.63 (m, 2H), 4.52-4.38 (m, 3H), 4.15-4.01 (m, 3H), 3.90-3.50 (m, 6H), 3.27 (m, 1H), 2.92-2.89 (m, 1H), 2.80 (m, 1H), 2.48-2.46 (m, 1H), 2.23-2.13 (m, 1H), 2.10-2.01 (m, 2H), 1.94 (m, 2H).

### Step 5B: Example 152, rel-3-(5-(((3S,4S)-1-((8-fluoro-2-(tetrahydro-2H-pyran-4-yl)quinolin-6-yl)methyl)-4-(oxetan-3-yloxy)pyrrolidin-3-yl)oxy)-1-oxoisoindolin-2-yl)piperidine-2,6-dione (from Intermediate in Step 4B)

This example was prepared according to Step 5A, using the intermediate from Step 4B. MS (ESI) m/z: 645.4 [M+H]⁺. ¹H NMR (400 MHz, MeOD-d₄) δ 8.32 (d, J=8.8 Hz, 1H), 7.91 (d, J=19.8 Hz, 1H), 7.77-7.63 (m, 3H), 7.19-7.05 (m, 2H), 5.24 (d, J=4.2 Hz, 1H), 5.14 (dd, J=13.3, 5.1 Hz, 1H), 4.85-4.74 (m, 3H), 4.72-4.59 (m, 3H), 4.55-4.40 (m, 3H), 4.17-4.02 (m, 3H), 3.93-3.59 (m, 6H), 3.27-3.25 (m, 1H), 2.93-2.90 (m, 1H), 2.80-2.77 (m, 1H), 2.55-2.44 (m, 1H), 2.19-2.18 (m, 1H), 2.04-2.01 (m, 2H), 1.99-1.90 (m, 2H).

### BIOLOGICAL EXAMPLES

### Example B1. WEE1-ePL Degradation Assay

To generate LentiX-WEE1-ePL-GSPT1-ND cells for the WEE1 degradation assay, LentiX-HEK293T (Invitrogen) were tranduced by lentivirus with a C-terminal ePL-tagged (DiscoverX, Fremont, CA) full-length WEE1 expression construct. A separate lentiviral construct was introduced to over-express a mutated form of GSPT1 (NP_002085), termed GSPT1-GN, which contains a G575N mutation and is further truncated in the first 158 amino acids. GSPT1-GN is functional but not degradable by Cereblon. Cells were selected and maintained in complete DMEM media (DMEM, 10% Heat Inactivated FBS, 400 μg/mL Geneticin, and 1 μg/mL puromycin at 37 C and 5% CO₂). Cells were passaged every 3-4 days by reseeding 1×10⁶ cells in a T150 flask.

Cells were dispensed into 384-well white plates (Corning #3570, NY) that were pre-spotted with compounds using an acoustic dispenser (Echo acoustic transfer system, Beckman Coulter Life Sciences, Carlsbad, CA) as a 10-point dose-response curve with a 3-fold dilution starting at either 1 uM or 10 uM and including a DMSO control. Twenty-five microliters of media containing 2500 cells for LentiX-oeWEE1-ePL was dispensed per well. Assay plates were incubated at 37° C. with 5% CO2 for four hours (WEE1) After incubation, 25 μL of the InCELL Hunter™ Detection Reagent Working Solution (DiscoverX, Fremont, CA) was added to each well and incubated at room temperature for 30 minutes protected from light. After 30 minutes, luminescence was read on a PHERAstar luminometer (Cary, NC).

To determine the half-maximal effective concentration (EC50) values for WEE1-ePL degradation, a four parameter logistic model was used: (Sigmoidal Dose-Response Model) (FIT=(A+((B−A)/1+((C/x)\hat{ }D)))) where C is the inflection point (EC50), D is the Hill slope, and A and B are the low and high limits of the fit respectively). The lower limit of the fit (value A) is referred to as Ymin. The Ymin was constrained by using a luciferase inhibitor at a concentration of 20 μM to fully inhibit the luciferase signal and was used as the Ymin=0 constraint value within each assay. The maximum limit constraint, Ymax, is similarly derived using DMSO control. The curves were processed and evaluated using Activity Base (IDBS, Alameda, CA) or using Dotmatics (Boston, MA). The results are shown in Table 2 below.

### Example B2. MKN45 and MDA-MB-231 Cell Proliferation Assays

MKN45 cells were obtained from Celgene internal cell banks and cultured in RPMI complete media (RPMI-1640, 1× Penicillin/Streptomycin, 1× glutamine, 1× Sodium Pyruvate, Non-essential Amino Acids, 10% Fetal Bovine serum, all component from Thermo-Fisher). MDA-MB-231 cells were obtained from the ATCC (HTB-26) and cultured in RPMI-1640 and 10% Fetal Bovine serum; MDA-MB-231 cells were expanded and stored as aliquots in liquid nitrogen in Celgene internal cell banks. Compounds were dispensed into 384-well plates using an HP DE300 compound printer in triplicate with 8 compounds on each plate. Compounds were dispensed into 384-well black clear bottom plates (Corning #3764BC) using an EDC acoustic dispenser at BMS. For each compound, a 10-point dose response was printed at half-log dilutions starting at either 1 uM or 10 uM in triplicate for MKN45 cells and in duplicate for MDA-MB-231 cells. The DMSO concentration was kept constant for a final assay concentration of 0.1% per well assuming 50 ul final media volume. Compound plates were sealed and frozen at −20 C until use.

For each assay batch, compound plates were thawed and allowed to reach room temperature, and cells were seeded at 400 MKN45 cells per well, or 800 MDA-MB-231 cells per well, in 50 ul media listed above for each cell line. After 120 hours of incubation at 37 C and 5% CO2, cells were lysed by addition of 25 μL of Cell-Titer-Glo Reagent (Promega Corporation, Madison, WI), as per manufacturer's instructions, protected from light, shaken for 45 minutes, and total luminescence read by a Perkin Elmer Envision plate reader. Total luminescence signal is linearly correlated with cell number remaining in the well.

Luminescence data was processed per plate by subtracting reagent-only “blank” well luminescence from all measured values and calculating a percent of DMSO control value for each treatment well. Then, to determine the half-maximal effective concentration (EC50) values for cell growth inhibition, a four parameter logistic model was fit to the data for each DMSO-normalized compound: Sigmoidal Dose-Response Model: (FIT=(A+((B−A)/1+((C/x)\hat{ }D)))) where C is the inflection point (EC52), D is the Hill slope, and A and B are the low and high limits of the fit respectively. The lower limit of the fit (value A) is referred to as Ymin-calculated. The minimal percent of DMSO control that is observed in the concentrations tested for each compound is labeled as “Ymin-obs”, and was recorded and reported in the tables. Sigmoidal fit curves were processed and evaluated using Activity Base (IDBS, Alameda, CA) or using Dotmatics (Boston, MA). The results are shown in Table 2 below. N/A=not available at the present time.

TABLE 2
MKN45 cell proliferation and WEE1-ePL degradation.

| | | | | | MDA-MB | MDA-MB |
|---|---|---|---|---|---|---|
| | 293T-LentiX | 293T-LentiX | MKN45 | MKN45 | 231 | 231 |
| | WEE1-ePL | WEE1-ePL | Cell | Cell | Cell | Cell |
| | Degradation | Degradation | Prolif. | Prolif. | Prolif. | Prolif. |
| | 4 hr | 4 hr | 120 hr | 120 hr | 120 hr | 120 hr |
| Ex. # | EC₅₀ (uM) | Y_{Min} (%) | EC₅₀ (uM) | Y_{Min} (%) | EC₅₀ (uM) | Y_{Min} (%) |
| 1 | 0.368 | 30.7 | 0.075 | 16.1 | 1.555 | 70.9 |
| 2 | 0.339 | 33.4 | 0.053 | 28.1 | 0.234 | 73.8 |
| 3 | 0.010 | 28.1 | 0.019 | 13.0 | 0.129 | 61.3 |
| 4 | 0.014 | 30.9 | 0.023 | 12.6 | 0.146 | 61.3 |
| 5 | 0.001 | 32.3 | 0.001 | 10.6 | 0.012 | 45.5 |
| 6 | 0.002 | 27.4 | 0.005 | 11.8 | 0.063 | 62.4 |
| 7 | 0.001 | 31.9 | 0.005 | 13.7 | 0.074 | 64.0 |
| 8 | 0.004 | 27.5 | 0.006 | 10.0 | 0.105 | 31.9 |
| 9 | 0.006 | 32.4 | 0.011 | 11.7 | 0.366 | 45.1 |
| 10 | 0.002 | 27.8 | 0.002 | 10.6 | 0.070 | 24.6 |
| 11 | 0.004 | 30.4 | 0.033 | 12.1 | 0.525 | 53.2 |
| 12 | N/A | N/A | 0.337 | 40.6 | 2.718 | 79.6 |
| 13 | 0.001 | 22.1 | 0.003 | 11.9 | 0.061 | 37.3 |
| 14 | 0.003 | 31.8 | 0.006 | 10.2 | 0.063 | 34.4 |
| 15 | 0.002 | 28.4 | 0.006 | 8.8 | 0.073 | 31.4 |
| 16 | 0.000 | 29.6 | 0.002 | 10.8 | 0.048 | 43.7 |
| 17 | 0.002 | 25.6 | 0.005 | 13.2 | 0.045 | 47.2 |
| 18 | 0.003 | 29.8 | 0.007 | 10.1 | 0.073 | 28.4 |
| 19 | 0.006 | 31.4 | 0.012 | 10.1 | 0.104 | 58.4 |
| 20 | 0.001 | 30.3 | 0.005 | 11.6 | 0.101 | 60.2 |
| 21 | 0.001 | 29.8 | 0.002 | 9.9 | 0.129 | 41.9 |
| 22 | 0.001 | 31.3 | 0.002 | 10.8 | 0.039 | 42.5 |
| 23 | 0.002 | 33.5 | 0.006 | 11.8 | 0.082 | 56.9 |
| 24 | 0.005 | 37.7 | 0.050 | 49.1 | 0.068 | 87.7 |
| 25 | 0.002 | 39.0 | 0.035 | 25.4 | 0.128 | 80.7 |
| 26 | 0.008 | 32.8 | 0.039 | 12.3 | 0.999 | 53.8 |
| 27 | N/A | N/A | 0.205 | 29.1 | 2.989 | 81.8 |
| 28 | 0.001 | 29.1 | 0.001 | 11.2 | 0.048 | 22.1 |
| 29 | 0.001 | 26.4 | 0.002 | 8.8 | 0.025 | 17.8 |
| 30 | 0.001 | 27.9 | 0.001 | 7.8 | 0.014 | 12.7 |
| 31 | 0.000 | 20.1 | N/A | N/A | N/A | N/A |
| 32 | 0.001 | 33.4 | 0.002 | 11.2 | 0.067 | 46.8 |
| 33 | 0.001 | 29.3 | 0.001 | 9.7 | 0.028 | 28.6 |
| 34 | 0.004 | 21.8 | 0.004 | 6.9 | 0.108 | 30.7 |
| 35 | 0.001 | 28.1 | 0.002 | 10.0 | 0.033 | 16.7 |
| 36 | 0.009 | 30.2 | 0.009 | 10.5 | 0.141 | 32.4 |
| 37 | 0.000 | 30.1 | 0.001 | 11.0 | 0.122 | 6.3 |
| 38 | 0.001 | 28.0 | 0.000 | 9.8 | 0.019 | 21.6 |
| 39 | 0.000 | 31.6 | 0.001 | 10.3 | 0.025 | 41.6 |
| 40 | 0.003 | 30.9 | 0.005 | 10.2 | 0.228 | 1.8 |
| 41 | 0.022 | 30.3 | 0.010 | 12.5 | 0.151 | 39.1 |
| 42 | 0.008 | 31.5 | 0.009 | 12.2 | 0.122 | 33.0 |
| 43 | 0.133 | 34.3 | 0.038 | 10.0 | 0.520 | 51.3 |
| 44 | 0.001 | 27.8 | 0.001 | 8.9 | 0.014 | 22.3 |
| 45 | 0.053 | 31.5 | 0.035 | 13.4 | 0.278 | 52.6 |
| 46 | 0.004 | 26.2 | 0.002 | 8.7 | 0.018 | 17.7 |
| 47 | 0.001 | 28.7 | 0.001 | 9.8 | 0.034 | 30.0 |
| 48 | 0.000 | 22.9 | 0.001 | 7.8 | 0.004 | 22.0 |
| 49 | 0.002 | 24.5 | N/A | N/A | N/A | N/A |
| 50 | 0.003 | 19.8 | 0.007 | 8.7 | 0.017 | 19.3 |
| 51 | 0.001 | 29.0 | 0.001 | 9.9 | 0.015 | 24.8 |
| 52 | 0.001 | 27.2 | 0.001 | 8.3 | 0.012 | 16.7 |
| 53 | 0.000 | 27.9 | 0.000 | 10.5 | 0.008 | 13.4 |
| 54 | 0.000 | 31.2 | 0.000 | 10.5 | 0.009 | 48.9 |
| 55 | 0.000 | 27.9 | 0.000 | 10.9 | 0.016 | 22.6 |
| 56 | 0.000 | 29.9 | 0.000 | 10.0 | 0.019 | 25.5 |
| 57 | 0.000 | 30.3 | 0.000 | 8.0 | 0.011 | 13.2 |
| 58 | 0.005 | 31.1 | 0.006 | 11.2 | 0.213 | 28.1 |
| 59 | 0.001 | 29.1 | 0.001 | 10.7 | 0.061 | 12.1 |
| 60 | 0.001 | 27.4 | 0.003 | 11.7 | 0.054 | 33.7 |
| 61 | 0.025 | 29.3 | 0.009 | 12.0 | 0.098 | 41.3 |
| 62 | 0.004 | 31.6 | 0.007 | 11.9 | 0.100 | 33.0 |
| 63 | 0.003 | 29.2 | 0.002 | 9.2 | 0.042 | 22.6 |
| 64 | 0.001 | 28.5 | 0.002 | 8.8 | 0.023 | 17.1 |
| 65 | 0.001 | 27.3 | 0.002 | 8.1 | N/A | 94.2 |
| 66 | 0.003 | 32.7 | 0.005 | 13.9 | 0.107 | 57.1 |
| 67 | 0.001 | 29.6 | 0.001 | 12.3 | 0.028 | 37.7 |
| 68 | 0.002 | 26.4 | 0.001 | 8.9 | 0.014 | 17.4 |
| 69 | 0.000 | 27.7 | 0.001 | 8.6 | 0.009 | 20.5 |
| 70 | 0.001 | 28.9 | 0.001 | 11.9 | 0.018 | 26.1 |
| 71 | 0.000 | 25.3 | 0.001 | 8.7 | 0.005 | 26.6 |
| 72 | 0.001 | 27.3 | 0.001 | 9.3 | 0.014 | 20.7 |
| 73 | 0.001 | 28.9 | 0.003 | 9.4 | 0.041 | 34.0 |
| 74 | 0.002 | 31.4 | 0.003 | 10.6 | 0.015 | 55.8 |
| 75 | 0.000 | 31.2 | 0.001 | 10.1 | 0.011 | 56.6 |
| 76 | 0.041 | 25.5 | 0.011 | 9.4 | 0.099 | 17.9 |
| 77 | 0.023 | 25.5 | 0.043 | 13.1 | 0.332 | 80.1 |
| 78 | 0.003 | 27.1 | 0.004 | 7.7 | 0.027 | 22.5 |
| 79 | 0.001 | 28.7 | 0.001 | 9.9 | 0.031 | 17.9 |
| 80 | 0.002 | 29.7 | 0.002 | 10.3 | 0.042 | 23.5 |
| 81 | 0.001 | 32.9 | 0.005 | 15.5 | 0.064 | 66.9 |
| 82 | 0.002 | 27.5 | 0.002 | 9.0 | 0.026 | 17.9 |
| 83 | 1.000 | 81.6 | 0.109 | 94.8 | 1.123 | 87.9 |
| 84 | 0.000 | 30.8 | 0.001 | 10.5 | 0.083 | 21.0 |
| 85 | 0.001 | 27.9 | 0.001 | 11.1 | 0.022 | 16.6 |
| 86 | 0.003 | 28.5 | 0.002 | 16.9 | 0.051 | 18.4 |
| 87 | 0.011 | 32.2 | 0.007 | 9.7 | 0.112 | 31.8 |
| 88 | 0.003 | 28.3 | 0.001 | 9.1 | 0.011 | 13.1 |
| 89 | 0.001 | 30.2 | 0.001 | 8.4 | 0.009 | 16.8 |
| 90 | 0.001 | 21.0 | 0.002 | 6.4 | 0.042 | 27.2 |
| 91 | 0.004 | 27.1 | 0.002 | 10.5 | 0.105 | 9.6 |
| 92 | 0.001 | 28.7 | 0.001 | 10.9 | 0.014 | 21.4 |
| 93 | 0.001 | 21.9 | 0.001 | 7.9 | 0.069 | 52.5 |
| 94 | 0.001 | 26.4 | 0.002 | 8.5 | 0.016 | 16.0 |
| 95 | 0.002 | 20.9 | N/A | N/A | N/A | N/A |
| 96 | 0.007 | 29.4 | 0.009 | 11.4 | 0.123 | 28.4 |
| 97 | 0.001 | 29.8 | 0.001 | 8.7 | 0.022 | 22.0 |
| 98 | 0.001 | 29.4 | 0.001 | 9.0 | 0.013 | 25.1 |
| 99 | 0.075 | 39.0 | 0.282 | 82.9 | 0.073 | 78.7 |
| 100 | 0.001 | 24.6 | N/A | N/A | N/A | N/A |
| 101 | 0.003 | 33.0 | N/A | N/A | N/A | N/A |
| 102 | 0.003 | 43.7 | 0.145 | 49.6 | 1.024 | 90.7 |
| 103 | 0.001 | 32.3 | 0.029 | 28.5 | 0.005 | 82.3 |
| 104 | 0.000 | 21.3 | 0.001 | 8.2 | 0.018 | 42.5 |
| 105 | 0.001 | 40.7 | 0.087 | 83.4 | 0.234 | 91.5 |
| 106 | 0.004 | 29.8 | 0.018 | 11.5 | 0.018 | 50.8 |
| 107 | 0.008 | 19.5 | 0.013 | 10.4 | 0.134 | 51.6 |
| 108 | 0.008 | 61.2 | 0.340 | 89.6 | 3.095 | 82.8 |
| 109 | 0.000 | 27.5 | 0.001 | 8.7 | 0.016 | 37.3 |
| 110 | 0.000 | 27.3 | 0.000 | 6.8 | 0.012 | 17.4 |
| 111 | 0.001 | 27.1 | 0.001 | 7.8 | 0.026 | 16.4 |
| 112 | 0.001 | 28.0 | 0.000 | 8.7 | 0.012 | 25.2 |
| 113 | 0.009 | 29.5 | 0.004 | 9.0 | 0.098 | 55.3 |
| 114 | 0.000 | 29.7 | 0.001 | 8.6 | 0.014 | 47.4 |
| 115 | 0.006 | 21.5 | 0.009 | 9.4 | 0.213 | 41.5 |
| 116 | 1.000 | 85.0 | 0.001 | 83.5 | 2.593 | 89.1 |
| 117 | 0.002 | 27.5 | 0.004 | 9.1 | 0.031 | 40.1 |
| 118 | 1.000 | 92.4 | 0.000 | 65.0 | 0.286 | 70.2 |
| 119 | 0.000 | 23.6 | 0.000 | 6.8 | 0.067 | 1.7 |
| 120 | 0.004 | 17.2 | 0.002 | 7.8 | 0.063 | 30.0 |
| 121 | 0.000 | 18.7 | N/A | 7.5 | 0.026 | 13.2 |
| 122 | 0.002 | 22.0 | 0.002 | 8.5 | 0.023 | 59.3 |
| 123 | 1.000 | 82.5 | 0.000 | 80.9 | 3.492 | 81.9 |
| 124 | 0.000 | 25.4 | N/A | N/A | N/A | N/A |
| 125 | 0.004 | 29.2 | N/A | N/A | N/A | N/A |
| 126 | 1.000 | 90.0 | N/A | N/A | N/A | N/A |
| 127 | 0.003 | 20.2 | 0.012 | 9.8 | 0.278 | 40.1 |
| 128 | 0.001 | 30.5 | 0.011 | 18.2 | 0.127 | 83.3 |
| 129 | 0.002 | 26.0 | 0.010 | 10.6 | 0.319 | 80.6 |
| 130 | 0.006 | 78.0 | 0.001 | 94.4 | 5.961 | 84.1 |
| 131 | 1.000 | 100.0 | N/A | N/A | N/A | N/A |
| 132 | 0.005 | 44.9 | N/A | N/A | N/A | N/A |
| 133 | 0.070 | 58.0 | N/A | N/A | N/A | N/A |
| 134 | 0.037 | 32.0 | 0.108 | 12.9 | 1.415 | 75.6 |
| 135 | 0.001 | 35.7 | 0.001 | 8.8 | 0.031 | 55.6 |
| 136 | 0.002 | 31.9 | 0.041 | 26.6 | 0.287 | 91.8 |
| 137 | 0.012 | 40.9 | 0.233 | 70.6 | 0.061 | 77.3 |
| 138 | 0.003 | 48.1 | 0.348 | 80.2 | 10.255 | 94.6 |
| 139 | 0.002 | 20.2 | 0.005 | 10.3 | 0.133 | 65.9 |
| 140 | 0.001 | 25.3 | 0.001 | 8.3 | 0.056 | 24.8 |
| 141 | 0.001 | 26.5 | 0.001 | 11.4 | 0.044 | 31.7 |
| 142 | 0.000 | 19.9 | 0.001 | 7.1 | 0.024 | 43.3 |
| 143 | 0.001 | 27.3 | 0.001 | 8.9 | 0.027 | 35.5 |
| 144 | 0.003 | 21.4 | N/A | N/A | N/A | N/A |
| 145 | 0.004 | 22.4 | 0.033 | 16.3 | 2.366 | 74.7 |
| 146 | 0.001 | 24.2 | 0.003 | 10.8 | 0.585 | 50.7 |
| 147 | 1.000 | 93.2 | 0.308 | 92.2 | 0.443 | 79.7 |
| 148 | 0.001 | 28.4 | 0.003 | 9.9 | 0.207 | 74.8 |
| 149 | 0.007 | 24.4 | 0.023 | 11.0 | 0.429 | 68.7 |
| 150 | 0.010 | 48.5 | N/A | N/A | N/A | N/A |
| 151 | 0.011 | 22.4 | N/A | N/A | N/A | N/A |
| 152 | 1.000 | 82.2 | N/A | N/A | N/A | N/A |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound selected from: 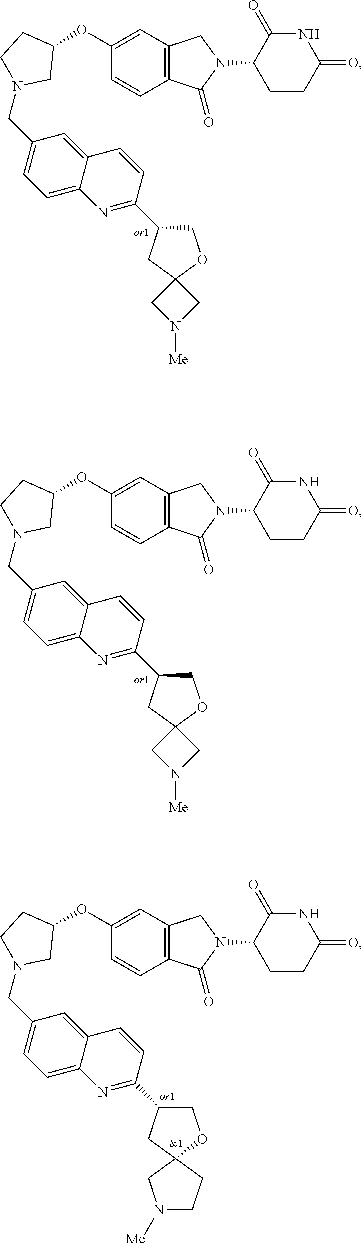 [SMILES: CN1CC2(C[C@H](c3ccc4cc(CN5CC[C@H](Oc6ccc7c(c6)CN([C@H]6CCC(=O)NC6=O)C7=O)C5)ccc4n3)CO2)C1 ; CN1CC2(C[C@@H](c3ccc4cc(CN5CC[C@H](Oc6ccc7c(c6)CN([C@H]6CCC(=O)NC6=O)C7=O)C5)ccc4n3)CO2)C1 ; CN1CC[C@]2(C[C@H](c3ccc4cc(CN5CC[C@H](Oc6ccc7c(c6)CN([C@H]6CCC(=O)NC6=O)C7=O)C5)ccc4n3)CO2)C1] 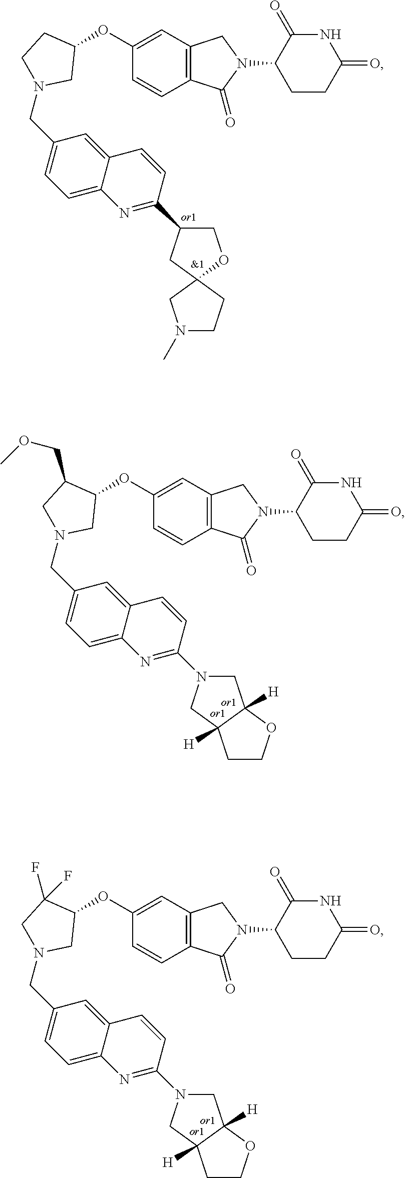 [SMILES: CN1CC[C@]2(C[C@@H](c3ccc4cc(CN5CC[C@H](Oc6ccc7c(c6)CN([C@H]6CCC(=O)NC6=O)C7=O)C5)ccc4n3)CO2)C1 ; COC[C@@H]1CN(Cc2ccc3nc(N4C[C@H]5CCO[C@H]5C4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5ccc6nc(N7C[C@H]8CCO[C@H]8C7)ccc6c5)CC4(F)F)ccc3C2=O)C(=O)N1] 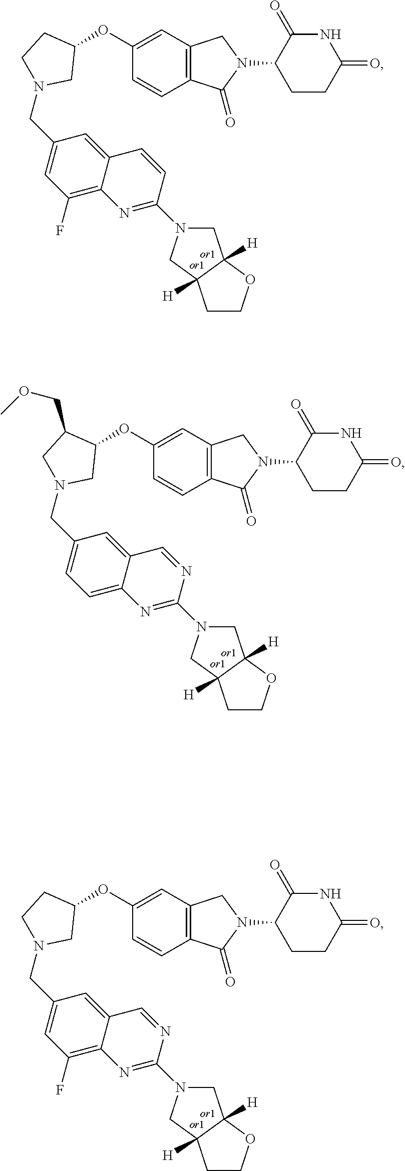 [SMILES: O=C1CC[C@H](N2Cc3cc(O[C@H]4CCN(Cc5cc(F)c6nc(N7C[C@H]8CCO[C@H]8C7)ccc6c5)C4)ccc3C2=O)C(=O)N1 ; COC[C@@H]1CN(Cc2ccc3nc(N4C[C@H]5CCO[C@H]5C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(N7C[C@H]8CCO[C@H]8C7)ncc6c5)C4)ccc3C2=O)C(=O)N1] 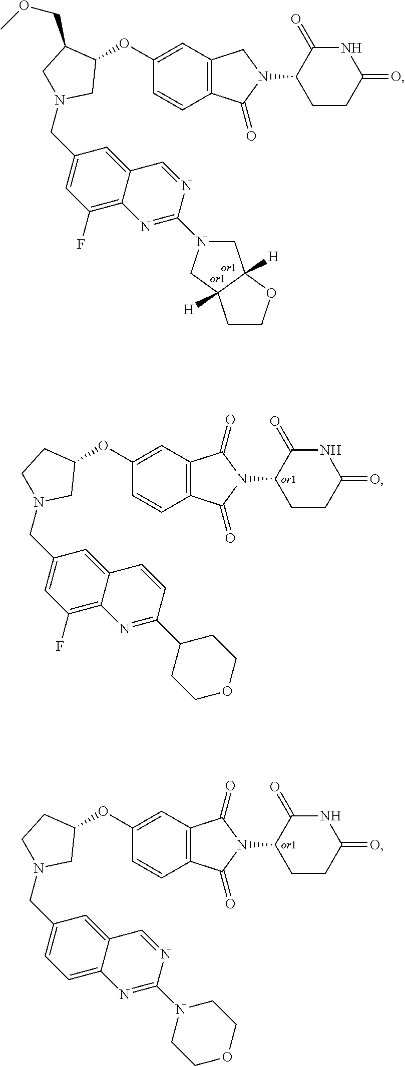 [SMILES: COC[C@@H]1CN(Cc2cc(F)c3nc(N4C[C@H]5CCO[C@H]5C4)ncc3c2)C[C@H]1Oc1ccc2c(c1)CN([C@H]1CCC(=O)NC1=O)C2=O ; O=C1CC[C@H](N2C(=O)c3ccc(O[C@H]4CCN(Cc5cc(F)c6nc(C7CCOCC7)ccc6c5)C4)cc3C2=O)C(=O)N1 ; O=C1CC[C@H](N2C(=O)c3ccc(O[C@H]4CCN(Cc5ccc6nc(N7CCOCC7)ncc6c5)C4)cc3C2=O)C(=O)N1] 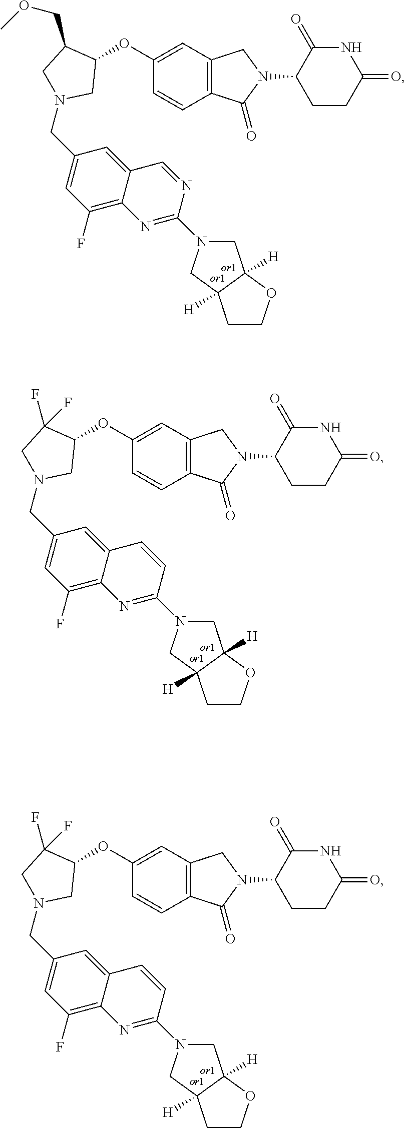 [SMILES: COC[C@@H]1CN(Cc2cc(F)c3nc(N4C[C@@H]5CCO[C@@H]5C4)ncc3c2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(N7C[C@H]8CCO[C@H]8C7)ccc6c5)CC4(F)F)ccc3C2=O)C(=O)N1 ; O=C1CC[C@H](N2Cc3cc(O[C@@H]4CN(Cc5cc(F)c6nc(N7C[C@@H]8CCO[C@@H]8C7)ccc6c5)CC4(F)F)ccc3C2=O)C(=O)N1] 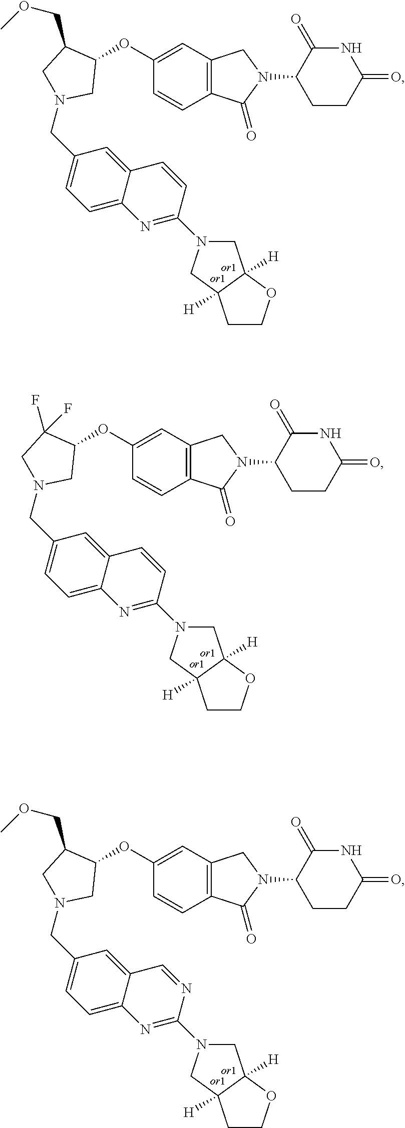 [SMILES: COC[C@@H]1CN(Cc2ccc3nc(N4C[C@@H]5CCO[C@@H]5C4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5ccc6nc(N7C[C@@H]8CCO[C@@H]8C7)ccc6c5)CC4(F)F)ccc3C2=O)C(=O)N1 ; COC[C@@H]1CN(Cc2ccc3nc(N4C[C@@H]5CCO[C@@H]5C4)ncc3c2)C[C@H]1Oc1ccc2c(c1)CN([C@H]1CCC(=O)NC1=O)C2=O] 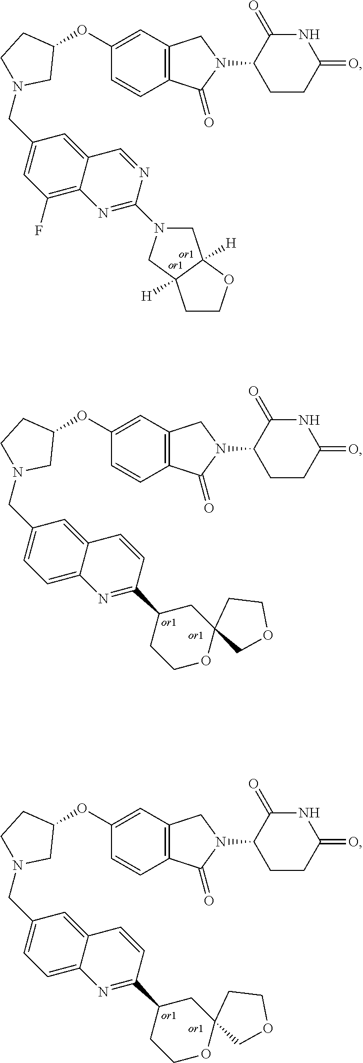 [SMILES: O=C1CC[C@H](N2Cc3cc(O[C@H]4CCN(Cc5cc(F)c6nc(N7C[C@@H]8CCO[C@@H]8C7)ncc6c5)C4)ccc3C2=O)C(=O)N1 ; O=C1CC[C@H](N2Cc3cc(O[C@H]4CCN(Cc5ccc6nc([C@@H]7CCO[C@@]8(CCOC8)C7)ccc6c5)C4)ccc3C2=O)C(=O)N1 ; O=C1CC[C@H](N2Cc3cc(O[C@H]4CCN(Cc5ccc6nc([C@@H]7CCO[C@]8(CCOC8)C7)ccc6c5)C4)ccc3C2=O)C(=O)N1] 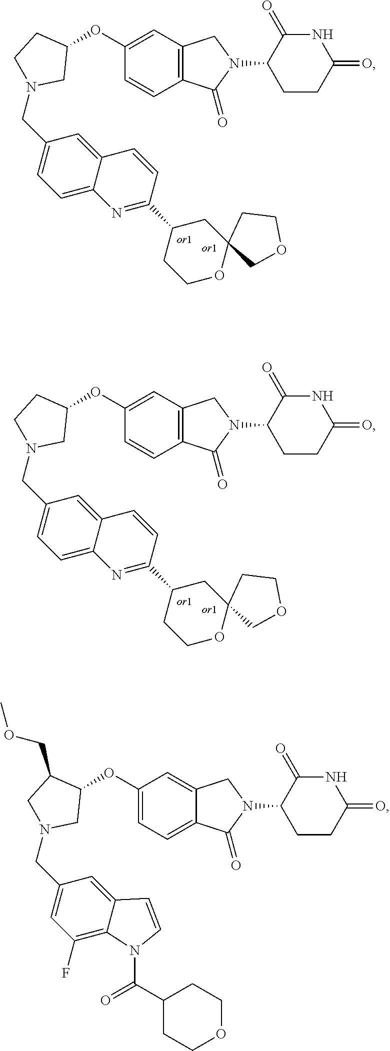 [SMILES: O=C1CC[C@H](N2Cc3cc(O[C@H]4CCN(Cc5ccc6nc([C@H]7CCO[C@@]8(CCOC8)C7)ccc6c5)C4)ccc3C2=O)C(=O)N1 ; O=C1CC[C@H](N2Cc3cc(O[C@H]4CCN(Cc5ccc6nc([C@H]7CCO[C@]8(CCOC8)C7)ccc6c5)C4)ccc3C2=O)C(=O)N1 ; COC[C@@H]1CN(Cc2cc(F)c3c(ccn3C(=O)C3CCOCC3)c2)C[C@H]1Oc1ccc2c(c1)CN([C@H]1CCC(=O)NC1=O)C2=O] 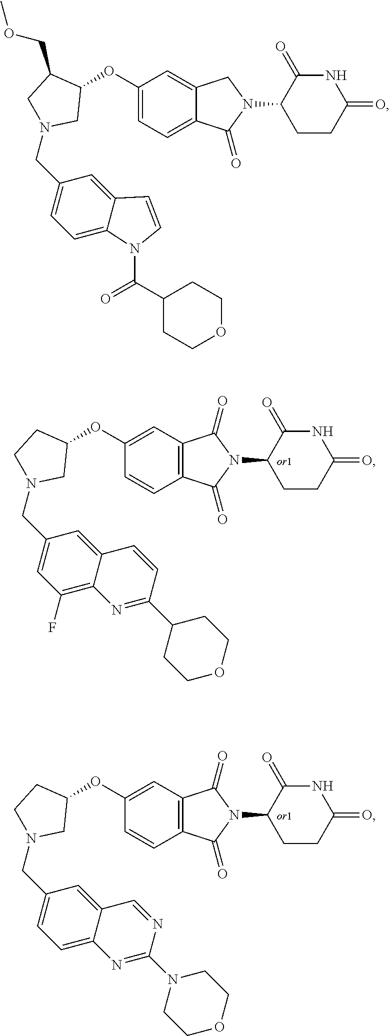 [SMILES: COC[C@@H]1CN(Cc2ccc3c(ccn3C(=O)C3CCOCC3)c2)C[C@H]1Oc1ccc2c(c1)CN([C@H]1CCC(=O)NC1=O)C2=O ; O=C1CC[C@@H](N2C(=O)c3ccc(O[C@H]4CCN(Cc5cc(F)c6nc(C7CCOCC7)ccc6c5)C4)cc3C2=O)C(=O)N1 ; O=C1CC[C@@H](N2C(=O)c3ccc(O[C@H]4CCN(Cc5ccc6nc(N7CCOCC7)ncc6c5)C4)cc3C2=O)C(=O)N1] 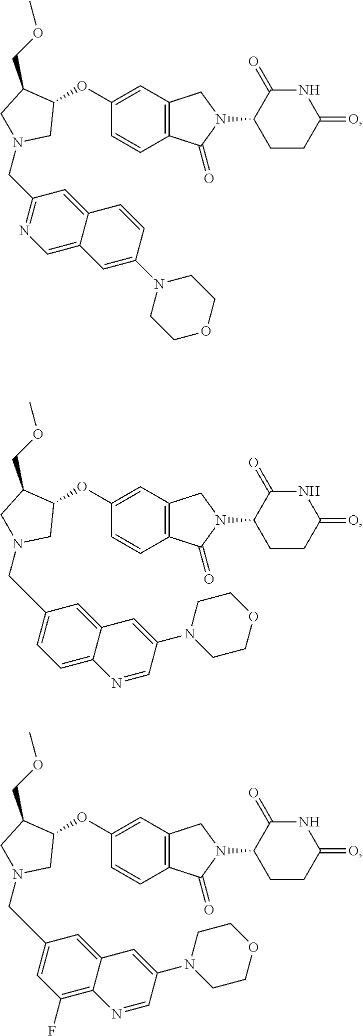 [SMILES: COC[C@@H]1CN(Cc2cc3ccc(N4CCOCC4)cc3cn2)C[C@H]1Oc1ccc2c(c1)CN([C@H]1CCC(=O)NC1=O)C2=O ; COC[C@@H]1CN(Cc2ccc3ncc(N4CCOCC4)cc3c2)C[C@H]1Oc1ccc2c(c1)CN([C@H]1CCC(=O)NC1=O)C2=O ; COC[C@@H]1CN(Cc2cc(F)c3ncc(N4CCOCC4)cc3c2)C[C@H]1Oc1ccc2c(c1)CN([C@H]1CCC(=O)NC1=O)C2=O] 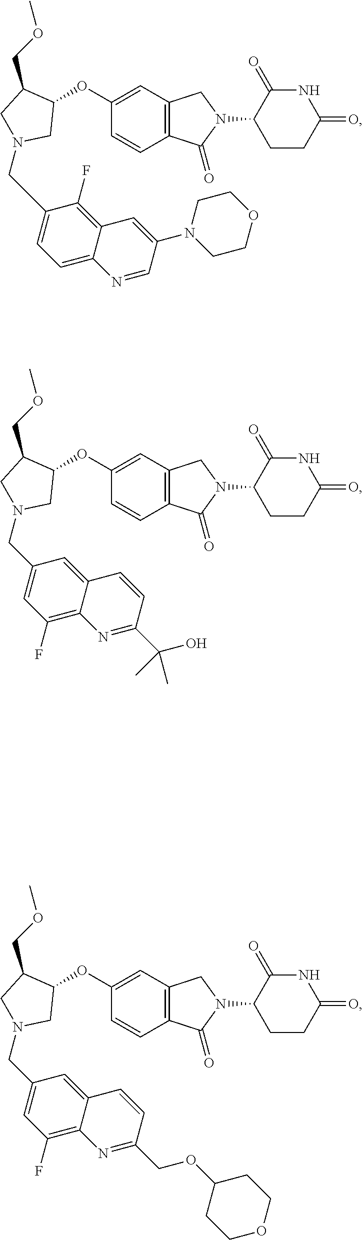 [SMILES: COC[C@@H]1CN(Cc2ccc3ncc(N4CCOCC4)cc3c2F)C[C@H]1Oc1ccc2c(c1)CN([C@H]1CCC(=O)NC1=O)C2=O ; COC[C@@H]1CN(Cc2cc(F)c3nc(C(C)(C)O)ccc3c2)C[C@H]1Oc1ccc2c(c1)CN([C@H]1CCC(=O)NC1=O)C2=O ; COC[C@@H]1CN(Cc2cc(F)c3nc(COC4CCOCC4)ccc3c2)C[C@H]1Oc1ccc2c(c1)CN([C@H]1CCC(=O)NC1=O)C2=O] 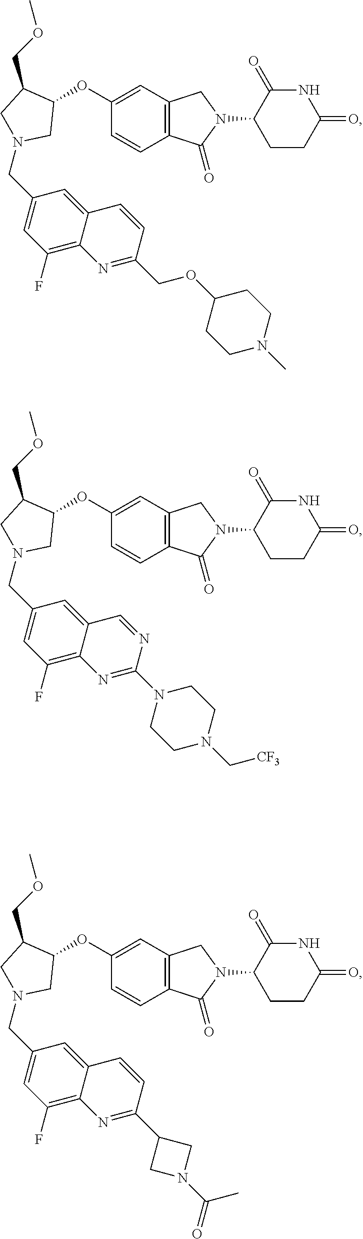 [SMILES: COC[C@@H]1CN(Cc2cc(F)c3nc(COC4CCN(C)CC4)ccc3c2)C[C@H]1Oc1ccc2c(c1)CN([C@H]1CCC(=O)NC1=O)C2=O ; COC[C@@H]1CN(Cc2cc(F)c3nc(N4CCN(CC(F)(F)F)CC4)ncc3c2)C[C@H]1Oc1ccc2c(c1)CN([C@H]1CCC(=O)NC1=O)C2=O ; COC[C@@H]1CN(Cc2cc(F)c3nc(C4CN(C(C)=O)C4)ccc3c2)C[C@H]1Oc1ccc2c(c1)CN([C@H]1CCC(=O)NC1=O)C2=O] 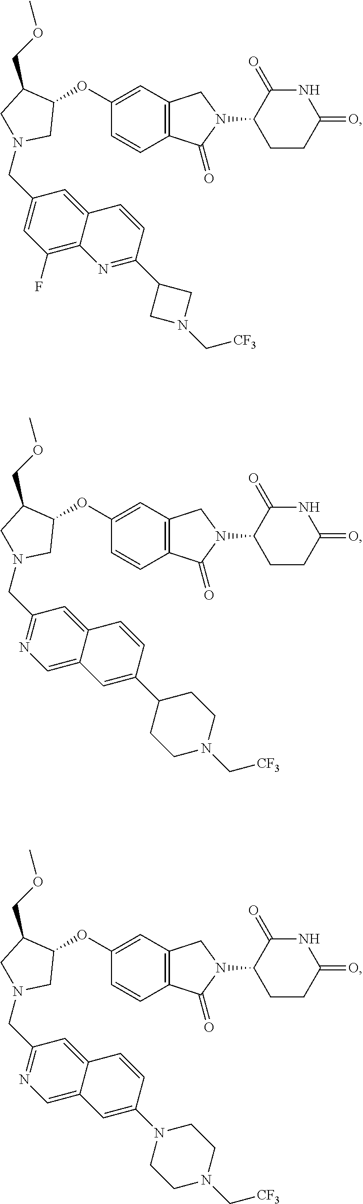 [SMILES: COC[C@@H]1CN(Cc2cc(F)c3nc(C4CN(CC(F)(F)F)C4)ccc3c2)C[C@H]1Oc1ccc2c(c1)CN([C@H]1CCC(=O)NC1=O)C2=O ; COC[C@@H]1CN(Cc2cc3ccc(C4CCN(CC(F)(F)F)CC4)cc3cn2)C[C@H]1Oc1ccc2c(c1)CN([C@H]1CCC(=O)NC1=O)C2=O ; COC[C@@H]1CN(Cc2cc3ccc(N4CCN(CC(F)(F)F)CC4)cc3cn2)C[C@H]1Oc1ccc2c(c1)CN([C@H]1CCC(=O)NC1=O)C2=O] 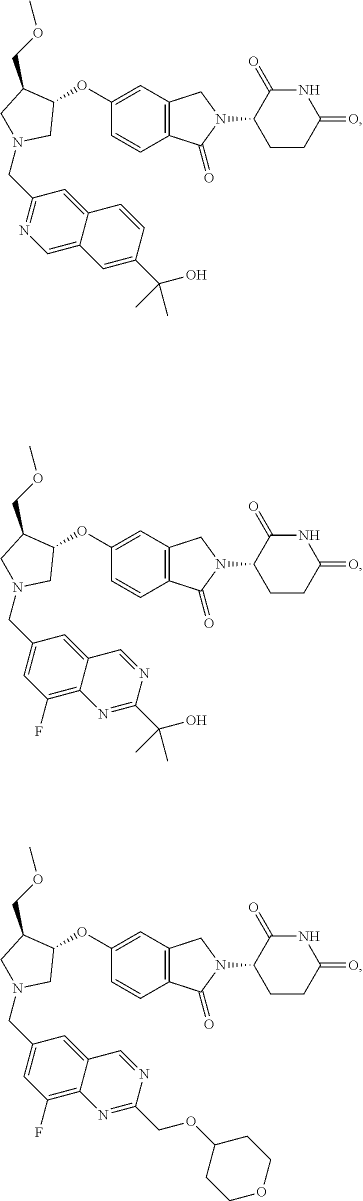 [SMILES: COC[C@@H]1CN(Cc2cc3ccc(C(C)(C)O)cc3cn2)C[C@H]1Oc1ccc2c(c1)CN([C@H]1CCC(=O)NC1=O)C2=O ; COC[C@@H]1CN(Cc2cc(F)c3nc(C(C)(C)O)ncc3c2)C[C@H]1Oc1ccc2c(c1)CN([C@H]1CCC(=O)NC1=O)C2=O ; COC[C@@H]1CN(Cc2cc(F)c3nc(COC4CCOCC4)ncc3c2)C[C@H]1Oc1ccc2c(c1)CN([C@H]1CCC(=O)NC1=O)C2=O] 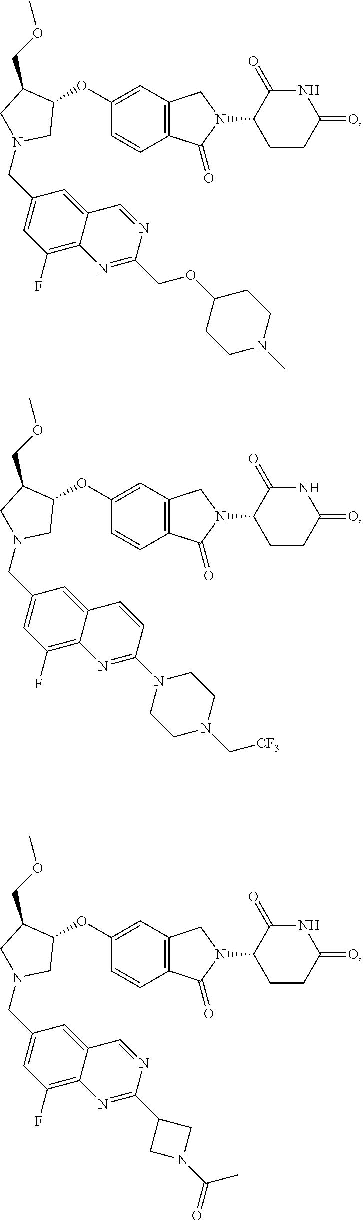 [SMILES: COC[C@@H]1CN(Cc2cc(F)c3nc(COC4CCN(C)CC4)ncc3c2)C[C@H]1Oc1ccc2c(c1)CN([C@H]1CCC(=O)NC1=O)C2=O ; COC[C@@H]1CN(Cc2cc(F)c3nc(N4CCN(CC(F)(F)F)CC4)ccc3c2)C[C@H]1Oc1ccc2c(c1)CN([C@H]1CCC(=O)NC1=O)C2=O ; COC[C@@H]1CN(Cc2cc(F)c3nc(C4CN(C(C)=O)C4)ncc3c2)C[C@H]1Oc1ccc2c(c1)CN([C@H]1CCC(=O)NC1=O)C2=O] 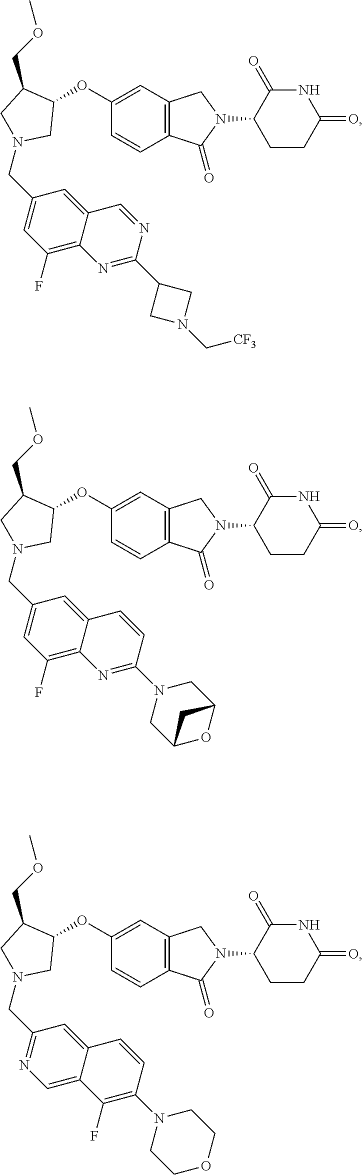 [SMILES: COC[C@@H]1CN(Cc2cc(F)c3nc(C4CN(CC(F)(F)F)C4)ncc3c2)C[C@H]1Oc1ccc2c(c1)CN([C@H]1CCC(=O)NC1=O)C2=O ; COC[C@@H]1CN(Cc2cc(F)c3nc(N4C[C@H]5C[C@@H](C4)O5)ccc3c2)C[C@H]1Oc1ccc2c(c1)CN([C@H]1CCC(=O)NC1=O)C2=O ; COC[C@@H]1CN(Cc2cc3ccc(N4CCOCC4)c(F)c3cn2)C[C@H]1Oc1ccc2c(c1)CN([C@H]1CCC(=O)NC1=O)C2=O] 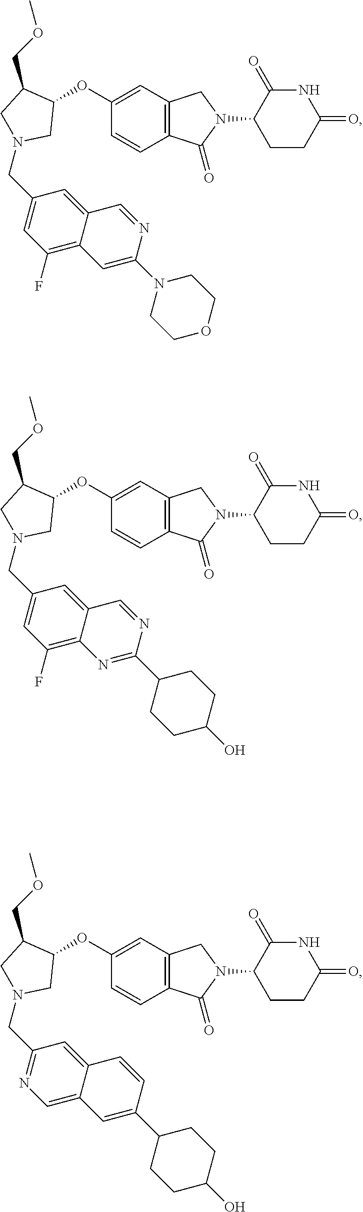 [SMILES: COC[C@@H]1CN(Cc2cc(F)c3cc(N4CCOCC4)ncc3c2)C[C@H]1Oc1ccc2c(c1)CN([C@H]1CCC(=O)NC1=O)C2=O ; COC[C@@H]1CN(Cc2cc(F)c3nc(C4CCC(O)CC4)ncc3c2)C[C@H]1Oc1ccc2c(c1)CN([C@H]1CCC(=O)NC1=O)C2=O ; COC[C@@H]1CN(Cc2cc3ccc(C4CCC(O)CC4)cc3cn2)C[C@H]1Oc1ccc2c(c1)CN([C@H]1CCC(=O)NC1=O)C2=O] 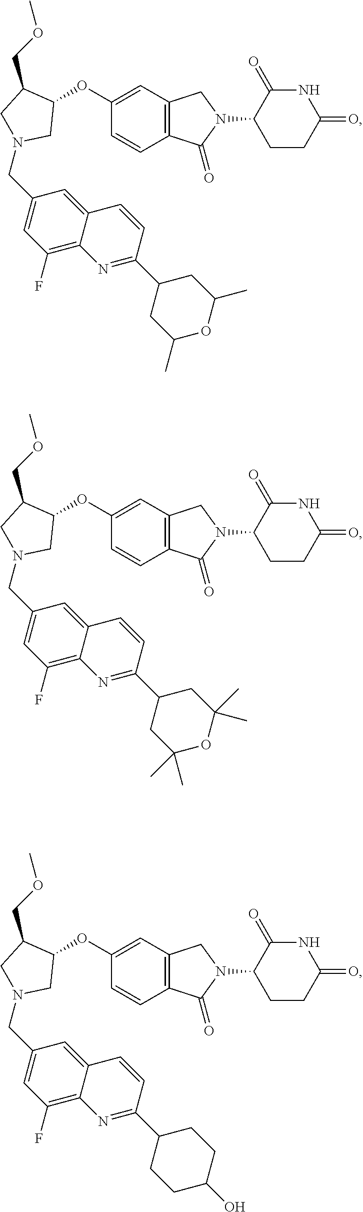 [SMILES: COC[C@@H]1CN(Cc2cc(F)c3nc(C4CC(C)OC(C)C4)ccc3c2)C[C@H]1Oc1ccc2c(c1)CN([C@H]1CCC(=O)NC1=O)C2=O ; COC[C@@H]1CN(Cc2cc(F)c3nc(C4CC(C)(C)OC(C)(C)C4)ccc3c2)C[C@H]1Oc1ccc2c(c1)CN([C@H]1CCC(=O)NC1=O)C2=O ; COC[C@@H]1CN(Cc2cc(F)c3nc(C4CCC(O)CC4)ccc3c2)C[C@H]1Oc1ccc2c(c1)CN([C@H]1CCC(=O)NC1=O)C2=O] 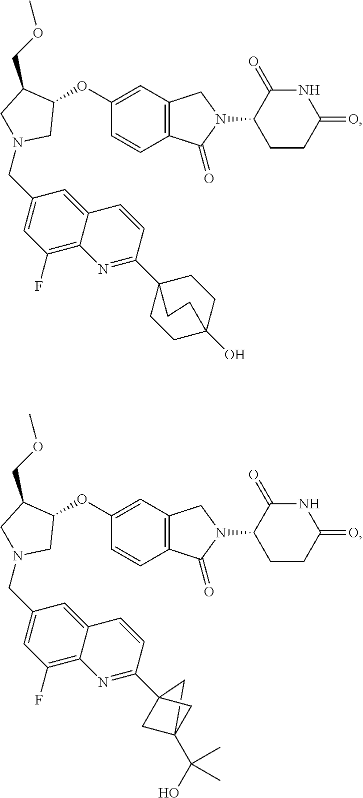 [SMILES: COC[C@@H]1CN(Cc2cc(F)c3nc(C45CCC(O)(CC4)CC5)ccc3c2)C[C@H]1Oc1ccc2c(c1)CN([C@H]1CCC(=O)NC1=O)C2=O ; COC[C@@H]1CN(Cc2cc(F)c3nc(C45CC(C(C)(C)O)(C4)C5)ccc3c2)C[C@H]1Oc1ccc2c(c1)CN([C@H]1CCC(=O)NC1=O)C2=O] 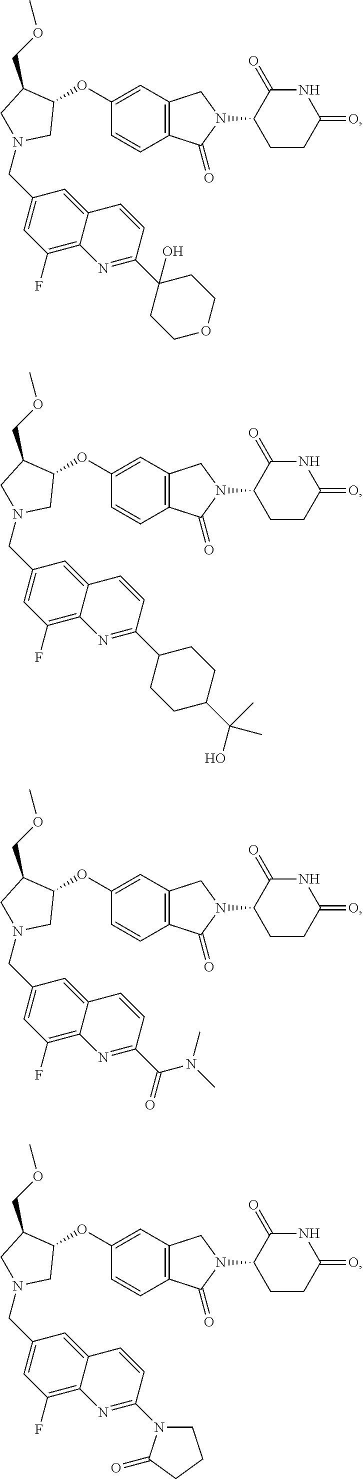 [SMILES: COC[C@@H]1CN(Cc2cc(F)c3nc(C4(O)CCOCC4)ccc3c2)C[C@H]1Oc1ccc2c(c1)CN([C@H]1CCC(=O)NC1=O)C2=O ; COC[C@@H]1CN(Cc2cc(F)c3nc(C4CCC(C(C)(C)O)CC4)ccc3c2)C[C@H]1Oc1ccc2c(c1)CN([C@H]1CCC(=O)NC1=O)C2=O ; COC[C@@H]1CN(Cc2cc(F)c3nc(C(=O)N(C)C)ccc3c2)C[C@H]1Oc1ccc2c(c1)CN([C@H]1CCC(=O)NC1=O)C2=O ; COC[C@@H]1CN(Cc2cc(F)c3nc(N4CCCC4=O)ccc3c2)C[C@H]1Oc1ccc2c(c1)CN([C@H]1CCC(=O)NC1=O)C2=O] 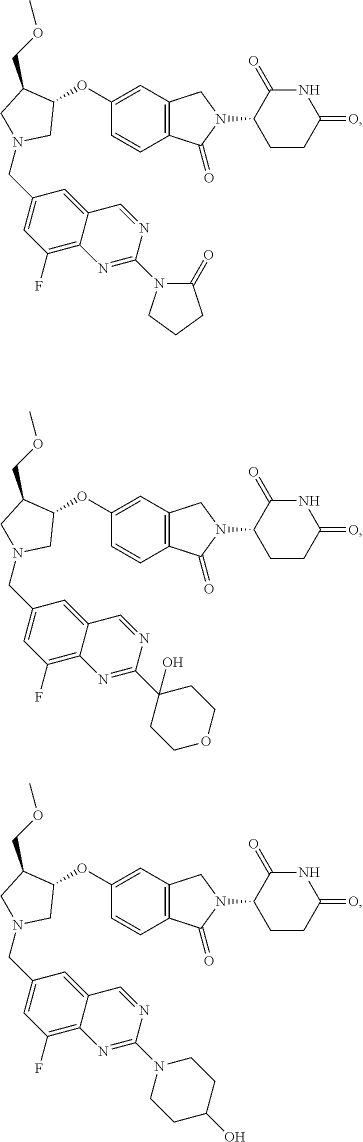 [SMILES: COC[C@@H]1CN(Cc2cc(F)c3nc(N4CCCC4=O)ncc3c2)C[C@H]1Oc1ccc2c(c1)CN([C@H]1CCC(=O)NC1=O)C2=O ; COC[C@@H]1CN(Cc2cc(F)c3nc(C4(O)CCOCC4)ncc3c2)C[C@H]1Oc1ccc2c(c1)CN([C@H]1CCC(=O)NC1=O)C2=O ; COC[C@@H]1CN(Cc2cc(F)c3nc(N4CCC(O)CC4)ncc3c2)C[C@H]1Oc1ccc2c(c1)CN([C@H]1CCC(=O)NC1=O)C2=O] 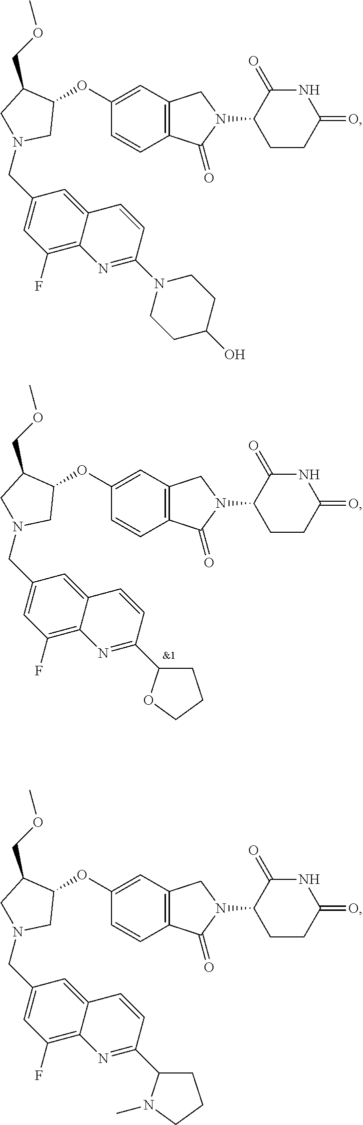 [SMILES: COC[C@@H]1CN(Cc2cc(F)c3nc(N4CCC(O)CC4)ccc3c2)C[C@H]1Oc1ccc2c(c1)CN([C@H]1CCC(=O)NC1=O)C2=O ; COC[C@@H]1CN(Cc2cc(F)c3nc(C4CCCO4)ccc3c2)C[C@H]1Oc1ccc2c(c1)CN([C@H]1CCC(=O)NC1=O)C2=O ; COC[C@@H]1CN(Cc2cc(F)c3nc(C4CCCN4C)ccc3c2)C[C@H]1Oc1ccc2c(c1)CN([C@H]1CCC(=O)NC1=O)C2=O] 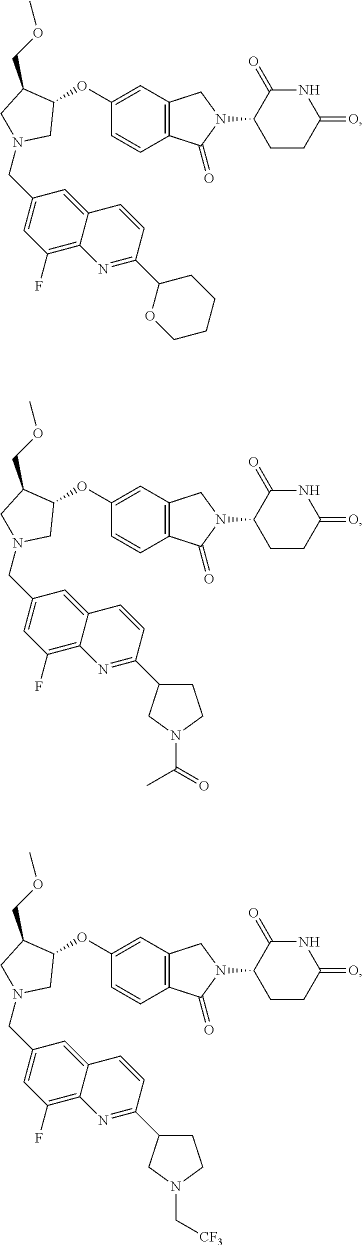 [SMILES: COC[C@@H]1CN(Cc2cc(F)c3nc(C4CCCCO4)ccc3c2)C[C@H]1Oc1ccc2c(c1)CN([C@H]1CCC(=O)NC1=O)C2=O ; COC[C@@H]1CN(Cc2cc(F)c3nc(C4CCN(C(C)=O)C4)ccc3c2)C[C@H]1Oc1ccc2c(c1)CN([C@H]1CCC(=O)NC1=O)C2=O ; COC[C@@H]1CN(Cc2cc(F)c3nc(C4CCN(CC(F)(F)F)C4)ccc3c2)C[C@H]1Oc1ccc2c(c1)CN([C@H]1CCC(=O)NC1=O)C2=O] 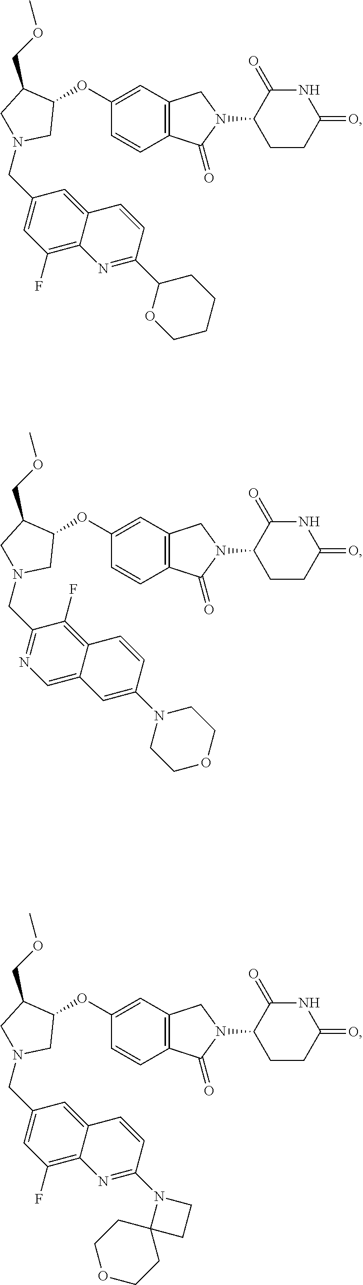 [SMILES: COC[C@@H]1CN(Cc2cc(F)c3nc(C4CCCCO4)ccc3c2)C[C@H]1Oc1ccc2c(c1)CN([C@H]1CCC(=O)NC1=O)C2=O ; COC[C@@H]1CN(Cc2ncc3cc(N4CCOCC4)ccc3c2F)C[C@H]1Oc1ccc2c(c1)CN([C@H]1CCC(=O)NC1=O)C2=O ; COC[C@@H]1CN(Cc2cc(F)c3nc(N4CCC45CCOCC5)ccc3c2)C[C@H]1Oc1ccc2c(c1)CN([C@H]1CCC(=O)NC1=O)C2=O] 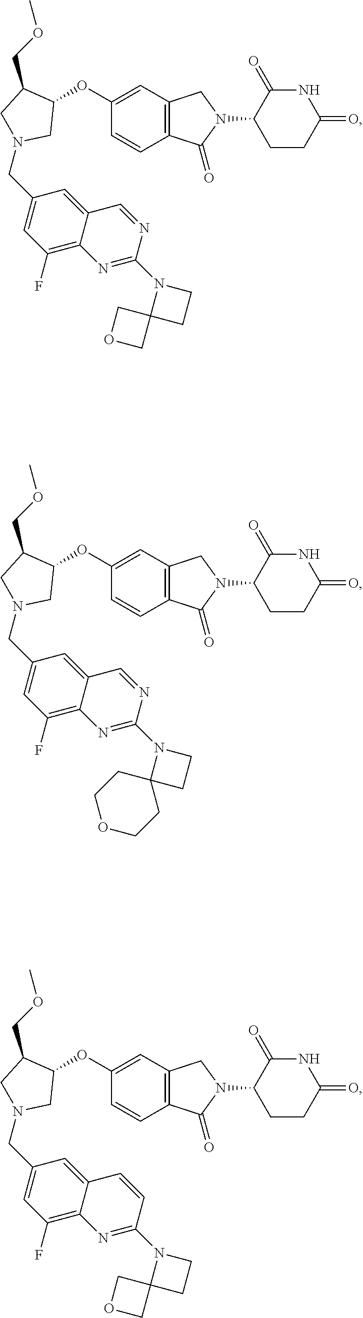 [SMILES: COC[C@@H]1CN(Cc2cc(F)c3nc(N4CCC45COC5)ncc3c2)C[C@H]1Oc1ccc2c(c1)CN([C@H]1CCC(=O)NC1=O)C2=O ; COC[C@@H]1CN(Cc2cc(F)c3nc(N4CCC45CCOCC5)ncc3c2)C[C@H]1Oc1ccc2c(c1)CN([C@H]1CCC(=O)NC1=O)C2=O ; COC[C@@H]1CN(Cc2cc(F)c3nc(N4CCC45COC5)ccc3c2)C[C@H]1Oc1ccc2c(c1)CN([C@H]1CCC(=O)NC1=O)C2=O] 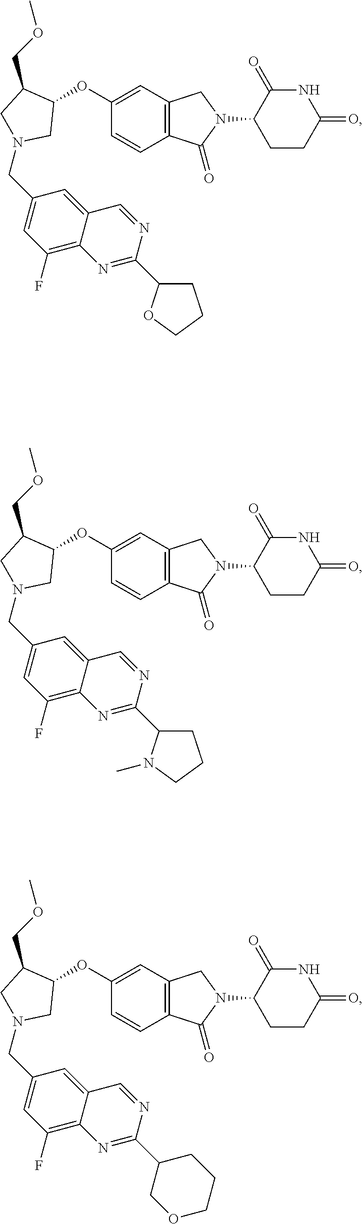 [SMILES: COC[C@@H]1CN(Cc2cc(F)c3nc(C4CCCO4)ncc3c2)C[C@H]1Oc1ccc2c(c1)CN([C@H]1CCC(=O)NC1=O)C2=O ; COC[C@@H]1CN(Cc2cc(F)c3nc(C4CCCN4C)ncc3c2)C[C@H]1Oc1ccc2c(c1)CN([C@H]1CCC(=O)NC1=O)C2=O ; COC[C@@H]1CN(Cc2cc(F)c3nc(C4CCCOC4)ncc3c2)C[C@H]1Oc1ccc2c(c1)CN([C@H]1CCC(=O)NC1=O)C2=O] 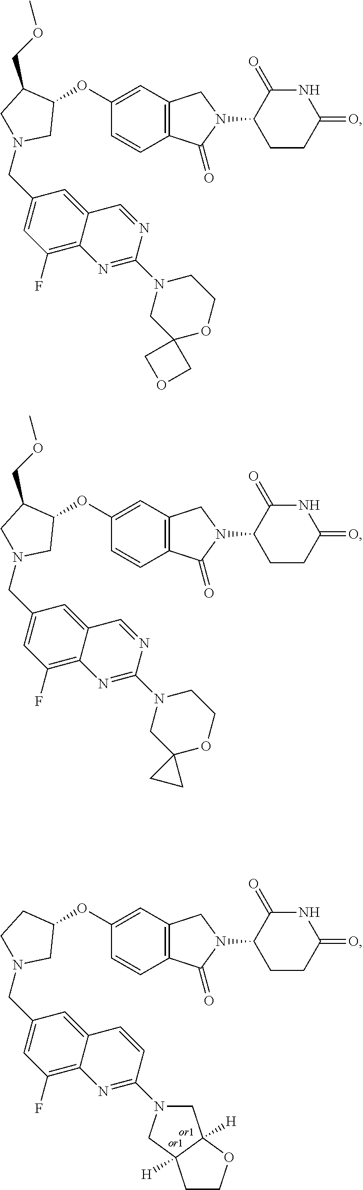 [SMILES: COC[C@@H]1CN(Cc2cc(F)c3nc(N4CCOC5(COC5)C4)ncc3c2)C[C@H]1Oc1ccc2c(c1)CN([C@H]1CCC(=O)NC1=O)C2=O ; COC[C@@H]1CN(Cc2cc(F)c3nc(N4CCOC5(CC5)C4)ncc3c2)C[C@H]1Oc1ccc2c(c1)CN([C@H]1CCC(=O)NC1=O)C2=O ; O=C1CC[C@H](N2Cc3cc(O[C@H]4CCN(Cc5cc(F)c6nc(N7C[C@@H]8CCO[C@@H]8C7)ccc6c5)C4)ccc3C2=O)C(=O)N1] 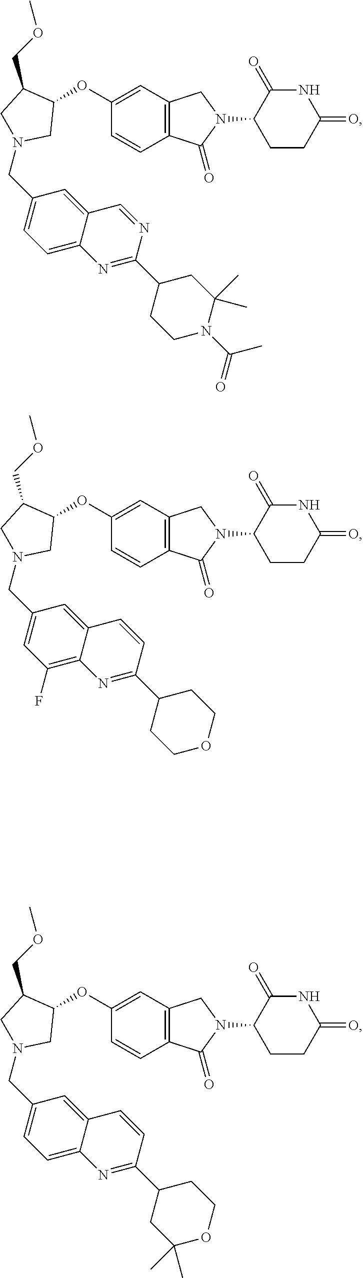 [SMILES: COC[C@@H]1CN(Cc2ccc3nc(C4CCN(C(C)=O)C(C)(C)C4)ncc3c2)C[C@H]1Oc1ccc2c(c1)CN([C@H]1CCC(=O)NC1=O)C2=O ; COC[C@H]1CN(Cc2cc(F)c3nc(C4CCOCC4)ccc3c2)C[C@H]1Oc1ccc2c(c1)CN([C@H]1CCC(=O)NC1=O)C2=O ; COC[C@@H]1CN(Cc2ccc3nc(C4CCOC(C)(C)C4)ccc3c2)C[C@H]1Oc1ccc2c(c1)CN([C@H]1CCC(=O)NC1=O)C2=O] 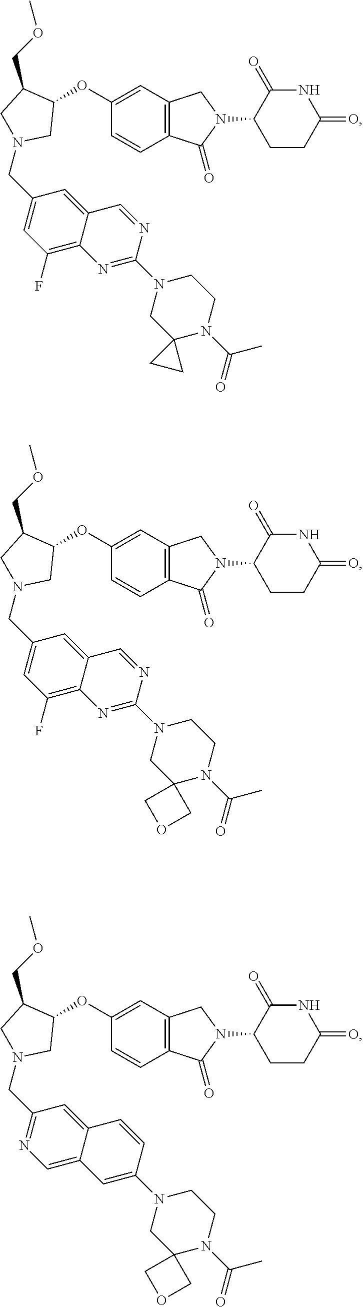 [SMILES: COC[C@@H]1CN(Cc2cc(F)c3nc(N4CCN(C(C)=O)C5(CC5)C4)ncc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(COC5)C4)ncc3c2)C[C@H]1Oc1ccc2c(c1)CN([C@H]1CCC(=O)NC1=O)C2=O ; COC[C@@H]1CN(Cc2cc3ccc(N4CCN(C(C)=O)C5(COC5)C4)cc3cn2)C[C@H]1Oc1ccc2c(c1)CN([C@H]1CCC(=O)NC1=O)C2=O] 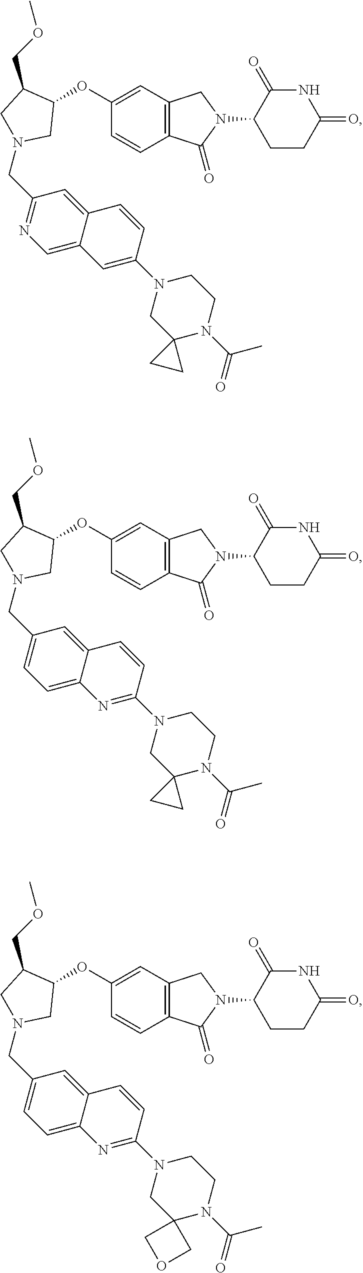 [SMILES: COC[C@@H]1CN(Cc2cc3ccc(N4CCN(C(C)=O)C5(CC5)C4)cc3cn2)C[C@H]1Oc1ccc2c(c1)CN([C@H]1CCC(=O)NC1=O)C2=O ; COC[C@@H]1CN(Cc2ccc3nc(N4CCN(C(C)=O)C5(CC5)C4)ccc3c2)C[C@H]1Oc1ccc2c(c1)CN([C@H]1CCC(=O)NC1=O)C2=O ; COC[C@@H]1CN(Cc2ccc3nc(N4CCN(C(C)=O)C5(COC5)C4)ccc3c2)C[C@H]1Oc1ccc2c(c1)CN([C@H]1CCC(=O)NC1=O)C2=O] 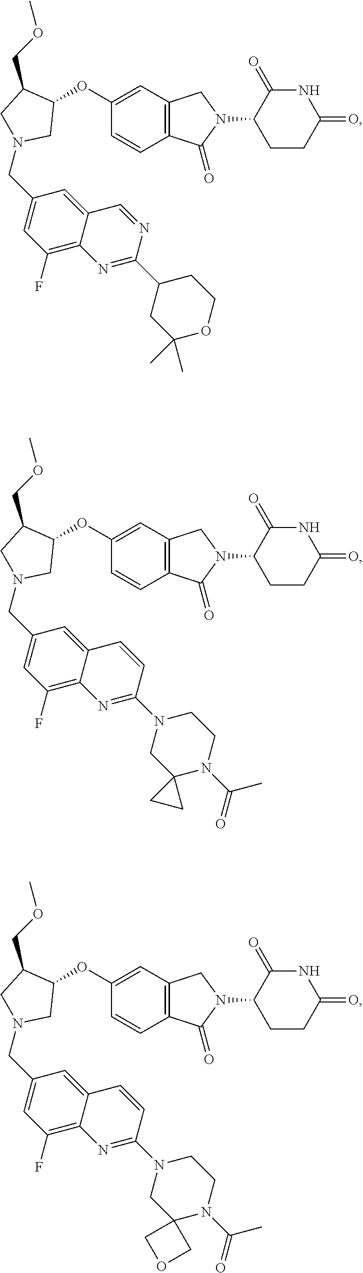 [SMILES: COC[C@@H]1CN(Cc2cc(F)c3nc(C4CCOC(C)(C)C4)ncc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(CC5)C4)ccc3c2)C[C@H]1Oc1ccc2c(c1)CN([C@H]1CCC(=O)NC1=O)C2=O ; COC[C@@H]1CN(Cc2cc(F)c3nc(N4CCN(C(C)=O)C5(COC5)C4)ccc3c2)C[C@H]1Oc1ccc2c(c1)CN([C@H]1CCC(=O)NC1=O)C2=O] 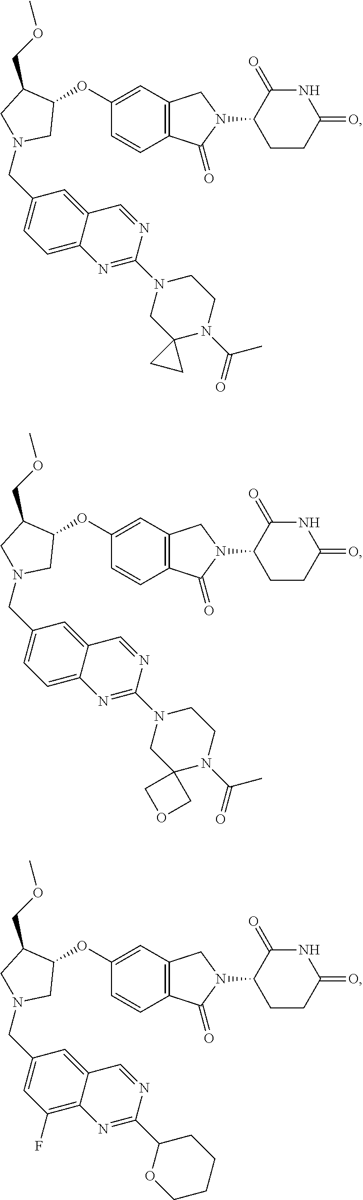 [SMILES: COC[C@@H]1CN(Cc2ccc3nc(N4CCN(C(C)=O)C5(CC5)C4)ncc3c2)C[C@H]1Oc1ccc2c(c1)CN([C@H]1CCC(=O)NC1=O)C2=O ; COC[C@@H]1CN(Cc2ccc3nc(N4CCN(C(C)=O)C5(COC5)C4)ncc3c2)C[C@H]1Oc1ccc2c(c1)CN([C@H]1CCC(=O)NC1=O)C2=O ; COC[C@@H]1CN(Cc2cc(F)c3nc(C4CCCCO4)ncc3c2)C[C@H]1Oc1ccc2c(c1)CN([C@H]1CCC(=O)NC1=O)C2=O] 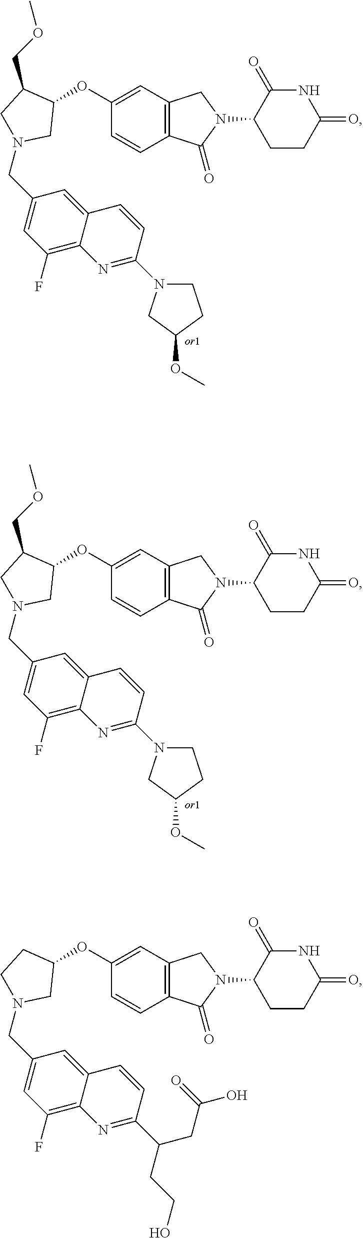 [SMILES: COC[C@@H]1CN(Cc2cc(F)c3nc(N4CC[C@@H](OC)C4)ccc3c2)C[C@H]1Oc1ccc2c(c1)CN([C@H]1CCC(=O)NC1=O)C2=O ; COC[C@@H]1CN(Cc2cc(F)c3nc(N4CC[C@H](OC)C4)ccc3c2)C[C@H]1Oc1ccc2c(c1)CN([C@H]1CCC(=O)NC1=O)C2=O ; O=C(O)CC(CCO)c1ccc2cc(CN3CC[C@H](Oc4ccc5c(c4)CN([C@H]4CCC(=O)NC4=O)C5=O)C3)cc(F)c2n1] 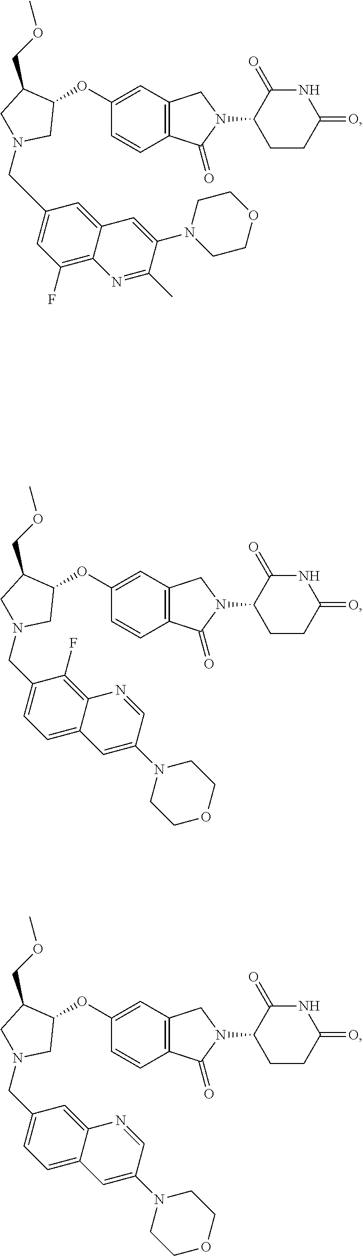 [SMILES: COC[C@@H]1CN(Cc2cc(F)c3nc(C)c(N4CCOCC4)cc3c2)C[C@H]1Oc1ccc2c(c1)CN([C@H]1CCC(=O)NC1=O)C2=O ; COC[C@@H]1CN(Cc2ccc3cc(N4CCOCC4)cnc3c2F)C[C@H]1Oc1ccc2c(c1)CN([C@H]1CCC(=O)NC1=O)C2=O ; COC[C@@H]1CN(Cc2ccc3cc(N4CCOCC4)cnc3c2)C[C@H]1Oc1ccc2c(c1)CN([C@H]1CCC(=O)NC1=O)C2=O] 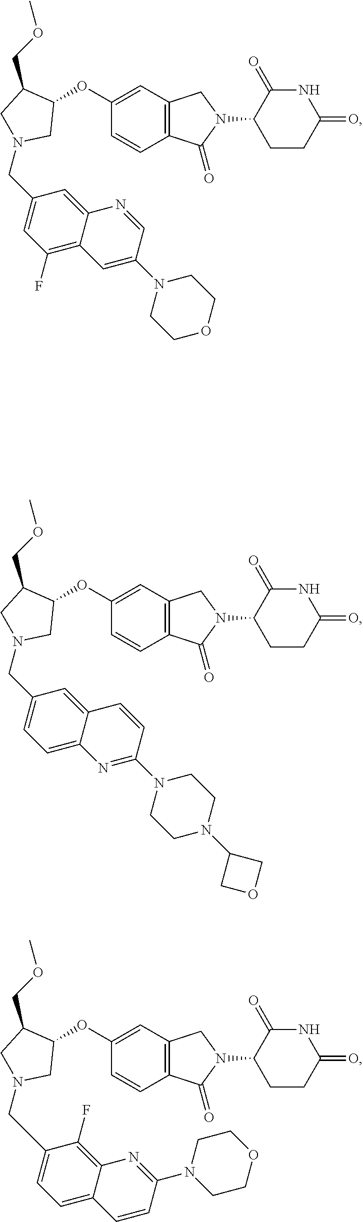 [SMILES: COC[C@@H]1CN(Cc2cc(F)c3cc(N4CCOCC4)cnc3c2)C[C@H]1Oc1ccc2c(c1)CN([C@H]1CCC(=O)NC1=O)C2=O ; COC[C@@H]1CN(Cc2ccc3nc(N4CCN(C5COC5)CC4)ccc3c2)C[C@H]1Oc1ccc2c(c1)CN([C@H]1CCC(=O)NC1=O)C2=O ; COC[C@@H]1CN(Cc2ccc3ccc(N4CCOCC4)nc3c2F)C[C@H]1Oc1ccc2c(c1)CN([C@H]1CCC(=O)NC1=O)C2=O] 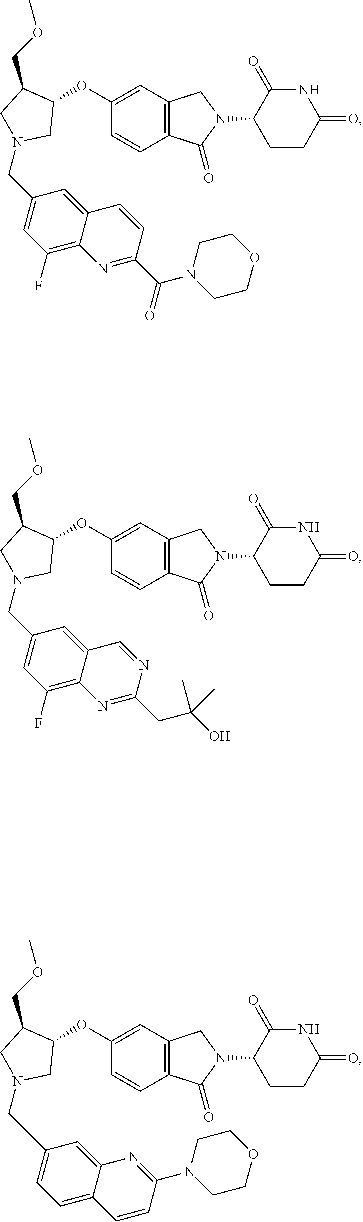 [SMILES: COC[C@@H]1CN(Cc2cc(F)c3nc(C(=O)N4CCOCC4)ccc3c2)C[C@H]1Oc1ccc2c(c1)CN([C@H]1CCC(=O)NC1=O)C2=O ; COC[C@@H]1CN(Cc2cc(F)c3nc(CC(C)(C)O)ncc3c2)C[C@H]1Oc1ccc2c(c1)CN([C@H]1CCC(=O)NC1=O)C2=O ; COC[C@@H]1CN(Cc2ccc3ccc(N4CCOCC4)nc3c2)C[C@H]1Oc1ccc2c(c1)CN([C@H]1CCC(=O)NC1=O)C2=O] 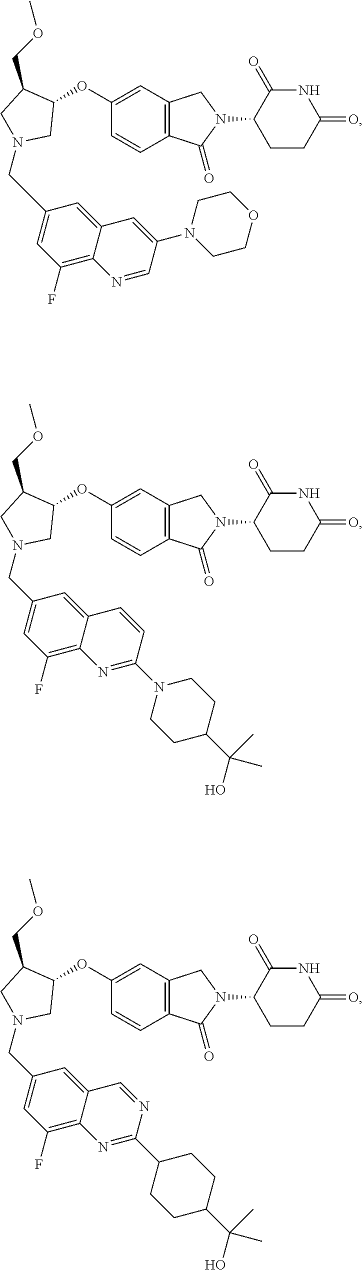 [SMILES: COC[C@@H]1CN(Cc2cc(F)c3ncc(N4CCOCC4)cc3c2)C[C@H]1Oc1ccc2c(c1)CN([C@H]1CCC(=O)NC1=O)C2=O ; COC[C@@H]1CN(Cc2cc(F)c3nc(N4CCC(C(C)(C)O)CC4)ccc3c2)C[C@H]1Oc1ccc2c(c1)CN([C@H]1CCC(=O)NC1=O)C2=O ; COC[C@@H]1CN(Cc2cc(F)c3nc(C4CCC(C(C)(C)O)CC4)ncc3c2)C[C@H]1Oc1ccc2c(c1)CN([C@H]1CCC(=O)NC1=O)C2=O] 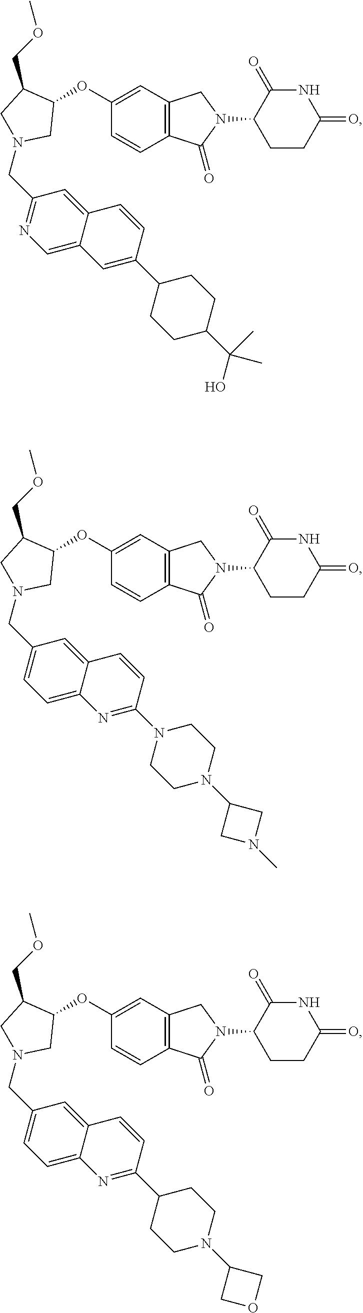 [SMILES: COC[C@@H]1CN(Cc2cc3ccc(C4CCC(C(C)(C)O)CC4)cc3cn2)C[C@H]1Oc1ccc2c(c1)CN([C@H]1CCC(=O)NC1=O)C2=O ; COC[C@@H]1CN(Cc2ccc3nc(N4CCN(C5CN(C)C5)CC4)ccc3c2)C[C@H]1Oc1ccc2c(c1)CN([C@H]1CCC(=O)NC1=O)C2=O ; COC[C@@H]1CN(Cc2ccc3nc(C4CCN(C5COC5)CC4)ccc3c2)C[C@H]1Oc1ccc2c(c1)CN([C@H]1CCC(=O)NC1=O)C2=O] 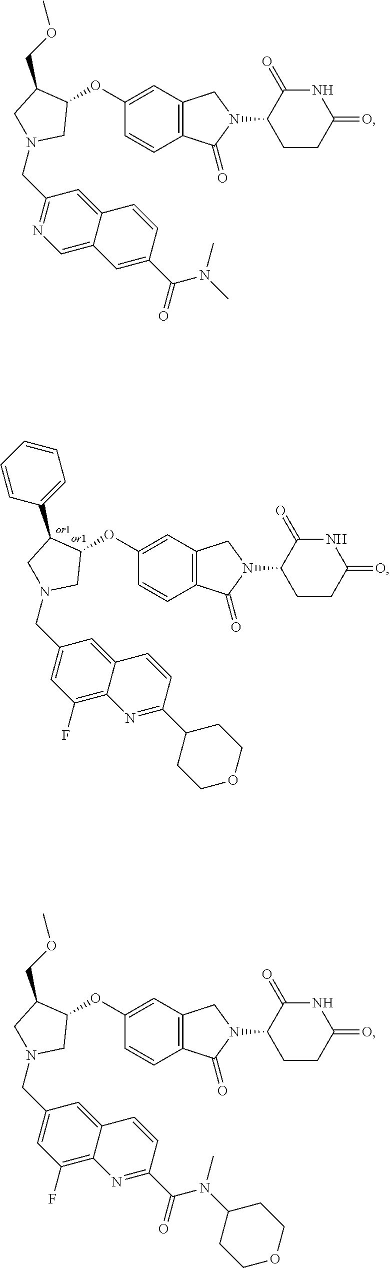 [SMILES: COC[C@@H]1CN(Cc2cc3ccc(C(=O)N(C)C)cc3cn2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4c4ccccc4)ccc3C2=O)C(=O)N1 ; COC[C@@H]1CN(Cc2cc(F)c3nc(C(=O)N(C)C4CCOCC4)ccc3c2)C[C@H]1Oc1ccc2c(c1)CN([C@H]1CCC(=O)NC1=O)C2=O] 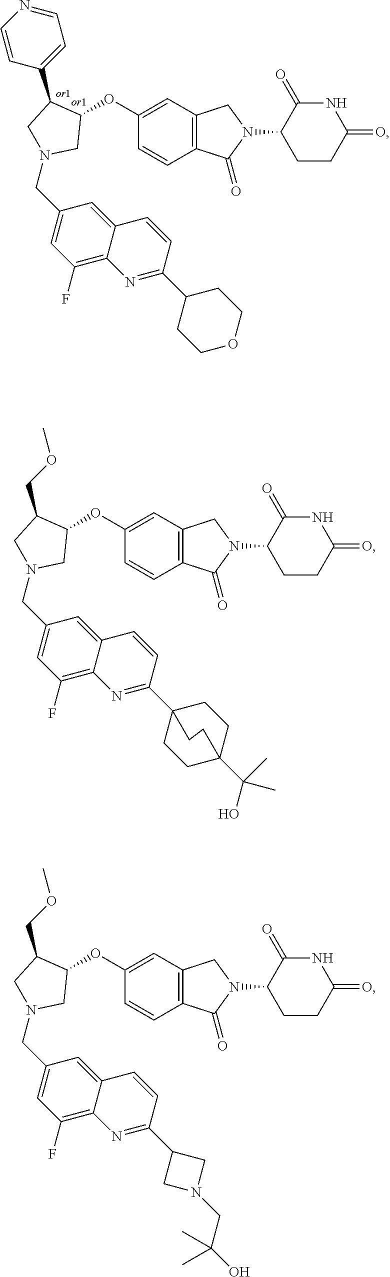 [SMILES: O=C1CC[C@H](N2Cc3cc(O[C@@H]4CN(Cc5cc(F)c6nc(C7CCOCC7)ccc6c5)C[C@H]4c4ccncc4)ccc3C2=O)C(=O)N1 ; COC[C@@H]1CN(Cc2cc(F)c3nc(C45CCC(C(C)(C)O)(CC4)CC5)ccc3c2)C[C@H]1Oc1ccc2c(c1)CN([C@H]1CCC(=O)NC1=O)C2=O ; COC[C@@H]1CN(Cc2cc(F)c3nc(C4CN(CC(C)(C)O)C4)ccc3c2)C[C@H]1Oc1ccc2c(c1)CN([C@H]1CCC(=O)NC1=O)C2=O] 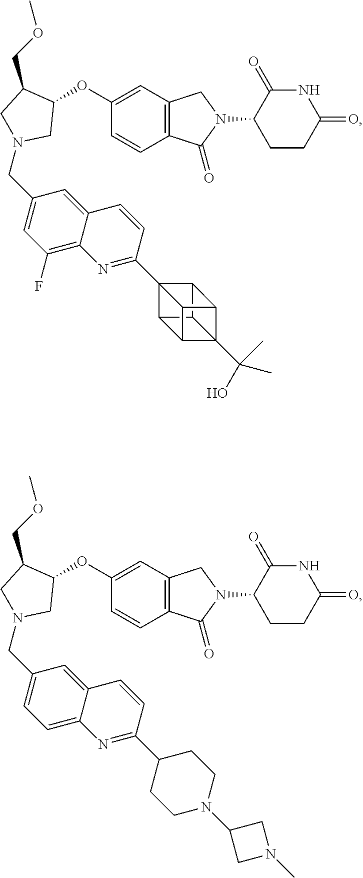 [SMILES: COC[C@@H]1CN(Cc2cc(F)c3nc(C45C6C7C4C4C5C6C74C(C)(C)O)ccc3c2)C[C@H]1Oc1ccc2c(c1)CN([C@H]1CCC(=O)NC1=O)C2=O ; COC[C@@H]1CN(Cc2ccc3nc(C4CCN(C5CN(C)C5)CC4)ccc3c2)C[C@H]1Oc1ccc2c(c1)CN([C@H]1CCC(=O)NC1=O)C2=O] 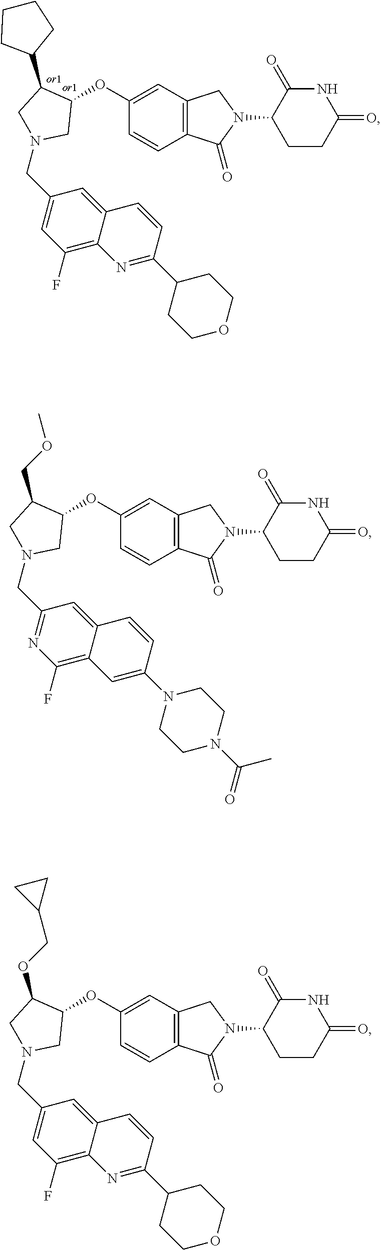 [SMILES: O=C1CC[C@H](N2Cc3cc(O[C@@H]4CN(Cc5cc(F)c6nc(C7CCOCC7)ccc6c5)C[C@H]4C4CCCC4)ccc3C2=O)C(=O)N1 ; COC[C@@H]1CN(Cc2cc3ccc(N4CCN(C(C)=O)CC4)cc3c(F)n2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4OCC4CC4)ccc3C2=O)C(=O)N1] 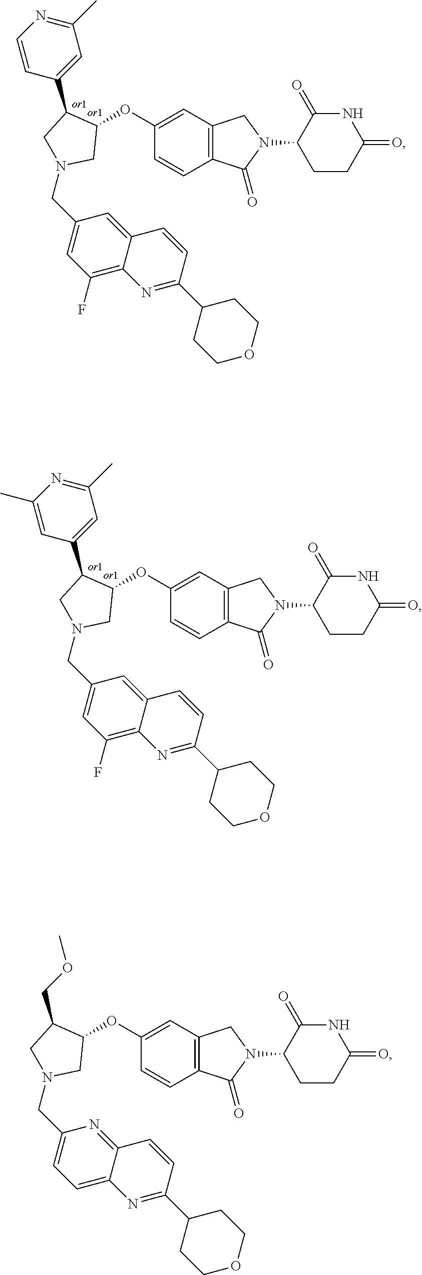 [SMILES: Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)ccn1 ; Cc1cc([C@@H]2CN(Cc3cc(F)c4nc(C5CCOCC5)ccc4c3)C[C@H]2Oc2ccc3c(c2)CN([C@H]2CCC(=O)NC2=O)C3=O)cc(C)n1 ; COC[C@@H]1CN(Cc2ccc3nc(C4CCOCC4)ccc3n2)C[C@H]1Oc1ccc2c(c1)CN([C@H]1CCC(=O)NC1=O)C2=O] 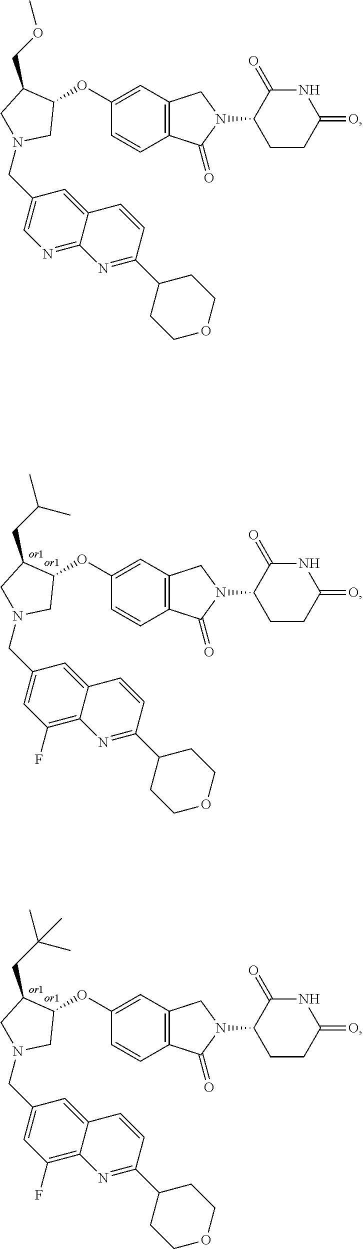 [SMILES: COC[C@@H]1CN(Cc2cnc3nc(C4CCOCC4)ccc3c2)C[C@H]1Oc1ccc2c(c1)CN([C@H]1CCC(=O)NC1=O)C2=O ; CC(C)C[C@@H]1CN(Cc2cc(F)c3nc(C4CCOCC4)ccc3c2)C[C@H]1Oc1ccc2c(c1)CN([C@H]1CCC(=O)NC1=O)C2=O ; CC(C)(C)C[C@@H]1CN(Cc2cc(F)c3nc(C4CCOCC4)ccc3c2)C[C@H]1Oc1ccc2c(c1)CN([C@H]1CCC(=O)NC1=O)C2=O] 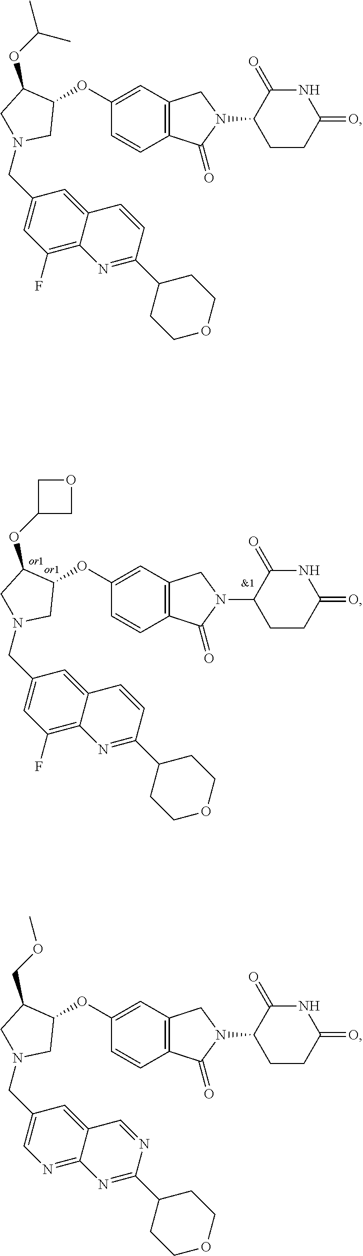 [SMILES: CC(C)O[C@@H]1CN(Cc2cc(F)c3nc(C4CCOCC4)ccc3c2)C[C@H]1Oc1ccc2c(c1)CN([C@H]1CCC(=O)NC1=O)C2=O ; O=C1CCC(N2Cc3cc(O[C@@H]4CN(Cc5cc(F)c6nc(C7CCOCC7)ccc6c5)C[C@H]4OC4COC4)ccc3C2=O)C(=O)N1 ; COC[C@@H]1CN(Cc2cnc3nc(C4CCOCC4)ncc3c2)C[C@H]1Oc1ccc2c(c1)CN([C@H]1CCC(=O)NC1=O)C2=O] 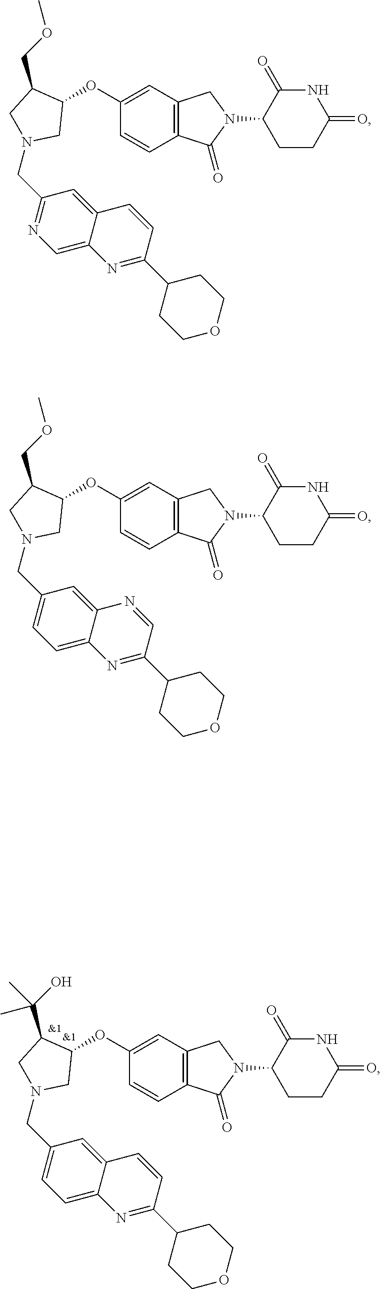 [SMILES: COC[C@@H]1CN(Cc2cc3ccc(C4CCOCC4)nc3cn2)C[C@H]1Oc1ccc2c(c1)CN([C@H]1CCC(=O)NC1=O)C2=O ; COC[C@@H]1CN(Cc2ccc3nc(C4CCOCC4)cnc3c2)C[C@H]1Oc1ccc2c(c1)CN([C@H]1CCC(=O)NC1=O)C2=O ; CC(C)(O)[C@@H]1CN(Cc2ccc3nc(C4CCOCC4)ccc3c2)C[C@H]1Oc1ccc2c(c1)CN([C@H]1CCC(=O)NC1=O)C2=O] 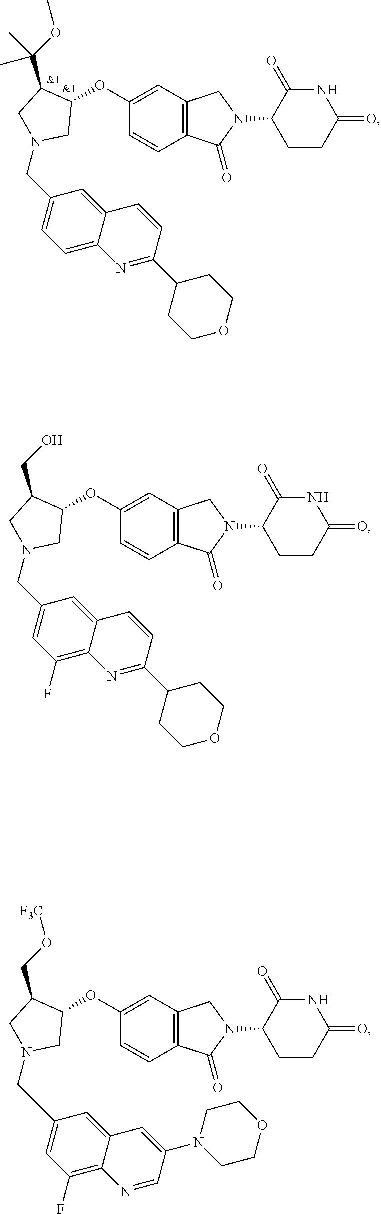 [SMILES: COC(C)(C)[C@@H]1CN(Cc2ccc3nc(C4CCOCC4)ccc3c2)C[C@H]1Oc1ccc2c(c1)CN([C@H]1CCC(=O)NC1=O)C2=O ; O=C1CC[C@H](N2Cc3cc(O[C@@H]4CN(Cc5cc(F)c6nc(C7CCOCC7)ccc6c5)C[C@H]4CO)ccc3C2=O)C(=O)N1 ; O=C1CC[C@H](N2Cc3cc(O[C@@H]4CN(Cc5cc(F)c6ncc(N7CCOCC7)cc6c5)C[C@H]4COC(F)(F)F)ccc3C2=O)C(=O)N1] 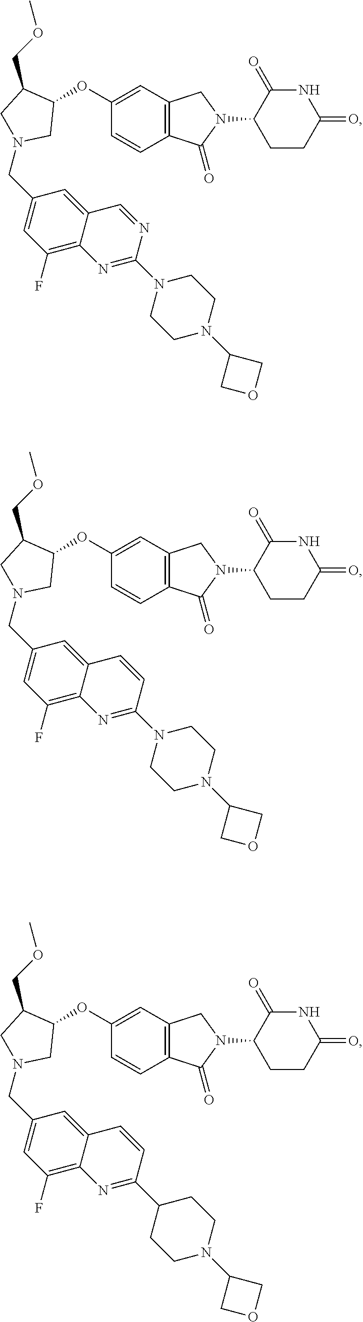 [SMILES: COC[C@@H]1CN(Cc2cc(F)c3nc(N4CCN(C5COC5)CC4)ncc3c2)C[C@H]1Oc1ccc2c(c1)CN([C@H]1CCC(=O)NC1=O)C2=O ; COC[C@@H]1CN(Cc2cc(F)c3nc(N4CCN(C5COC5)CC4)ccc3c2)C[C@H]1Oc1ccc2c(c1)CN([C@H]1CCC(=O)NC1=O)C2=O ; COC[C@@H]1CN(Cc2cc(F)c3nc(C4CCN(C5COC5)CC4)ccc3c2)C[C@H]1Oc1ccc2c(c1)CN([C@H]1CCC(=O)NC1=O)C2=O] 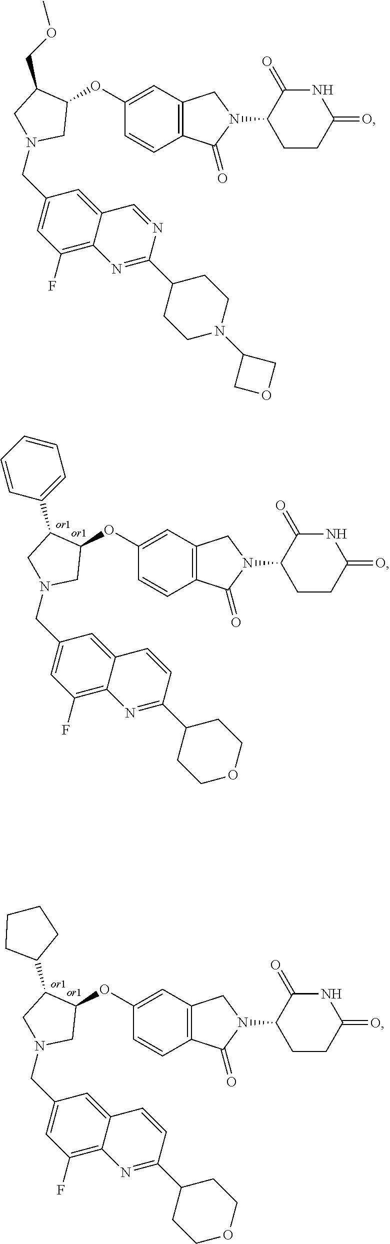 [SMILES: COC[C@@H]1CN(Cc2cc(F)c3nc(C4CCN(C5COC5)CC4)ncc3c2)C[C@H]1Oc1ccc2c(c1)CN([C@H]1CCC(=O)NC1=O)C2=O ; O=C1CC[C@H](N2Cc3cc(O[C@H]4CN(Cc5cc(F)c6nc(C7CCOCC7)ccc6c5)C[C@@H]4c4ccccc4)ccc3C2=O)C(=O)N1 ; O=C1CC[C@H](N2Cc3cc(O[C@H]4CN(Cc5cc(F)c6nc(C7CCOCC7)ccc6c5)C[C@@H]4C4CCCC4)ccc3C2=O)C(=O)N1] 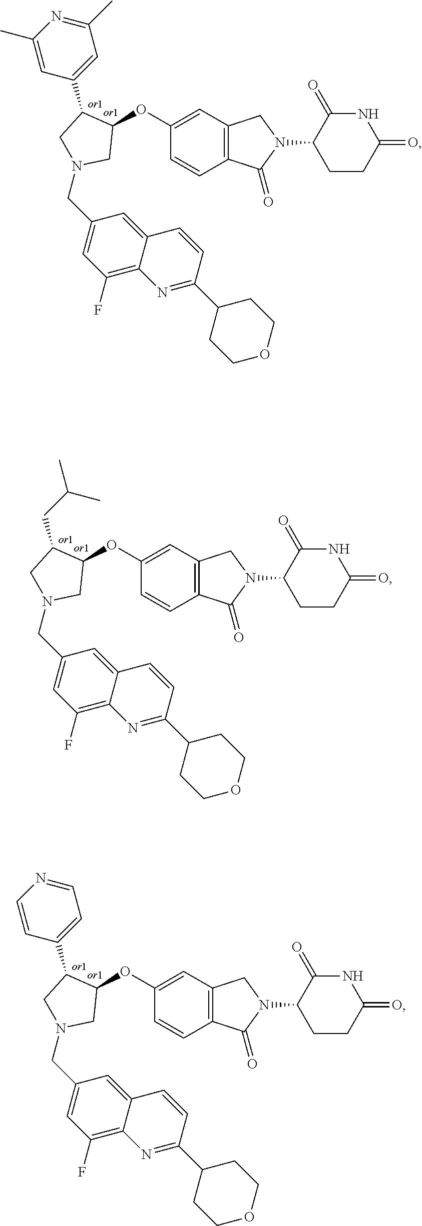 [SMILES: Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)cc(C)n1 ; CC(C)C[C@H]1CN(Cc2cc(F)c3nc(C4CCOCC4)ccc3c2)C[C@@H]1Oc1ccc2c(c1)CN([C@H]1CCC(=O)NC1=O)C2=O ; O=C1CC[C@H](N2Cc3cc(O[C@H]4CN(Cc5cc(F)c6nc(C7CCOCC7)ccc6c5)C[C@@H]4c4ccncc4)ccc3C2=O)C(=O)N1] 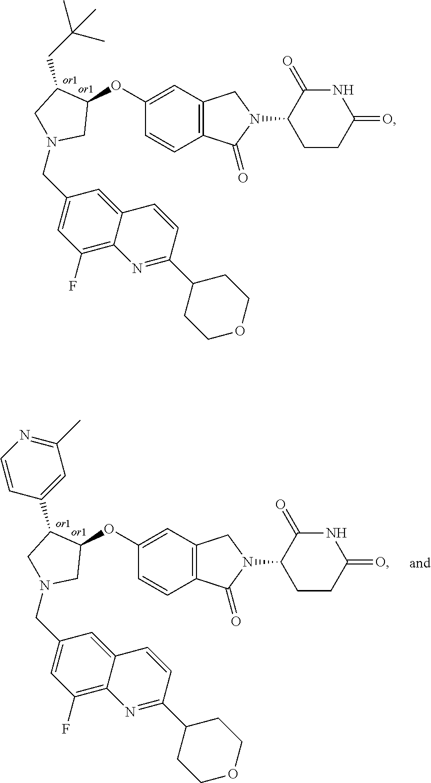 [SMILES: CC(C)(C)C[C@H]1CN(Cc2cc(F)c3nc(C4CCOCC4)ccc3c2)C[C@@H]1Oc1ccc2c(c1)CN([C@H]1CCC(=O)NC1=O)C2=O ; Cc1cc([C@H]2CN(Cc3cc(F)c4nc(C5CCOCC5)ccc4c3)C[C@@H]2Oc2ccc3c(c2)CN([C@H]2CCC(=O)NC2=O)C3=O)ccn1] 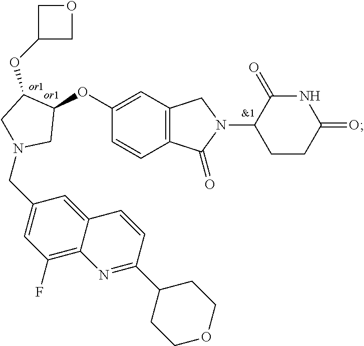 [SMILES: O=C1CCC(N2Cc3cc(O[C@H]4CN(Cc5cc(F)c6nc(C7CCOCC7)ccc6c5)C[C@@H]4OC4COC4)ccc3C2=O)C(=O)N1]
or a pharmaceutically acceptable salt, tautomer, isotopologue, or stereoisomer thereof.

2. A method for reducing WEE1 kinase protein levels, the method comprising contacting a cell with an effective amount of a compound of claim 1 or a pharmaceutically acceptable salt, tautomer, isotopologue, or stereoisomer thereof.

3. The method of claim 2, wherein the cell is in a subject.

4. A method of preventing or treating cancer in a subject comprising administering to a subject in need thereof an effective amount of a compound of claim 1 or a pharmaceutically acceptable salt, tautomer, isotopologue, or stereoisomer thereof.

5. The method according to claim 4, wherein the cancer is selected from gastric, lung, pancreatic, ovarian, breast, skin, colon, neuroblastoma, osteosarcoma, uterine, rectal, and kidney.

6. The method according to claim 5, wherein the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).

7. The use of a compound of claim 1 or a pharmaceutically acceptable salt, tautomer, isotopologue, or stereoisomer thereof, in the manufacture of a medicament for reducing WEE1 kinase protein levels.

8. The use of a compound of claim 1 or a pharmaceutically acceptable salt, tautomer, isotopologue, or stereoisomer thereof, in the manufacture of a medicament for the prevention or treatment of cancer.

9. The use according to claim 8, wherein the cancer is selected from gastric, lung, pancreatic, ovarian, breast, skin, colon, neuroblastoma, osteosarcoma, uterine, rectal, and kidney.

10. The use according to claim 9, wherein the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).
